SEQUENCE LISTING (33 sequences)

SEQ ID NO: 1 - DNA, 2173 bp, Homo sapiens
  source 1..2173: mol_type = genomic DNA
gatatcttgattccctcagaaccaggtggaaacatgttctgaataaaagccagtcctgggtattcttgctctactcaccggcttggttgtgttgggctgagccttatagaacacagtgctggagaccttctcagggtcacttcctgtacccttgactttgagctgcagccctctggttttctcaggtgagttttcttgactctttccactactcttcagccgataaagtgaccattccactcatgtgctttcccctggggtaactcctagtggatgcagtgttgggaccccttagtttcactgggcagtgtcacagggcaggctctttagaagcagaatttcagctggagtttggggcacaggaggtttatcagttgatgtaaagggaaggaggaggaagcaggatcgggcagggggagagctgagtatgatgcaagccacacaaagccttggccagcccagcggggagctctggagcagggatggcgtggcagagtgttttggggtaaagggcatgagcttggggaagtgactttctgcagactctgatggagctgacatctggaggctgcctgcccaccgcactccctgcagctgtgcagcgagcctttccttgaaaaggggtctcagtggtgcacatttttttttgagacagagtcttgctctgtcgctcaggctggagtatagtggcatgatctctgctcattgcaacctctgcctcccgggttcaagcgattctcacgcctcagccacccaagtagctgagattacaggcgtgcaccaccacacgtctctactgtttttagtagagacggggtttcagcatgttgaccaggctggtctcgaactcccgatctcaagtgatccgcccgcctcggcctcccaaagtgctgggattccaggtgtgagccaccgcacccagccagtggttcacatctttgtctcccactgtcaggaaaggtatggggaggagctgtgaaaagaaatgggtggccactgggtaagtaactggggacccagggaaaatcctgttactcccatgactcgcccacatggtcctgagccagtcactttaccactctgtgcctcagtttcctcaagactcagttgagggttggggagtatatccattcattcttctgacctatattgactgagtacctacatgtgccaggcactgtgccaagttataacagtcaacaaggcacacaaaacctcgccctcagggagctgacattgccaagaacccctcaaatctacccagtgacccagactgagggcagatgtcacccctggccttgcttgcaatgatcatgacgcacagtgagcaccgtggcctcgttgcatattcagacagatgacagcctgacatggaagctgaggatctaatcagtggcatccacatggtaacaggaaggaaggagccactgtgggtccttgagcaggagcacggagatatcagtgcaaaacttcagaagggcaattccagtggcaatcaatagaaaaagacctttttactgcatgtcaccctgccccttaggtgagtcaaattaaggcaaatagccgaggccaggccaccctgtctgtcccctgttatatttttggttttggtttgtttatagctgagttattccctagtggtggcagtgatgattgggaatttggctttctgatctgaccatttggtgacttctgggaatgaatgaacggatgttggcagagaacagcaggagtcaggcatgttatcctgtgtctcttggatctcttgccctgtggctaacggtgtgaaaatgaagcattaagctccctctgtggctgcagtcagactcctggggtgagtgagtaatgttttcccatctctggagggtgtctatacattattttgtaatgtcacttgaattaaaggaactcagttctgattggctgatagagataaataagcacttatacaaatcaaatattcctaaaatttaggaaaatgaggaaatcagactttttaaatggctgtcctctgtagtccaggaagaaactgaacttttagtaccgtaaatgcactagtcttgaaaaagattcctccttcagaaactaattcagagactttaagaatccaagttctcataattaagataaatctttggcaaatgacattagtttaattatttgagtttaataa

SEQ ID NO: 2 - DNA, 555 bp, Homo sapiens
  source 1..555: mol_type = genomic DNA
gaataactcagctataaacaaaccaaaaccaaaaatataacaggggacagacagggtggcctggcctcggctatttgccttaatttgactcacctaaggggcagggtgacatgcagtaaaaaggtctttttctattgattgccactggaattgcccttctgaagttttgcactgatatctccgtgctcctgctcaaggacccacagtggctccttccttcctgttaccatgtggatgccactgattagatcctcagcttccatgtcaggctgtcatctgtctgaatatgcaacgaggccacggtgctcactgtgcgtcatgatcattgcaagcaaggccaggggtgacatctgccctcagtctgggtcactgggtagatttgaggggttcttggcaatgtcagctccctgagggcgaggttttgtgtgccttgttgactgttataacttggcacagtgcctggcacatgtaggtactcagtcaatataggtcagaagaatgaatggatatactccccaaccctcaactgagtcttgaggaaactgaggcacagag

SEQ ID NO: 3 - DNA, 235 bp, Homo sapiens
  source 1..235: mol_type = genomic DNA
ccactggaattgcccttctgaagttttgcactgatatctccgtgctcctgctcaaggacccacagtggctccttccttcctgttaccatgtggatgccactgattagatcctcagcttccatgtcaggctgtcatctgtctgaatatgcaacgaggccacggtgctcactgtgcgtcatgatcattgcaagcaaggccaggggtgacatctgccctcagtctgggtcactgggta

SEQ ID NO: 4 - DNA, 9817 bp, synthetic construct
  source 1..9817: mol_type = other DNA
catgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggaataactcagctataaacaaaccaaaaccaaaaatataacaggggacagacagggtggcctggcctcggctatttgccttaatttgactcacctaaggggcagggtgacatgcagtaaaaaggtctttttctattgattgccactggaattgcccttctgaagttttgcactgatatctccgtgctcctgctcaaggacccacagtggctccttccttcctgttaccatgtggatgccactgattagatcctcagcttccatgtcaggctgtcatctgtctgaatatgcaacgaggccacggtgctcactgtgcgtcatgatcattgcaagcaaggccaggggtgacatctgccctcagtctgggtcactgggtagatttgaggggttcttggcaatgtcagctccctgagggcgaggttttgtgtgccttgttgactgttataacttggcacagtgcctggcacatgtaggtactcagtcaatataggtcagaagaatgaatggatatactccccaaccctcaactgagtcttgaggaaactgaggcacagaggacctgaagggtaagggggtgtggaggttgtgaaggcgggaaggggggtagccccttcaccaatgtaaacaaggatgtgggttctgcggccacactctcccccgccctccccagcggcatttccagcaagtcacatgtcctgcgcacaggctgggggccccctgctgctgcctttcttagaagccaggaccacagagcccgcacagtgagctacttggggagctatttctgtagactgagcttggggtactgggaggcagtgctgttggcctccaactctctgctccacgttccttccactattcaacacatggcaggaagtcagcctgtagggcttcacactacagtctaagctgactgccatctgtccccatccaggcgaggctggaaacgggggcagcaaaaagcccagggacaaagtccccttcccaggcacatgtattatgcactctgcaccaaggaaacctccagataaggtctagagaccaagggccatgtacttcgggggagaggccagaggacttctgaggttttacagagaaaagccaaaggcagcccagtcaggggaaatgtgtagccatagtgccgataaggaaaggccttcaacctgccctggtcagctcttcctgtaagtagaggccccttaccgaaggccccagtgggaggaagggtcgggagtactcgagtcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgtctggcggacctatgggaggtagacctggtggaagaggtgcccccgccgtgcagcagaacatccccagcaccctgctgcaggaccacgagaaccagcgcctgttcgagatgctgggccgcaagtgcctgaccctggccaccgccgtggtgcagctgtacctggccctgccccccggcgccgagcactggaccaaggagcactgcggcgccgtgtgcttcgtgaaggacaacccccagaagagctacttcatccgcctgtacggcctgcaggccggccgcctgctgtgggagcaggagctgtacagccagctggtgtacagcacccccacccccttcttccacaccttcgccggcgacgactgccaggccggcctgaacttcgccgacgaggacgaggcccaggccttccgcgccctggtgcaggagaagatccagaagcgcaaccagcgccagagcggcgacagaaggcaactgcctcctccacctacaccagccaacgaggaaagaagaggcggactgccccccctgcctcttcatcctggcggagatcaaggtggacctcctgtgggaccactgtctcttggcctggccaccgtggacatccagaaccccgacatcaccagcagccgctaccgcggcctgcccgcccccggccccagccccgccgacaagaagcgcagcggcaagaagaagatcagcaaggccgacatcggcgcccccagcggcttcaagcacgtgagccacgtgggctgggacccccagaacggcttcgacgtgaacaacctggaccccgacctgcgcagcctgttcagccgcgccggcatcagcgaggcccagctgaccgacgccgagaccagcaagctgatctacgacttcatcgaggaccagggcggcctggaggccgtgcgccaggagatgcgccgccaggagcccctgccaccacctccacctccatctagaggcggcaaccagctgccccgccctcctatcgttggcggcaacaagggaagatctggccctctgcctcctgtgcctctgggaatcgctccaccaccaccaacacctagaggcccgcctccaccaggcagaggtggtcctccgccgccacctcctccagccaccggccgcagcggcccccttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactgctggaccagatccgccagggcatccagctgaacaagacccctggcgctcctgagagttctgctctgcaaccgccaccacagtctagcgaaggacttgtgggagccctgatgcacgtgatgcagaagcgcagccgcgccatccacagcagcgacgagggcgaggaccaggccggcgacgaggacgaggacgacgagtgggacgactaactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagta

SEQ ID NO: 5 - DNA, 9497 bp, synthetic construct
  source 1..9497: mol_type = other DNA
catgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagccactggaattgcccttctgaagttttgcactgatatctccgtgctcctgctcaaggacccacagtggctccttccttcctgttaccatgtggatgccactgattagatcctcagcttccatgtcaggctgtcatctgtctgaatatgcaacgaggccacggtgctcactgtgcgtcatgatcattgcaagcaaggccaggggtgacatctgccctcagtctgggtcactgggtagacctgaagggtaagggggtgtggaggttgtgaaggcgggaaggggggtagccccttcaccaatgtaaacaaggatgtgggttctgcggccacactctcccccgccctccccagcggcatttccagcaagtcacatgtcctgcgcacaggctgggggccccctgctgctgcctttcttagaagccaggaccacagagcccgcacagtgagctacttggggagctatttctgtagactgagcttggggtactgggaggcagtgctgttggcctccaactctctgctccacgttccttccactattcaacacatggcaggaagtcagcctgtagggcttcacactacagtctaagctgactgccatctgtccccatccaggcgaggctggaaacgggggcagcaaaaagcccagggacaaagtccccttcccaggcacatgtattatgcactctgcaccaaggaaacctccagataaggtctagagaccaagggccatgtacttcgggggagaggccagaggacttctgaggttttacagagaaaagccaaaggcagcccagtcaggggaaatgtgtagccatagtgccgataaggaaaggccttcaacctgccctggtcagctcttcctgtaagtagaggccccttaccgaaggccccagtgggaggaagggtcgggagtactcgagtcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgtctggcggacctatgggaggtagacctggtggaagaggtgcccccgccgtgcagcagaacatccccagcaccctgctgcaggaccacgagaaccagcgcctgttcgagatgctgggccgcaagtgcctgaccctggccaccgccgtggtgcagctgtacctggccctgccccccggcgccgagcactggaccaaggagcactgcggcgccgtgtgcttcgtgaaggacaacccccagaagagctacttcatccgcctgtacggcctgcaggccggccgcctgctgtgggagcaggagctgtacagccagctggtgtacagcacccccacccccttcttccacaccttcgccggcgacgactgccaggccggcctgaacttcgccgacgaggacgaggcccaggccttccgcgccctggtgcaggagaagatccagaagcgcaaccagcgccagagcggcgacagaaggcaactgcctcctccacctacaccagccaacgaggaaagaagaggcggactgccccccctgcctcttcatcctggcggagatcaaggtggacctcctgtgggaccactgtctcttggcctggccaccgtggacatccagaaccccgacatcaccagcagccgctaccgcggcctgcccgcccccggccccagccccgccgacaagaagcgcagcggcaagaagaagatcagcaaggccgacatcggcgcccccagcggcttcaagcacgtgagccacgtgggctgggacccccagaacggcttcgacgtgaacaacctggaccccgacctgcgcagcctgttcagccgcgccggcatcagcgaggcccagctgaccgacgccgagaccagcaagctgatctacgacttcatcgaggaccagggcggcctggaggccgtgcgccaggagatgcgccgccaggagcccctgccaccacctccacctccatctagaggcggcaaccagctgccccgccctcctatcgttggcggcaacaagggaagatctggccctctgcctcctgtgcctctgggaatcgctccaccaccaccaacacctagaggcccgcctccaccaggcagaggtggtcctccgccgccacctcctccagccaccggccgcagcggcccccttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactgctggaccagatccgccagggcatccagctgaacaagacccctggcgctcctgagagttctgctctgcaaccgccaccacagtctagcgaaggacttgtgggagccctgatgcacgtgatgcagaagcgcagccgcgccatccacagcagcgacgagggcgaggaccaggccggcgacgaggacgaggacgacgagtgggacgactaactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagta

SEQ ID NO: 6 - DNA, 9262 bp, synthetic construct
  source 1..9262: mol_type = other DNA
catgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgaggacctgaagggtaagggggtgtggaggttgtgaaggcgggaaggggggtagccccttcaccaatgtaaacaaggatgtgggttctgcggccacactctcccccgccctccccagcggcatttccagcaagtcacatgtcctgcgcacaggctgggggccccctgctgctgcctttcttagaagccaggaccacagagcccgcacagtgagctacttggggagctatttctgtagactgagcttggggtactgggaggcagtgctgttggcctccaactctctgctccacgttccttccactattcaacacatggcaggaagtcagcctgtagggcttcacactacagtctaagctgactgccatctgtccccatccaggcgaggctggaaacgggggcagcaaaaagcccagggacaaagtccccttcccaggcacatgtattatgcactctgcaccaaggaaacctccagataaggtctagagaccaagggccatgtacttcgggggagaggccagaggacttctgaggttttacagagaaaagccaaaggcagcccagtcaggggaaatgtgtagccatagtgccgataaggaaaggccttcaacctgccctggtcagctcttcctgtaagtagaggccccttaccgaaggccccagtgggaggaagggtcgggagtactcgagtcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatgtctggcggacctatgggaggtagacctggtggaagaggtgcccccgccgtgcagcagaacatccccagcaccctgctgcaggaccacgagaaccagcgcctgttcgagatgctgggccgcaagtgcctgaccctggccaccgccgtggtgcagctgtacctggccctgccccccggcgccgagcactggaccaaggagcactgcggcgccgtgtgcttcgtgaaggacaacccccagaagagctacttcatccgcctgtacggcctgcaggccggccgcctgctgtgggagcaggagctgtacagccagctggtgtacagcacccccacccccttcttccacaccttcgccggcgacgactgccaggccggcctgaacttcgccgacgaggacgaggcccaggccttccgcgccctggtgcaggagaagatccagaagcgcaaccagcgccagagcggcgacagaaggcaactgcctcctccacctacaccagccaacgaggaaagaagaggcggactgccccccctgcctcttcatcctggcggagatcaaggtggacctcctgtgggaccactgtctcttggcctggccaccgtggacatccagaaccccgacatcaccagcagccgctaccgcggcctgcccgcccccggccccagccccgccgacaagaagcgcagcggcaagaagaagatcagcaaggccgacatcggcgcccccagcggcttcaagcacgtgagccacgtgggctgggacccccagaacggcttcgacgtgaacaacctggaccccgacctgcgcagcctgttcagccgcgccggcatcagcgaggcccagctgaccgacgccgagaccagcaagctgatctacgacttcatcgaggaccagggcggcctggaggccgtgcgccaggagatgcgccgccaggagcccctgccaccacctccacctccatctagaggcggcaaccagctgccccgccctcctatcgttggcggcaacaagggaagatctggccctctgcctcctgtgcctctgggaatcgctccaccaccaccaacacctagaggcccgcctccaccaggcagaggtggtcctccgccgccacctcctccagccaccggccgcagcggcccccttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactgctggaccagatccgccagggcatccagctgaacaagacccctggcgctcctgagagttctgctctgcaaccgccaccacagtctagcgaaggacttgtgggagccctgatgcacgtgatgcagaagcgcagccgcgccatccacagcagcgacgagggcgaggaccaggccggcgacgaggacgaggacgacgagtgggacgactaactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagta

SEQ ID NO: 7 - DNA, 222 bp, Homo sapiens
  source 1..222: mol_type = genomic DNA
caaaccttgttatctgagagaaagctagagcttccttctttcagcccccagagacaatgtggccaggctccggagggctgggaagatgagcaatgcttgtgagtcaccatgataggaagcagaagggtcaggaagtccctgggagcaaggcttagggttagggttagggagaaaacacttgggcctggaggctcggggcaggcttcctagaggggaggggta

SEQ ID NO: 8 - DNA, 245 bp, Homo sapiens
  source 1..245: mol_type = genomic DNA
ctaaaagccaccaaaaccaagacagcaatgaaagtaacctctggtcttcctaactgctcattatacactaattatgatgcactagcatgctaagagacacttccaccagcgccatgacagtgccatggcaacatcaagaagtcaccccacatggtctaaaaaggggaagaaccttcagttcccggaattgcccacccctttcctggaaaactcatgaataatccaccccttgtttagcatataat

SEQ ID NO: 9 - DNA, 371 bp, Homo sapiens
  source 1..371: mol_type = genomic DNA
gatctgggggaaaaccccctgagggtcccatttctctgccactgacccctttgcctggcagaaaggctctgctgcctcctagctgtgtagcaatggataaggcctttaacctctctaagctgaaatgtcctcatgcataaggtggagacagtaactcctcttccatctgctgggtgttgtcaaaactacacaggaggtttgccaagcacctaaggcacagtaggtggtcaagaaacaggaacacaatttgtcatgacgacaatgattccttcatatgctagttacccacgagtcagccagaatgttgccataaccacttatgaagcccttcctcatttctgttgcaattcctcagccagaggaaagcccca

SEQ ID NO: 10 - DNA, 245 bp, Homo sapiens
  source 1..245: mol_type = genomic DNA
cacagcccttgccactcccccatggcccatgtagaaacaaccatgtggccttcactctgcccacagaagctagcaccaggaccctggtcagggttagaggtttctgctgagtcaaagccacatggagggagggagcaagggagagatgcagagtcatgtttccaggaggaggttatctgagcataacagggacagggtgggccacaggatacctctgaggctcaggttcccacctccactccacc

SEQ ID NO: 11 - DNA, 1633 bp, Homo sapiens
  source 1..1633: mol_type = genomic DNA
aaagggctgttacaggagaatatggacacccaggctgcacatgcacaccatggaatgctgtatggcagtggaaataaatgaacagctaccactataggcaaacaggaatcacagcaacagccaagagtgaaggcgtggagggacgagaccatgcactcacacctggcctgcctggctcgcactccgggcaaaggggtcagaacagtgactggcacacacgttaagtgctatgtgagtgttaagataaaactaggatgtccagtggggagaaagcaagcctttgaagattatgtgcttttacaaacttcaagtgcaatgaaaactaaacaagatgttgttcaggcattcatatatgatataaagttcctttctttaaaaaagggatgggctgggcacggtggctcacgcctgtaattctaatactttgggaggccgaggcaggtggatcacgaggtcgagaaatcgagaccatcctggccaacatggtgaaaccctgtctctactaaaaatacaaaaaaattagctgggcgtggtggcgtgtgcctgtagtcccagctacttgggaggctgaggcaggagagtcacttgaacccgggaggcaaaggttgcagtgagccgagatcgtgccaccgcactccagcctggcgacagagtgagactccatctcaaaaaaaaaaagaaaaaaaaaagtatgacaagcagaaagtaatttgggagctgcggggaggcaagggtaagggatggggaagtggaccagaggcatatgcgtcattggcagtgtctaagcactcacgataggcgtggatcacaggggctcgctctgtaattaaaaggaaaagggtttttgttgtgttgttgttgttgctgtttttgagacaagggtcttgctctgtcatcatccaggctggagtgcagtggtgcagtctcagctcactgcaacctccgcctcctgggttcaagcgattctcctgcctcagcctcctgagcagctaggactacaggtgtgtgccaccatgcctggctaatttttgtattttttagtggaaatggggttttgccatgttgcccaggctcgtcttgaactcctgacctcaagtgatccactcgtctcggcctcccaaagtgctgggattacaggtgtgagctattgtccccagccaaaaggaaaagttttactgtagtaacccttccggactagggacctcgggccctcgagtcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcacc

SEQ ID NO: 12 - DNA, 417 bp, Homo sapiens
  source 1..417: mol_type = genomic DNA
tcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcacc

SEQ ID NO: 13 - DNA, 1794 bp, Homo sapiens
  source 1..1794: mol_type = genomic DNA
gacctgaagggtaagggggtgtggaggttgtgaaggcgggaaggggggtagccccttcaccaatgtaaacaaggatgtgggttctgcggccacactctcccccgccctccccagcggcatttccagcaagtcacatgtcctgcgcacaggctgggggccccctgctgctgcctttcttagaagccaggaccacagagcccgcacagtgagctacttggggagctatttctgtagactgagcttggggtactgggaggcagtgctgttggcgatctgggggaaaaccccctgagggtcccatttctctgccactgacccctttgcctggcagaaaggctctgctgcctcctagctgtgtagcaatggataaggcctttaacctctctaagctgaaatgtcctcatgcataaggtggagacagtaactcctcttccatctgctgggtgttgtcaaaactacacaggaggtttgccaagcacctaaggcacagtaggtggtcaagaaacaggaacacaatttgtcatgacgacaatgattccttcatatgctagttacccacgagtcagccagaatgttgccataaccacttatgaagcccttcctcatttctgttgcaattcctcagccagaggaaagccccaccacctctgtcctccagaaagccctcctcttcccagaaggctggcacacagggctgggggccaggcttgacttccctgtggaggggtctgaggttggggggatgacctgcagaggtaggggaattcagagaacttgttttgatagaaactgaaactcctcctcttgctgacgctgcacttgtgggcaatctgcacctgttctcccctcccagaggccagctggaagggggtcccccaccattctacctctgttcccatctcaaacagggctcagaaatcctcagagagcctggattatcttcctatagggtcctaccatttgctttagagacatctgagaactgcctactccccaggtagctgcctgcctcccagaagagccacagcaagttccgctaagggcaaaaaaggaagctaggtgtctacagggaacctaaaaacaaaccacactaacgtgtgtacacacagctgcagagggagacagggacctggtctgctcacagcccttgccactcccccatggcccatgtagaaacaaccatgtggccttcactctgcccacagaagctagcaccaggaccctggtcagggttagaggtttctgctgagtcaaagccacatggagggagggagcaagggagagatgcagagtcatgtttccaggaggaggttatctgagcataacagggacagggtgggccacaggatacctctgaggctcaggttcccacctccactccaccctccaactctctgctccacgttccttccactattcaacacatggcaggaagtcagcctgtagggcttcacactacagtctaagctgactgccatctgtccccatccaggcgaggctggaaacgggggcagcaaaaagcccagggacaaagtccccttcccaggcacatgtattatgcactctgcaccaaggaaacctccagataaggtctagagaccaagggccatgtacttcgggggagaggccagaggacttctgaggttttacagagaaaagccaaaggcagcccagtcaggggaaatgtgtagccatagtgccgataaggaaaggccttcaacctgccctggtcagctcttcctgtaagtagaggccccttaccgaaggccccagtgggaggaagggtcgggagta

SEQ ID NO: 14 - DNA, 270 bp, Homo sapiens
  source 1..270: mol_type = genomic DNA
gacctgaagggtaagggggtgtggaggttgtgaaggcgggaaggggggtagccccttcaccaatgtaaacaaggatgtgggttctgcggccacactctcccccgccctccccagcggcatttccagcaagtcacatgtcctgcgcacaggctgggggccccctgctgctgcctttcttagaagccaggaccacagagcccgcacagtgagctacttggggagctatttctgtagactgagcttggggtactgggaggcagtgctgttggc

SEQ ID NO: 15 - DNA, 283 bp, Homo sapiens
  source 1..283: mol_type = genomic DNA
ccacctctgtcctccagaaagccctcctcttcccagaaggctggcacacagggctgggggccaggcttgacttccctgtggaggggtctgaggttggggggatgacctgcagaggtaggggaattcagagaacttgttttgatagaaactgaaactcctcctcttgctgacgctgcacttgtgggcaatctgcacctgttctcccctcccagaggccagctggaagggggtcccccaccattctacctctgttcccatctcaaacagggctcagaaatcctca

SEQ ID NO: 16 - DNA, 215 bp, Homo sapiens
  source 1..215: mol_type = genomic DNA
gagagcctggattatcttcctatagggtcctaccatttgctttagagacatctgagaactgcctactccccaggtagctgcctgcctcccagaagagccacagcaagttccgctaagggcaaaaaaggaagctaggtgtctacagggaacctaaaaacaaaccacactaacgtgtgtacacacagctgcagagggagacagggacctggtctgct

SEQ ID NO: 17 - DNA, 410 bp, Homo sapiens
  source 1..410: mol_type = genomic DNA
ctccaactctctgctccacgttccttccactattcaacacatggcaggaagtcagcctgtagggcttcacactacagtctaagctgactgccatctgtccccatccaggcgaggctggaaacgggggcagcaaaaagcccagggacaaagtccccttcccaggcacatgtattatgcactctgcaccaaggaaacctccagataaggtctagagaccaagggccatgtacttcgggggagaggccagaggacttctgaggttttacagagaaaagccaaaggcagcccagtcaggggaaatgtgtagccatagtgccgataaggaaaggccttcaacctgccctggtcagctcttcctgtaagtagaggccccttaccgaaggccccagtgggaggaagggtcgggagta

SEQ ID NO: 18 - DNA, 531 bp, Homo sapiens
  source 1..531: mol_type = genomic DNA
ctaaaagccaccaaaaccaagacagcaatgaaagtaacctctggtcttcctaactgctcattatacactaattatgatgcactagcatgctaagagacacttccaccagcgccatgacagtgccatggcaacatcaagaagtcaccccacatggtctaaaaaggggaagaaccttcagttcccggaattgcccacccctttcctggaaaactcatgaataatccaccccttgtttagcatataattaagaaataactgtaagtatccttagtccagcagcccaagctgctgctctgcctatggagtagccattctttattcctttactttcttaataaacttgctttcacattaaaaacaacaacaacaacaccaacaaccagcaaatatatcagaggcttagcatcccctattcaaggggacagttctggaggccgagaaagaagtagaaaggtggagaataaaagacacaaaggagcagaagaattttatcttggcctccatgtggcacctcacagcttgggaacaaca

SEQ ID NO: 19 - DNA, 555 bp, Homo sapiens
  source 1..555: mol_type = genomic DNA
gggatctgtgtgcagatttacctctacctacacctggctggggatcataaagaaaatcaagggatgcgctacctccatgaatccaggtttcagcagagctaagggagtgaaatttggggccattttacttgtctcaaaccttgttatctgagagaaagctagagcttccttctttcagcccccagagacaatgtggccaggctccggagggctgggaagatgagcaatgcttgtgagtcaccatgataggaagcagaagggtcaggaagtccctgggagcaaggcttagggttagggttagggagaaaacacttgggcctggaggctcggggcaggcttcctagaggggaggggtaggaagaggcagtgacagggcccacagcaatggagaggagttggactgcagggatggggtagggggacagacgacaagggacactcagaagactaatgtctgggagtgggaaaacagtgtttgctcagccaggaagctgcatccagctcttttatcatttgtagaagaccaagtacccaggcctgggaggagcccagaga

SEQ ID NO: 20 - DNA, 1509 bp, Homo sapiens
  source 1..1509: mol_type = genomic DNA
atgagtgggggcccaatgggaggaaggcccgggggccgaggagcaccagcggttcagcagaacataccctccaccctcctccaggaccacgagaaccagcgactctttgagatgcttggacgaaaatgcttgacgctggccactgcagttgttcagctgtacctggcgctgccccctggagctgagcactggaccaaggagcattgtggggctgtgtgcttcgtgaaggataacccccagaagtcctacttcatccgcctttacggccttcaggctggtcggctgctctgggaacaggagctgtactcacagcttgtctactccacccccacccccttcttccacaccttcgctggagatgactgccaagcggggctgaactttgcagacgaggacgaggcccaggccttccgggccctcgtgcaggagaagatacaaaaaaggaatcagaggcaaagtggagacagacgccagctacccccaccaccaacaccagccaatgaagagagaagaggagggctcccacccctgcccctgcatccaggtggagaccaaggaggccctccagtgggtccgctctccctggggctggcgacagtggacatccagaaccctgacatcacgagttcacgataccgtgggctcccagcacctggacctagcccagctgataagaaacgctcagggaagaagaagatcagcaaagctgatattggtgcacccagtggattcaagcatgtcagccacgtggggtgggacccccagaatggatttgacgtgaacaacctcgacccagatctgcggagtctgttctccagggcaggaatcagcgaggcccagctcaccgacgccgagacctctaaacttatctacgacttcattgaggaccagggtgggctggaggctgtgcggcaggagatgaggcgccaggagccacttccgccgcccccaccgccatctcgaggagggaaccagctcccccggccccctattgtggggggtaacaagggtcgttctggtccactgccccctgtacctttggggattgccccacccccaccaacaccccggggacccccacccccaggccgagggggccctccaccaccaccccctccagctactggacgttctggaccactgccccctccaccccctggagctggtgggccacccatgccaccaccaccgccaccaccgccaccgccgcccagctccgggaatggaccagcccctcccccactccctcctgctctggtgcctgccgggggcctggcccctggtgggggtcggggagcgcttttggatcaaatccggcagggaattcagctgaacaagacccctggggccccagagagctcagcgctgcagccaccacctcagagctcagagggactggtgggggccctgatgcacgtgatgcagaagagaagcagagccatccactcctccgacgaaggggaggaccaggctggcgatgaagatgaagatgatgaatgggatgactga

SEQ ID NO: 21 - DNA, 1509 bp, synthetic construct
  source 1..1509: mol_type = other DNA
atgtctggcggacctatgggaggtagacctggtggaagaggtgcccccgccgtgcagcagaacatccccagcaccctgctgcaggaccacgagaaccagcgcctgttcgagatgctgggccgcaagtgcctgaccctggccaccgccgtggtgcagctgtacctggccctgccccccggcgccgagcactggaccaaggagcactgcggcgccgtgtgcttcgtgaaggacaacccccagaagagctacttcatccgcctgtacggcctgcaggccggccgcctgctgtgggagcaggagctgtacagccagctggtgtacagcacccccacccccttcttccacaccttcgccggcgacgactgccaggccggcctgaacttcgccgacgaggacgaggcccaggccttccgcgccctggtgcaggagaagatccagaagcgcaaccagcgccagagcggcgacagaaggcaactgcctcctccacctacaccagccaacgaggaaagaagaggcggactgccccccctgcctcttcatcctggcggagatcaaggtggacctcctgtgggaccactgtctcttggcctggccaccgtggacatccagaaccccgacatcaccagcagccgctaccgcggcctgcccgcccccggccccagccccgccgacaagaagcgcagcggcaagaagaagatcagcaaggccgacatcggcgcccccagcggcttcaagcacgtgagccacgtgggctgggacccccagaacggcttcgacgtgaacaacctggaccccgacctgcgcagcctgttcagccgcgccggcatcagcgaggcccagctgaccgacgccgagaccagcaagctgatctacgacttcatcgaggaccagggcggcctggaggccgtgcgccaggagatgcgccgccaggagcccctgccaccacctccacctccatctagaggcggcaaccagctgccccgccctcctatcgttggcggcaacaagggaagatctggccctctgcctcctgtgcctctgggaatcgctccaccaccaccaacacctagaggcccgcctccaccaggcagaggtggtcctccgccgccacctcctccagccaccggccgcagcggcccccttcctcctccaccacctggtgctggtggacctccaatgccaccgccaccgcctccgccacctccgcctccaagttctggaaatggacctgctcctcctcctttgcctcctgctttggttcctgctggcggattggctccaggcggaggaagaggcgcactgctggaccagatccgccagggcatccagctgaacaagacccctggcgctcctgagagttctgctctgcaaccgccaccacagtctagcgaaggacttgtgggagccctgatgcacgtgatgcagaagcgcagccgcgccatccacagcagcgacgagggcgaggaccaggccggcgacgaggacgaggacgacgagtgggacgactaa

SEQ ID NO: 22 - DNA, 529 bp, Human betaherpesvirus 5
  source 1..529: mol_type = genomic DNA
agttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccg

SEQ ID NO: 23 - DNA, 180 bp, Human immunodeficiency virus 1
  source 1..180: mol_type = genomic DNA
gggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagc

SEQ ID NO: 24 - DNA, 138 bp, Human immunodeficiency virus 1
  source 1..138: mol_type = genomic DNA
tcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggag

SEQ ID NO: 25 - DNA, 205 bp, Human immunodeficiency virus 1
  source 1..205: mol_type = genomic DNA
tccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacct

SEQ ID NO: 26 - DNA, 656 bp, synthetic construct
  source 1..656: mol_type = other DNA
cccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctgga

SEQ ID NO: 27 - DNA, 22 bp, Human immunodeficiency virus 1
  source 1..22: mol_type = genomic DNA
tttttaaaagaaaaggggggac

SEQ ID NO: 28 - DNA, 233 bp, synthetic construct
  source 1..233: mol_type = other DNA
tggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagc

SEQ ID NO: 29 - DNA, 140 bp, Simian virus 40
  source 1..140: mol_type = genomic DNA
atcccgcccctaactccgcccagttccgcccattctccgccccatggctgactaattttttttatttatgcagaggccgaggccgcctcggcctctgagctattccagaagtagtgaggaggcttttttggaggcctagg

SEQ ID NO: 30 - DNA, 792 bp, synthetic construct
  source 1..792: mol_type = other DNA
attgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 31 - DNA, 620 bp, synthetic construct
  source 1..620: mol_type = other DNA
agatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcg

SEQ ID NO: 32 - DNA, 680 bp, Homo sapiens
  source 1..680: mol_type = genomic DNA
gacctgaagggtaagggggtgtggaggttgtgaaggcgggaaggggggtagccccttcaccaatgtaaacaaggatgtgggttctgcggccacactctcccccgccctccccagcggcatttccagcaagtcacatgtcctgcgcacaggctgggggccccctgctgctgcctttcttagaagccaggaccacagagcccgcacagtgagctacttggggagctatttctgtagactgagcttggggtactgggaggcagtgctgttggcctccaactctctgctccacgttccttccactattcaacacatggcaggaagtcagcctgtagggcttcacactacagtctaagctgactgccatctgtccccatccaggcgaggctggaaacgggggcagcaaaaagcccagggacaaagtccccttcccaggcacatgtattatgcactctgcaccaaggaaacctccagataaggtctagagaccaagggccatgtacttcgggggagaggccagaggacttctgaggttttacagagaaaagccaaaggcagcccagtcaggggaaatgtgtagccatagtgccgataaggaaaggccttcaacctgccctggtcagctcttcctgtaagtagaggccccttaccgaaggccccagtgggaggaagggtcgggagta

SEQ ID NO: 33 - DNA, 9556 bp, synthetic construct
  source 1..9556: mol_type = other DNA
agttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagcaaaccttgttatctgagagaaagctagagcttccttctttcagcccccagagacaatgtggccaggctccggagggctgggaagatgagcaatgcttgtgagtcaccatgataggaagcagaagggtcaggaagtccctgggagcaaggcttagggttagggttagggagaaaacacttgggcctggaggctcggggcaggcttcctagaggggaggggtactaaaagccaccaaaaccaagacagcaatgaaagtaacctctggtcttcctaactgctcattatacactaattatgatgcactagcatgctaagagacacttccaccagcgccatgacagtgccatggcaacatcaagaagtcaccccacatggtctaaaaaggggaagaaccttcagttcccggaattgcccacccctttcctggaaaactcatgaataatccaccccttgtttagcatataatgacctgaagggtaagggggtgtggaggttgtgaaggcgggaaggggggtagccccttcaccaatgtaaacaaggatgtgggttctgcggccacactctcccccgccctccccagcggcatttccagcaagtcacatgtcctgcgcacaggctgggggccccctgctgctgcctttcttagaagccaggaccacagagcccgcacagtgagctacttggggagctatttctgtagactgagcttggggtactgggaggcagtgctgttggcgatctgggggaaaaccccctgagggtcccatttctctgccactgacccctttgcctggcagaaaggctctgctgcctcctagctgtgtagcaatggataaggcctttaacctctctaagctgaaatgtcctcatgcataaggtggagacagtaactcctcttccatctgctgggtgttgtcaaaactacacaggaggtttgccaagcacctaaggcacagtaggtggtcaagaaacaggaacacaatttgtcatgacgacaatgattccttcatatgctagttacccacgagtcagccagaatgttgccataaccacttatgaagcccttcctcatttctgttgcaattcctcagccagaggaaagccccacacagcccttgccactcccccatggcccatgtagaaacaaccatgtggccttcactctgcccacagaagctagcaccaggaccctggtcagggttagaggtttctgctgagtcaaagccacatggagggagggagcaagggagagatgcagagtcatgtttccaggaggaggttatctgagcataacagggacagggtgggccacaggatacctctgaggctcaggttcccacctccactccaccctccaactctctgctccacgttccttccactattcaacacatggcaggaagtcagcctgtagggcttcacactacagtctaagctgactgccatctgtccccatccaggcgaggctggaaacgggggcagcaaaaagcccagggacaaagtccccttcccaggcacatgtattatgcactctgcaccaaggaaacctccagataaggtctagagaccaagggccatgtacttcgggggagaggccagaggacttctgaggttttacagagaaaagccaaaggcagcccagtcaggggaaatgtgtagccatagtgccgataaggaaaggccttcaacctgccctggtcagctcttcctgtaagtagaggccccttaccgaaggccccagtgggaggaagggtcgggagtactcgagtcagcctcaggctacctaggtgctttagaaaggaggccacccaggcccatgactactccttgccacagggagccctgcacacagatgtgctaagctctcgctgccagccagagggaggagggtctgagccagtcagaaggagatgggccccagagagtaagaaagggggaggaggacccaagctgatccaaaaggtgggtctaagcagtcaagtggaggagggttccaatctgatggcggagggcccaagctcagcctaacgaggaggccaggcccaccaaggggcccctggaggacttgtttcccttgtcccttgtggttttttgcatttcctgttcccttgctgctcattgcggaagttcctcttcttaccctgcacccagagcctcgccagagaagacaagggcagaaagcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtgactgcaggaattcgagcatcttaccgccatttattcccatatttgttctgtttttcttgatttgggtatacatttaaatgttaataaaacaaaatggtggggcaatcatttacatttttagggatatgtaattactagttcaggtgtattgccacaagacaaacatgttaagaaactttcccgttatttacgctctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacctactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatagcacctagatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatcaggtggcacttttcggggaaatgtgcgcggaacccctatttgtttatttttctaaatacattcaaatatgtatccgctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatgg

(19) United States
(12) Patent Application Publication
(10) Pub. No.: US 2024/0287546 A1
(43) Pub. Date: Aug. 29, 2024

(54) ENHANCERS AND VECTORS

(71) Applicant: ImmunoVec, Inc., Los Angeles, CA (US)

(72) Inventor: Ryan Lupman Wong, Los Angeles, CA (US)

(21) Appl. No.: 18/605,713

(22) Filed: Mar. 14, 2024

Related U.S. Application Data
(63) Continuation of application No. PCT/US2023/025435, filed on Jun. 15, 2023.
(60) Provisional application No. 63/352,935, filed on Jun. 16, 2022.

(51) Int. Cl. C12N 15/86

(52) U.S. Cl. CPC C12N 15/86; C12N 2740/15043; C12N 2830/48

(57) ABSTRACT

In some aspects, described herein are improved enhancer elements, effective fragments thereof, and their use in vectors. In some aspects, described herein is a minimal backbone enhancer element structure, and its use in vectors. In some aspects, described herein is expression of a gene product (e.g., WAS protein) using such improved enhancer elements, for example, in a vector having the described minimal backbone enhancer element structure. In some aspects, the vectors described herein are lentiviral vectors. In some aspects, the described enhancer elements, minimal backbone enhancer element structure and vectors lead to improved gene transfer, viral titer, expression of the gene product encoded by the vector, and/or improved biological or clinical effects. Also described herein are uses of such enhancer elements, minimal backbone enhancer element structure and vectors for the treatment of diseases, for example for the treatment of diseases associated with deficient WASp expression such as Wiskott-Aldrich Syndrome (WAS).

### FIELD

The present disclosure relates to gene expression elements, in particular enhancers, and transgene delivery vehicles (e.g., vectors) comprising the same. The present disclosure also relates to the treatment or prevention of disorders associated with defective expression of certain genes, such as genes encoding a Wiskott-Aldrich syndrome (WAS) protein, using transgene delivery vehicles.

### SEQUENCE LISTING

The instant application contains a Sequence Listing which has been submitted electronically in XML file format and is hereby incorporated by reference in its entirety. Said XML copy, created on Mar. 13, 2024, is named 21278_001 US1_SL.xml and is 84,700 bytes in size.

### BACKGROUND

The WAS protein (WASp), expressed from hematopoietic cells, is critical to organizing the actin cytoskeleton and the absence of functional WASp disrupts cell motility, endocytosis (and thus antigen recognition), cell-to-cell adhesion, and other cellular factors.

WAS gene mutations can result in three distinct clinical manifestations: Wiskott-Aldrich syndrome (WAS), X-linked thrombocytopenia (XLT) and X-linked neutropenia (XLN). Mutations completely inhibiting WASp expression usually result in Wiskott-Aldrich Syndrome (WAS). Wiskott-Aldrich Syndrome (WAS) is an X-linked primary immunodeficiency caused by one or more mutations in or absence of the WAS gene and affects between 1 and 10 males per million.

Gene mutations leading to expression of defective WASp usually result in X-linked thrombocytopenia (XLT). X-linked neutropenia can be caused by gain of function WASp mutations (constitutively activated WAS). See Albert et al., 2010, Blood 115(16):3231-3228.

Symptoms of WAS include thrombocytopenia, severe eczema, bloody diarrhea, and recurrent otitis media (in male infants). In addition to thrombocytopenia, WAS patients display reduced platelet size (i.e., microthrombocytes). 30% of WAS patients display elevated eosinophil counts (i.e., eosinophilia). WAS is characterized by an increased susceptibility to viral and bacterial infections, and an increased risk of autoimmune disease and cancer (due to defects in adaptive and innate immune responses). Recurrent bacterial infections typically develop by three months after birth and children with WAS typically develop at least one autoimmune disorder. Up to one-third of WAS patients develop cancers (mainly lymphoma and leukemia). Patients with WAS demonstrate altered immunoglobulin levels, wherein immunoglobulin G (IgG) levels can be normal, reduced, or elevated: IgM levels are typically reduced, and the levels of IgA and IgE are typically elevated.

If left untreated, WAS usually leads to death in early childhood or adolescence.

Some patients with a mutation in the WAS protein exhibit a milder phenotype, termed X-linked thrombocytopenia (XLT). XLT is an inherited clotting disorder, which is a variant of WAS. XLT is also caused by mutations in the WAS gene. The symptoms of XLT are milder than those of WAS, and may include thrombocytopenia, eczema and infections.

Allogeneic stem cell transplantation is a common treatment of WAS and can be curative. However, this therapy requires availability of HLA matched donors, and may not be available to many patients due to unavailability of a suitable (HLA matched) donor.

An alternative to allogeneic stem cell transplantation is an autologous hematopoietic stem cell (HSC) transplantation by in vivo or ex vivo gene therapy. Previous viral-based therapies include CMMP-WAS γ-retroviral vector; however, use of such vector led to development of acute leukemia (due to insertional oncogenesis) in 7/9 patients. See Braun, 2014, Sci Transl. Med. 6(227):227ra33. Current gene therapy trials are utilizing a SIN lentiviral vector driven by a 1.6 kb promoter fragment of the endogenous WAS gene. Although patients showed some clinical improvement post such gene therapy, variable improvements in platelet counts were observed and majority of patients remained thrombocytopenia (likely due to the low expression of the LV in the megakaryocyte lineage or low gene transfer into hematopoetic stem cells). See Magnani, 2022, Nature Medicine, 28, 71-80, doi: 10.1038/s41591-021-01641-x; Ferrua, 2019, Lancet Hacmatol, 6(5):e239-c253, doi: 10.1016/S2352-3026(19)30021-3; Abina, 2015, JAMA, 313(15):1550-63, doi: 10.1001/jama.2015.3253.

WO2021/096887 describes lentiviral vector(s) (LVs) for the treatment of WAS, in particular WASVec1.0, incorporated by reference here in its entirety.

However, there is a need for more therapies for disorders associated with defective WAS protein expression, in particular gene therapies that can be used to circumvent dependence on suitable donors. There also remains a need for improved vectors that can be used for efficacious and safe gene therapy of WAS-related disorders.

### SUMMARY

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

In some aspects, the invention describes an improved enhancer element, element 14 (SEQ ID NO:1), as well as effective fragments thereof, e.g., element 14 core (SEQ ID NO: 2) and element 14 ultra-core (SEQ ID NO: 3). In other aspects, the disclosure provides additional regulatory sequences for improving gene expression, e.g., uCore E2 element SEQ ID NO: 32. In certain aspects, the invention describes regulatory elements that support transgene gene expression in certain blood cell types (e.g., megakaryocytes and platelets). In certain aspects the disclosure provides vectors for expressing a WAS protein in a cell.

Also described herein are uses of such enhancer elements, minimal backbone enhancer element structure and vectors for the treatment of diseases, for example for the treatment of diseases associated with deficient WASp expression such as Wiskott-Aldrich Syndrome (WAS).

In some aspects, provided herein are recombinant vectors comprising: a nucleic acid sequence of an enhancer comprising a nucleic acid sequence of enhancer element 14 or an effective fragment thereof, wherein the nucleic acid sequence of enhancer element 14 comprises, or consists of, SEQ ID NO: 1; a nucleic acid sequence of a promoter or an effective fragment thereof; and a nucleic acid that encodes a gene product operably linked to said nucleic acid sequence of an enhancer and said nucleic acid sequence of a promoter.

In some aspects, provided herein are recombinant vectors comprising: a nucleic acid sequence of an enhancer comprising a nucleic acid sequence of enhancer element 14 or an effective fragment thereof, wherein the nucleic acid sequence of enhancer element 14 comprises, or consists of, a sequence having at least 70%, 75%, 80%, 85%, 90%, 95%, 97%, 98% or 99% sequence identity to SEQ ID NO:1; a nucleic acid sequence of a promoter or an effective fragment thereof; and a nucleic acid that encodes a gene product operably linked to said nucleic acid sequence of an enhancer and said nucleic acid sequence of a promoter.

In some embodiments, a recombinant vector can be any transgene delivery vehicle. In some embodiments, the recombinant vector is a viral vector (e.g., a lentiviral vector). In some embodiments, the recombinant vector is a non-viral vector (e.g., a plasmid). In some embodiments, the nucleic acid that encodes a gene product is a nucleic acid sequence that encodes WASp (which can be cDNA and/or codon-optimized sequence).

In some embodiments of the vectors provided herein, the nucleic acid sequence of enhancer element 14 or an effective fragment thereof is a nucleic acid sequence of a core fragment of element 14 comprising, or consisting of, SEQ ID NO:2. In some embodiments of the vectors provided herein, the nucleic acid sequence of enhancer element 14 or an effective fragment thereof is a nucleic acid sequence of a core fragment of element 14 comprising, or consisting of, a sequence having at least 70%, 75%, 80%, 85%, 90%, 95%, 97%, 98%, 99% or 100% sequence identity to SEQ ID NO:2.

In some embodiments of the vectors provided herein, the nucleic acid sequence of enhancer element 14 or an effective fragment thereof is a nucleic acid sequence of an ultra-core fragment of element 14 comprising, or consisting of, SEQ ID NO:3. In some embodiments of the vectors provided herein, the nucleic acid sequence of enhancer element 14 or an effective fragment thereof is a nucleic acid sequence of an ultra-core fragment of element 14 comprising, or consisting of, a sequence having at least 70%, 75%, 80%, 85%, 90%, 95%, 97%, 98%, 99% or 100% sequence identity to SEQ ID NO:3.

In some embodiments of the vectors provided herein, the enhancer comprises the first half of enhancer element 2 core sub-element 1 of SEQ ID NO: 14, and/or enhancer element 2 core sub-element 5 of SEQ ID NO:17. In some embodiments of the vectors provided herein, the enhancer comprises the first half of enhancer element 2 core sub-element 1 of a sequence having at least 70%, 75%, 80%, 85%, 90%, 95%, 97%, 98% or 99% sequence identity to SEQ ID NO:14, and/or enhancer element 2 core sub-element 5 of a sequence having at least 70%, 75%, 80%, 85%, 90%, 95%, 97%, 98% or 99% sequence identity to SEQ ID NO: 17.

In some embodiments of the vectors provided herein, the vector does not comprise the nucleic acid sequence of sub-sub-element 1 of element 2 of SEQ ID NO:9 or an effective fragment thereof. In some embodiments of the vectors provided herein, the vector does not comprise the nucleic acid sequence of sub-sub-element 1 of element 2 of a sequence having at least 70%, 75%, 80%, 85%, 90%, 95%, 97%, 98% or 99% sequence identity to SEQ ID NO:9.

In some embodiments of the vectors provided herein, the vector does not comprise the nucleic acid sequence of sub-element 4 of enhancer element 2 of SEQ ID NO: 10 or an effective fragment thereof. In some embodiments of the vectors provided herein, the vector does not comprise the nucleic acid sequence of sub-element 4 of enhancer element 2 of a sequence having at least 70%, 75%, 80%, 85%, 90%, 95%, 97%, 98% or 99% sequence identity to SEQ ID NO:10.

In some embodiments of the vectors provided herein, the vector does not comprise the nucleic acid sequence of enhancer element 9 slim of SEQ ID NO:7 or an effective fragment thereof. In some embodiments of the vectors provided herein, the vector does not comprise the nucleic acid sequence of enhancer element 9 slim of a sequence having at least 70%, 75%, 80%, 85%, 90%, 95%, 97%, 98% or 99% sequence identity to SEQ ID NO:7.

In some embodiments of the vectors provided herein, the vector does not comprise the nucleic acid sequence of hypersensitive site 3 (HS3) core of SEQ ID NO:8 or an effective fragment thereof. In some embodiments of the vectors provided herein, the vector does not comprise the nucleic acid sequence of hypersensitive site 3 (HS3) core of a sequence having at least 70%, 75%, 80%, 85%, 90%, 95%, 97%, 98% or 99% sequence identity to SEQ ID NO:8.

In some embodiments of the vectors provided herein, the nucleic acid sequence of an enhancer consists of, or substantially consists of: (i) a nucleic acid sequence of enhancer element 14 or an effective fragment thereof, wherein the nucleic acid sequence of enhancer element 14 comprises, or consists of, SEQ ID NO: 1, and/or (ii) a nucleic acid sequence of uCore E2 element of SEQ ID NO:32. In some embodiments of the vectors provided herein, the nucleic acid sequence of an enhancer consists of, or substantially consists of: (i) a nucleic acid sequence of enhancer element 14 or an effective fragment thereof, wherein the nucleic acid sequence of enhancer element 14 comprises, or consists of, a sequence having at least 70%, 75%, 80%, 85%, 90%, 95%, 97%, 98% or 99% sequence identity to SEQ ID NO:1, and/or (ii) a nucleic acid sequence of uCore E2 element of a sequence having at least 70%, 75%, 80%, 85%, 90%, 95%, 97%, 98% or 99% sequence identity to SEQ ID NO:32.

In some aspects, provided herein are recombinant vectors comprising: a nucleic acid sequence of an enhancer comprising, or consisting of, the first half of enhancer element 2 core sub-element 1 of SEQ ID NO: 14, and enhancer element 2 core sub-element 5 of SEQ ID NO:17; a nucleic acid sequence of a promoter or an effective fragment thereof; and a nucleic acid that encodes a gene product operably linked to said nucleic acid sequence of an enhancer and said nucleic acid sequence of a promoter, optionally wherein such vector does not comprise: (i) the nucleic acid sequence of sub-sub-element 1 of element 2 of SEQ ID NO:9 or an effective fragment thereof; (ii) the nucleic acid sequence of sub-element 4 of enhancer element 2 of SEQ ID NO: 10 or an effective fragment thereof; (iii) the nucleic acid sequence of enhancer element 9 slim of SEQ ID NO:7 or an effective fragment thereof, and/or (iv) the nucleic acid sequence of hypersensitive site 3 (HS3) core of SEQ ID NO:8 or an effective fragment thereof. In some aspects, provided herein are recombinant vectors comprising: a nucleic acid sequence of an enhancer comprising, or consisting of, the first half of enhancer element 2 core sub-element 1 of SEQ ID NO: 14, and enhancer element 2 core sub-element 5 of SEQ ID NO: 17; a nucleic acid sequence of a promoter or an effective fragment thereof; and a nucleic acid that encodes a gene product operably linked to said nucleic acid sequence of an enhancer and said nucleic acid sequence of a promoter, wherein such vector does not comprise: (i) the nucleic acid sequence of sub-sub-element 1 of element 2 of SEQ ID NO:9 or an effective fragment thereof; (ii) the nucleic acid sequence of sub-element 4 of enhancer element 2 of SEQ ID NO: 10 or an effective fragment thereof; (iii) the nucleic acid sequence of enhancer element 9 slim of SEQ ID NO:7 or an effective fragment thereof, and/or (iv) the nucleic acid sequence of hypersensitive site 3 (HS3) core of SEQ ID NO:8 or an effective fragment thereof.

In some aspects, provided herein are recombinant vectors comprising: a nucleic acid sequence of an enhancer comprising, or consisting of, the first half of enhancer element 2 core sub-element 1 of a sequence having at least 70%, 75%, 80%, 85%, 90%, 95%, 97%, 98% or 99% sequence identity to SEQ ID NO:14, and enhancer element 2 core sub-element 5 of a sequence having at least 70%, 75%, 80%, 85%, 90%, 95%, 97%, 98% or 99% sequence identity to SEQ ID NO:17; a nucleic acid sequence of a promoter or an effective fragment thereof; and a nucleic acid that encodes a gene product operably linked to said nucleic acid sequence of an enhancer and said nucleic acid sequence of a promoter, optionally wherein such vector does not comprise: (i) the nucleic acid sequence of sub-sub-element 1 of element 2 of SEQ ID NO:9 or an effective fragment thereof (e.g., a sequence having at least 70%, 75%, 80%, 85%, 90%, 95%, 97%, 98% or 99% sequence identity to SEQ ID NO:9); (ii) the nucleic acid sequence of sub-element 4 of enhancer element 2 of SEQ ID NO: 10 or an effective fragment thereof (e.g., a sequence having at least 70%, 75%, 80%, 85%, 90%, 95%, 97%, 98% or 99% sequence identity to SEQ ID NO:10); (iii) the nucleic acid sequence of enhancer element 9 slim of SEQ ID NO:7 or an effective fragment thereof (e.g., a sequence having at least 70%, 75%, 80%, 85%, 90%, 95%, 97%, 98% or 99% sequence identity to SEQ ID NO:7), and/or (iv) the nucleic acid sequence of hypersensitive site 3 (HS3) core of SEQ ID NO:8 or an effective fragment thereof (e.g., a sequence having at least 70%, 75%, 80%, 85%, 90%, 95%, 97%, 98% or 99% sequence identity to SEQ ID NO:8).

In some embodiments, a recombinant vector can be any transgene delivery vehicle. In some embodiments, the recombinant vector is a viral vector (e.g., a lentiviral vector). In some embodiments, the recombinant vector is a non-viral vector (e.g., a plasmid or episome). In some embodiments, the nucleic acid that encodes a gene product is a nucleic acid sequence that encodes WASp (which can be cDNA and/or codon-optimized sequence).

In some embodiments of the vectors provided herein, the vector comprises a nucleic acid sequence of enhancer element 14 or an effective fragment thereof, wherein the nucleic acid sequence of enhancer element 14 comprises, or consists of, SEQ ID NO:1. In some embodiments of the vectors provided herein, the vector comprises a nucleic acid sequence of enhancer element 14 or an effective fragment thereof, wherein the nucleic acid sequence of enhancer element 14 comprises, or consists of, a sequence having at least 70%, 75%, 80%, 85%, 90%, 95%, 97%, 98% or 99% sequence identity to SEQ ID NO:1.

In some embodiments of the vectors provided herein, the vector comprises a nucleic acid sequence of a core fragment of element 14 comprising, or consisting of, SEQ ID NO:2. In some embodiments of the vectors provided herein, the vector comprises a nucleic acid sequence of a core fragment of element 14 comprising, or consisting of, a sequence having at least 70%, 75%, 80%, 85%, 90%, 95%, 97%, 98% or 99% sequence identity to SEQ ID NO:2.

In some embodiments of the vectors provided herein, the vector comprises a nucleic acid sequence of an ultra-core fragment of element 14 comprising, or consisting of, SEQ ID NO:3. In some embodiments of the vectors provided herein, the vector comprises a nucleic acid sequence of an ultra-core fragment of element 14 comprising, or consisting of, a sequence having at least 70%, 75%, 80%, 85%, 90%, 95%, 97%, 98% or 99% sequence identity to SEQ ID NO:3.

In some embodiments of the vectors provided herein, the nucleic acid sequence of an enhancer consists of, or substantially consists of: a nucleic acid sequence of uCore E2 element of SEQ ID NO:32. In some embodiments of the vectors provided herein, the nucleic acid sequence of an enhancer consists of, or substantially consists of: a nucleic acid sequence of uCore E2 element of a sequence having at least 70%, 75%, 80%, 85%, 90%, 95%, 97%, 98% or 99% sequence identity to SEQ ID NO:32.

In some embodiments of the vectors provided herein, the nucleic acid sequence of an enhancer consists of, or substantially consists of: (i) a nucleic acid sequence of enhancer element 14 or an effective fragment thereof, wherein the nucleic acid sequence of enhancer element 14 comprises, or consists of, SEQ ID NO:1, and/or (ii) a nucleic acid sequence of uCore E2 element of SEQ ID NO:32. In some embodiments of the vectors provided herein, the nucleic acid sequence of an enhancer consists of, or substantially consists of: (i) a nucleic acid sequence of enhancer element 14 or an effective fragment thereof, wherein the nucleic acid sequence of enhancer element 14 comprises, or consists of, SEQ ID NO:1, and (ii) a nucleic acid sequence of uCore E2 element of SEQ ID NO:32.

In some embodiments of the vectors provided herein, the nucleic acid sequence of an enhancer consists of, or substantially consists of: (i) a nucleic acid sequence of enhancer element 14 or an effective fragment thereof, wherein the nucleic acid sequence of enhancer element 14 comprises, or consists of, a sequence having at least 70%, 75%, 80%, 85%, 90%, 95%, 97%, 98% or 99% sequence identity to SEQ ID NO: 1, and/or (ii) a nucleic acid sequence of uCore E2 element of a sequence having at least 70%, 75%, 80%, 85%, 90%, 95%, 97%, 98% or 99% sequence identity to SEQ ID NO:32.

In some embodiments of the vectors provided herein, the translated gene product is Wiskott-Aldrich Syndrome protein (WASp).

In some embodiments of the vectors provided herein, the nucleic acid that encodes the WASp is a codon-optimized WAS nucleic acid sequence, optionally wherein the codon-optimized WAS comprises or consists of SEQ ID NO:21. In some embodiments of the vectors provided herein, the nucleic acid that encodes the WASp is a codon-optimized WAS nucleic acid sequence, wherein the codon-optimized WAS nucleic acid sequence comprises or consists of SEQ ID NO:21. In some embodiments of the vectors provided herein, the nucleic acid that encodes the WASp is a codon-optimized WAS nucleic acid sequence, wherein the codon-optimized WAS nucleic acid sequence comprises or consists of a sequence having at least 70%, 75%, 80%, 85%, 90%, 95%, 97%, 98% or 99% sequence identity to SEQ ID NO:21.

In some embodiments of the vectors provided herein, the codon-optimized WAS nucleic acid sequence is selected from the group consisting of jCAT codon-optimized WAS nucleic acid sequence(s), GeneArt-optimized WAS nucleic acid sequence(s), and IDT-optimized WAS nucleic acid sequence(s). In some embodiments, the WAS nucleic acid sequence is jCAT codon-optimized WAS nucleic acid sequence(s). In some embodiments, the WAS nucleic acid sequence is a GeneArt-optimized WAS nucleic acid sequence. In some embodiments, the WAS nucleic acid sequence is IDT-optimized WAS nucleic acid sequence.

In some embodiments of the vectors provided herein, the promoter is a human promoter.

In some embodiments of the vectors provided herein, the promoter is the endogenous promoter of the WAS gene, e.g., endogenous human promoter of the WAS gene.

In some embodiments of the vectors provided herein, the promoter is the WAS gene promoter of SEQ ID NO:11. In some embodiments of the vectors provided herein, the promoter is the WAS gene promoter of a sequence having at least 70%, 75%, 80%, 85%, 90%, 95%, 97%, 98% or 99% sequence identity to SEQ ID NO:11.

In some embodiments of the vectors provided herein, the promoter or the effective fragment thereof is an effective fragment of the endogenous promoter of a WAS gene which has maximum length of 600 bp and contains the nucleic acid sequence of HS1pro of SEQ ID NO:12. In some embodiments of the vectors provided herein, the promoter or the effective fragment thereof is an effective fragment of the endogenous promoter of the WAS gene which has maximum length of 600 bp and contains the nucleic acid sequence of HS1pro of a sequence having at least 70%, 75%, 80%, 85%, 90%, 95%, 97%, 98% or 99% sequence identity to SEQ ID NO:12.

In some embodiments of the vectors provided herein, the promoter or the effective fragment thereof is an effective fragment of the endogenous promoter of the WAS gene consisting of the sequence of HS1pro (SEQ ID NO: 12). In some embodiments of the vectors provided herein, the promoter or the effective fragment thereof is an effective fragment of the endogenous promoter of the WAS gene consisting of a sequence having at least 70%, 75%, 80%, 85%, 90%, 95%, 97%, 98% or 99% sequence identity to SEQ ID NO: 12.

In some embodiments of the vectors provided herein, the vector is a recombinant lentiviral vector. In some embodiments, the vector comprises a Y′ packaging signal. In some embodiments, the vector comprises a Rev Responsive Element (RRE). In some embodiments, the vector comprises a central polypurine tract. In some embodiments, the vector comprises a posttranscriptional regulatory element. In some embodiments, the posttranscriptional regulatory element is a Woodchuck Post-transcriptional Regulatory Element (WPRE).

In some embodiments, provided herein are vectors comprising or consisting of a nucleic acid sequence having at least 70%, at least 75%, at least 80%, at least 85%, at least 90%, at least 95%, at least 97%, at least 98%, at least 99%, or 100% nucleic acid sequence identity to SEQ ID NO:4. In some embodiments, provided herein are vectors comprising a nucleic acid sequence having at least 70%, at least 75%, at least 80%, at least 85%, at least 90%, at least 95%, at least 97%, at least 98%, at least 99%, or 100% nucleic acid sequence identity to any element or sequence in the vector of SEQ ID NO:4.

In some embodiments, provided herein are vectors comprising or consisting of a nucleic acid sequence having at least 70%, at least 75%, at least 80%, at least 85%, at least 90%, at least 95%, at least 97%, at least 98%, at least 99%, or 100% nucleic acid sequence identity to SEQ ID NO:5. In some embodiments, provided herein are vectors comprising a nucleic acid sequence having at least 70%, at least 75%, at least 80%, at least 85%, at least 90%, at least 95%, at least 97%, at least 98%, at least 99%, or 100% nucleic acid sequence identity to any element or sequence in the vector of SEQ ID NO:5.

In some embodiments, provided herein are vectors comprising or consisting of a nucleic acid sequence having at least 70%, at least 75%, at least 80%, at least 85%, at least 90%, at least 95%, at least 97%, at least 98%, at least 99%, or 100% nucleic acid sequence identity to SEQ ID NO:6. In some embodiments, provided herein are vectors comprising a nucleic acid sequence having at least 70%, at least 75%, at least 80%, at least 85%, at least 90%, at least 95%, at least 97%, at least 98%, at least 99%, or 100% nucleic acid sequence identity to any element or sequence in the vector of SEQ ID NO:6.

In some embodiments of the vectors provided herein, the vectors are capable of expressing a gene product encoded by the transgene (e.g., WASp) in a cell (e.g., a stem cell and/or a progenitor cell, such as a hematopoietic stem and/or progenitor cell). In some of these embodiments, the vector is capable of expressing the gene product at or near its physiologic level. In some of these embodiments, the vector is capable of expressing the gene product at a high level (e.g., at a level above the endogenous or physiologic level of the corresponding native gene in a healthy subject). In some embodiments of the vectors provided herein, introduction of the vectors into a cell leads to expression of the gene product within about 60%, 50%, 40%, 30%, or 20% of the endogenous, physiologic level of expression of the corresponding native gene in a healthy subject. In some embodiments, the vector is effective to express WASp in a cell within about 60%, 50%, 40%, 30%, or 20% of the endogenous, physiologic levels of WASp expression in a healthy subject.

In some aspects, the vectors provided herein are encapsulated within a viral particle, e.g., a viral capsid.

In some aspects, a cell is transduced with any vector or viral particle provided herein. In some embodiments, the cell is a stem cell or a progenitor cell. In some embodiments, the cell is a CD34+ hematopoietic stem and/or progenitor cell. In some embodiments, the cell is a cell derived from bone marrow, umbilical cord blood, and/or peripheral blood. In some embodiments, the cell is a dendritic cell, a CD4⁺ T cell, or a peripheral blood B or T cell. In some embodiments, the cell is a human cell.

In some aspects, provided herein is a pharmaceutical composition comprising any vector provided herein, any viral particle provided herein, or any cell provided herein. In some embodiments, the pharmaceutical composition further comprises a pharmaceutically acceptable carrier.

In some aspects, provided herein are methods of treating or preventing a disease or disorder associated with a deficient expression of a gene product in a subject in need thereof, said methods comprising: transducing a cell (e.g., a stem cell and/or progenitor cell) with any vector described herein or any viral particle described herein; and transplanting the cell into the subject (e.g., wherein the cell or a derivative thereof expresses the gene product encoded by the vector or viral particle). In some embodiments, prior to transducing, the cell is derived from the subject (i.e., autologous to the subject to be treated). In some embodiments, the transduced cell is not derived from the subject to be treated.

In some aspects, provided herein are uses of any vector or viral particle described herein for treating or preventing a disease or disorder associated with a deficient expression of a gene product encoded by the vector or viral particle in a subject in need thereof, comprising: transducing a cell (e.g., a stem cell and/or progenitor cell) with any vector described herein or any viral particle described herein; and transplanting the cell into the subject (e.g., wherein the cell or a derivative thereof expresses the gene product encoded by the vector or viral particle). In some embodiments, prior to transducing, the cell is derived from the subject (i.e., autologous to the subject to be treated). In some embodiments, the transduced cell is not derived from the subject to be treated.

In some embodiments, the methods and uses provided herein are methods and uses for treating (rather than preventing) a disease or disorder.

In some embodiments, syndrome, disease, or disorder is any syndrome, disease, or disorder associated with deficient expression of WASp. In some embodiments, syndrome, disease or disorder is any disease or disorder associated with abnormal expression of WASp. In some embodiments, syndrome, disease or disorder is Wiskott-Aldrich Syndrome (WAS). In some embodiments, disease or disorder is X-linked thrombocytopenia (XLT) or X-linked congenital neutropenia (XLN). In some embodiments, the gene product expressed by the vector or viral particle is WASp. In some embodiments, syndrome, disease, or disorder is any syndrome, disease, or disorder associated with deficient expression of WASp, and the gene product expressed by the vector or viral particle is a functional WASp.

In some embodiments, the stem cell and/or a progenitor cell is a human hematopoietic stem and/or progenitor cell. In some embodiments, the stem cell and/or a progenitor cell is a human hematopoietic stem and/or progenitor cell that is derived from bone marrow. In some embodiments, the cell is a CD34+ cell. In some embodiments, the cell is a megakaryocyte. In some embodiments, the cell is derived from mPBSCs.

In some aspects, provided herein are methods of treating or preventing a disease or disorder associated with a deficient expression of a gene product in a subject in need thereof, said methods comprising: administering to the subject any vector described herein, any viral particle described herein, any cell transduced with the vector or viral particle described herein, or any pharmaceutical composition comprising a vector, a viral particle, or a cell transduced with the same as described herein.

In some aspects, provided herein are uses of any vector, viral particle, cell or pharmaceutical composition as described herein for treating or preventing a disease or disorder associated with a deficient expression of a gene product in a subject in need thereof, comprising: administering to the subject such vector, viral particle, cell or pharmaceutical composition, as described herein.

In some embodiments, the methods and uses provided herein are methods and uses for treating (rather than preventing) a disease or disorder.

In some embodiments, the subject being treated using any methods or nucleic acids (e.g., viral vector) described herein is a mammal. In some embodiments, the subject is a human. In some embodiments, the subject is male. In some embodiments, the subject to be treated using any methods or uses described herein is under the age of 21, 18, 16, 14, 12, 10, 8, 6, 5, 4, 3, 2, or 1. In some embodiments, the subject is an infant. In some embodiments, the subject is a toddler.

In some embodiments, the treating comprises a single administration of a vector, a viral particle, a transduced cell or a pharmaceutical composition described herein.

In some embodiments, the treating comprises parenteral (e.g., intravenous) administration of a vector, a viral particle, a transduced cell or a pharmaceutical composition described herein. In some embodiments, the treating is by intravenous infusion. In some embodiments, the treating comprises local administration. In some embodiments, the treating comprises intramuscular administration.

In some embodiments, the vectors or viral particles described herein are administered in a dose in the range of about 1×10⁵ TU/ml to about 1×10⁸ TU/ml. In some instances, the vectors are administered in doses of no more than 1×10⁵ TU/ml. In some instances, the vectors are administered in doses of 1×10¹-1×10¹² TU/m, 1×10¹-1×10¹¹ TU/mL, 1×10¹-1×10¹⁰ TU/mL, 1×10¹-1×10⁹ TU/mL, 1×10¹-1×10⁸ TU/mL, 1×10¹-1×10⁷ TU/mL, 1×10¹-1×10⁶ TU/mL, 1×10¹-1×10⁵ TU/mL, or 1×10¹-1×10⁴ TU/mL.

In some aspects, provided herein is a recombinant nucleic acid or an expression cassette comprising a nucleic acid sequence of enhancer element 14 comprising, or consisting of, SEQ ID NO:1 or an effective fragment thereof. In some aspects, provided herein is a recombinant nucleic acid or an expression cassette comprising a nucleic acid sequence of enhancer element 14 comprising, or consisting of, a sequence having at least 70%, 75%, 80%, 85%, 90%, 95%, 97%, 98% or 99% sequence identity to SEQ ID NO: 1 or an effective fragment thereof.

In some embodiments, the recombinant nucleic acid comprises a nucleic acid sequence of a core fragment of element 14 comprising, or consisting of, SEQ ID NO:2. In some embodiments, the recombinant nucleic acid comprises a nucleic acid sequence of a core fragment of element 14 comprising, or consisting of, a sequence having at least 70%, 75%, 80%, 85%, 90%, 95%, 97%, 98% or 99% sequence identity to SEQ ID NO:2.

In some embodiments, the recombinant nucleic acid comprises a nucleic acid sequence of ultra-core fragment of element 14 comprising, or consisting of, SEQ ID NO:3. In some embodiments, the recombinant nucleic acid comprises a nucleic acid sequence of ultra-core fragment of element 14 comprising, or consisting of, a sequence having at least 70%, 75%, 80%, 85%, 90%, 95%, 97%, 98% or 99% sequence identity to SEQ ID NO:3.

In some embodiments, the recombinant nucleic acid comprises a nucleic acid sequence of the first half of enhancer element 2 core sub-element 1 of SEQ ID NO: 14, and/or enhancer element 2 core sub-element 5 of SEQ ID NO:17. In some embodiments, the recombinant nucleic acid comprises a nucleic acid sequence of the first half of enhancer element 2 core sub-element 1 of a sequence having at least 70%, 75%, 80%, 85%, 90%, 95%, 97%, 98% or 99% sequence identity to SEQ ID NO: 14, and/or enhancer element 2 core sub-element 5 of a sequence having at least 70%, 75%, 80%, 85%, 90%, 95%, 97%, 98% or 99% sequence identity to SEQ ID NO:17.

In some embodiments, the recombinant nucleic acid comprises or consists of a nucleic acid sequence of uCore E2 element of SEQ ID NO:32. In some embodiments, the recombinant nucleic acid comprises or consists of a nucleic acid sequence of uCore E2 element of a sequence having at least 70%, 75%, 80%, 85%, 90%, 95%, 97%, 98% or 99% sequence identity to SEQ ID NO:32.

In some embodiments, the recombinant nucleic acid comprises or consists of a combination of (i) a nucleic acid sequence of enhancer element 14 comprising, or consisting of, SEQ ID NO: 1 or an effective fragment thereof, optionally comprising or consisting of SEQ ID NO:2 or SEQ ID NO:3, and/or (ii) a nucleic acid sequence of uCore E2 element of SEQ ID NO:32. In some embodiments, the recombinant nucleic acid comprises or consists of a combination of (i) a nucleic acid sequence of enhancer element 14 comprising, or consisting of, a sequence having at least 70%, 75%, 80%, 85%, 90%, 95%, 97%, 98%, 99% or 100% sequence identity to SEQ ID NO:1 or an effective fragment thereof, optionally comprising or consisting of SEQ ID NO:2 or SEQ ID NO:3, and (ii) a nucleic acid sequence of uCore E2 element of a sequence having at least 70%, 75%, 80%, 85%, 90%, 95%, 97%, 98%, 99% or 100% sequence identity to SEQ ID NO:32.

In some embodiments, the recombinant nucleic acid comprises a nucleic acid sequence of any human promoter or an effective fragment thereof.

In some embodiments, the recombinant nucleic acid comprises a nucleic acid sequence of an endogenous promoter of the WAS gene. In some embodiments, the recombinant nucleic acid comprises a nucleic acid sequence of a WAS gene promoter comprising, or consisting of, SEQ ID NO:11. In some embodiments, the recombinant nucleic acid comprises a nucleic acid sequence of a WAS gene promoter comprising, or consisting of, at least 70%, 75%, 80%, 85%, 90%, 95%, 97%, 98%, 99% or 100% sequence identity to SEQ ID NO:11.

In some embodiments, the promoter or the effective fragment thereof is an effective fragment of the endogenous promoter of the WAS gene which has maximum length of 600 bp and contains the nucleic acid sequence of HS1pro of SEQ ID NO: 12. In some embodiments, the promoter or the effective fragment thereof is an effective fragment of the endogenous promoter of the WAS gene which has maximum length of 600 bp and contains the nucleic acid sequence of HS1pro of at least 70%, 75%, 80%, 85%, 90%, 95%, 97%, 98%, 99% or 100% sequence identity to SEQ ID NO:12. In some embodiments, the promoter or the effective fragment thereof is an effective fragment of the endogenous promoter of the WAS gene consisting of the sequence of SEQ ID NO: 12. In some embodiments, the promoter or the effective fragment thereof is an effective fragment of the endogenous promoter of the WAS gene consisting of a sequence of at least 70%, 75%, 80%, 85%, 90%, 95%, 97%, 98%, 99% or 100% sequence identity to SEQ ID NO:12.

In some embodiments, the recombinant nucleic acid does not comprise: (i) the nucleic acid sequence of sub-sub-element 1 of element 2 of SEQ ID NO:9 or an effective fragment thereof; (ii) the nucleic acid sequence of sub-element 4 of enhancer element 2 of SEQ ID NO:10 or an effective fragment thereof; (iii) the nucleic acid sequence of enhancer element 9 slim of SEQ ID NO:7 or an effective fragment thereof; and/or (v) the nucleic acid sequence of hypersensitive site 3 (HS3) of SEQ ID NO:8 or an effective fragment thereof.

In some embodiments, the recombinant nucleic acid does not comprise: (i) the nucleic acid sequence of sub-sub-element 1 of element 2 of at least 70%, 75%, 80%, 85%, 90%, 95%, 97%, 98%, 99% or 100% sequence identity to SEQ ID NO:9 or an effective fragment thereof; (ii) the nucleic acid sequence of sub-element 4 of enhancer element 2 of at least 70%, 75%, 80%, 85%, 90%, 95%, 97%, 98%, 99% or 100% sequence identity to SEQ ID NO: 10 or an effective fragment thereof; (iii) the nucleic acid sequence of enhancer element 9 slim of at least 70%, 75%, 80%, 85%, 90%, 95%, 97%, 98%, 99% or 100% sequence identity to SEQ ID NO:7 or an effective fragment thereof; and/or (v) the nucleic acid sequence of hypersensitive site 3 (HS3) of at least 70%, 75%, 80%, 85%, 90%, 95%, 97%, 98%, 99% or 100% sequence identity to SEQ ID NO:8 or an effective fragment thereof.

In some embodiments, the recombinant nucleic acid comprises a transgene. In some embodiments of the nucleic acids described herein, the recombinant nucleic acid comprises a transgene operably linked to any enhancer element described herein (e.g., enhancer element 14 or an effective fragment thereof) and/or any promoter described herein. In some embodiments, the recombinant nucleic acid comprises a transgene which encodes WASp. In some embodiments, the recombinant nucleic acid is comprised within an expression cassette. In some embodiments, said expression cassette is capable of expressing a gene product encoded by the transgene (e.g., WASp) in a cell (e.g., a stem cell and/or a progenitor cell, such as a hematopoietic stem and/or progenitor cell). In some embodiments, the expression cassette drives expression of the gene product at or near its physiologic level in a cell of a healthy subject. In some embodiments, the expression cassette drives expression of the gene product at a high level (e.g., at a level above the endogenous or physiologic level of the corresponding native gene). In some embodiments, said expression cassette is effective to express WASp at a physiologic or high level in a healthy subject when transduced into a cell (e.g., a megakaryocyte). In some embodiments, said expression cassette is effective to express WASp within about 60%, 50%, 40%, 30%, or 20% of endogenous, physiologic levels of WASp in a healthy subject.

### Terminology

As used herein, the term “about” and the term “approximately,” when used to modify a numeric value, indicate that deviations of up to 10% above and below the numeric value remain within the intended meaning of the recited value.

A “promoter” refers to a nucleic acid sequence capable of initiating transcription of a gene (e.g., a gene operably linked to the promoter). The term “promoter,” as used herein, has a meaning commonly known in the art.

An “enhancer” generally refers to a nucleic acid sequence that, when bound by one or more specific proteins called transcription factors, regulates (e.g., enhances by increases the rate of or likelihood of) transcription of an operably linked gene. The specification describes novel enhancers that are not commonly known in the art. Generally, enhancers may act by increasing the activity of the promoter operably linked to the same gene. Enhancers can be located away from the gene, upstream or downstream from the start site, e.g., up to 1,000,000 bp away from the gene). In some instances, the instant application describes new enhancer element and its properties. In some aspects, the invention provides new enhancer element(s), e.g., element 14. The invention also describes functional fragments of element 14: element 14 core and element 14 ultra-core. The examples presented herein show that the use of such enhancer elements improved gene transfer and viral titers in viral vectors.

As used herein, the term “effective fragment” when used with respect to a promoter (e.g., an effective fragment of a WAS promoter) refers to a fragment of the full-length promoter that is sufficient for the promoter activity, i.e., capable of initiating transcription of a gene operably linked to that promoter. In some embodiments, the effective fragment provides the same, substantially the same, or similar, expression level and/or pattern of an operably linked gene relative to the full-length promoter. In some embodiments, the effective fragment provides better expression level of an operably linked gene relative to the full-length promoter.

As used herein, the term “effective fragment” when used with respect to an enhancer (e.g., an effective fragment of a WAS enhancer) refers to a fragment of the full-length enhancer that is sufficient for the enhancer activity, i.e., capable of enhancing transcription of an operably linked gene when bound by a transcription factor. In some embodiments, the effective fragment provides the same, substantially the same, or similar, expression level and/or pattern of an operably linked gene relative to the full-length enhancer. In some embodiments, the effective fragment provides better expression level of an operably linked gene relative to the full-length enhancer.

The term “operably linked” refers to a nucleic acid sequence placed into a functional relationship with another nucleic acid sequence. The term “operably linked,” as used herein, has a meaning commonly known in the art. For example, a promoter is operably linked to a gene when that promoter is placed in a location that permits that promoter to initiate transcription of that gene. An enhancer is operably linked to a gene when that enhancer, when bound by an appropriate transcription factor, can regulate (e.g., enhance) expression of that gene.

“Recombinant” is used consistently with its usage in the art to refer to a nucleic acid sequence that is not naturally occurring, e.g., comprises portions that do not naturally occur together as part of a single sequence or that have been rearranged relative to a naturally occurring sequence. A recombinant nucleic acid is created by a process that involves human intervention and manipulation and/or is generated from a nucleic acid that was so created. A recombinant virus is one that comprises a recombinant nucleic acid. A recombinant cell is one that comprises a recombinant nucleic acid.

As used herein, the term “recombinant vector” refers to an artificially created polynucleotide vector.

As used herein, the term “percent sequence identity” with respect to a reference nucleic acid or amino acid sequence is the percentage of nucleic acid bases or amino acid residues in a candidate sequence that are identical with the nucleic acid bases or amino acid residues in the reference sequence, respectively, after aligning the sequences and introducing gaps, if necessary, to achieve the maximum percent sequence identity. Methods of sequence alignment are well known in the art. Optimal alignment of sequences can be conducted by methods described in Needleman and Wunsch, 1970, J. Mol. Biol. 48:443; Pearson and Lipman, 1988, PNAS 85:2444, by computerized implementations of these algorithms. Alignments can be made using publicly available computer software such as BLASTp, BLASTn, BLAST-2, ALIGN or MegAlign Pro (DNASTAR) software.

As used herein, the term “an effective amount” refers to the amount of an agent or composition comprising the agent required to result in a particular physiological effect, e.g., to ameliorate or eliminate symptoms of a disease relative to an untreated patient. The effective amount of a particular agent may be represented in a variety of ways based on the nature of the agent, such as mass/volume, number of cells/volume, particles/volume, (mass of the agent)/(mass of the subject), number of cells/(mass of subject), or particles/(mass of subject). The effective amount of an agent or a composition described herein for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

### BRIEF DESCRIPTION OF THE DRAWINGS

The foregoing and other features and advantages of the present invention will be more fully understood from the following detailed description of illustrative embodiments taken in conjunction with the accompanying drawings in which:

FIG. 1 shows the expression of mCitrine in fetal liver (FL) CD34+ megakaryocytes transfected with mCitrine-expressing vectors containing different enhancer elements.

FIG. 2 illustrates the minimal backbone LV construct, WASVec2.0 V3.

FIG. 3 shows a schematic demonstrating vector constructs generated as part of a LV library for a screen to identify novel enhancers of the endogenous WAS gene.

FIG. 4 shows the results of the single element enhancer screen in megakaryocytes and the identification of new WAS regulatory elements.

FIG. 5A illustrates a LV construct wherein the E14 core fragment was incorporated into WASVec2.0 V3, named WASVec2.0 V1. It has E14core-E2(1^{st} half of 1 and 5 slim)-HS1pro-WASp(jCAT)-WPRE.

FIG. 5B illustrates an LV construct wherein the E14 ultra-core fragment was incorporated into WASVec2.0 V3, named WASVec2.0 V2. It has E14 ultra-core-E2(1^{st} half of 1 and 5 slim)-HS1pro-WASp(jCAT)-WPRE.

FIG. 5C shows the key structures of WASVec2.0 V2.

FIG. 6 shows the viral titers in HT-29 cells transduced with WAS vectors.

FIG. 7 shows the vector copy number (VCN) with increasing vector dose (TU/ml) for tested WAS vectors in human CD34+ mobilized PBSCs.

FIG. 8A shows a schematic which details the strategy for evaluating the in vitro rescue of WAS protein expression by WASVec2.0 V1, WASVec2.0 V2, WASVec2.0 V3, WASVec1.0, and WASVec1.6 (WAS1.6) in megakaryocytes differentiated from FL CD34+ cells in which the endogenous WAS gene was deleted.

FIGS. 8B to 8D show the in vitro restoration of WAS protein expression using the tested WAS vectors, in megakaryocytes in which endogenous WAS deleted.

FIG. 8E shows that WASVec2.0 V2 restores WAS protein (WASp) expression to HD levels at a VCN of 1.29 while WAS1.6 needs a VCN of 2.61 to restore HD of WASp in WAS knockout (KO) megakaryocytes. WASp expression was measured by mean fluorescence intensity.

FIG. 9A shows a schematic which details the strategy for evaluating the in vitro rescue of WAS protein expression by WASVec2.0 V2 or other vectors, in T cells in which the endogenous WAS gene was knocked out.

FIG. 9B shows the in vitro restoration of WAS protein levels with WASVec2.0 V2 and WASVec1.6 in T-cells in which the endogenous WAS gene was knocked out.

FIG. 10A shows the restoration of WAS protein levels with WASVec2.0 and WASVec1.6 in megakaryocytes derived from CD34+ mPBSCs in which the endogenous WAS gene was knocked out. WAS Vec2.0 is labeled as “WASVec” in the figure. WASVec1.6 is labeled as “WAS1.6pro” and “WAS1.6pro-WASp-WPRE” in the figure.

FIG. 10B is a replicate of the experiment shown in FIG. 10A, but does not show the results with WASVec1.6.

FIG. 11 shows a schematic which details the strategy for evaluating the in vivo restoration of WAS protein expression by WASVec2.0 V2 or other vectors in NBSWG neonatal mice.

FIG. 12 compares the expression levels of WAS protein in T cells, B cells, and myeloid cells derived from healthy donor NBSWG neonatal mice, WAS−/− NBSWG neonatal mice, and WAS−/− NBSWG neonatal mice transduced with WASVec2.0 V2 (labeled “WASVec” in the figure).

FIG. 13 compares the levels of platelet engraftment and WAS protein expression in platelets collected from NBSWG neonatal mice transplanted with healthy donor cells, NBSWG neonatal mice transplanted with WAS−/− knockout cells, and NBSWG neonatal mice transplanted with WAS−/− knockout cells that were transduced with WASVec2.0 V2 (labeled “WASVec” in the figure).

FIG. 14 shows the restoration of WAS protein levels in B cells, myeloid cells, T cells, and platelets of NBSWG neonatal mice transplanted with WAS−/− knockout cells that were transduced with low or high doses of WASVec2.0 V2 (labeled “WASVec” in the figure).

FIG. 15 demonstrates that WASVec2.0 V2 restores WAS protein expression to healthy donor levels in B cells, myeloid cells, T cells, and platelets of NBSWG neonatal mice transplanted with WAS−/− knockout cells that were transduced with low or high doses of WASVec2.0 V2 (labeled “WASVec” in the figure).

FIG. 16 shows the platelet levels in NBSWG neonatal mice transplanted with healthy donor cells, NBSWG neonatal mice transplanted with WAS−/− knockout cells, and NBSWG neonatal mice transplanted with WAS−/− knockout cells that were transduced with WASVec2.0 V2 (labeled “WASVec” in the figure) at 8-weeks post-transplant.

FIG. 17 shows the platelet activation response to thrombin stimulation in platelets derived from NBSWG neonatal mice transplanted with healthy donor cells, NBSWG neonatal mice transplanted with WAS−/− knockout cells, and NBSWG neonatal mice transplanted with WAS−/− knockout cells that were transduced with WASVec2.0 V2 (labeled “WASVec” in the figure) at 8-weeks post-transplant. CD61p: Integrin expressed in CD41/61 complex. CD62P: (P-selectin) is expressed on activated platelets and MKs.

FIG. 18 is a schematic representation of a transfer plasmid of FIG. 5B comprising the uCore E14 and uCore E2 megakaryocyte enhancers upstream of a coding sequence, in this case the novel platelet and megakaryocyte enhancers are located upstream of a minimal WAS promoter driving expression of a WASp. FIG. 18 underscores the presence of uCoreE14, uCore E2, and minimal WAS promoter as elements for selectively driving gene expression in certain blood cell lines.

FIG. 19A is a schematic of an experimental set up used to validate the activity of the novel enhancers in a NOD.Cg-Kit^{W-41J}Tyr⁺ Prkde^{scid} Il2rg^{tm1Wjl}/ThomJ (NBSGW) humanized mouse model in order to restore healthy donor levels of WASp expression in the MK/platelet lineage in order to successfully correct platelet counts and function.

FIG. 19B is a chart depicting numbers of CD45+ human cells over total CD45+ mouse and human cells in the blood used to calculate human chimerism for all experimental arms.

FIG. 19C is a chart depicting a calculation of WAS KO frequency by measuring the INDEL frequency compared to unedited WT DNA using the ICE tool (Synthego).

FIGS. 20A-20C are charts illustrating that enhancer elements uCore E14 and uCore E2 successfully improved expression of WASp in megakaryocytes and platelets and had superior performance compared to control vectors that did not include the novel enhancer elements(s).

FIGS. 20D-20F are histograms depicting WASp expression as measured by mean fluorescence intensity (MFI).

FIGS. 21A and 21B are charts representing the vector of FIG. 18 produced healthy donor levels of functional platelets.

FIGS. 22A-22C are charts representing restoration of WASp expression in multiple cell lineages in vivo with a vector of FIG. 18.

FIG. 23A is an schematic of an experimental set up for validating a vector encoding WASp. FIG. 23B depicts the results of the WAS KO indel frequency compared to unedited WT DNA. FIG. 23C depicts the average vector copy number (VCP) per cell for each experimental arm.

FIGS. 24A-24E collectively illustrate that a vector of the disclosure restores WASp expression and IL2 production in WASp knockout T cells.

FIG. 25 is a chart illustrating in vitro vector dose response in murine lin-cells.

FIGS. 26A-26C illustrate the results of the analysis of peripheral blood platelets for WASp expression.

FIG. 27 illustrates the results of platelet counts from CBC analysis of peripheral blood at 15 weeks post-transplant.

FIGS. 28A-28H depict the results of the CBC analysis of peripheral blood at 15 weeks post-transplant.

FIGS. 29A-29C depict the results of a vector copy number (VCN) analysis in the bone marrow (A), thymus (B), and spleen (C) at 20 weeks post-transplant.

FIGS. 30A-30C depict the results of engraftment as measured by ddPCR to determine the percentage of donor cells in the bone marrow (A), thymus(B), and spleen (C) at 20 weeks post-transplant.

FIG. 31 is a graft showing the bone marrow lineage distribution at 20 weeks post-transplant.

FIG. 32 is a graft showing the spleen lineage distribution at 20 weeks post-transplant.

FIG. 33 is a graft showing the thymus lineage distribution at 20 weeks post-transplant.

FIGS. 34A-34B depict hWASP expression in the bone marrow by lineage. (A) Percentage of hWASP+ cells within each defined hematopoietic lineage in the bone marrow. (B) Mean Fluorescence intensity (MFI) of hWASP in each defined lineage in the bone marrow.

FIGS. 35A-35B depict hWASP expression in the spleen by lineage. (A) Percentage of hWASP+ cells within each defined hematopoietic lineage in the spleen. (B) Mean Fluorescence intensity (MFI) of hWASP in each defined lineage in the spleen.

FIGS. 36A-36B depict hWASP expression in the thymus by lineage. (A) Percentage of hWASP+ cells within each defined hematopoietic lineage in the thymus. (B) Mean Fluorescence intensity (MFI) of hWASP in each defined lineage in the thymus.

FIG. 37 is a graph depicting serum levels of total IgE at 20 weeks post-transplant.

FIG. 38A depicts total serum levels of anti-dsDNA IgG measured by ELISA at 20 weeks post-transplant. FIG. 38B is a chart depicting the proportion of mice in each arm with positive dsDNA antibodies.

FIG. 39 depicts antibody responses (ELISA O.D. values) for anti-pneumococcal IgM to Pneumo Vax23.

It should be understood that the drawings are not necessarily to scale (e.g., schematics), and that like reference numbers refer to like features.

### DETAILED DESCRIPTION

In some aspects, the invention is based on elucidation of a new enhancer element, element 14, as well as effective fragments thereof, element 14 core and element 14 ultra-core. The examples presented herein show that the use of such enhancer elements in WAS-expressing vectors yielded improved gene transfer and viral titers.

In some aspects, the invention is based on elucidation of a minimal enhancer element, which can be placed in a vector backbone to support gene expression in certain cell types, including certain blood cell types. Such enhancers can be optimized to minimize vector size (such as the enhancer element backbone in WASVec2.0 V3). The examples presented herein show that the use of such minimal enhancer element vector backbone in WAS-expressing vectors yielded improved gene transfer and viral titers.

In some aspects, provided herein are vectors comprising the element 14 or an effective fragment thereof (such as element 14 core and element 14 ultra-core). Vectors described herein may further comprise a minimal enhancer element vector backbone (such as that of WASVec2.0 V3). The examples presented herein demonstrate that vectors having any one of such enhancer elements achieve superior hematopoietic stem and progenitor cell (HPSC) gene transfer and improved viral titer. The examples also demonstrate that such vectors can maintain the ability to restore physiologic levels of WASp expression in WAS−/− cells.

The examples presented herein also show that the in vivo use of the enhancer elements identified by the inventors, in WASp-expressing vectors, yielded improved WASp expression in all affected hematopoietic lineages, as well as restoration of platelet counts and platelet function to healthy subject levels.

Accordingly, in some embodiments, provided herein is an enhancer element 14, having nucleic acid sequence of SEQ ID NO: 1, or an effective fragment thereof. In some embodiments, provided herein is an enhancer element having at least or more than 70%, at least 75%, at least 80%, at least 85%, at least 90%, at least 95%, at least 97%, at least 98%, at least 99%, or 100% nucleic acid sequence identity to SEQ ID NO:1, or an effective fragment thereof. In some embodiments, provided herein is a vector comprising any such enhancer, for example, enhancer element 14 of SEQ ID NO:1, an enhancer element having at least or more than 70% (or any of the % identity noted above) nucleic acid sequence identity to SEQ ID NO:1, or an effective fragment thereof. In some embodiments, the vector is a viral vector, e.g., a lentiviral vector. In some embodiments, the vector is for expression of WAS protein (i.e., the vector drives expression of a gene that encodes WASp).

In some embodiments, provided herein is an enhancer element 14 core, having nucleic acid sequence of SEQ ID NO:2, or an effective fragment thereof. In some embodiments, provided herein is an enhancer element having at least or more than 70%, at least 75%, at least 80%, at least 85%, at least 90%, at least 95%, at least 97%, at least 98%, at least 99%, or 100% nucleic acid sequence identity to SEQ ID NO:2, or an effective fragment thereof. In some embodiments, provided herein is an enhancer comprising, or consisting of, the nucleic acid sequence of the enhancer from WASVec2.0 VI (SEQ ID NO:4), or the nucleic acid sequence having at least or more than 70% (or any of the % identity noted above) nucleic acid sequence identity to the enhancer from SEQ ID NO:4, or an effective fragment thereof. In some embodiments, provided herein is a vector comprising any such enhancer, for example, enhancer element 14 core of SEQ ID NO:2, an enhancer element having at least or more than 70% (or any of the % identity noted above) nucleic acid sequence identity to SEQ ID NO:2, or an effective fragment thereof. In some embodiments, the vector is a viral vector, e.g., a lentiviral vector. In some embodiments, the vector is for expression of WAS protein (i.e., the vectors drive expression of a gene that encodes WASp). In some embodiments, provided herein is vector WASVec2.0 VI (SEQ ID NO:4), or a vector substantially identical to WASVec2.0 VI (SEQ ID NO:4). In some embodiments, provided herein is vector having at least or more than 50%, at least 60%, at least 70%, at least 75%, at least 80%, at least 85%, at least 90%, at least 95%, at least 97%, at least 98%, at least 99%, or 100% nucleic acid sequence identity to SEQ ID NO:4.

In some embodiments, provided herein is an enhancer element 14 ultra-core, having nucleic acid sequence of SEQ ID NO:3, or an effective fragment thereof. In some embodiments, provided herein is an enhancer element having at least or more than 70%, at least 75%, at least 80%, at least 85%, at least at least 90%, at least 95%, at least 97%, at least 98%, or at least 99%, or 100% nucleic acid sequence identity to SEQ ID NO:3, or an effective fragment thereof. In some embodiments, provided herein is an enhancer comprising, or consisting of, the nucleic acid sequence of the enhancer from WASVec2.0 V2 (SEQ ID NO:5), or the nucleic acid sequence having at least or more than 70% (or any of the % identity noted above) nucleic acid sequence identity to the enhancer from SEQ ID NO:5, or an effective fragment thereof. In some embodiments, provided herein is a vector comprising any such enhancer, for example, enhancer element 14 ultra-core of SEQ ID NO:3, an enhancer element having at least or more than 70% (or any of the % identity noted above) nucleic acid sequence identity to SEQ ID NO:3, or an effective fragment thereof. In some embodiments, the vector is a viral vector, e.g., a lentiviral vector. In some embodiments, the vector is for expression of WAS protein (i.e., the vectors drive expression of a gene that encodes WASp). In some embodiments, provided herein is vector WASVec2.0 V2 (SEQ ID NO:5), or a vector substantially identical to WASVec2.0 V2 (SEQ ID NO:5). In some embodiments, provided herein is vector having at least or more than 50%, at least 60%, at least 70%, at least 75%, at least 80%, at least 85%, at least 90%, at least 95%, at least 97%, at least 98%, at least 99%, or 100% nucleic acid sequence identity to SEQ ID NO:5.

In some embodiments where a sequence having a percent identity to a given sequence is specified, the sequence is effective to perform the function of the given sequence. In some embodiments where a sequence having a percent identity to a given enhancer sequence is specified, the sequence is effective to perform the enhancer function of the given sequence.

In some embodiments, the enhancer element 14 or an effective fragment thereof (such as enhancer element 14 core or enhancer element 14 ultra-core) is used in combination with an additional enhancer element or elements. In some embodiments, the additional enhancer element or elements comprise: (i) the first half of enhancer element 2 core sub-element 1 comprising nucleic acid sequence of SEQ ID NO: 14 (or an effective fragment thereof), and/or (ii) enhancer element 2 core sub-element 5 comprising nucleic acid sequence of SEQ ID NO: 17 (or an effective fragment thereof). In some embodiments, the additional enhancer element or elements comprise or consist of the uCore E2 element consisting of the first half of enhancer element 2 core sub-element 1 SEQ ID NO: 14 and enhancer element 2 core sub-element 5 of SEQ ID NO:17. In some embodiments, the additional enhancer element or elements comprise or consist of the uCore E2 element of SEQ ID NO:32. In some embodiments, the additional enhancer element comprises a sequence having at least or more than 50%, at least 60%, at least 70%, at least 75%, at least 80%, at least 85%, at least 90%, at least 95%, at least 97%, at least 98%, at least 99%, or 100% nucleic acid sequence identity to SEQ ID NOs: 14, 17 or 32. In some embodiments, the additional enhancer element comprises a sequence having at least or more than 50%, at least 60%, at least 70%, at least 75%, at least 80%, at least 85%, at least 90%, at least 95%, at least 97%, at least 98%, at least 99%, or 100% nucleic acid sequence identity to SEQ ID NOs: 14, 17 or 32, and effective to perform the function of SEQ ID NOs: 14, 17 or 32.

In some embodiments, the vectors provided herein comprise the enhancer element 14 or an effective fragment thereof (such as enhancer element 14 core or enhancer element 14 ultra-core).

In some embodiments, the vectors provided herein comprise the enhancer element 14 or an effective fragment thereof (such as enhancer element 14 core or enhancer element 14 ultra-core), the first half of enhancer element 2 core sub-element 1 comprising nucleic acid sequence of SEQ ID NO: 14 (or an effective fragment thereof), and enhancer element 2 core sub-element 5 comprising nucleic acid sequence of SEQ ID NO: 17 (or an effective fragment thereof). In some embodiments, the vectors provided herein comprise the enhancer element 14 or an effective fragment thereof (such as enhancer element 14 core or enhancer element 14 ultra-core), and the uCore E2 element consisting of the first half of enhancer element 2 core sub-element 1 SEQ ID NO:14 and enhancer element 2 core sub-element 5 of SEQ ID NO:17. In some embodiments, the vectors provided herein comprise an enhancer element wherein the enhancer element comprises or substantially consists of the enhancer element 14 or an effective fragment thereof (such as enhancer element 14 core or enhancer element 14 ultra-core), and the uCore E2 element of SEQ ID NO:32.

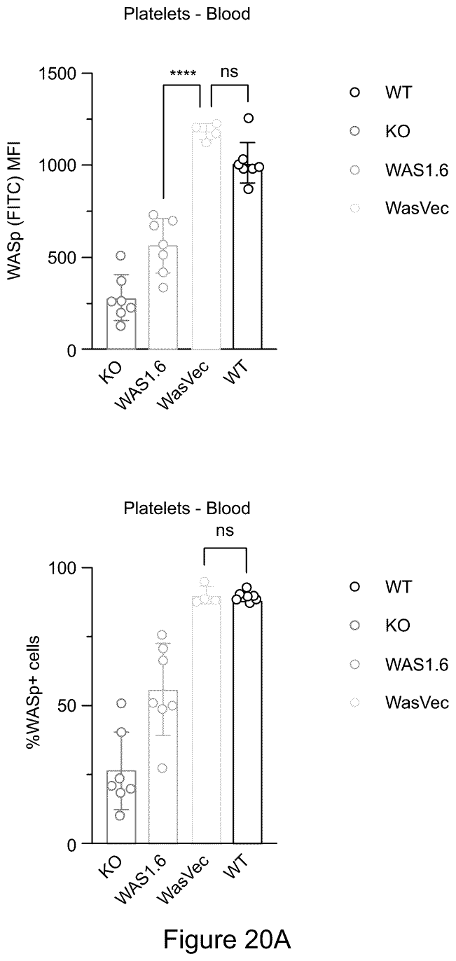

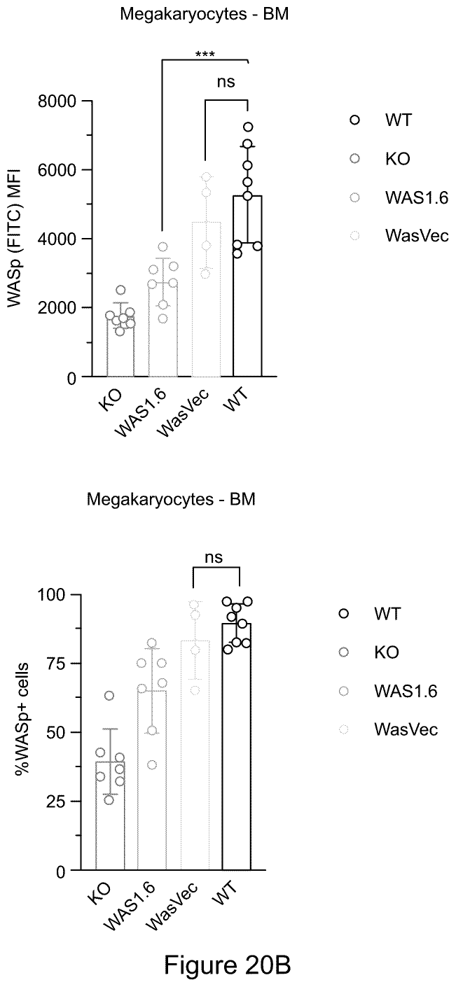

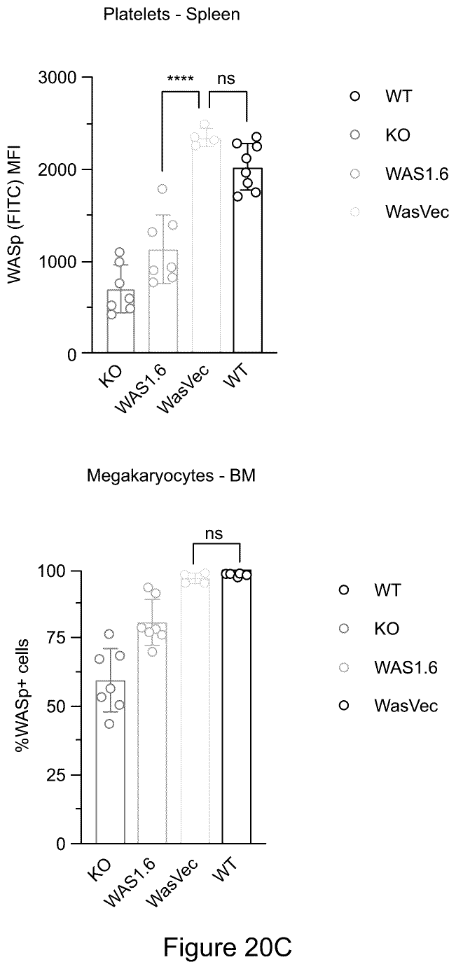

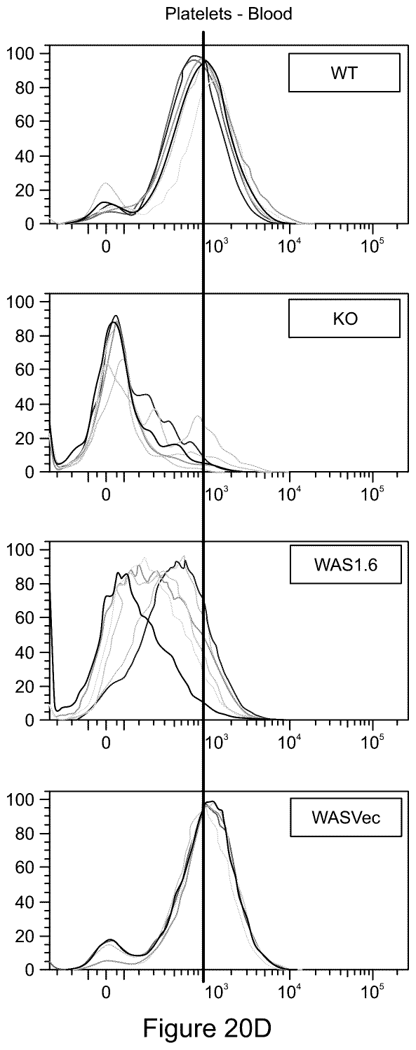

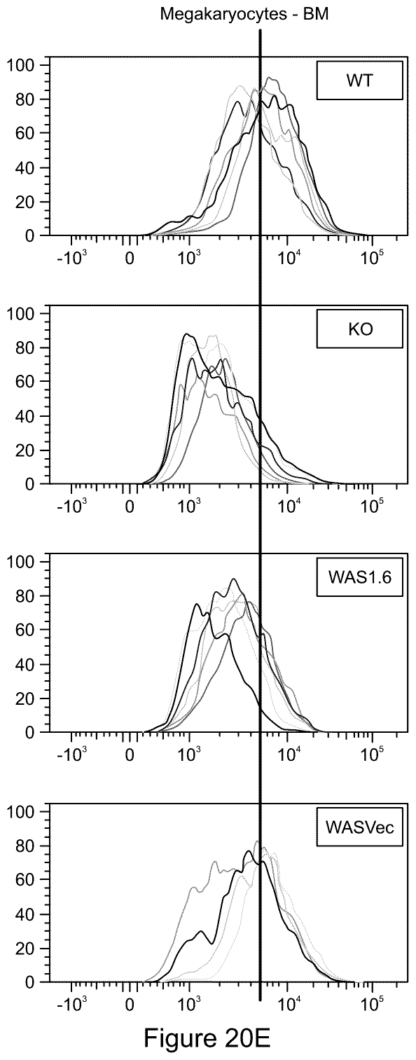

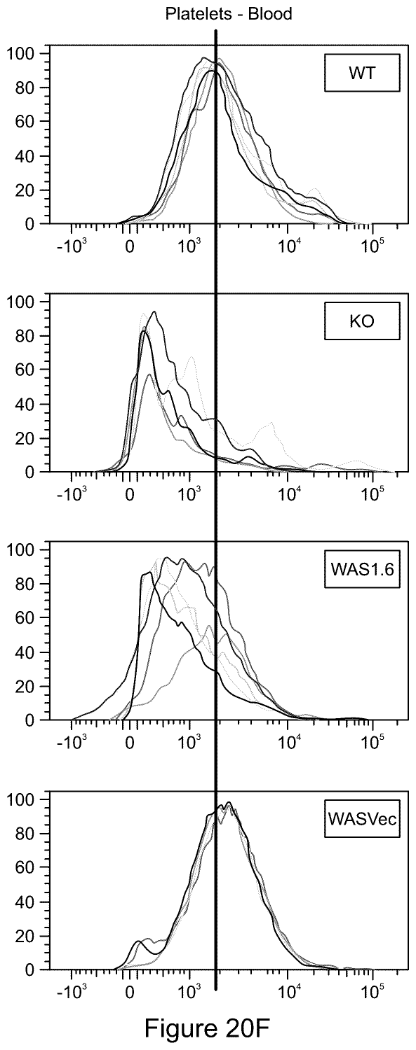

WASVec 1.0 is described in WO2021096887A (referenced as FIG. 20, SEQ ID NO: 17 in WO2021096887A), the entire disclosure of which is hereby incorporated by reference herein in its entirety, and in particular its disclosure relating to the enhancer, promoter, WAS and vector elements described therein, such as those of WASVec1.0. The enhancer elements used in WASVec 1.0 are described in WO2021096887A.

In some embodiments, the enhancers provided herein have been reduced to a minimal backbone relative to the enhancer in WASVec1.0. In some embodiments, a minimal backbone enhancer provided herein lacks: (i) “element 9 slim” (E9s) having the nucleic acid sequence of SEQ ID NO:7, an effective fragment thereof, or an element substantially identical thereto, (ii) “Hypersensitive site 3 slim” (HS3s) having the nucleic acid sequence of SEQ ID NO:8, an effective fragment thereof, or an element substantially identical thereto, (iii) sub-sub element 1 of element 2 having the nucleic acid sequence of SEQ ID NO:9, an effective fragment thereof, or an element substantially identical thereto, and/or (iv) sub element 4 of E2 having the nucleic acid sequence of SEQ ID NO: 10, an effective fragment thereof, or an element substantially identical thereto. In some embodiments, a minimal backbone enhancer provided herein lacks “element 9 slim” (E9s) having the nucleic acid sequence of SEQ ID NO:7 and/or an effective fragment thereof. In some embodiments, a minimal backbone enhancer provided herein lacks “Hypersensitive site 3 slim” (HS3s) having the nucleic acid sequence of SEQ ID NO:8 and/or an effective fragment thereof. In some embodiments, a minimal backbone enhancer provided herein lacks sub-sub element 1 of element 2 having the nucleic acid sequence of SEQ ID NO:9 and/or an effective fragment thereof. In some embodiments, a minimal backbone enhancer provided herein lacks sub element 4 of E2 having the nucleic acid sequence of SEQ ID NO: 10 and/or an effective fragment thereof.

In some embodiments, the enhancers provided herein, that have been reduced to a minimal backbone relative to the enhancer in WASVec1.0, lack the elements described in the preceding paragraph (such as “element 9 slim” (E9s), “Hypersensitive site 3 slim” (HS3s), sub element 4 of E2, and sub-sub element 1 of element 2, of the sequences specified above, or effective fragments thereof), but comprise the first half of enhancer element 2 core sub-element 1 comprising nucleic acid sequence of SEQ ID NO:14, and comprise enhancer element 2 core sub-element 5 comprising nucleic acid sequence of SEQ ID NO: 17 (such as in WASVec2.0 V3 provided herein).

In some embodiments, the enhancers provided herein, that have been reduced to a minimal backbone relative to the enhancer in WASVec1.0, lack the elements described in the preceding paragraph (such as “element 9 slim” (E9s), “Hypersensitive site 3 slim” (HS3s), sub element 4 of E2, and sub-sub element 1 of element 2, of the sequences specified above, or effective fragments thereof), but comprise the uCore E2 element consisting of the first half of enhancer element 2 core sub-element 1 of SEQ ID NO: 14 and enhancer element 2 core sub-element 5 of SEQ ID NO: 17 (such as in WASVec2.0 V3 provided herein). In some embodiments, the enhancers provided herein, that have been reduced to a minimal backbone relative to the enhancer in WASVec1.0, lack the elements described in the preceding paragraph (such as “element 9 slim” (E9s), “Hypersensitive site 3 slim” (HS3s), sub element 4 of E2, and sub-sub element 1 of element 2, of the sequences specified above, or effective fragments thereof), but comprise the uCore E2 element of SEQ ID NO:32. In some embodiments, the enhancers provided herein comprise or substantially consist of the uCore E2 element of SEQ ID NO:32. In some embodiments, the enhancers provided herein comprise or substantially consist of the combination of SEQ ID NO: 14 and SEQ ID NO: 17.

In some embodiments, the vectors provided herein comprise an enhancer comprising, or consisting of, the nucleic acid sequence of the enhancer in WASVec2.0 V3. In some embodiments, the vectors provided herein comprise an enhancer comprising, or consisting of, the nucleic acid sequence of the enhancer in WASVec2.0 V2.

In some embodiments, an enhancer provided herein comprises the first half of enhancer element 2 core sub-element 1 comprising nucleic acid sequence of SEQ ID NO:14, an effective fragment thereof, or a sequence substantially identical thereto. In some embodiments, an enhancer provided herein comprises an enhancer element having at least 70%, at least 75%, at least 80%, at least 85%, at least 90%, at least 95%, at least 97%, at least 98%, at least 99%, or 100% nucleic acid sequence identity to SEQ ID NO:14, or an effective fragment thereof. Vectors comprising such enhancers are also contemplated.

In some embodiments, an enhancer provided herein comprises enhancer element 2 core sub-element 5 comprising nucleic acid sequence of SEQ ID NO:17, an effective fragment thereof, or a sequence substantially identical thereto. In some embodiments, an enhancer provided herein comprises an enhancer element having at least 70%, at least 75%, at least 80%, at least 85%, at least 90%, at least 95%, at least 97%, at least 98%, at least 99%, or 100% nucleic acid sequence identity to SEQ ID NO: 17, or an effective fragment thereof. Vectors comprising such enhancers are also contemplated.

In some embodiments, an enhancer provided herein comprises uCoreE2 element comprising nucleic acid sequence of SEQ ID NO:32, an effective fragment thereof, or a sequence substantially identical thereto. In some embodiments, an enhancer provided herein comprises an enhancer element having at least 70%, at least 75%, at least 80%, at least 85%, at least 90%, at least 95%, at least 97%, at least 98%, at least 99%, or 100% nucleic acid sequence identity to SEQ ID NO:32, or an effective fragment thereof. Vectors comprising such enhancers are also contemplated.

In some embodiments, provided herein is an expression construct (e.g., a vector) comprising: a nucleic acid sequence of an enhancer comprising, or consisting of, the first half of enhancer element 2 core sub-element 1 of SEQ ID NO: 14, and enhancer element 2 core sub-element 5 of SEQ ID NO: 17; a nucleic acid sequence of a promoter or an effective fragment thereof; and a nucleic acid that encodes the Wiskott-Aldrich Syndrome protein (WASp) operably linked to said nucleic acid sequence of the enhancer and said nucleic acid sequence of the promoter; wherein said expression construct does not comprise: (i) the nucleic acid sequence of enhancer element 2 of SEQ ID NO:13, (ii) the nucleic acid sequence of sub-sub-element 1 of element 2 of SEQ ID NO:9 or an effective fragment thereof; (iii) the nucleic acid sequence of sub-element 4 of enhancer element 2 of SEQ ID NO: 10 or an effective fragment thereof; (iv) the nucleic acid sequence of enhancer element 9 slim of SEQ ID NO:7 or an effective fragment thereof; and/or (v) the nucleic acid sequence of hypersensitive site 3 (HS3) of SEQ ID NO:8 or an effective fragment thereof. In some embodiments, the vector is a lentiviral vector.

In some embodiments, provided herein is an expression construct (e.g., a vector) comprising: a nucleic acid sequence of an enhancer comprising, or consisting of, uCore E2 element of SEQ ID NO:32; a nucleic acid sequence of a promoter or an effective fragment thereof; and a nucleic acid that encodes the Wiskott-Aldrich Syndrome protein (WASp) operably linked to said nucleic acid sequence of the enhancer and said nucleic acid sequence of the promoter; wherein said expression construct does not comprise: (i) the nucleic acid sequence of enhancer element 2 of SEQ ID NO:13, (ii) the nucleic acid sequence of sub-sub-element 1 of element 2 of SEQ ID NO:9 or an effective fragment thereof; (iii) the nucleic acid sequence of sub-element 4 of enhancer element 2 of SEQ ID NO: 10 or an effective fragment thereof; (iv) the nucleic acid sequence of enhancer element 9 slim of SEQ ID NO:7 or an effective fragment thereof; and/or (v) the nucleic acid sequence of hypersensitive site 3 (HS3) of SEQ ID NO:8 or an effective fragment thereof. In some embodiments, the vector is a lentiviral vector.

In some embodiments, provided herein is an expression construct (e.g., a vector) comprising: a nucleic acid sequence of an enhancer comprising: enhancer element 14 of SEQ ID NO: 1, enhancer element 14 core of SEQ ID NO:2 or enhancer element 14 ultra-core of SEQ ID NO3; a nucleic acid sequence of a promoter or an effective fragment thereof; and a nucleic acid that encodes the Wiskott-Aldrich Syndrome protein (WASp) operably linked to said nucleic acid sequence of the enhancer and said nucleic acid sequence of the promoter. In some embodiments, the vector is a lentiviral vector.

In some embodiments, provided herein is an expression construct (e.g., a vector) comprising: a nucleic acid sequence of an enhancer comprising: enhancer element 14 of SEQ ID NO: 1, enhancer element 14 core of SEQ ID NO:2 or enhancer element 14 ultra-core of SEQ ID NO3; a nucleic acid sequence of a promoter or an effective fragment thereof; and a nucleic acid that encodes the Wiskott-Aldrich Syndrome protein (WASp) operably linked to said nucleic acid sequence of the enhancer and said nucleic acid sequence of the promoter; wherein said expression construct does not comprise: (i) the nucleic acid sequence of enhancer element 2 of SEQ ID NO:13, (ii) the nucleic acid sequence of sub-sub-element 1 of element 2 of SEQ ID NO:9 or an effective fragment thereof; (iii) the nucleic acid sequence of sub-element 4 of enhancer element 2 of SEQ ID NO: 10 or an effective fragment thereof; (iv) the nucleic acid sequence of enhancer element 9 slim of SEQ ID NO:7 or an effective fragment thereof; and/or (v) the nucleic acid sequence of hypersensitive site 3 (HS3) of SEQ ID NO:8 or an effective fragment thereof. In some embodiments, the vector is a lentiviral vector.

In some embodiments of the expression constructs (e.g., vectors) described herein, the promoter is any human promoter (such as any human promoter known in the art). In some embodiments of the expression constructs (e.g., vectors) described herein, the promoter is the endogenous promoter of the WAS gene, e.g., a human endogenous WAS gene promoter. In some embodiments, the promoter comprises nucleic acid sequence of SEQ ID NO: 11, or a sequence having at least 70%, at least 75%, at least 80%, at least 85%, at least 90%, at least 95%, at least 97%, at least 98%, at least 99%, or 100% nucleic acid sequence identity to SEQ ID NO:11. In some embodiments, the promoter has maximum length of 600 bp and comprises the sequence of HS1pro (SEQ ID NO:12). In some embodiments, the promoter comprises nucleic acid sequence of SEQ ID NO: 12, or a sequence having at least 70%, at least 75%, at least 80%, at least 85%, at least 90%, at least 95%, at least 97%, at least 98%, at least 99%, or 100% nucleic acid sequence identity to SEQ ID NO:12. In some embodiments, the promoter is the effective fragment of the endogenous promoter of the WAS gene consisting of, or substantially consisting of, the sequence of HS1pro (SEQ ID NO:12).

In some embodiments where a sequence having a percent identity to a given promoter sequence is specified, the sequence is effective to perform the promoter function of the given sequence.

In some embodiments, the vectors described herein comprise a transgene operably linked to any of the enhancer and/or promoter elements described herein. In some embodiments, the transgene encodes a protein (e.g., WASp).

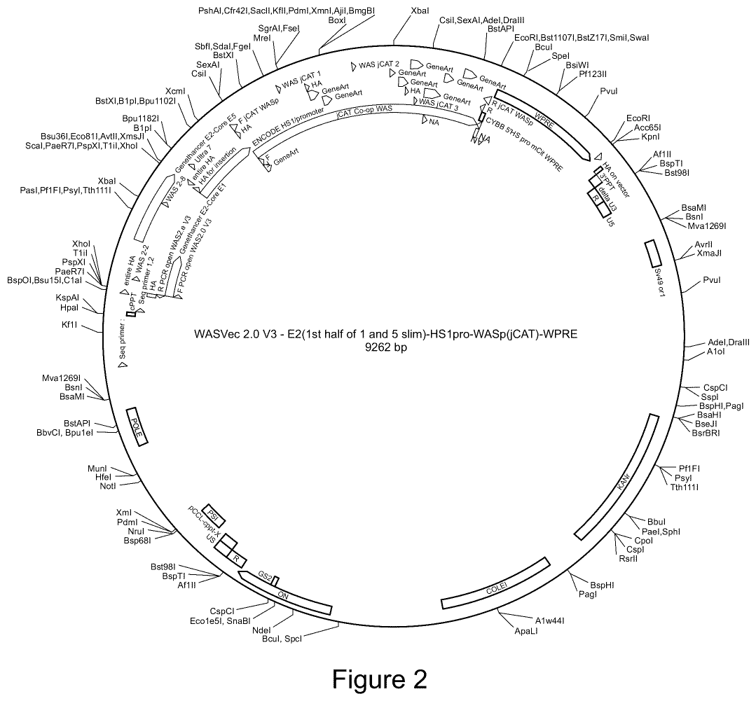

In some embodiments, the vectors described herein comprise any or all of the features of the vector shown in FIG. 2.

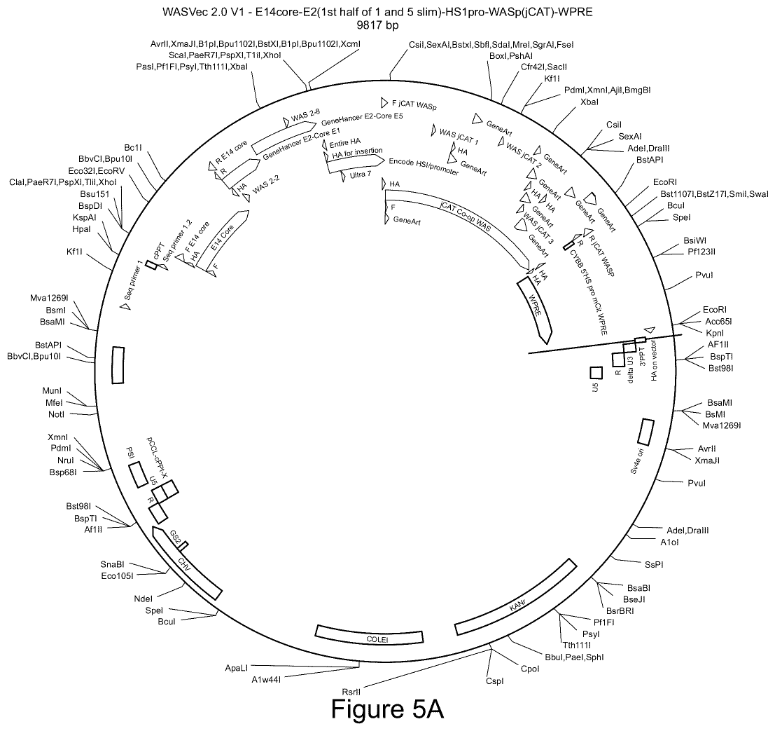

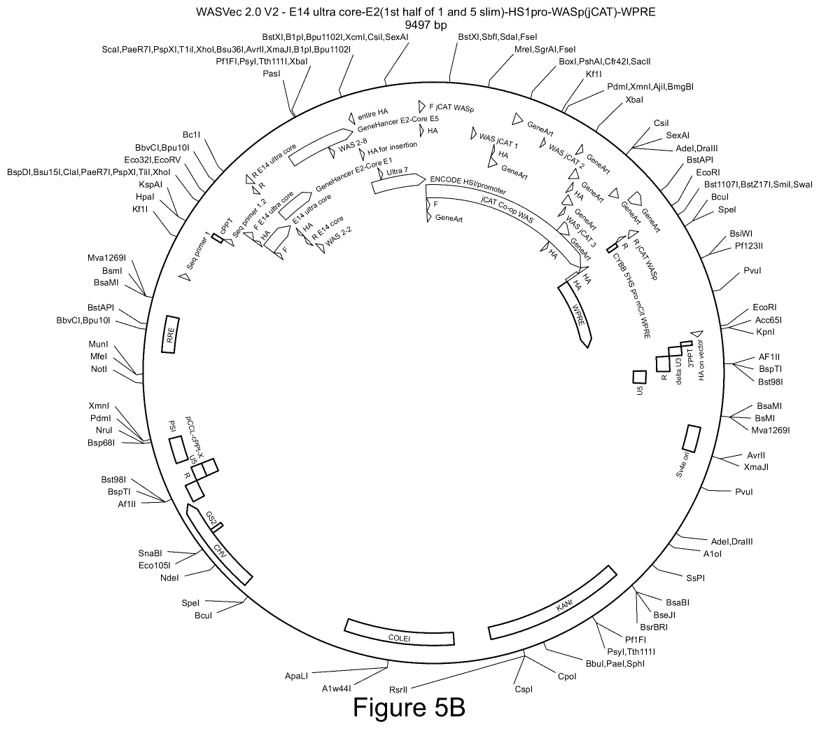

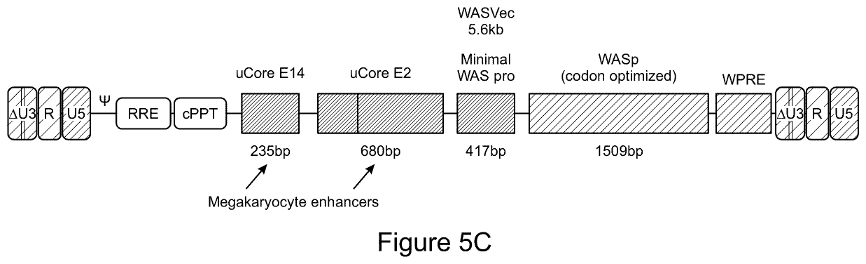

In some embodiments, the vectors described herein comprise any or all of the features of the vector shown in FIG. 5A.

In some embodiments, the vectors described herein comprise any or all of the features of the vector shown in FIG. 5B.

In some embodiments, described herein is a vector comprising a nucleic acid sequence of SEQ ID NO:4. In some embodiments, described herein is a vector comprising a nucleic acid sequence having at least 70%, at least 75%, at least 80%, at least 85%, at least 90%, at least 95%, at least 97%, at least 98%, at least 99%, or 100% nucleic acid sequence identity to SEQ ID NO:4.

In some embodiments, described herein is a vector comprising a nucleic acid sequence of SEQ ID NO:5. In some embodiments, described herein is a vector comprising a nucleic acid sequence having at least 70%, at least 75%, at least 80%, at least 85%, at least 90%, at least 95%, at least 97%, at least 98%, at least 99%, or 100% nucleic acid sequence identity to SEQ ID NO:5.

In some embodiments, described herein is a vector comprising a nucleic acid sequence of SEQ ID NO:6. In some embodiments, described herein is a vector comprising a nucleic acid sequence having at least 70%, at least 75%, at least 80%, at least 85%, at least 90%, at least 95%, at least 97%, at least 98%, at least 99%, or 100% nucleic acid sequence identity to SEQ ID NO:6.

In some embodiments, the vectors provided herein increase expression level and titer of the operably linked transgene, e.g., WAS. In some embodiments, the vectors provided herein are capable of capable of expressing a transgene, e.g., WAS, at a physiologic level in a cell (e.g., at or near the level of expression of a native gene corresponding to the transgene). In some embodiments, the vectors provided herein are capable of capable of expressing a transgene, e.g., WAS, in a cell at a level that is at least or more than 25%, 30%, 40%, 50%, 60%, 70%, 75%, 80%, 85%, 90% or 95% of the level of expression of a native gene corresponding to the transgene. In some embodiments, the vectors provided herein are capable of capable of expressing a transgene, e.g., WAS, at a high level in a cell (e.g., higher than the level of expression of a native gene corresponding to the transgene). In some embodiments, the vectors are lentiviral vectors.

In some embodiments, the vectors provided herein are optimized to reduce vector size. In some embodiments, the vectors provided herein are less than 6 kb in size. In some embodiments, the vectors provided herein are about 5.9 kb or less than 5.9 kb in size. In some embodiments, the vectors provided herein are about 5.8 kb or less than 5.8 kb in size. In some embodiments, the vectors provided herein are about 5.7 kb or less than 5.7 kb in size. In some embodiments, the vectors provided herein are about 5.6 kb or less than 5.6 kb in size. In some embodiments, the vectors provided herein are about 5.5 kb or less than 5.5 kb in size. In some embodiments, the vectors provided herein are about 5.4 kb in size. In some of these embodiments, the vectors are lentiviral vectors. In some of these embodiments, the vectors are lentiviral vectors and the transgene encodes WASp.

In some embodiments, the vectors provided herein are for the treatment of any disorder associated with a deficient expression of a protein encoded by the transgene. In some embodiments, the vectors provided herein are for the treatment of any disorder associated with a deficient expression of WAS protein. In some embodiments, the vectors provided herein are for the treatment of Wiskott-Aldrich Syndrome (WAS). In some embodiments, the vectors provided herein are for the treatment of XLT.

Where WAS transgene and/or expression of WASp are mentioned herein, use of other transgenes and/or expression of other gene products (such as those described herein) are also contemplated.

Where vectors are mentioned herein, use of other transgene delivery vehicles (such as those described herein) are also contemplated.

### Additional Descriptions of Enhancer Elements

The bioinformatic analysis and enhancer screen described in the examples of this application revealed an approximately 2173 bp sequence, named element 14, which increased WAS expression 1.7-fold over the endogenous WAS promoter alone in human HSPC derived megakaryocytes. The disclosure contemplates that element 14 and functional fragments thereof, when driving expression at least from a WAS promoter, a WAS minimal promoter, or a similar promoter, can be used to drive the expression of various other genes in certain blood cell types. In some embodiments, described herein is the 2173 bp nucleic acid sequence of SEQ ID NO:1, herein referred to as “element 14 (E14)”.

A functional 555 bp region within E14 was identified by the inventors. In some embodiments, described herein is the 555 bp nucleic acid sequence of SEQ ID NO:2, herein referred to as “element 14 core.”

A functional 234 bp region within the E14 core was further identified by the inventors. In some embodiments, described herein is the 234 bp nucleic acid sequence of SEQ ID NO:3, herein referred to as “element 14 ultra-core”.

In some embodiments, the E14 core fragment is incorporated into WASVec2.0 V3 to generate WASVec2.0 V1. In some embodiments, the E14 ultra-core fragment is incorporated into WASVec2.0 V3 to generate WASVec2.0 V2.

In some embodiments, WASVec2.0 V1, WASVec2.0 V2, and WASVec2.0 V3 show increased viral titer relative to WASVec 1.0 after transfection into human cells.

In some embodiments, WASVec2.0 V1, WASVec2.0 V2, and WASVec2.0 V3 show increased gene transfer relative to WASVec 1.0 after transfection into human cells.

In some embodiments, the expression cassette comprises a slim enhancer element 2 (SEQ ID NO:13) or an effective fragment thereof. In some embodiments, the expression cassette comprises an effective fragment of enhancer element 2 where the fragment comprises enhancer element 2 core sub-element 1 (SEQ ID NO:14+SEQ ID NO:9). In some embodiments, the expression cassette comprises an effective fragment of enhancer element 2 where the fragment comprises enhancer element 2 core sub-element 4 (SEQ ID NO:10). In some embodiment, the expression cassette comprises enhancer element 2 core sub-element 5 (SEQ ID NO:17). In some embodiments, the expression cassette comprises an effective fragment of enhancer element 2 where the fragment comprises enhancer element 2 core sub-element 1 (SEQ ID NO:14+SEQ ID NO:9), enhancer element 2 core sub-element 4 (SEQ ID NO: 10), and enhancer element 2 core sub-element 5 (SEQ ID NO:17).

In some embodiments, the expression cassette does not comprise a slim enhancer element 2 (SEQ ID NO:13). In some embodiments, the expression cassette does not comprise SEQ ID NO:9). In some embodiments, the expression cassette does not comprise enhancer element 2 core sub-element 4 (SEQ ID NO:10).

In some embodiments the expression cassette comprises an effective fragment of enhancer element 2 where the fragment comprises the first half of enhancer element 2 core sub-element 1 (SEQ ID NO:14), and enhancer element 2 core sub-element 5 (SEQ ID NO:17). In some embodiments the expression cassette comprises an effective fragment of enhancer element 2 where the fragment consists of the first half of enhancer element 2 core sub-element 1 (SEQ ID NO:14), and enhancer element 2 core sub-element 5 (SEQ ID NO:17).

In some embodiments, the expression cassette comprises enhancer element E9 core sequence (SEQ ID NO:7). In some embodiments, the expression cassette comprises enhancer element HS3 core sequence (SEQ ID NO:8).

In some embodiments, the expression cassette does not comprise enhancer element E9 core sequence (SEQ ID NO:7). In some embodiments, the expression cassette does not comprise enhancer element HS3 core sequence (SEQ ID NO:8).

In some embodiments, the expression cassette comprises enhancer element E9 core sequence (SEQ ID NO:7), enhancer element HS3 core sequence (SEQ ID NO:8), and a slim enhancer element 2 sequence (SEQ ID NO:13).

In some embodiments, the expression cassette does not comprise the combination of enhancer element E9 core sequence (SEQ ID NO:7), enhancer element HS3 core sequence (SEQ ID NO:8), and a slim enhancer element 2 sequence (SEQ ID NO:13).

In some embodiments, the expression cassette does not comprise 2^{nd} half of core sub-element 1 of enhancer element 2 (SEQ ID NO:9). In some embodiments, the expression cassette does not comprise core sub-element 4 of enhancer element 2 (SEQ ID NO:10).

In some embodiments, the expression cassette does not comprise the combination of enhancer element E9 core sequence (SEQ ID NO:7), enhancer element HS3 core sequence (SEQ ID NO:8), 2^{nd} half of core sub-element 1 of enhancer element 2 (SEQ ID NO:9), and core sub-element 4 of enhancer element 2 (SEQ ID NO:10).

In some embodiments the expression cassette comprises an enhancer element E9 core sequence (SEQ ID NO:7), enhancer element HS3 core sequence (SEQ ID NO: 8), enhancer element 2 core sub-element 1 (SEQ ID NO:14+SEQ ID NO:9), enhancer element 2 core sub-element 4 (SEQ ID NO:10), and enhancer element 2 core sub-element 5 (SEQ ID NO: 17).

In some embodiments the expression cassette does not comprise the combination of an enhancer element E9 core sequence (SEQ ID NO:7), enhancer element HS3 core sequence (SEQ ID NO: 8), enhancer element 2 core sub-element 1 (SEQ ID NO:14+SEQ ID NO:9), enhancer element 2 core sub-element 4 (SEQ ID NO:10), and enhancer element 2 core sub-element 5 (SEQ ID NO:17).

In some embodiments, the expression cassette comprises enhancer element E9 core sequence (SEQ ID NO:7), enhancer element HS3 core sequence (SEQ ID NO: 8), a first half of enhancer element 2 core sub-element 1 (SEQ ID NO:14), and enhancer element 2 core sub-element 5 (SEQ ID NO:17).

In some embodiments, the expression cassette does not comprise the combination of enhancer element E9 core sequence (SEQ ID NO:7), enhancer element HS3 core sequence (SEQ ID NO: 8), a first half of enhancer element 2 core sub-element 1 (SEQ ID NO: 14), and enhancer element 2 core sub-element 5 (SEQ ID NO:17).

In some embodiments, the expression cassette comprises an enhancer comprising or substantially consisting of the uCore E2 element of SEQ ID NO:32.

In some embodiments, the expression cassette comprises an enhancer comprising or substantially consisting of the combination of the element 14 or an effective fragment thereof (e.g., element 14 core or element 14 ultra-core) and the uCore E2 element of SEQ ID NO:32.

In some embodiments, the expression cassette comprises an enhancer comprising or substantially consisting of the combination of (i) SEQ ID NO:1, SEQ ID NO:2 or SEQ ID NO:3, and/or (ii) SEQ ID NO:32.

In some embodiments, the enhancer is capable of expressing an operably linked transgene (e.g., in a megakaryocyte) at a level at least or more than 25%, 30%, 35%, 40%, 45%, 50%, 55%, 60%, 65%, 70%, 75%, 80%, 85%, 90%, 95% or 100% of the wild-type expression of the gene or expression of the gene in a healthy subject. In some embodiments, the transgene encodes WASp.

In some embodiments, the enhancer is capable of expressing an operably linked transgene at or about the physiologic or endogenous level (the normal level of expression of the corresponding wild type gene) in a cell, e.g., a megakaryocyte. About the normal level of expression may be within 5%, within 10%, within 15%, within 20%, within 25%, or within 30%, of the wild type expression level of the gene (e.g., 70% to 100% of the wild type level, 80-100% of the wild type level, 90-100% of the wild type level, 100% to 130% of the wild type level, or 75% to 125% of the wild type level, or any range in between). In some embodiments, the transgene encodes WASp.

In some embodiments, the enhancer is capable of expressing an operably linked transgene at a physiologic level in all affected cell lineages. In some embodiments, the enhancer is capable of expressing an operably linked transgene at a physiologic level in megakaryocytes. In some embodiments, the transgene encodes WASp. In some embodiments, the enhancer is capable of expressing WASp at a level that increases platelet counts. In some embodiments, the enhancer is capable of expressing WASp at a level that restores platelet counts in vivo to healthy subject levels. In some embodiments, the enhancer is capable of expressing WASp at a level that, in vivo, increases platelet engraftment and/or improves or restores platelet function to healthy subject levels.

In some embodiments, the enhancer is capable of expressing an operably linked transgene at a high level (i.e., capable of overexpressing the transgene) in a cell, e.g., a megakaryocyte, a platelet.

The enhancers described herein can be used with any of the promoters described herein.

The enhancers described herein can be used in any vectors described herein. In some embodiments, the enhancers described herein are for use in a viral vector (e.g., an LV). In some embodiments, the enhancers described herein are for use in a non-viral vector (e.g., a plasmid or a transposon).

In some embodiments, the enhancers described herein can be used in a single stranded oligonucleotide and/or a double stranded DNA homology directed repair template for CRISPR gene editing.

### Promoters

In some embodiments, the promoter described herein is any promoter (e.g., any human promoter).

In some embodiments, the promoter is the minimal CMV promoter. In some embodiments, the promoter is a constitutively active promoter such an as an elongation factor alpha short (EFS) or a phosphoglycerate kinase (PGK) promoter.

Recent systematic analysis of the biochemical compatibility of 1000 enhancer and 1000 promoter sequences revealed broad compatibility among human enhancer and promoter elements. See Bergman et al., 2022; Nature, doi: 10.1038/s41586-022-04877-w. Accordingly, in some embodiments, any expression cassette (e.g., a vector) described herein may comprise any human promoter (such as an endogenous promoter of any human gene). In some embodiments, an expression cassette (e.g., a vector) described herein comprises an endogenous promoter for any human gene. In some embodiments, the promoter is a human CMV promoter, a human phosphoglycerate kinase gene promoter, a human elongation factor 1 alpha (EF1-alpha) promoter, a human U6 promoter, a human ubiquitin promoter (e.g., a human ubiquitin C promoter).

In some embodiments, the promoter described herein is an endogenous promoter (e.g., a human promoter) for the transgene (e.g., a human gene) or an effective fragment thereof.

In some embodiments, the promoter described herein is an endogenous promoter of the WAS gene. In some embodiments, the promoter described herein is an endogenous promoter of the human WAS gene. In some embodiments, the promoter described herein is genome fragment of about 1.6 kb from the human WAS promoter. In some embodiments, the promoter described herein is a genome fragment of (or about) 1.6 kb from the human WAS promoter as disclosed in Dupre et al., 2005; Mol. Ther, 10(5):903-15, doi: 10.1016/j.ymthe.2004.08.008, which disclosure is hereby incorporated by reference herein in its entirety. In some embodiments, the promoter described herein comprises nucleic acid sequence of SEQ ID NO:11. In some embodiments, the promoter described herein comprises or consists of nucleic acid sequence of SEQ ID NO:11, or a sequence having at least 70%, at least 75%, at least 80%, at least 85%, at least at least 90%, at least 95%, at least 97%, at least 98%, or at least 99%, or 100% nucleic acid sequence identity to SEQ ID NO:11.

In some embodiments, an expression cassette (e.g., a vector) described herein comprises a WAS promoter (such as an endogenous promoter of the human WAS gene). In some embodiments, an expression cassette (e.g., a vector) described herein comprises a promoter comprising about 1.6 kb genome fragment from the human WAS promoter (such as the 1.6 kb WAS promoter disclosed in Dupre et al., 2005; Mol. Ther, 10(5):903-15, doi: 10.1016/j.ymthe.2004.08.008). In some embodiments, an expression cassette (e.g., a vector) described herein comprises a promoter comprising or consisting of nucleic acid sequence of SEQ ID NO: 11, or a sequence having at least 70%, at least 75%, at least 80%, at least 85%, at least at least 90%, at least 95%, at least 97%, at least 98%, or at least 99%, or 100% nucleic acid sequence identity to SEQ ID NO:11.

In some embodiments, the promoter described herein is an effective fragment of an endogenous promoter of the WAS gene. In some embodiments, the promoter described herein is an effective fragment of an endogenous promoter of the human WAS gene. In some embodiments, the promoter described herein is a minimal effective fragment of an endogenous promoter of the WAS gene (e.g., human WAS gene). In some embodiments, the promoter described herein is a minimal effective fragment of an endogenous promoter of the WAS gene, wherein said promoter has maximum length of 600 bp and contains the sequence of HS1pro (e.g., as described in WO2021/096887A1, the disclosure of which relating to promoters is hereby incorporated by reference herein in its entirety). In some embodiments, the promoter described herein is a minimal effective fragment of an endogenous promoter of the WAS gene, wherein said promoter comprises the sequence of HS1pro (e.g., as described in WO2021/096887A1). In some embodiments, the promoter described herein is HS1pro (e.g., as described in WO2021/096887A1), e.g., consists or substantially consists of the sequence of HS1pro. In some embodiments, the promoter described herein comprises or consists of SEQ ID NO:12. In some embodiments, the promoter described herein comprises or consists of nucleic acid sequence of SEQ ID NO:12, or a sequence having at least 70%, at least 75%, at least 80%, at least 85%, at least 90%, at least 95%, at least 97%, at least 98%, at least 99%, or 100% nucleic acid sequence identity to SEQ ID NO:12.

In some embodiments, an expression cassette (e.g., a vector) described herein comprises an effective fragment of an endogenous promoter of the WAS gene. In some embodiments, an expression cassette (e.g., a vector) described herein comprises an endogenous promoter of the human WAS gene. In some embodiments, an expression cassette (e.g., a vector) described herein comprises a minimal effective fragment of an endogenous promoter of the WAS gene (e.g., human WAS gene). In some embodiments, an expression cassette (e.g., a vector) described herein comprises a minimal effective fragment of an endogenous promoter of the WAS gene, wherein said promoter has maximum length of 600 bp and contains the sequence of HS1pro (e.g., as described in WO2021/096887A1, the disclosure of which relating to promoters is hereby incorporated by reference herein in its entirety). In some embodiments, an expression cassette (e.g., a vector) described herein comprises a minimal effective fragment of an endogenous promoter of the WAS gene, wherein said minimal effective fragment comprises the sequence of HS1pro (e.g., as described in WO2021/096887A1). In some embodiments, an expression cassette (e.g., a vector) described herein comprises a promoter wherein the promoter is HS1pro (e.g., as described in WO2021/096887A1), e.g., consists or substantially consists of the sequence of HS1pro. In some embodiments, an expression cassette (e.g., a vector) described herein comprises a promoter which comprises or consists of SEQ ID NO:12. In some embodiments an expression cassette (e.g., a vector) described herein comprises a promoter which comprises or consists of nucleic acid sequence of SEQ ID NO: 12, or a sequence having at least 70%, at least 75%, at least 80%, at least 85%, at least 90%, at least 95%, at least 97%, at least 98%, at least 99%, or 100% nucleic acid sequence identity to SEQ ID NO:12.

The promoters described herein can be used with any enhancer elements described herein. The promoters described herein can be used in any vectors described herein. In some embodiments, the promoters described herein are for use in a viral vector (e.g., an LV). In some embodiments, the promoters described herein are for use in a non-viral vector (e.g., a plasmid or a transposon).

### Transgenes

In some embodiments, any vector, expression construct, enhancer and/or promoter described herein can be used for expression of any transgene. In some embodiments, the transgene encodes a polypeptide, e.g., a polypeptide that has a therapeutic benefit. In some embodiments, the expression of the polypeptide encoded by the transgene (using any vector, expression construct, enhancer and/or promoter described herein) supplements deficient or absent expression of an endogenous polypeptide in a cell. An artisan would know or can determine the appropriateness of any particular transgene for use with the vectors, expression constructs, enhancers and/or promoters described herein.

In some embodiments, the transgene encodes a therapeutic peptide or protein. In some embodiments, the transgene encodes a chimeric antigen receptor. In some embodiments, the transgene encodes a clotting factor.

In some embodiments, the transgene encodes a WAS protein. In some embodiments, the transgene encodes a human WAS protein. In some embodiments, the transgene comprises a nucleic acid, such as DNA, of WAS gene (e.g., DNA of human WAS). In some embodiments, the transgene comprises a WAS cDNA (e.g., cDNA of a human WAS gene). In some embodiments, the transgene is or comprises a WAS cDNA having the nucleic acid sequence of SEQ ID NO:20. In some embodiments, the transgene is or comprises a WAS cDNA of a nucleic acid sequence having at least 70%, at least 75%, at least 80%, at least 85%, at least at least 90%, at least 95%, at least 97%, at least 98%, or at least 99%, or 100% nucleic acid sequence identity to SEQ ID NO:20. In some embodiments, the transgene comprises a codon-optimized WAS cDNA (e.g., codon-optimized cDNA of a human WAS gene). In some embodiments, the transgene is or comprises a codon-optimized WAS cDNA having the nucleic acid sequence of SEQ ID NO:21. In some embodiments, the transgene is or comprises a WAS cDNA of a nucleic acid sequence having at least 70%, at least 75%, at least 80%, at least 85%, at least 90%, at least 95%, at least 97%, at least 98%, at least 99%, or 100% nucleic acid sequence identity to SEQ ID NO:21.

### Polynucleotides or Nucleic Acids

In some embodiments, provided herein is a polynucleotide or a nucleic acid comprising any enhancer described herein, any promoter described herein, and any transgene described herein. In some embodiments, provided herein is a polynucleotide or a nucleic acid comprising any enhancer described herein and any promoter described herein, which are operably linked to any transgene described herein.

In some embodiments, the polynucleotides or nucleic acids described herein are capable of expressing the gene product encoded by a transgene.

In some embodiments, the polynucleotides or nucleic acids described herein are codon optimized (e.g., for human codon selection).

In some embodiments, the polynucleotides or nucleic acids described herein further comprise an untranslated region (UTR), a signal sequence, a Kozak sequence, a transcription start site, a polyadenylation sequence, a termination codon, and/or a transcriptional termination signal.

In some embodiments, the polynucleotides or nucleic acids are recombinant polynucleotides or nucleic acids.

### Vectors

In some embodiments, the delivery of a nucleic acid using any expression cassette, enhancer and/or promoter described herein is by use of a vector. The vector can be any viral or non-viral vector known in the art or described herein. For example, viral and non-viral vectors and delivery systems are described in Sung & Kim 2019, Biomaterials Research 23:8, doi: 10.1186/s40824-019-0156-z; Mali, 2013, Indian Journal of Human Genetics, 19(1):3-8; Hardee et al., 2017, Genes 8:65; Bulcha et al., 2020, Signal Transduction and Targeted Therapy; Ghosh et al., 2020, Applied Biosafety: Journal of ABSA International 25(1):7-18, the disclosures of each of which are hereby incorporated by reference herein in their entireties.

In some embodiments, the vector is a viral vector. In some embodiments the viral vector is a lentiviral vector (LV), a retroviral vector (RV), an adenoviral vector (AV), an adeno-associated virus vector (AAV), a herpes simplex virus vector (HSV), or a poxvirus vector.

In some embodiments, provided herein is an LV (such as a recombinant LV) comprising any expression cassette, enhancer and/or promoter described herein.

In some embodiments, provided herein is an RV comprising any expression cassette, enhancer and/or promoter described herein.

In some embodiments, provided herein is a gamma-retroviral vector comprising any expression cassette, enhancer and/or promoter described herein.

In some embodiments, provided herein is an AV comprising any expression cassette, enhancer and/or promoter described herein.

In some embodiments, provided herein is an AAV comprising any expression cassette, enhancer and/or promoter described herein.

In some embodiments, provided herein is an HSV comprising any expression cassette, enhancer and/or promoter described herein.

In some embodiments, provided herein is a poxvirus-based vector comprising any expression cassette, enhancer and/or promoter described herein.

In some embodiments, the viral vectors described herein are engineered for safety by making them replication incompetent. In some embodiments, the viral vectors described herein are replication-incompetent.

In some embodiments, the viral vectors described herein are replication-competent.

In some embodiments, the viral vectors described herein have no or low toxicity (i.e., have no effect on the physiology of the normal host cell).

In some embodiments, the viral vectors described herein are stable (such as no rearrangement of the genome occurs).

In some embodiments, the vector is a non-viral vector. In some embodiments, the non-viral vector is a naked DNA (e.g., a DNA plasmid). In some embodiments, the non-viral vector is a plasmid. In some embodiments, the non-viral vector is delivered in a lipid composition, in a chromosome, with a cationic polymer, or as a conjugate complex. In some embodiments, the non-viral vector is a liposome or lipid vector comprising plasmid DNA and a lipid solution. In some embodiments, the non-viral vector is a transposon vector.

Non-viral vectors or plasmid DNA can be transfected into cells, e.g., by chemical or physical transfection. Chemical transfection can be achieved by calcium phosphate, lipid or protein complexes. Physical transfection can be achieved by electroporation or microinjection.

In some embodiments, the vector described herein show high expression in MEG-01 cells (megakaryocyte cell line), and/or in Jurkat cells (T-cell line), and/or in RAMOs cells (B-cell line). In some embodiments, the vectors described herein show high expression in CB CD34+ differentiated megakaryocytes including “pro-megakaryocytes”, megakaryocytes, and platelets.

The expression cassettes, enhancers and/or promoters described herein with respect to lentiviral vectors need not be limited to their use in lentiviral vectors and can be incorporated in essentially any other construct where expression of a transgene (such as WASp) is desired. Thus, in some embodiments, nucleic acid constructs comprising any of the expression cassette components described herein (e.g., enhancers, promoters, and/or combinations thereof) are contemplated.

In some embodiments, the vectors described herein are capable of being delivered to both dividing and non-dividing cells. In some embodiments, the vectors described herein are capable of being delivered to non-dividing cells. In some embodiments, the vectors described herein are capable of being delivered to dividing cells.

In some embodiments, any of the vectors described herein are recombinant vectors.

Any vector described herein can be transduced or introduced into cells.

### Lentiviral Vectors

In some embodiments, the vector described herein is a lentiviral vector (LV).

In some embodiments, the LVs described herein contain any one or more of the elements typically found in lentiviral vectors. Such elements may comprise any one or more of, but need not be limited to, a vector genome packaging signal, a Rev Responsive Element (RRE), a polypurine tract (e.g., a central polypurine tract, a 3′ polypurine tract, etc.), a post-translational regulatory element (e.g., a modified Woodchuck Post-transcriptional Regulatory Element (WPRE)), an insulator, e.g., as described below.

In some embodiments, the LVs described herein can comprise various safety features. In some embodiments, the LV described herein is self-inactivating (SIN). In some embodiments, the LV described herein is TAT-independent. The various “‘safety’” features can include, for example, the presence of an insulator (e.g., an PB insulator in the 3′LTR; long terminal repeat). In some embodiments, an insulator (e.g., the PB insulator) is introduced into the 3′LTR for safety. In some embodiments, the HIV LTR is substituted with an alternative promoter (e.g., a CMV) to yield a higher titer vector without the inclusion of the HIV TAT protein during packaging.

Methods of constructing self-inactivating, replication-deficient, and/or TAT-independent LVs are known in the art.

In some embodiments, the LV provided herein is constructed to provide efficient transduction and high titer. Methods of constructing LVs that achieve efficient transductions and/or high titer are known in the art.

In some embodiments, the lentiviral vectors described herein comprise a self-inactivating (SIN) and TAT-independent configuration. This self-inactivating ability serves as a biosafety feature. In SIN vectors, the production of full-length vector RNA in transduced cells is greatly reduced or abolished altogether. This feature reduces the chance that replication-competent recombinants (RCRs) will emerge. Furthermore, it reduces the chances of aberrant expression of cellular coding sequences located adjacent to the vector integration site.

In some embodiments, an LTR region that has reduced promoter activity relative to wild-type LTR is employed in the LVs described herein.

In some embodiments, the LV is a SIN vector substantially incapable of reconstituting a wild-type lentivirus through recombination.

In some embodiments, a SIN design reduces the possibility of interference between the LTR and the promoter that is driving the expression of the transgene.

In some embodiments, self-inactivation is achieved through a deletion in the U3 region of the 3′ LTR of the LV DNA, i.e., the DNA used to produce the vector RNA. During RT, this deletion is transferred to the 5′ LTR of the proviral DNA.

In some embodiments, it is desirable to eliminate as many of the transcriptionally important motifs from the LTR as possible while sparing the polyadenylation determinants.

In some embodiments, the LVs described herein comprise a Rev response element (RRE) to enhance nuclear export of unspliced RNA. Illustrative RREs comprise, but are not limited to RREs such as that located at positions 7622-8459 in the HIV NL4-3 genome (Genbank accession number AF003887) as well as RREs from other strains of HIV or other retroviruses. Such sequences are readily available from Genbank or from the database with URL hiv-web.lanl.gov/content/index. One illustrative, but non-limiting RRE is shown in SEQ ID NO:25). In some embodiments, the LVs described herein comprise SEQ ID NO:25.

In some embodiments, the LVs described herein comprise a polypurine tract (e.g., central polypurine tract (cPPT), or 3′ poplypurine tract (3′PPT)). One illustrative, but non-limiting 3′PPT is shown in SEQ ID NO:27). In some embodiments, the LVs described herein comprise SEQ ID NO:27.

In some embodiments, insertion of a fragment containing the 3′PPT (see, e.g., SEQ ID NO:27) or the central polypurine tract (cPPT) in lentiviral (e.g., HIV-1) vector constructs is known to enhance transduction efficiency.

### Posttranscriptional Regulatory Elements (PRE)

In some embodiments, the vectors described herein comprise a post-transcriptional regulatory element (PRE). In some embodiments, the vectors described herein comprise one or more post-transcriptional regulatory elements (PREs) which increase expression of a heterologous nucleic acid (e.g., a nucleic acid that encodes WASp) at the protein level.

In some embodiments, the LV described herein comprise one or more post-transcriptional regulatory elements (PREs) which increase expression of a heterologous nucleic acid (e.g., a nucleic acid that encodes WASp) at the protein level. In some embodiments, PREs may be particularly useful in lentiviral constructs with modest promoters.

Posttranscriptional regulatory elements that do not rely on splicing events are not excised during the viral life cycle. Some examples are the post-transcriptional processing element of herpes simplex virus, the posttranscriptional regulatory element of the hepatitis B virus (HPRE) and the woodchuck hepatitis virus (WPRE). WPRE contains an additional cis-acting element not found in the HPRE. In some embodiments, the post-transcriptional regulatory element is WPRE.

The WPRE is characterized and described in U.S. Pat. No. 6,136,597, which is hereby incorporated by reference herein its entirety and in particular in regard to its description of WPRE. As described therein, the WPRE is an RNA export element. WPRE promotes transport of RNA from the nucleus to the cytoplasm. It inserts a cis-acting nucleic acid sequence, such that the element and the transgene are contained within a single transcript to enhance the expression of transgenes. The presence of the WPRE in the sense orientation was shown to increase transgene expression by up to 7- to 10-fold.

In some embodiments, the inclusion of the WPRE in a vector results in enhanced expression of transgenes.

One illustrative, but non-limiting WPRE is provided by SEQ ID NO:26. In some embodiments, the vectors described herein comprise SEQ ID NO:26. In some embodiments, the LVs described herein comprise SEQ ID NO:26.

### Packaging Signals

In some embodiments, the vectors described herein comprise a packaging signal. A “packaging signal,” “packaging sequence,” or “PSI sequence” is any nucleic acid sequence sufficient to direct packaging of a nucleic acid (the sequence of which comprises the packaging signal) into a retroviral particle. The term includes naturally occurring packaging sequences and engineered variants thereof. Packaging signals of several different retroviruses, including lentiviruses, are known in the art. One illustrative, but non-limiting PSI is provided by SEQ ID NO:24. In some embodiments, the vectors described herein comprise SEQ ID NO:24. In some embodiments, the LVs described herein comprise SEQ ID NO:24.

### Packaging Cells

In some embodiments, the vectors described herein do not encode certain virion proteins and a suitable packaging cell line is needed to package the genome of the viral vector into a virion. In some embodiments, the vectors described herein are used in conjunction with a suitable packaging cell line or co-transfected into cells in vitro along with other vector plasmids containing the necessary genes (e.g., necessary retroviral genes such as gag and pol) to form replication incompetent virions capable of packaging the vectors described herein and infecting cells.

In some embodiments, the vectors are transfected into a packaging cell line that produces viral particles which contain the vector genome. The recombinant virus can be recovered from the culture media and titered by standard methods after co-transfection of the packaging vectors and the transfer vector to the packaging cell line. Production of virions (such as replication incompetent virions) and transfection methodologies are well known in the art.

In some embodiments, the packaging construct is introduced into a mammalian (e.g., human) cell line by calcium phosphate transfection, lipofection or electroporation. In some embodiments, the packaging construct is introduced into a mammalian (e.g., human) cell lines with a dominant selectable marker, such as neomycin, DHFR, kanamycin, or Glutamine synthetase, and subsequent selection is performed in the presence of the appropriate drug to isolate marker-expressing clones. In some embodiments, the selectable marker gene is physically linked to a packaging gene in the construct.

Stable cell lines wherein the packaging functions are configured to be expressed by a suitable packaging cell are known (see, e.g., U.S. Pat. No. 5,686,279, which describes packaging cells, which is hereby incorporated by reference herein in its entirety, and its disclosure relating to stable packaging cell lines is specifically incorporated by reference herein).

To produce lentiviral particles, one may employ any cell that is compatible with the expression of lentiviral Gag and Pol genes, or any cell that can be engineered to support such expression. For example, producer cells such as 293T cells and HT1080 cells may be used.

To produce viral particles any suitable cell can be used. For example, producer cells such as HEK293, 293T cells or HT1080 cells may be used.

To produce lentiviral particles, any cell that is compatible with the expression of lentiviral Gag and Pol genes, or any cell that can be engineered to support such expression, can be used. For example, producer cells such as 293T cells or HT1080 cells may be used.

### Ex Vivo Cell Transduction and Gene Therapy

In some embodiments, methods are provided for transducing a cell (e.g., a human cell).

The vectors and other delivery vehicles described herein can transfer a heterologous nucleic acid sequence (e.g., a nucleic acid encoding WASp) into a mammalian cell (e.g., a human cell).

In some embodiments, the methods provided herein comprise contacting a population of cells with any of the viral vectors and other delivery vehicles described herein (e.g., an LV) under conditions suitable to affect the transduction of the cell.

In some embodiments, methods are provided of delivering a transgene to a cell which is then integrated into the genome of the cell, comprising contacting the cell with a viral vector or another delivery vehicle described herein.

In some embodiments, the cells are stem and/or progenitor cells (e.g., human stem and/or progenitor cells). In some embodiments, the cells are hematopoietic stem and/or progenitor cells (e.g., human hematopoietic stem and/or progenitor cells). In some embodiments, the cells are hematopoietic stem cells (e.g., human hematopoietic stem cells). In some embodiments, the cells are hematopoictic progenitor cells (e.g., human hematopoictic progenitor) cells.

In some embodiments, the cells to be transduced are human CD4+ T cells. In some embodiments, the cells to be transduced are peripheral blood B or T lymphocyte cells. In some embodiments, the cells to be transduced are CD34+ cells. In some embodiments, the cells to be transduced are CD34+ hematopoietic stem and/or progenitor cells (e.g., human cells).

In some embodiments, the cells are induced pluripotent stem cells (iPSCs).

In some embodiments, the cells are transduced in vitro or ex vivo. Methods of introducing vectors or nucleic acids described herein into a cell are known in the art. In some embodiments, vectors or nucleic acids are introduced by, for example and without limitation, viral or bacteriophage infection, transfection, conjugation, lipofection, electroporation, calcium phosphate precipitation, polyethyleneimine (PEI)-mediated transfection, DEAE-dextran mediated transfection, liposome-mediated transfection, microinjection, nanoparticle-mediated nucleic acid delivery, or any other method known in the art. The suitable method depends on the specific delivery vehicle used, as in known in the art.

In some embodiments, the cells are transduced with a vector in a dose in the range of about 1×10⁵ TU/ml to about 1×10⁸ TU/ml. In some embodiments, the cells are transduced using a vector dose in the range of about 1×10⁷ TU/ml to about 1×10⁸ TU/ml. In some embodiments, the cells are transduced using a vector dose in the range of about 1×10⁶ TU/ml to about 1×10⁷ TU/ml. In some embodiments, the cells are transduced using a vector dose in the range of about 1×10⁵ TU/ml to about 1×10⁶ TU/ml. In some embodiments, the cells are transduced using a vector dose in the range of about 1×10⁵ TU/ml to about 1×10⁷ TU/ml. In some embodiments, the cells are transduced using a vector dose of equal to or less than 1×10⁸ TU/ml, 1×10⁷ TU/ml, 1×10⁶ TU/ml, or 1×10⁵ TU/ml.

In some embodiments, the cells transduced ex vivo are then administered to a subject (e.g., a human subject). In some embodiments, the cells are transduced ex vivo and then the transduced cells are infused into a human subject.

In some embodiments, the cell is autologous to the subject (from the subject to be treated).

In some embodiments, the cell is non-autologous (i.e., allogeneic or xenogenic) to the subject (the subject to be treated).

In some embodiments, the cells (e.g., human hematopoietic stem and/or progenitor cells progenitor cells) can be removed from a human using methods known in the art and transduced as described. In some embodiments, the transduced cells are then reintroduced into the same or a different human. In some embodiments, the human is a human having a deficient or absent expression of a gene product, and the transgene delivered by the vector encodes the gene product. In some embodiments, the human is a human having a deficient or absent expression of WAS protein, and the transgene delivered by the vector comprises a human WAS gene.

Where stem cells are to be used, it will be recognized that such cells can be derived from a number of sources including bone marrow (BM), cord blood (CB), mobilized peripheral blood stem cells (mPBSC), and the like. In some embodiments, the cells are derived from BM. In some embodiments, the cells are derived from CB. In some embodiments, the cells are derived from mPBSC. Methods of isolating any such cells, transducing such cells and introducing them into a mammalian subject are well known in the art. Methods that are commonly used for, e.g., bone marrow transplant, peripheral blood stem cell transplant (e.g., in patients undergoing chemotherapy) can be used in this context. In some embodiments, cells from a cell line or from an individual other than the subject, can be used.

In some embodiments, the vectors described herein are introduced into bone marrow cells, mesenchymal stem cells (e.g., obtained from adipose tissue), or other primary cells derived from a mammalian (e.g., human) source.

In some embodiments, the cells to be transduced are human hematopoietic stem cells and/or human hematopoietic progenitor cells obtained from the bone marrow, the peripheral blood, or the umbilical cord blood.

In some embodiments, cell-based therapy comprises providing stem cells and/or progenitor cells (such as human hematopoietic stem cells and/or hematopoietic progenitor cells), transducing the cells with a vector (e.g., an LV) comprising a transgene encoding a gene product, and then introducing the transduced cells into a subject in need thereof (e.g., a subject with a mutation in the gene product resulting in its deficient expression).

In some embodiments, cell-based therapy comprises providing stem cells and/or progenitor cells (such as human hematopoietic stem cells and/or hematopoietic progenitor cells), transducing the cells with a vector (e.g., an LV) comprising a nucleic acid that encodes WASp, and then introducing the transduced cells into a subject in need thereof (e.g., a subject with a mutation in the WAS gene resulting in deficient expression of WASp).

In some embodiments, the administration of a vector (e.g., an LV) described herein to cells results in production of a normal copy of transgene (e.g., normal WASp) in the cells in vitro or ex vivo. In some embodiments, the administration of a vector described herein to cells in vitro results in production of endogenous or wild-type levels of transgene (e.g., wild type levels of WASp) in cells deficient in transgene expression (e.g., having a loss-of-function mutation in or deletion of the WAS gene). In some embodiments, the cells are first expanded in tissue culture before administration of the vector (e.g., an LV). After administration of the vector (e.g., an LV), the cells are then returned to the subject, where they may provide a population of cells (such as red blood cells) that produce the gene product (such as WASp).

In some embodiments, an LV described herein is used in gene therapy (using stem and/or progenitor cells) for WAS (or another disease associated with a deficient WASp expression) by introducing a nucleic acid that encodes WASp into the cells of patients with WAS followed by autologous transplantation.

In some embodiments, the transduced cells described herein are administered to a subject (e.g., parenterally such as by an intravenous infusion). In some embodiments, the transduced cells are administered to a localized area of a subject (e.g., bone marrow).

In some embodiments, the transduced cells described herein are administered to a subject in a therapeutically effective amount.

A therapeutically effective amount is an amount capable of achieving a therapeutic effect. In some embodiments, therapeutic effects may include, without limitation, increase in or restoration of a normal (wild-type or physiologic) expression levels of the protein encoded by the transgene in a subject, treatment or prevention of a disorder caused by deficient expression of the transgene, improvement in or amelioration of any symptom of a disorder caused by deficient expression of the transgene, improvement in survival or life-span of the subject being treated.

In some embodiments, the cells are administered to a subject in a dose in the range of about 1×10⁵ to about 1×10⁷ of cells per kg of body weight. In some embodiments, the cells are administered to a subject in a dose in the range of about 1×10⁶ to about 50×10⁶ of cells per kg of body weight. In some embodiments, the cells are administered to a subject in a dose in the range of about 1×10⁶ to about 20×10⁶ of cells per kg of body weight. In some embodiments, the cells are administered to a subject in a dose equal to or less than 50×10⁶ of cells per kg of body weight, 30×10⁶ of cells per kg of body weight, 20×10⁶ of cells per kg of body weight, 10×10⁶ of cells per kg of body weight, or 5×10⁶ of cells per kg of body weight.

In some embodiments, administration of transduced cells described herein to a subject achieves a therapeutic effect. In some embodiments, administration of transduced cells described herein to a subject increases or restores normal or wild-type expression levels of the protein encoded by the transgene in a subject. In some embodiments, administration of transduced cells described herein to a subject is effective to treat or improve the symptoms of a disorder caused by deficient expression of the transgene. In some embodiments, administration of transduced cells described herein to a subject is effective to prevent a disorder caused by deficient expression of the transgene. In some embodiments, the transgene comprises nucleic acid of the WAS gene, and the protein encoded by the transgene is WASp. In some embodiments, the disorder to be treated is WAS. In some embodiments, the disorder to be treated is XLT.

In some embodiments, administration of transduced cells described herein to a subject leads to physiologic or near physiologic level of expression of transgene in all affected cell lineages. In some embodiments, administration of transduced cells described herein to a subject leads to physiologic or near physiologic level of expression of transgene in megakaryocytes. In some embodiments, the transgene encodes WASp. In some embodiments, administration of transduced cells described herein, wherein the transgene encodes WASp, to a subject leads to increased platelet counts in the subject. In some embodiments, administration of transduced cells described herein, wherein the transgene encodes WASp, to a subject leads to rescue of platelet counts in the subject to healthy subject levels. In some embodiments, administration of transduced cells described herein, wherein the transgene encodes WASp, to a subject leads to an improved platelet engraftment and/or improves or restores platelet function to healthy subject levels.

The transduced cells described herein can be administered to a subject once or a number of times, at various intervals and over different periods of time as required.

In some embodiments, the transduced cells described herein are administered to a subject once, in a single administration. In some embodiments, single administration achieves a therapeutic effect. In some embodiments, single administration significantly increases or restores normal or wild-type expression levels of the protein encoded by the transgene in a subject. In some embodiments, single administration is effective to treat or improve the symptoms of a disorder caused by deficient expression of the transgene. In some embodiments, single administration is effective to prevent a disorder caused by deficient expression of the transgene. In some embodiments, the transgene comprises nucleic acid of the WAS gene, and the protein encoded by the transgene is WASp. In some embodiments, the disorder to be treated is WAS. In some embodiments, the disorder to be treated is XLT. In some embodiments, the disorder to be treated is XLN.

In some embodiments, the transduced cells are administered to a subject once in 10 years, once in 5 years, once in 3 years, once a year, once every 6 months, once every 3 months, or once a month. In some embodiments, the transduced cells are administered to a subject for an appropriate period of time, e.g., for at least or less than 1 year, 2 years, 3 years, 5 years, 10 years or 20 years or as needed.

In some embodiments, the transduced cells are administered to a subject for a number of times needed to achieve the desired effect.

In some embodiments, treatment of a subject with a LV may include a single treatment. In some embodiments, treatment of a subject with a LV may include a series of treatments.

As is known in the art, certain factors can influence the dosage and timing required to effectively treat a subject, including but not limited to the general health and/or age of the subject, the severity of the disease or disorder, previous treatments, and other diseases present.

### In Vivo Cell Transduction and Gene Therapy

In some embodiments, the cells are transduced in vivo. In some embodiments, subjects are treated via direct, in vivo introduction of a vector, viral particle or virion described herein. In some embodiments, a vector, viral particle or virion described herein is directly administered to a subject. In some embodiments, a vector, viral particle or virion described herein is directly administered to a localized area of a subject (e.g., bone marrow).

In some embodiments, a vector, viral particle or virion described herein is administered to a subject in a therapeutically effective amount. A therapeutically effective amount is an amount capable of achieving a therapeutic effect. In some embodiments, therapeutic effects may include, without limitation, increase in or restoration of a normal (wild-type or physiologic) expression levels of the protein encoded by the transgene in a subject, treatment or prevention of a disorder caused by deficient expression of the transgene, improvement in or amelioration of any symptom of a disorder caused by deficient expression of the transgene, improvement in survival or life-span of the subject being treated.

In some embodiments, a vector, viral particle or virion described herein is administered to a subject in a dose in the range of about 1×10⁵ TU/ml to about 1×10⁸ TU/ml. In some embodiments, a vector, viral particle or virion described herein is administered to a subject in a dose in the range of about 1×10⁷ TU/ml to about 1×10⁸ TU/ml. In some embodiments, a vector, viral particle or virion described herein is administered to a subject in a dose in the range of about 1×10⁶ TU/ml to about 1×10⁷ TU/ml. In some embodiments, a vector, viral particle or virion described herein is administered to a subject in a dose in the range of about 1×10⁵ TU/ml to about 1×10⁶ TU/ml. In some embodiments, a vector, viral particle or virion described herein is administered to a subject in a dose in the range of about 1×10⁵ TU/ml to about 1×10⁷ TU/ml. In some embodiments, a vector, viral particle or virion described herein is administered to a subject in a dose equal to or less than 1×10⁸ TU/ml, 1×10⁷ TU/ml, 1×10⁶ TU/ml, or 1×10⁵ TU/ml. In some embodiments, a vector, viral particle or virion described herein is administered to a subject in a dose equal to or less than about 1×10⁸ TU/ml or about 1×10⁷ TU/ml.

In some embodiments, in vivo administration of a vector, virion or a pharmaceutical composition described herein achieves a therapeutic effect. In some embodiments, in vivo administration of a vector, virion or a pharmaceutical composition described herein increases expression levels or restores normal or wild-type expression levels of the protein encoded by the transgene in a subject. In some embodiments, in vivo administration of a vector, virion or a pharmaceutical composition described herein is effective to treat or improve the symptoms of a disorder caused by deficient expression of the transgene. In some embodiments, in vivo administration of a vector, virion or a pharmaceutical composition described herein is effective to prevent a disorder caused by deficient expression of the transgene. In some embodiments, the transgene comprises nucleic acid of the WAS gene, and the protein encoded by the transgene is WASp. In some embodiments, the disorder to be treated is WAS. In some embodiments, the disorder to be treated is XLT.

In some embodiments, in vivo administration of a vector, virion or a pharmaceutical composition described herein to a subject leads to physiologic or near physiologic level of expression of transgene in a cell (e.g., in all cell lineages). In some embodiments, the transgene encodes WASp. In some embodiments, in vivo administration of a vector, virion or a pharmaceutical composition described herein leads to increased platelet counts in the subject. In some embodiments, in vivo administration of a vector, virion or a pharmaceutical composition described herein leads to rescue of platelet counts in the subject to healthy subject levels. In some embodiments, in vivo administration of a vector, virion or a pharmaceutical composition described herein leads to an improved platelet engraftment and/or improves or restores platelet function to healthy subject levels.

Vectors, virions, and pharmaceutical compositions described herein can be administered once or a number of times, at various intervals and over different periods of time as required.

In some embodiments, the vectors, virions, and pharmaceutical compositions described herein are administered to a subject once, in a single administration. In some embodiments, single administration achieves a therapeutic effect. In some embodiments, single administration significantly increases or restores normal or wild-type expression levels of the protein encoded by the transgene in a subject. In some embodiments, single administration is effective to treat or improve the symptoms of a disorder caused by deficient expression of the transgene. In some embodiments, single administration is effective to prevent a disorder caused by deficient expression of the transgene.

In some embodiments, the vectors, virions, and pharmaceutical compositions described herein are administered to a subject once in 10 years, once in 5 years, once in 3 years, once a year, once every 6 months, once every 3 months, or once a month. In some embodiments, the vectors, virions, and pharmaceutical compositions described herein are administered to a subject for an appropriate period of time, e.g., for at least or less than 1 year, 2 years, 3 years, 5 years, 10 years or 20 years or as needed.

In some embodiments, the vectors, virions, and pharmaceutical compositions described herein are administered to a subject for a number of times needed to achieve the desired effect.

In some embodiments, treatment of a subject with a LV may include a single treatment. In some embodiments, treatment of a subject with a LV may include a series of treatments.

As is known in the art, certain factors can influence the dosage and timing required to effectively treat a subject, including but not limited to the general health and/or age of the subject, the severity of the disease or disorder, previous treatments, and other diseases present.

### Pharmaceutical Compositions and Methods of Treatment/Prevention

The cells, vectors, viral particles and virions (and other delivery vehicles) described herein can be formulated into a pharmaceutically acceptable composition (e.g., any composition suitable for human administration known in the art). In some embodiments, pharmaceutical compositions include a cell, a vector, a viral particle or a virion (e.g., an LV) in combination with a pharmaceutically acceptable carrier. Suitable pharmaceutically acceptable carriers for delivery of a cell, a vector, a viral particle or a virion (e.g., an LV) are known in the art. Suitable pharmaceutically acceptable carriers are determined by the particular composition being administered and the particular method of administration used.

In some embodiments, a pharmaceutical composition comprises one or more cells transduced with a vector described herein.

In some embodiments, a pharmaceutical composition comprises a vector described herein.

In some embodiments, a pharmaceutical composition comprises a viral particle or a virion capable of infecting cells, wherein the infected cells express the transgene as described herein.

The cells, vectors, viral particles and virions described herein, and pharmaceutically acceptable compositions comprising the same, can be formulated for delivery by any available route including, but not limited to, parenteral (e.g., intravenous), intramuscular, intradermal, subcutaneous, transdermal (topical), transmucosal, vaginal, and rectal. In some embodiments, the cells, vectors, viral particles and virions described herein, and pharmaceutically acceptable compositions comprising the same, are administered parenterally (e.g., intravenously such as by an infusion, e.g., continuous infusion). In some embodiments, the cells, vectors, viral particles and virions described herein, and pharmaceutically acceptable compositions comprising the same, are administered intravenously, intra-arterially or intraperitoneally. In some embodiments, the cells, vectors, viral particles and virions described herein, and pharmaceutically acceptable compositions comprising the same, are administered locally to a tissue or organ.

In some embodiments, LV gene therapy vectors described herein can be delivered to a subject by, for example, intravenous injection, local administration, or by stereotactic injection (see, e.g., Chen et al. (1994) Proc. Natl. Acad. Sci. USA, 91: 3054). Pharmaceutical preparations can include a LV in an acceptable diluent or can comprise a slow-release matrix in which a LV is imbedded.

Pharmaceutical compositions for parenteral delivery may comprise an isotonic sterile injection solution comprising, e.g., a buffer and/or an antioxidant. Pharmaceutical compositions for parenteral delivery may comprise a sterile suspension comprising, e.g., a suspending agent, a thickening agent, a solubilizer, a stabilizer, and/or a preservative.

In some embodiments, liposomes are used as pharmaceutically acceptable carriers. These can be prepared according to methods known in the art, for example, as described in U.S. Pat. No. 4,522,811, which is hereby incorporated by reference herein in its entirety.

In some embodiments, the cells, vectors, viral particles and virions described herein, and pharmaceutically acceptable compositions comprising the same, may be encapsulated or otherwise manipulated to protect them from degradation, rapid elimination from the body, enhance uptake into tissues or cells, etc.

In some embodiments, the pharmaceutical compositions described herein comprise a microencapsulated delivery system with, e.g., biodegradable and/or biocompatible polymers (such as collagen, ethylene vinyl acetate, polyanhydrides, polyglycolic acid, polylactic acid, and polyorthoesters).

Pharmaceutical compositions can be in dosage unit form for ease of administration and uniformity of dosage. Dosage unit form as used herein refers to physically discrete units suited as unitary dosages for the subject to be treated; each unit comprising a predetermined quantity of a cell, a vector or a virion (e.g., an LV) calculated to produce the desired therapeutic effect in association with a pharmaceutical carrier.

Pharmaceutical compositions described herein can be in a unit dose container such as an ample or a vial, or a multi-unit dose container or a pack, optionally together with instructions for administration.

A unit dose may be for continuous infusion over a set time period. Unit dose of the vector or virion (e.g., LV) described herein may be described in terms of transducing units (T.U.), as defined by titering the vector on a cell line such as HeLa or HEK293. In some embodiments, unit doses can range from 10⁴ to 10¹⁰ T.U. In some embodiments, unit doses can range from about 10⁵ to about 10⁹ T.U. In some embodiments, unit doses can range from about 10⁵ to about 10⁸ T.U. In some embodiments, unit doses can range from about 10⁶ to about 10⁸ T.U. In some embodiments, unit doses can range from about 10⁷ to about 10⁸ T.U.

In some embodiments, the pharmaceutical composition is targeted to specific cell types. In some embodiments, compositions are targeted to specific cell types using monoclonal antibodies to cell surface markers, e.g., endogenous markers or viral antigens expressed on the surface of infected cells.

In some embodiments, a pharmaceutical composition described herein is administered to a subject in a therapeutically effective amount. The pharmaceutically acceptable compositions can be used in a method of treating of a subject having a deficient expression of a gene product encoded by the transgene, treating a disorder caused by the deficient expression, or preventing a disorder caused by the deficient expression. Treating refers to, for example: (i) obtaining a desired biological result (such as an increased expression of a gene product), (ii) obtaining a desired clinical result (such as the reduction of symptoms caused or known to be caused by a deficient expression of a gene product), (iii) causing reduced development or regression of the disease or disorder caused or known to be caused by a deficient expression of a gene product. Preventing a disorder or disease refers to, for example: causing clinical symptoms of the disease or disorder not to develop in a subject that may be predisposed to or at risk of the disease or disorder (such due to a deficient expression of a gene product). In some embodiments, the gene product is WASp. In some embodiments, the disease or disorder is WAS. In some embodiments, the disease or disorder is XLT.

A therapeutically effective amount is an amount capable of achieving a therapeutic effect. In some embodiments, therapeutic effects may include, without limitation, increase in or restoration of a normal (wild-type or physiologic, or nearly wild-type/physiologic) expression levels of the protein encoded by the transgene in a subject, treatment or prevention of a disorder caused by deficient expression of the transgene, improvement in or amelioration of any symptom of a disorder caused by deficient expression of the transgene, improvement in survival or life-span of the subject being treated.

In some embodiments, where the transgene is WAS encoding WASp, therapeutic effects include, but are not limited to: (i) increase in or restoration of physiologic WASp expression (or restoration of WASp expression to within 60%, within 50%, within 40%, within 30%, within 20%, within 20% or within 10% below or above its physiologic or wild-type levels); and (ii) alleviation of one, two, three or more symptoms of a WASp-related disorder such as thrombocytopenia, microthrombocytopenia, eczema, and/or symptoms of autoimmunity. In some embodiments, where the transgene is WAS encoding WASp, treatment in accordance with the methods described herein improves or eliminates thrombocytopenia in the subject. In some embodiments, where the transgene is WAS encoding WASp, treatment in accordance with the methods described herein increases platelet counts of the treated subject, e.g., at least or more than 20%, 30%, 40%, 50%, 60%, 70%, 80%, 90% or 100% relative to the platelet counts in the subject prior to treatment. In some embodiments, where the transgene is WAS encoding WASp, treatment in accordance with the methods described herein increases platelet counts of the treated subject, e.g., at least or more than 2-fold, 3-fold, 4-fold, 5-fold, 6-fold, 7-fold, 8-fold, 9-fold, 10-fold, 15-fold or 20-fold relative to the platelet counts in the subject prior to treatment. In some embodiments, where the transgene is WAS encoding WASp, treatment in accordance with the methods described herein increases platelet counts of the treated subject, e.g., to within at least or more than 60%, 50%, 40%, 30%, 20%, 15%, 10% or 5% of the platelet count in a healthy subject or a subject having wild-type WASp expression. In some embodiments, where the transgene is WAS encoding WASp, treatment in accordance with the methods described herein restores platelet counts of the treated subject to physiologic or nearly physiologic level (such as the level in a healthy subject or a subject having wild-type WASp expression). In some embodiments, where the transgene is WAS encoding WASp, treatment in accordance with the methods described herein leads to a high level or improved platelet engraftment and/or improves or restores platelet function to healthy subject levels. In some embodiments, where the transgene is WAS encoding WASp, treatment in accordance with the methods described herein improves or eliminates thrombocytopenia in the subject. In some embodiments, where the transgene is WAS encoding WASp, treatment in accordance with the methods described herein prevents development of thrombocytopenia in the subject. In some embodiments, where the transgene is WAS encoding WASp, treatment in accordance with the methods described herein improves or eliminates microthrombocytopenia in the subject. In some embodiments, where the transgene is WAS encoding WASp, treatment in accordance with the methods described herein prevents development of microthrombocytopenia in the subject. In some embodiments, where the transgene is WAS encoding WASp, treatment in accordance with the methods described herein improves or eliminates eczema in the subject. In some embodiments, where the transgene is WAS encoding WASp, treatment in accordance with the methods described herein prevents development of eczema in the subject. In some embodiments, where the transgene is WAS encoding WASp, treatment in accordance with the methods described herein improves or eliminates symptoms of autoimmune disease in the subject. In some embodiments, where the transgene is WAS encoding WASp, treatment in accordance with the methods described herein prevents development of symptoms of autoimmune disease in the subject. In some embodiments, where the transgene is WAS encoding WASp, treatment in accordance with the methods described herein prevents development of cancer thrombocytopenia in the subject such as, e.g., the subject does not develop cancer within 5, 10, 15, 20, 25 or 30 years after administration.

### Patient Populations

The patients or subjects treated in accordance with the methods described herein include, but are not limited to, humans and non-human mammals. In some embodiments, the subject being treated is a primate. In some embodiments, the subject being treated is a human. In certain embodiments, the subject being treated is a livestock animal (e.g., cattle, sheep, horses, goats, cows, swine, and the like) or a domesticated animal (e.g., a dog or a cat). In some embodiments, the subject being treated is a laboratory animal (e.g., used in research), such as a rodent, a rabbit, or a primate.

In some embodiments, the subject is a male. In some embodiments, the subject is a female.

In some embodiments, the subject is an infant or a toddler. In some embodiments, the subject is less than 1 years old, less than 2 years old, less than 3 years old, less than 4 years old, or less than 5 years old. In some embodiments, the subject is less than 6 years old, less than 7 years old, less than 8 years old, less than 9 years old, or less than 10 years old. In some embodiments the subject is less than 16 years old or less than 18 years old.

In some embodiments, the subject being treated in accordance with the methods described herein has a deficiency in expression of a gene product encoded by the transgene. In some embodiments, the subject being treated in accordance with the methods described herein has a mutation in or a deletion of the gene that the transgene is used to replace. In some embodiments, the subject being treated in accordance with the methods described herein has a loss-of-function mutation in the gene that the transgene is used to replace.

In some embodiments, the subject being treated in accordance with the methods described herein has a deficiency in expression of WAS protein. In some embodiments, the subject being treated in accordance with the methods described herein has a mutation in the WAS gene or a deletion of the WAS gene. In some embodiments, the subject being treated in accordance with the methods described herein has a loss-of-function mutation in the WAS gene.

In some embodiments, the subject being treated in accordance with the methods described herein has WAS (e.g., has been diagnosed with WAS). In some embodiments, the subject being treated in accordance with the methods described herein has XLT (e.g., has been diagnosed with XLT).

In some embodiments, the subject being treated has not been treated with an allogeneic stem cell transplantation.

### EXAMPLES

### Example 1—Identification of Minimal Enhancer Elements for a Vector/Plasmid Expressing WASp

This example shows identification of minimal enhancer elements for WASp (WAS protein) expression, the use of which leads to superior hematopoietic stem and progenitor cell (HPSC) gene transfer and improved viral titer, while maintaining the ability to restore physiologic levels of WASp expression in WAS−/− cells.

The redesign of a lentiviral vector WASVec1.0 described in WO 2021/096887 (referenced as FIG. 20, SEQ ID NO:17 in WO2021096887A) was performed using an improved and refined bioinformatics-based approach. The goal of the redesign was to create a superior hematopoietic stem and progenitor cell (HPSC) gene transfer and improved viral titer, while maintaining the ability to restore physiologic levels of WASp expression in WAS−/− cells (such as at a VCN ˜1).

In this study, new endogenous elements of the WAS locus were identified and utilized, and the minimal functional boundaries of the key enhancer elements were redefined to decrease proviral length (in order to increase gene transfer and titer).

To design the new lead candidate LV, WASVec1.0 was systematically deconstructed to retain only the key functional elements. The following elements were removed: “element 9 slim” (E9s), “Hypersensitive site 3 slim” (HS3s), “sub-sub-element 1 of element 2”, and “sub-element 4 of element 2”, totaling 1.1 kb of sequence removed from WASVec1.0 to create “WASVec2.0 V3” which served as the minimal backbone for the redesign.

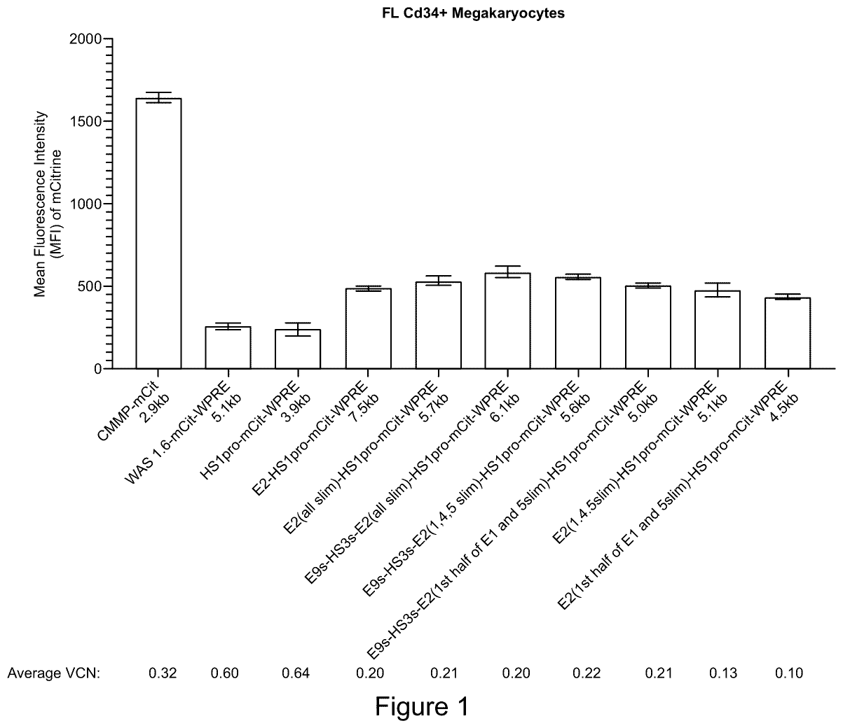

FIG. 1 demonstrates the expression of mCitrine driven from a series of LV. A series of lentiviral LV containing either the original or “slim” fragments of key enhancer constructs were cloned to define the minimal functional boundaries. For example, a “slim” version of the element 2 enhancer (E2) was cloned upstream of the endogenous minimal WAS promoter (HS1) driving expression of an mCitrine reporter gene (mCit) (5th from the left) and was compared the original E2 containing LV. Key element within WASVec1.0 (7th from the left) were also removed or “slim” to define the minimal functional boundaries of key elements within WASVec1.0 and to see which enhancer elements could be removed without significantly decreasing expression. 3rd from the right, we removed sub element 4 of element 2.

These constructs were packaged into LV particles in 293T cells as previously described in Cooper, 2011, J Virol Methods, 177(1): 1-9 (doi: 10.1016/j.jviromet.2011.06.019, https://pubmed.ncbi.nlm.nih.gov/21784103/). The packaged constructs were used to transduce fetal liver CD34+ hematopoietic stem and progenitor cells (HSPCs) cultured in X-VIVO15 media supplemented with 50 ng/ml of hSCF, hTPO, and hFL3L (as previously described Masiuk, 2019, Cell Stem Cell, 24(2):309-317.e7 (doi: 10.1016/j.stem.2018.12.003, https://pubmed.ncbi.nlm.nih.gov/30639036/), which were subsequently differentiated into megakaryocytes through a 12-day culture in StemSpan media+50 ng/ml hTPO (as previously described by Perdomo et al., 2017; J Vis Exp (130), c56420, doi: 10.3791/56420). After 12 days of differentiation, the expression of mCitrine with CD41+CD42+ megakaryocytes were evaluated.

The data revealed that “element 9 slim” (E9s), “Hypersensitive site 3 slim” (HS3s), sub element 4 of E2, and sub-sub element 1 of element 2 are not main drivers of expression. Accordingly, these four elements were removed from WASVec1.0 to generate WASVec2.0 V3 which served as the minimal backbone for the vector redesign. FIG. 2 shows a schematic of WASVec2.0 V3.

### Example 2-Development of Improved Expression Vectors for Superior Hematopoietic Stem, Progenitor Cell, and Certain Blood Lineages

This example shows identification of new regulatory enhancer elements regulating gene expression, e.g., the WAS, in cells of the hematopoietic lineage.

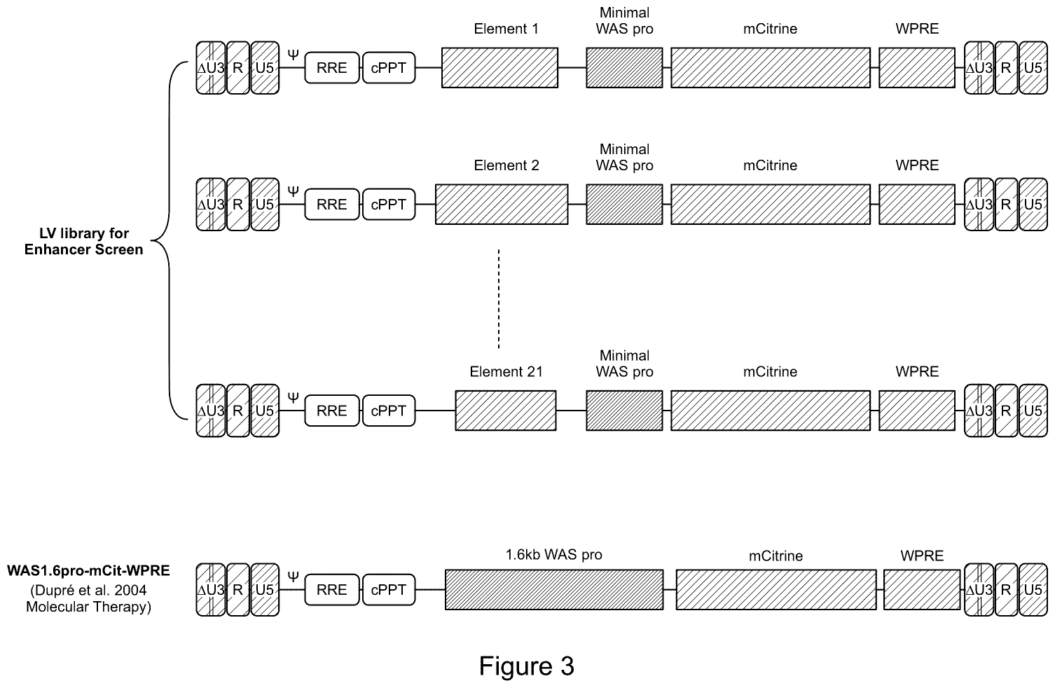

After defining the minimal LV backbone, WASVec 2.0 V3, the WAS locus was reanalyzed using a refined bioinformatics approach to identify new endogenous regulatory elements regulating the WAS gene expression. FIG. 3 shows a schematic demonstrating vector constructs generated as part of a LV library for a screen to identify new enhancers of the endogenous WAS gene.

To elucidate the function of each of the putative regulatory elements, a series of lentiviral vectors were made, each with a single putative enhancer element cloned upstream of the endogenous WAS promoter driving expression of mCitrine. Since the main goal was to identify the endogenous megakaryocyte enhancer responsible for controlling expression of the WAS gene in the human genome, the LV library was transduced into human HSPCs which were subsequently differentiated into megakaryocytes in-vitro to identify strong megakaryocyte enhancers.

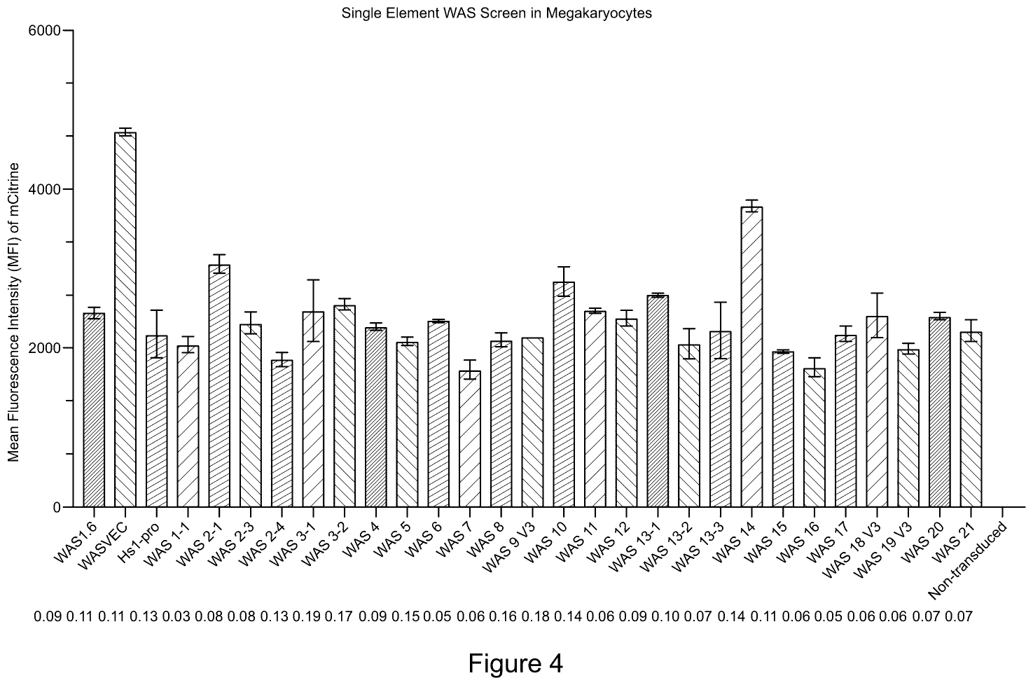

In particular, a series of LVs were cloned, each with a single putative regulatory/enhancer element cloned upstream of the minimal WAS promoter driving expression of an mCitrine reporter gene. As a bar of comparison, the LV currently being evaluated in clinical trials (WAS1.6) and WASVec1.0 (labeled as WASVEC in FIG. 4) were also tested. WASVec1.6 is currently in clinical trials and is driven by a 1.6 kb promoter fragment of the WAS gene (Abina, 2015, JAMA, 313(15):1550-63, doi: 10.1001/jama.2015.3253). These LV constructs were packaged into LV particles (as previously described by Cooper et al., 2011, J Virol Methods, 177(1): 1-9, doi: 10.1016/j.jviromet.2011.06.019) and used to transduce fetal liver CD34+ hematopoietic stem and progenitor cells (HSPCs) cultured in X-VIVO15 media supplemented with 50 ng/ml of hSCF, hTPO, and hFL3L (as previously described) and subsequently differentiated into megakaryocytes through a 12-day culture in StemSpan media+50 ng/ml hTPO (as previously described by Perdomo et al. (2017; J Vis Exp, (130), e56420, doi: 10.3791/56420). After 12 days of differentiation, the expression of mCitrine with CD41+CD42+ megakaryocytes were evaluated.

FIG. 4 shows the results of this refined single element WAS screen, which revealed 26 new putative regulatory elements. The identified new endogenous elements were located within a 1100 kb topologically associated domain or TAD spanning 261 kb upstream to 839 kb downstream of the WAS gene.

This enhancer screen revealed a 2173 bp sequence, element 14, which increased expression 1.7-fold over the endogenous WAS promoter alone in human HSPC derived megakaryocytes. The identified element 14 (WAS 14) drove high level expression in the megakaryocyte lineage, expressing almost 2-fold higher than the minimal WAS promoter (HS1-pro) alone.

The enhancer screen also revealed that elements 2 and 10 contributed to driving expression, but when all elements were combined together, element 10 did not contribute much to expression. To decrease proviral size element 10 was removed from certain vectors being evaluated.

Using further bioinformatic data such as DNaseI hypersensitivity, Transcription Factor binding via ChIP-seq and sequence conservation, the core and ultra-core fragments of E14 (next 2 slides) were identified. In particular, using further bioinformatic data, a 555 bp “core” fragment of element 14 and 235 bp “ultra-core” fragment of element 14 were identified.

The identified “core” (E14core) and “ultra-core” (E14ultra-core) fragments of element 14 were incorporated into WASVec2.0 V3 (i.e., the minimal backbone vector, as shown in FIG. 2) to create WASVec2.0 V1 and WASVec2.0 V2, respectively, which are shown in FIGS. 5A and 5B, respectively. FIG. 5C shows key elements of WASVec2.0 V2. The vectors express a codon optimized variant of WASp. The goal of this redesign was to decrease proviral length in an effort to increase titer and gene transfer while maintaining expression.

To illustrate the design of the previously known and newly designed vectors:
WASVec 1.0 is 6.4 kb (with WASp in ORF): E9 slim-HS3-slim-(1,4,5 of E2slim)-HS1-WASp^{(JCAT co-op)}-WRPE.
WASVec2.0 V1 is 5.9 kb (increased maximal expression, smaller proviral size): E14core-(1^{st} half of 1 and 5 of E2slim)-HS1-WASp(JCAT co-op)-WRPE.
WASVec2.0 V2 is 5.6 kb (increased/maintain expression, even smaller proviral size): E14ultra-core-(1^{st} half of 1 and 5 of E2slim)-HS1-WASp(JCAT co-op)-WRPE.
WASVec2.0 V3 is 5.3 kb (slight decrease in expression, smallest proviral size): (1^{st} half of 1 and 5 of E2slim)-HS1-WASp^{(JCAT co-op)}-WRPE.

### Example 3-Viral Titer and Dose Response in Human Cells Transfected with the New WAS Vectors

This example shows that the new vectors WASVec2.0 V1, V2, and V3 show an increased viral titer and gene transfer relative to previously known WASVec1.0.

WASVec1.0, described in WO2021/096887, had a proviral genome length of 6.4 kb and subsequently poor HPSC gene transfer and viral titer. There is a negative correlation between proviral length to both HSPC gene transfer and viral titer (see Morgan, 2020, 28(1):328-340. doi: 10.1016/j.ymthe.2019.09.020).

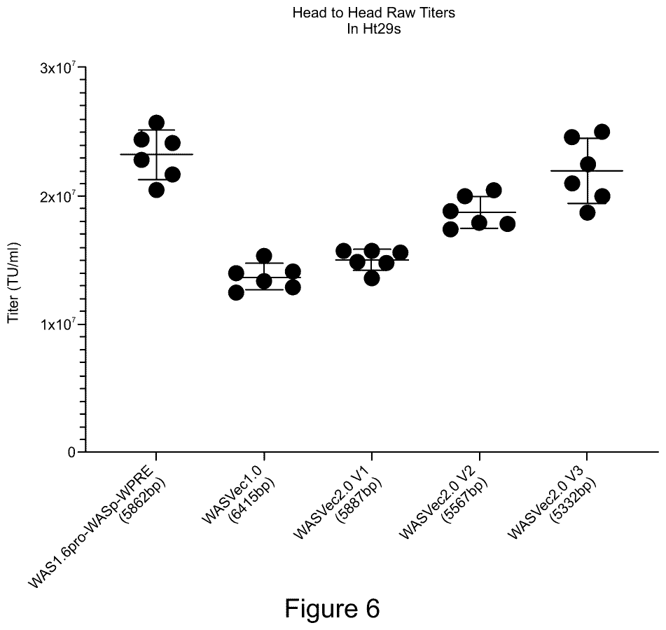

The newly designed LVs, WASVec2.0 V1, V2, and V3 have proviral lengths of 5.9 kb, 5.6 kb, and 5.4 kb respectively. When these LVs were packaged and titered head to head in HT-29 cells (as previously described in Cooper, 2011, J Virol Methods, 177(1): 1-9, doi: 10.1016/j.jviromet.2011.06.019, https://pubmed.ncbi.nlm.nih.gov/21784103/), a gradual increase in titer was observed as proviral length decreased. In particular, WASVec2.0 V1, V2 and V3 displayed a 1.097-fold, 1.36-fold, and 1.6-fold increase in titer compared to WASVec 1.0, respectively. See FIG. 6.

To evaluate the improvement in gene transfer, human CD34+ mobilized peripheral blood stem cells (PBSC) were transduced with increasing doses of WASVec1.0, or WASVec2.0 V1, V2, or V3, along with WAS1.6 and WASVec1.0, and cultured for 14 days to measure stable vector copy numbers.

In particular, as previously described (Masiuk, 2019, Cell Stem Cell, 24(2):309-317.c7. doi: 10.1016/j.stem.2018.12.003, https://pubmed.ncbi.nlm.nih.gov/30639036/), human CD34+ PBSCs were pre-stimulated in X-VIVO15 media supplemented with 50 ng/ml hSCF, hTPO and hFLT3L for 24 hrs before addition of LV supernatant for an additional 24 hrs. Cells were then cultured for an additional 14 days in basal bone marrow media (BBMM) containing: IMDM supplemented with 20% FBS, 0.52% BSA, 5 ng/m hIL3, 10 ng/mL IL6, and 25 ng/ml hSCF. After 14 days in culture genomic DNA (gDNA) was extracted from the cells and vector copy number was evaluated via ddPCR as previously described (Masiuk, 2019, Cell Stem Cell, 24(2):309-317.c7. doi: 10.1016/j.stem.2018.12.003).

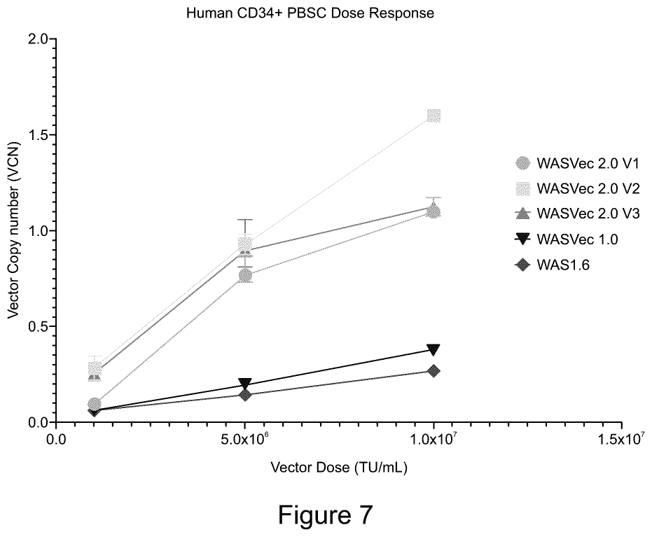

It was found that at an equal vector dose of 4.8×10\hat{ }7 TU/mL, WASVec2.0 V1, V2, and V3 had a 2.9-fold, 4.2-fold, 3.0-fold improvement in gene transfer compared to WASVec1.0, respectively. See FIG. 7. WASVec2.0 V2 has the highest gene transfer into HSPCs, having a 4.2-fold increased gene transfer over WASVec1.0, at an equal vector dose of 1×10\hat{ }7 TU/mL.

### Example 4-New Vectors Restore WAS Protein to Endogenous Levels in WAS−/− Cells

This example shows that the new vectors having the new designed enhancer elements are able to restore wildtype levels of WASp per transduced cell.

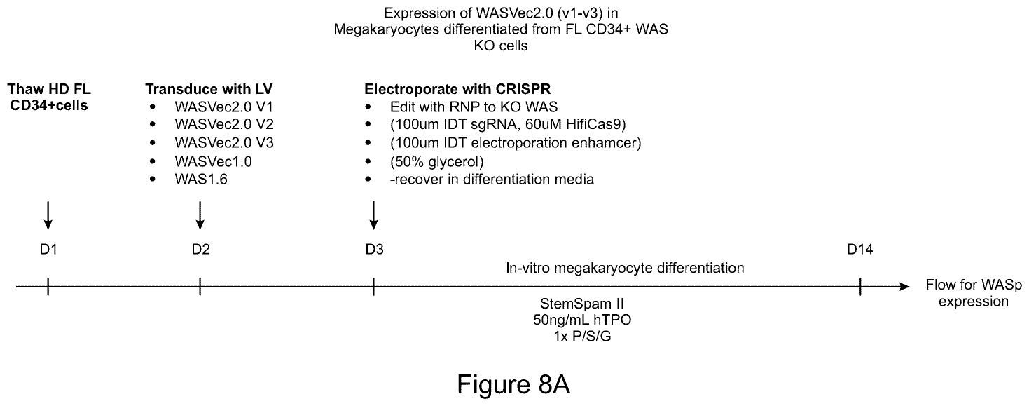

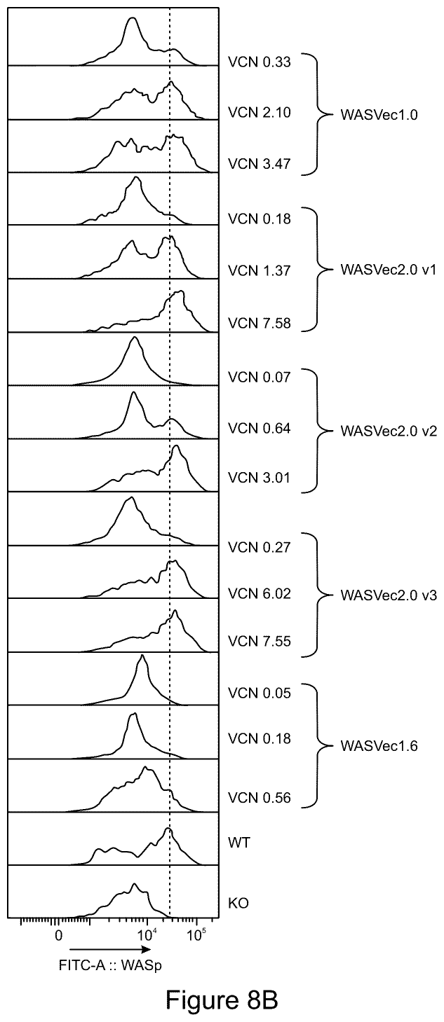

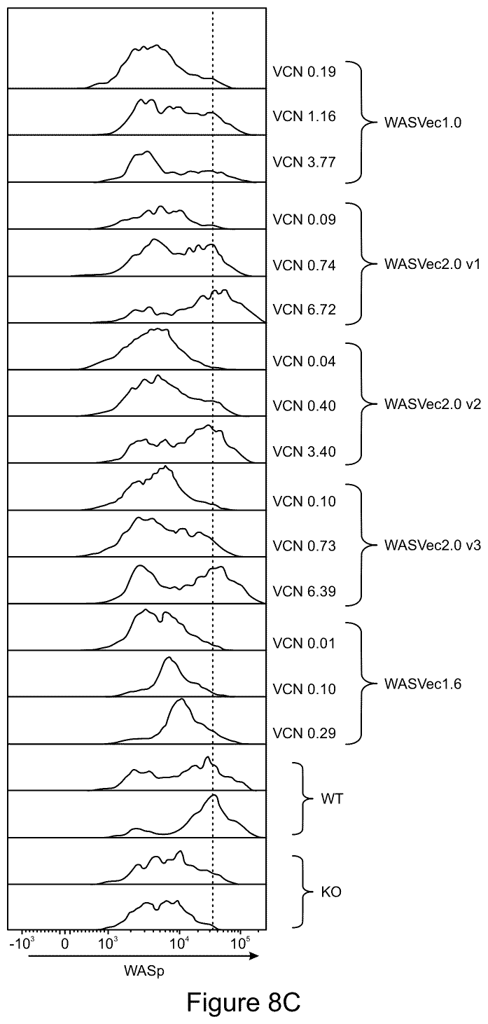

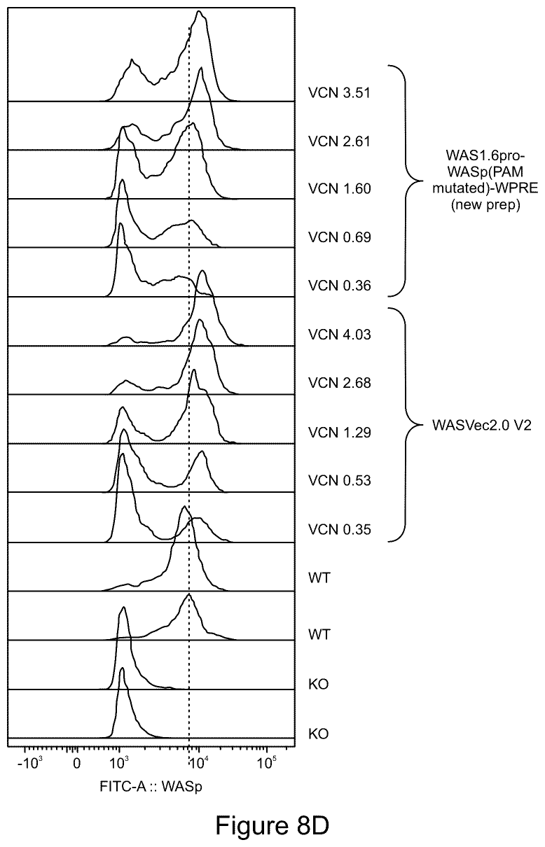

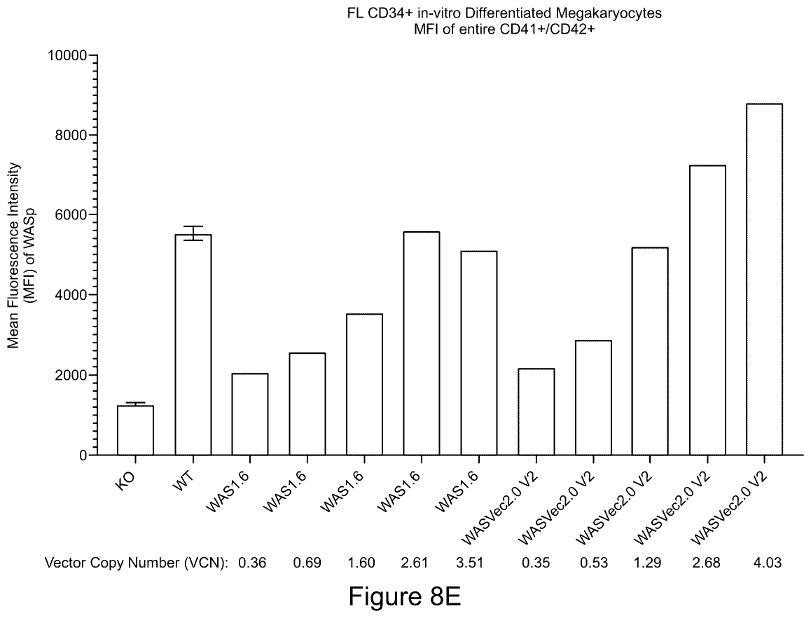

To evaluate expression from each LV construct, human WAS−/− HSPCs generated by CRISPR knockout were transduced with WASVec2.0 V1, V2, V3, WASVec1.0, or WAS1.6 at a range of vector doses and differentiated into megakaryocytes in-vitro. FIG. 8A is a schematic which details the strategy used for evaluating the rescue of WAS expression with the new vectors in megakaryocytes differentiated from FL CD34+ WAS KO cells.

In particular, to evaluate the expression from each construct, healthy donor (HD) fetal liver (FL) CD34+ HSPCs were transduced as previously described (Masiuk, 2019, Cell Stem Cell, 24(2):309-317.c7. doi: 10.1016/j.stem.2018.12.003) with the above-noted LV constructs, and 24 hrs later, electroporated with CRISPR-Cas9 targeting the endogenous WAS gene (using the conditions specified in FIG. 8A) to knockout the endogenous WAS gene in order to measure the ability of each LV to restore WAS levels. Cells were maintained for 11-days in megakaryocyte differentiation media through a 12-day culture in StemSpan media+50 ng/ml hTPO (as previously described in Perdomo, 2017, J Vis Exp, (130), c56420, doi: 10.3791/56420).

Restoration of WASp expression was measured at day 14 in the CD41+CD42+ megakaryocyte population. See FIGS. 8B to 8E.

FIG. 8B shows restoration of the WASp (Wiskott Aldrich Syndrome protein) in WAS knockout megakaryocytes (from 3 mixed CD34+ donors), where each LV was transduced to achieve varying VCNs for a comparison of expression. It was found that WASVec1.0, WASVec2.0 v1, v2, and v3 are all able to restore WASp to HD levels. WASVec2.0 V2 expressed WASp similar to WASVec1.0 (even though WASVec2.0 has a much smaller proviral length) and both restored WASp to HD levels. No WASp expression was detected in the WAS KO control arm without LV transduction.

FIG. 8C shows a 2^{nd} replicate of the experiment shown in FIG. 8B, where cells from 2 mixed CD34+ donors were used. Again, it was found that WASVec1.0, WASVec2.0 v1, v2, and v3 are all able to restore WASp to HD levels. WASVec2.0 V2 expressed WASp similar to WASVec 1.0 (even though WASVec2.0 has a much smaller proviral length) and both restored WASp to HD levels. No WASp expression was detected in the WAS KO control arm without LV transduction.

FIG. 8D shows a 3rd replicate of the experiment shown in FIG. 8B, where cells from 2 mixed CD34+ donors were used. At equivalent VCNs, WASVec2.0 V2 was found to express WASp better (greater amount) than WAS1.6.

FIG. 8E shows results obtained in a 3^{rd} replicate of the experiments described above. It shows that WASVec2.0 V2 was able to restore WASp to HD levels at a VCN of 1.29 while WAS1.6 needed a VCN of 2.61 to restore HD of WASp in WAS knockout megakaryocytes.

Overall, it was found that, at a VCN of 1.29, WASVec2.0 V2 was able to restore WASp expression to wildtype levels in the WAS−/− HSPC derived megakaryocytes while WAS1.6 was only able to restore WASp expression to 64% of wildtype levels. It was also found that both WASVec2.0 V2 and WASVec1.0 were able to restore wildtype levels of WASp in WAS−/− megakaryocytes.

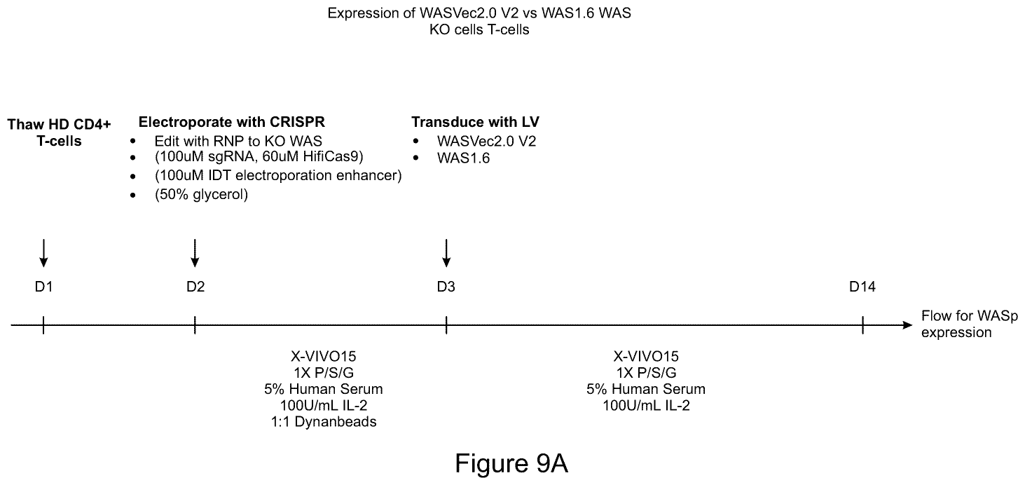

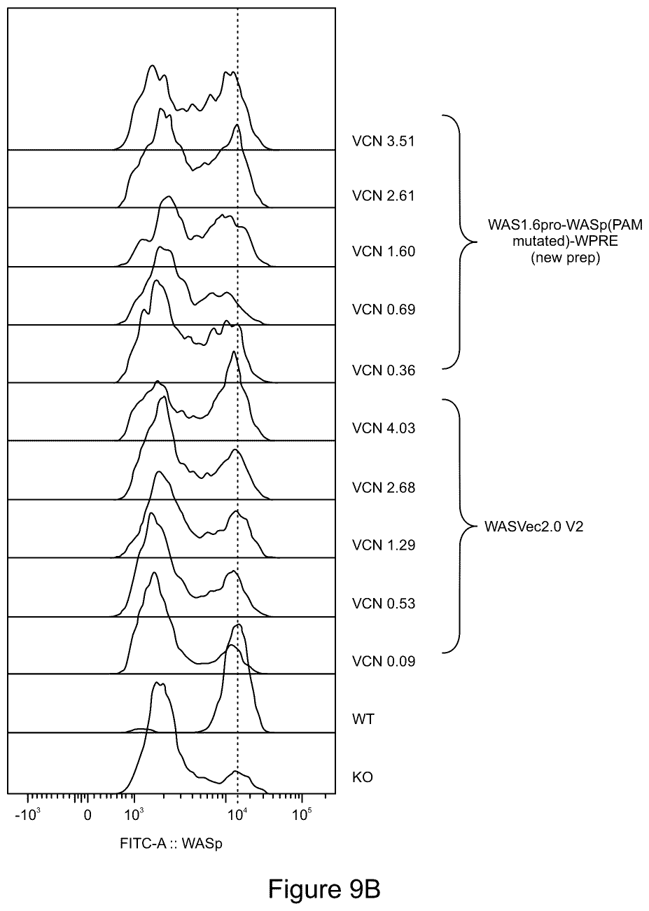

To evaluate the expression in T-cells, human WAS−/− T-cells generated by CRISPR knockout were transduced with WASVec2.0 V2 or WAS1.6 at a range of vector doses. FIG. 9A is a schematic which details the strategy used for evaluating the rescue of WAS expression with WASVec2.0 V2 vs. with WAS1.6 in WAS gene knockout (KO) T-cells.

In particular, to evaluate the ability of WASVec2.0 to restore WASp expression in WAS−/− T-cells, HD T-cells were electroporated with CRISPR to knockout the endogenous WAS gene and subsequently transduced with either WASVec2.0 V2 or WAS1.6, both expressing WASp. T-cells were cultured for an additional 11 days in X-VIVO15 media supplemented with 5% Human Serum and 100U/mL hIL-2. After 14 days, restoration of WASp and VCN was measured.

It was found that WASVec2.0 V2 was able to restore wildtype levels of WASp per transduced cell in WAS KO T-cells.

In particular, WASVec2.0 V2 was found to display a 1.37-fold improvement in viral titer and 4.2-fold improvement in gene transfer in HSPCs (human peripheral blood stem cells) over the previously known vector, WASVec1.0 (such as at a vector dose of 4.8×10⁷ TU/mL). Notably, WASVec2.0 V2 was also found to restore physiologic levels of WASp expression in transduced WAS−/− cells. See FIG. 9B. Thus, it was found that WASVec2.0 V2 maintains physiologic expression per integrated copy.

To evaluate the expression of WASp from WASVec1.6 and WASVec2.0 V2, CD34+-mobilized peripheral blood stem cells (PBSCs) were transduced with either WASVec2.0 or WAS1.6 (as previously described in Masiuk, 2019, Cell Stem Cell, 24(2):309-317.c7, doi: 10.1016/j.stem.2018.12.003). 24 hrs later, the cells were electroporated with CRISPR-Cas9 targeting the endogenous WAS gene (using the conditions from FIG. 8A) to knockout the endogenous WAS gene so to enable measurement of the ability of each LV to restore WAS levels. Cells were incubated for 13-days in the megakaryocyte differentiation media StemSpan media+50 ng/mL hTPO (as previously described in Perdomo, 2017, J Vis Exp, (130), c56420, doi: 10.3791/56420).

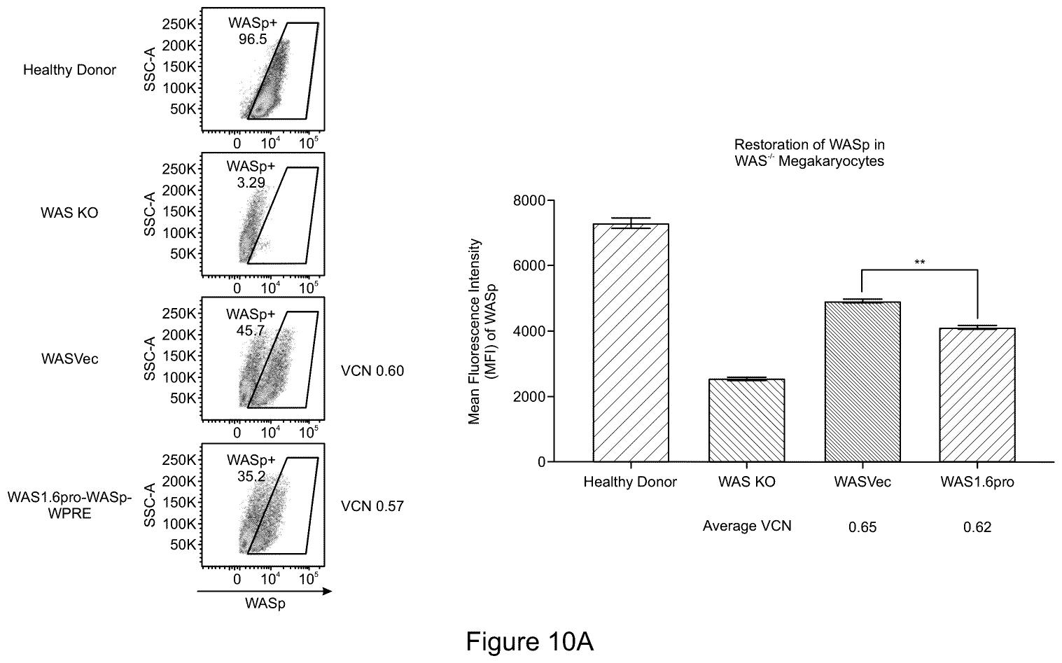

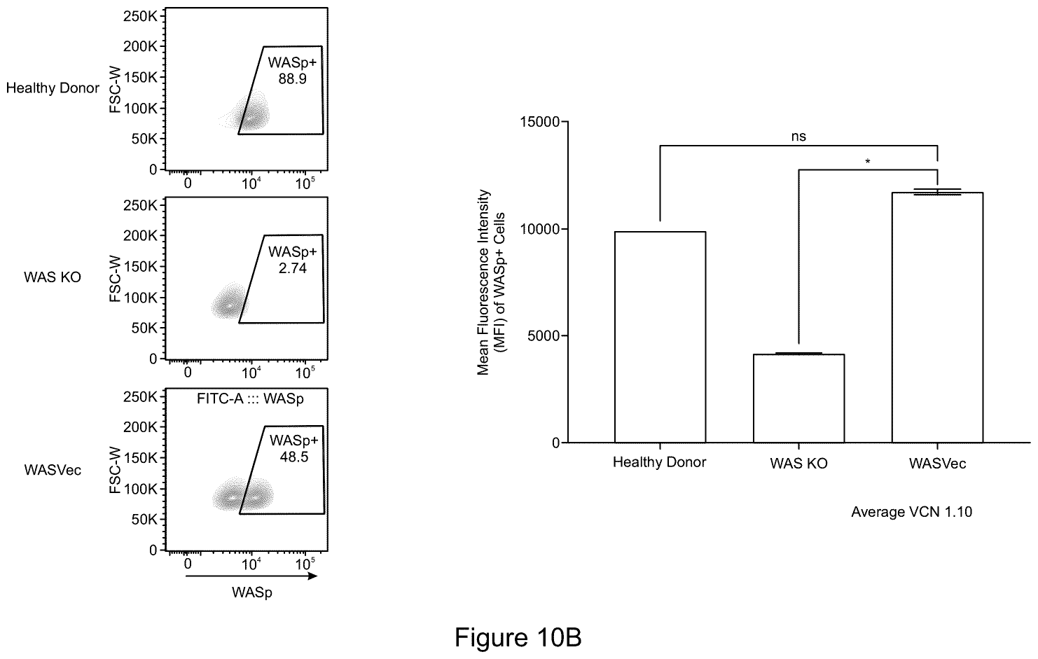

FIGS. 10A and 10B display the expression levels of intracellular WASp as measured via flow cytometry. The CRISPR-Cas9 targeting construct was able to knockout WAS with high efficiency. Furthermore, within an average VCN of 1.1, WASVec2.0 V2 was able to restore WASp to healthy donor levels in gene-corrected cells.

### Example 5—Restoration of WASp Levels In Vivo

This example shows that the new vectors having the new enhancer elements described herein are able to restore WASp expression across different hematopoietic lineages (including megakaryocytes) in vivo. Furthermore, this example shows that the level of WASp expression achieved using such vectors led to restoration of platelet counts and platelet function to healthy donor levels.

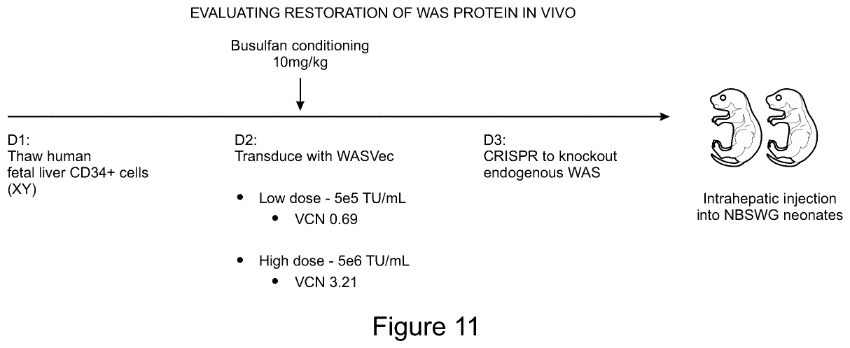

In order to evaluate the ability of WASVec2.0 V2 to restore WAS expression across the different hematopoietic lineages in-vivo, the protocol was optimized to create WAS−/− knockout HSPCs to transplant into NBSWG mice to. FIG. 11 demonstrates a schematic of the optimized protocol.

Healthy donor (HD) male fetal liver (FL) CD34 HPSCs were thawed and pre-stimulated in X-VIVO15 medium containing 100 ng/ml hSCF, 100 ng/mL hTPO, and 100 ng/mL Flt3L for 24 hours before transduction with WASVec2.0 V2 at a low or high vector dose of 5e5 TU/mL or 5e6 TU/L, respectively, for an additional 24 hours. The following day, a CRISPR-based method was employed to knockout the endogenous WAS gene in the transduced cells: the cells were electroplated with ribonuclear protein complex (RNP) composed of 100 uM of sgRNA targeting the endogenous WAS gene, 60 uM of HifiCas9 protein, and 100 uM of IDT electroporation enhancer. 2-hours post-electroporation, the WAS−/− knockout cells transduced with WASVec2.0 V2 were transplanted into busulfan-conditioned NBSWG mice via intrahepatic injection. WASVec2.0 V2 construct was codon-optimized, making it unrecognizable to the sgRNA targeting the endogenous WAS gene. The NBSWG model was chosen because it had been previously shown to support human platelet reconstitution over irradiated NSG mice. As a positive control, non-modified HD FL HSPCs were transplanted and WAS−/− knockout, non-transduced FL HSPCs were transplanted as a negative control. The two cell products that went into the mice kept in culture for 14 days to measure VCN of 0.69 and 3.21, for the low and high dose, respectively.

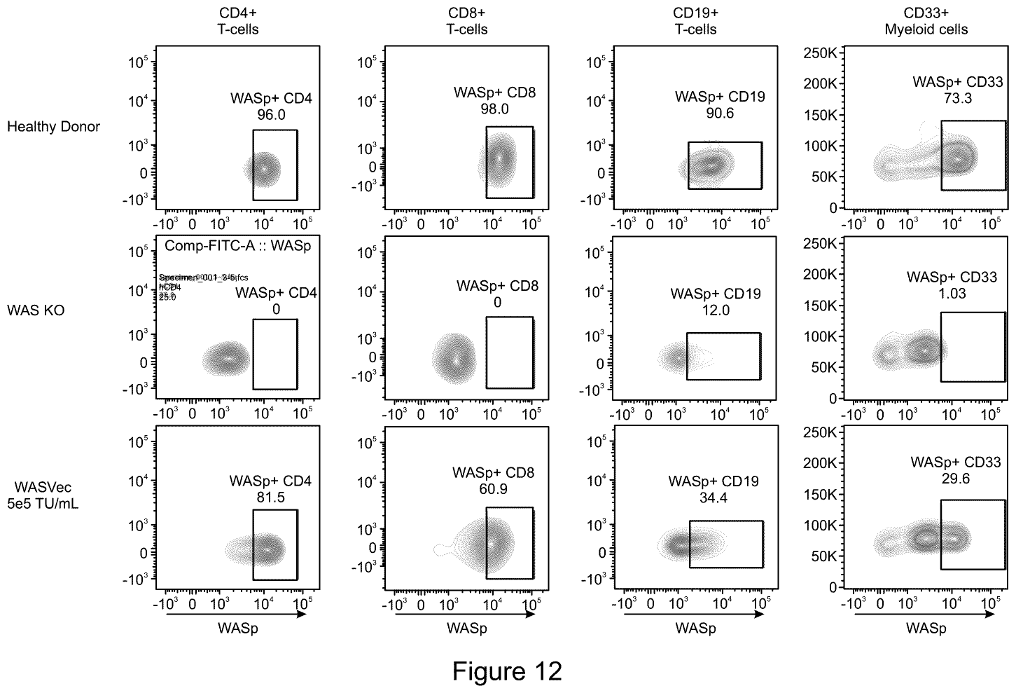

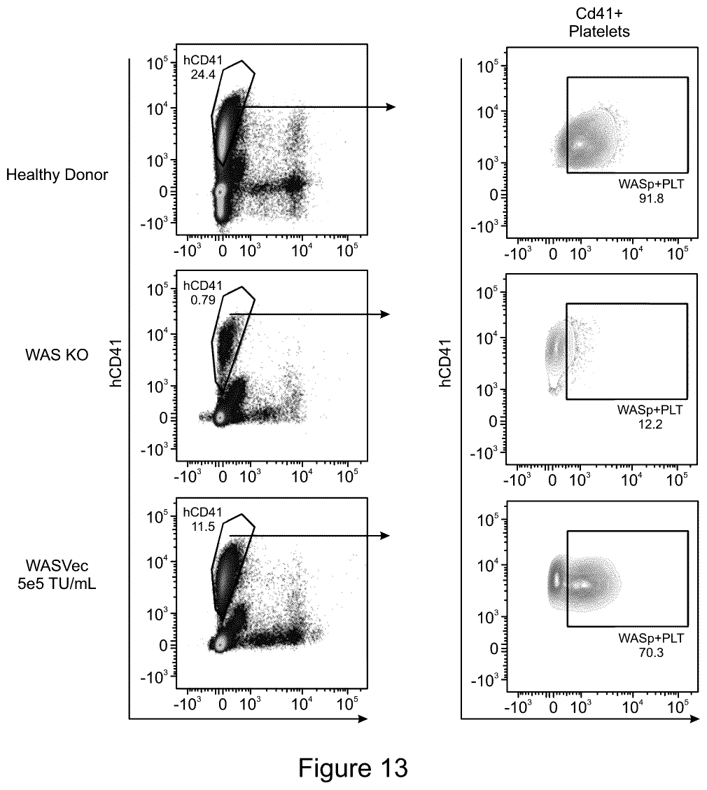

At 8 weeks post-transplant, the peripheral blood of the mice was analyzed to evaluate the restoration of WASp across the different affected hematopoietic lineages. FIG. 12 reveals that the knockout (KO) strategy had high efficiency resulting in a complete absence or very low levels of WASp across the reconstituted T-cells, B-cells, and myeloid cells in the engrafted mice compared to mice transplanted with unmodified HD HSPCs. Even at a low vector dose of 5e5 TU/mL, WASVec2.0 V2 restored WASp across the different hematopoietic lineages to physiologic levels in the WASp+ population. As demonstrated in FIG. 13, surprisingly high levels of platelet engraftment were observed in the transplanted mice with the presence of a CD41+CD45-population, which allowed to evaluate the expression of WASVec2.0 V2 in the platelet compartment. Mice transplanted with CRISPR WAS KO cells had very low levels of WASp expression in the peripheral blood (PB) platelets compared to mice transplanted with unmodified HD HSPCs. Mice transplanted with WASVec2.0 V2 transduced WAS KO HPSCs were able to restore WASp expression to HD levels in the WASp+ platelet population.

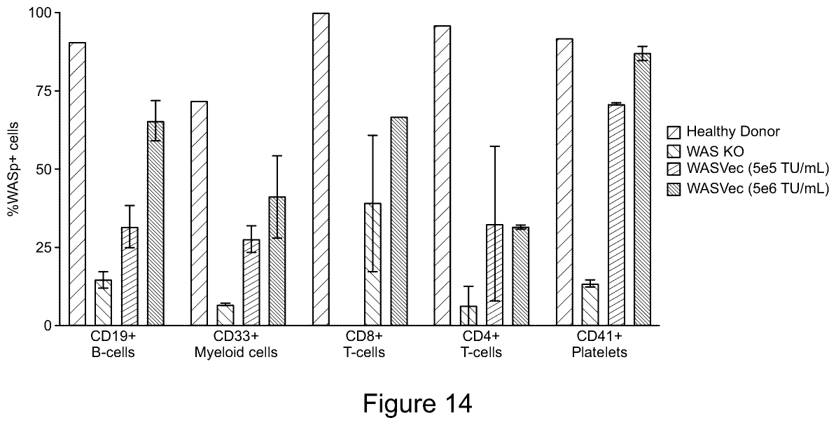

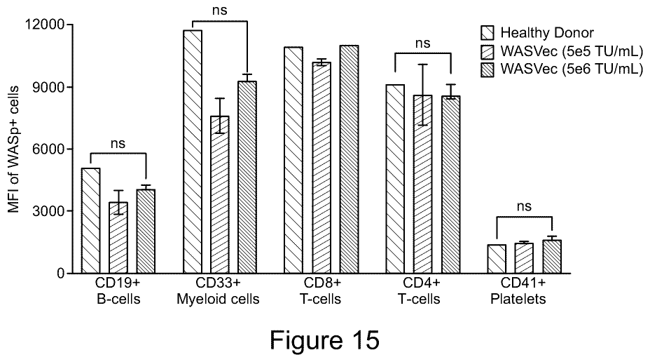

FIG. 14 demonstrates a clear increase of WASp+ cells among the different cell lineages of mice transplanted with cells transduced with a low (5e5 TU/mL) or higher (5e6 TU/mL) dose of WASVec2.0 V2. FIGS. 14 and 15 show that at a higher vector dose of 5e6 TU/mL, mice transplanted with WASVec2.0 V2 transduced cells had 87% HD levels of WASp+ platelets, compared to 92% seen in mice transplanted with WT HSPCs and thus a clear dose response was observed. This suggests that WAS levels correlate with platelet reconstitution in the model. Looking at the expression across different lineages, WASVec2.0 V2 was able to restore WASp expression to physiologic levels.

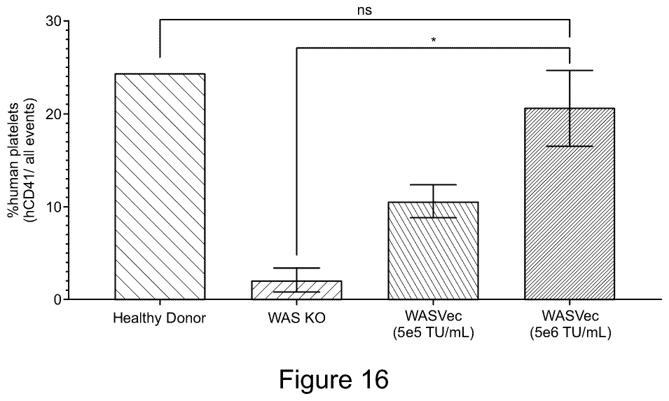

Since a robust platelet engraftment at 8 weeks post-transplant was observed, it was evaluated whether mice transplanted with WAS KO HSPSCs displayed thrombocytopenia compared to mice transplanted with WT HPSCs and if WASVec2.0 V2 could rescue this platelet defect. FIG. 16 demonstrates that mice transplanted with WAS KO HPSCs displayed much lower levels of circulating platelets compared to mice transplanted with unmodified HD HPSCs. Transduction of the WAS KO HPSCs with WASVec2.0 V2 at a vector dose of 5e6 TU/mL corrected the platelet counts to healthy donor levels. Partial correction in platelet counts was observed in mice transplanted with WAS KO cells transduced with WASVecV2 2.0 at a low dose of 5e5 TU/mL, which suggests that the correction of platelet counts is dependent on the expression of WASp and number of gene-corrected cells. To then evaluate the function of these platelets, the platelet responsiveness to thrombin stimulation and CD62p expression post-stimulation were measured.

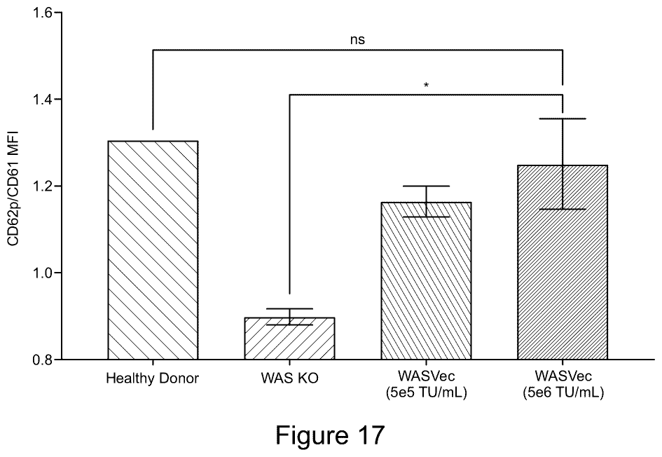

The results presented in FIG. 17 indicate that WT platelets and platelets derived from WASVec2.0 V2 transduced HPSCs at a higher vector dose had similar responsiveness to activation while WAS KO platelets had a blunted response.

Example 6-uCore E14 and uCore E2 Regulatory Elements Support Superior Expression of a Gene Encoding a WASp Protein from a Minimal WAS Promoter in Megakaryocytes and Restores Platelet Counts to a Healthy Donor Level In Vivo

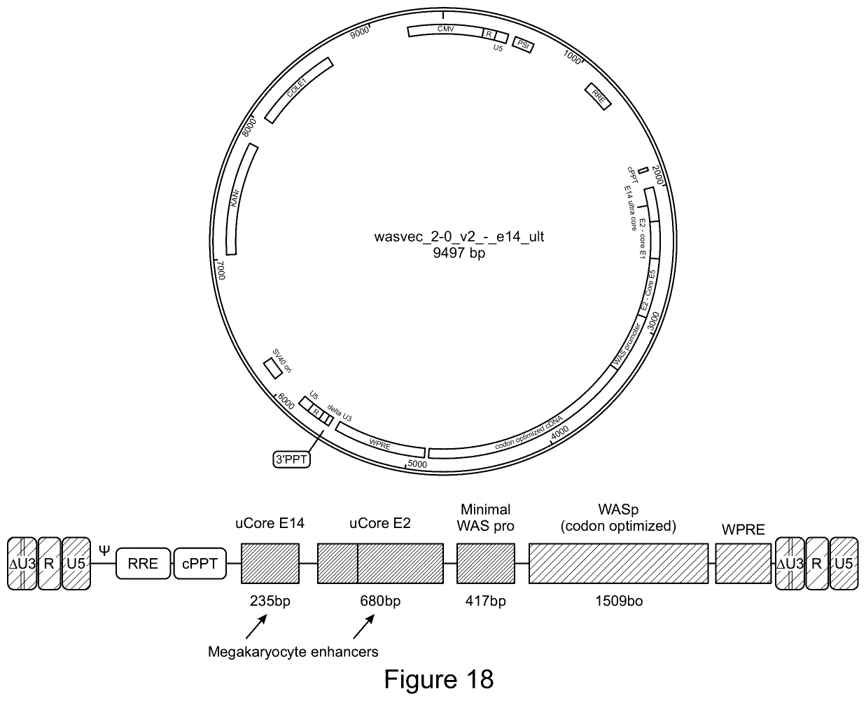

A transfer plasmid (FIG. 18) comprising novel megakaryocyte enhancers (uCore E14 and uCore E2) was cloned upstream of the minimal WAS promoter to drive expression of a codon optimized version of WASp. The vector was evaluated for its ability to restore healthy donor levels of WASp expression in the MK/platelet lineage in order to successfully correct platelet counts and function. This example demonstrates the ability of the claimed vector to restore healthy donor levels of WASp expression in WASp knockout (KO) MKs and platelets, and restore platelet counts in preclinical models.

### Development of WAS Animal Model and Study Design

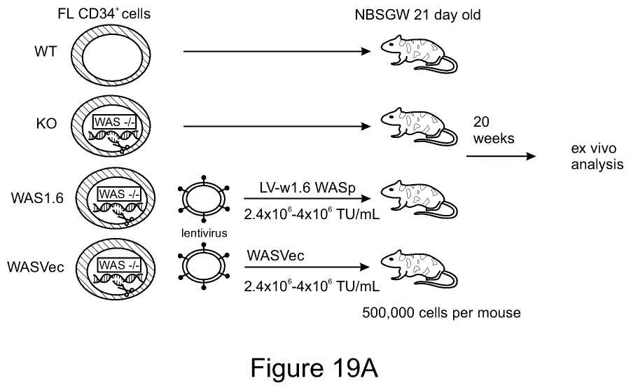

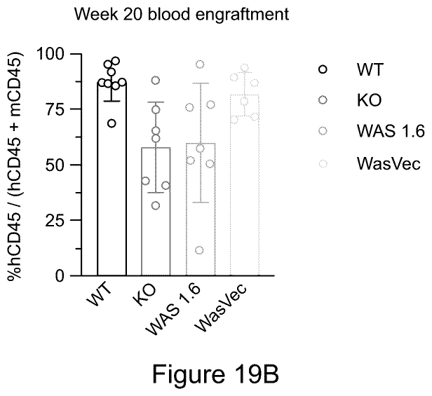

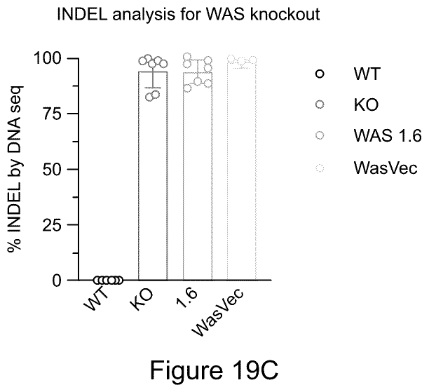

IMVC-003 (FIG. 18) was evaluated in a humanized mouse model to assess in vivo WASp expression and correction of thrombocytopenia and platelet function. NOD.Cg-Kit^{W-41J} Tyr⁺ Prkde^{scid} Il2rg^{tm1Wjl}/ThomJ (NBSGW) mice support multi-lineage human HSPC engraftment and human platelet development, providing a suitable model to test platelet count. As a surrogate product for WAS patient HSPC, WASp KO fetal liver CD34⁺ HSPCs were generated by electroporation of CRISPR/Cas9 ribonucleoprotein (RNP) with a guide RNA targeting exon 1 of the WAS gene. WT cells were mock electroporated. WASp KO CD34⁺ cells were transduced with WAS1.6 and IMVC-003 at equal vector dose and transplanted into 21-day old NBSGW mice by IV injection (FIG. 19A).

FIG. 19A depicts a schematic of an experimental set up used to validate the activity of the novel enhancers in a NOD.Cg-Kit^{W-41J} Tyr⁺ Prkdc^{scid} Il2rg^{tmlWjl}/ThomJ (NBSGW) humanized mouse model in order to restore healthy donor levels of WASp expression in the MK/platelet lineage in order to successfully correct platelet counts and function. WT arms received mock electroporation, KO arms were electroporated and edited using CRISPR/Cas9 with guide RNA targeting the WAS locus, WAS1.6 and WASVec2.0 V2 (referred to as Was Vec in the Figure) arms were transduced with IMVC-003 and Was1.6 lentiviral vectors and edited to KO endogenous WASp. 500,000 fetal liver CD34+ cells per mouse were injected into 21d old NBSGW adult mice via IV retro-orbital injection.

At 20 weeks post-transplant, the average stable vector copy number (VCN) in the bone marrow (BM) was 3.6 in the IMVC-003 arm and 1.35 in the WAS1.6 arm, reflecting superior gene transfer from IMVC-003. All mice had stable engraftment with the average engraftment for all arms being over 50% (FIG. 19B). FIG. 19B shows human chimerism calculated for all experimental arms using flow cytometry, the ratio of CD45+ human cells over total CD45+ mouse and human cells in the blood is shown. Successful knockout of WASp in vivo was confirmed by sequencing of the WAS gene from gDNA from the BM of all mice; The frequency of frameshift insertion/deletions was 94% on average and greater than 84% for all samples (FIG. 19C). FIG. 19C shows results of an analysis of genomic DNA extracted from the BM of all mice, PCR was performed to amplify the edited region of the WAS locus, WAS KO frequency was calculated by measuring the INDEL frequency compared to unedited WT DNA using the ICE tool (Synthego). The abbreviations used in FIGS. 19B and 19C are as follows: BM: bone marrow; INDEL: insertion deletion; IV: intravenous; KO: knockout; NBSGW: NOD.Cg-Kit^{W-41J} Tyr⁺ Prkdc^{scid} Il2rg^{tm1Wjl}/ThomJ; PCR: polymerase chain reaction; WAS: Wiskott-Aldrich syndrome gene; WASp: Wiskott-Aldrich syndrome protein; WASVec: IMVC-003, also known as WASVec2.0 V2; WT: wildtype.

### Example 7-IMVC-003 Corrects Megakaryocytes and Platelet Lineage in WAS Animal Model

The abbreviations used in FIGS. 20A-20F are as follows: BM: bone marrow; KO: knockout; MFI: mean fluorescence intensity; WASp: Wiskott-Aldrich syndrome protein; WASVec: IMVC-003 also known as WASVec2.0 V2; WT: wildtype.

Briefly, at 20 weeks post-transplant, the BM, spleen, and blood were harvested from all mice and immune cells were isolated from each tissue. Immune cells were analyzed for WASp expression using flow cytometry with intracellular staining. (20A-20C) WASp expression was measured by (top) MFI and by (bottom) % WASp cells in platelets and megakaryocytes. (20D-20F) Histograms are shown for each individual mouse, WASp MFI is measured on the x-axis. The vertical line represents the average MFI of the WT cells for each cell type. Data represents pooled data from 3 different donors with (n=4-7/arm). One way analysis of variance with Tukey's multiple comparisons.

Platelets are formed and released in the peripheral blood (PB) by MKs residing in the BM. In order to assess correction of the MK/platelet lineage, WASp expression was assessed in platelets in the blood and spleen, as well as the MK lineage in the BM. IMVC-003 platelets had WASp expression equivalent to WT platelets and at levels 2-fold higher than WAS1.6 platelets on average when measured by mean fluorescence intensity (MFI) (FIGS. 20A-20C, top charts). Notably, IMVC-003 platelets were nearly all WASp+ (90% on average) equivalent to WT platelets and WAS1.6 platelets were only 50% WASp+ on average (FIGS. 20A-20C, bottom charts).

IMVC-003 MKs in the BM also had WT levels of WASp expression measured by MFI and nearly all MKs in the IMVC-003 arm were WASp+ with no significant difference compared to WT (FIG. 20B). When WASp expression was examined using histograms measuring MFI, IMVC-003 platelets and MKs had WT levels of WASp expression. In addition, WAS1.6 WASp expression was highly variable and consistently below WT levels (FIGS. 20D-20F).

Methods: at 20 weeks post-transplant, the BM, spleen, and blood were harvested from all mice and immune cells were isolated from each tissue. Immune cells were analyzed for WASp expression using flow cytometry with intracellular staining. (20A-20C) WASp expression was measured by (top) MFI and by (bottom) % WASp cells in platelets and megakaryocytes. (20D-20F) Histograms are shown for each individual mouse, WASp MFI is measured on the x-axis. The vertical line represents the average MFI of the WT cells for each cell type. Data represents pooled data from 3 different donors with (n=4-7/arm). One way analysis of variance with Tukey's multiple comparisons.

### Example 8-IMVC-003 Produces Healthy Donor Levels of Functional Platelets

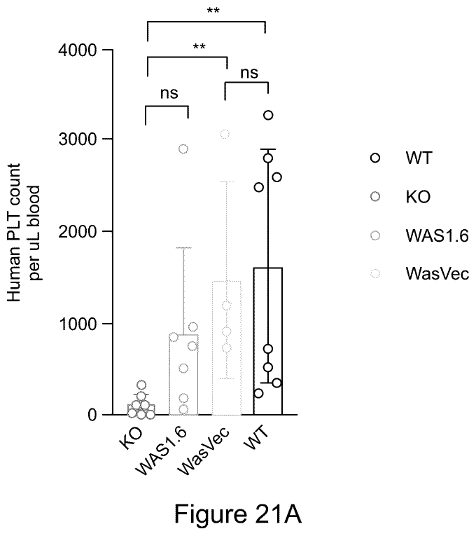

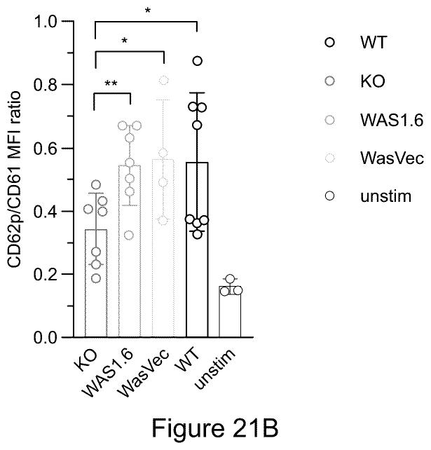

The abbreviations used in FIGS. 21A-21B are as follows: BM: bone marrow; KO: knockout; PB: peripheral blood; PLT: platelet; WASp: Wiskott-Aldrich syndrome protein; WASVec2.0V2: IMVC-003; WT: wildtype.

Briefly, absolute platelet counts in peripheral blood were assessed by flow cytometry and normalized by engraftment. IMVC-003 restored platelet counts to WT levels while WAS1.6 platelet counts remained 1.5-fold lower than WT and IMVC-003 platelet counts (FIG. 21A). Although there was an increase in WAS1.6 platelet counts compared to KO, that difference was insignificant (FIG. 21A). In addition to thrombocytopenia, WAS patients have impaired platelet function which can be quantified by decreased expression of CD62p (a platelet activation marker) upon activation with adenosine diphosphate (Sereni et al. 2019, Rai et al. 2020). The MFI ratio of CD62p/CD61 (a platelet lineage marker) was examined for all arms compared to unstimulated platelets and IMVC-003 platelet activation was found to be equal to WT platelets. WAS1.6 and IMVC-003 platelets were both significantly more activated compared to KO platelets. We also examined the relationship of WASp expression in the MK/platelet lineages and platelet count restoration. We found that the number of platelets in the PB was positively correlated with WASp expression in both PB platelets and BM MKs (FIG. 21B).

Methods: PB was collected through the retro orbital vein and PLTs were quantified using flow cytometry. (A) The bar graph represents the platelet count per μL of blood. (B) PB PLTs were activated with adenosine diphosphate (10 μM) for 10 minutes and immediately stained for flow cytometry analysis. CD62p/CD61 ratio was calculated using mean fluorescence intensity values for each marker. Data represents pooled data from 3 different donors with (n=4-7/arm). Unpaired t test with Welch's correction was performed.

### Example 9-WASp Expression is Restored in Multiple Cell Lineages In Vivo with IMVC-003

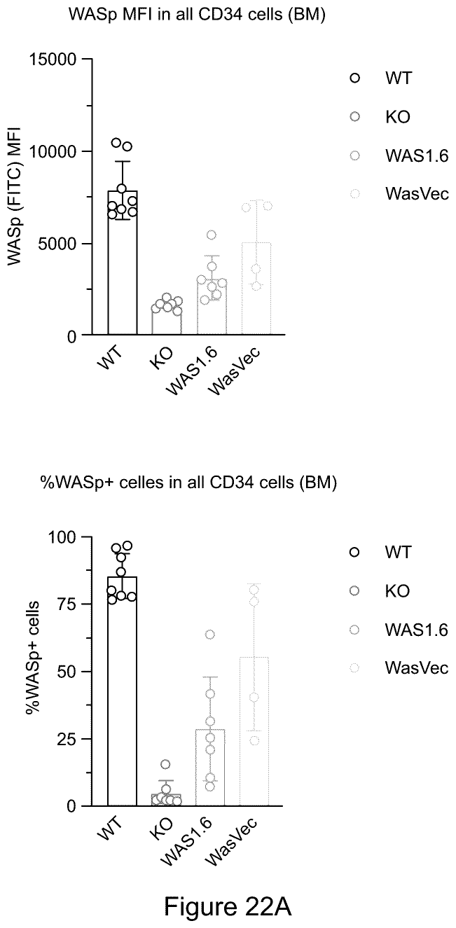

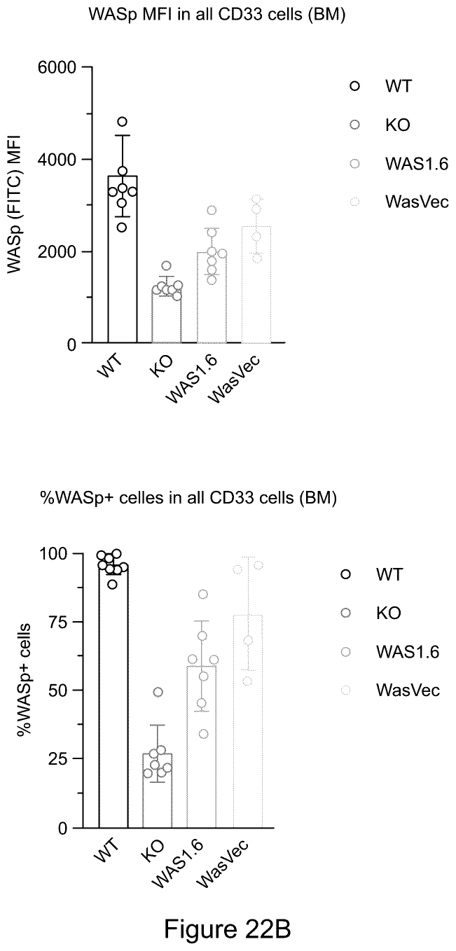

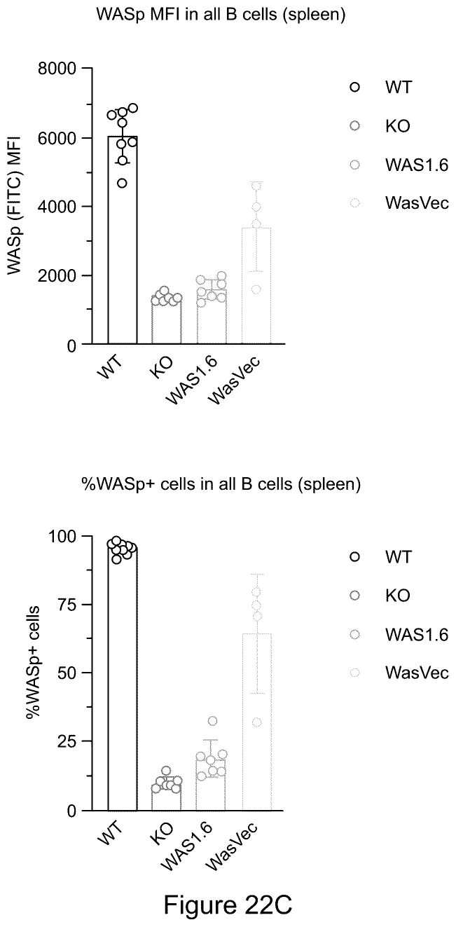

We also considered restoration of WASp expression in multiple lineages. IMVC-003 demonstrated superior WASp expression compared to WAS1.6 in preclinical studies. Briefly, at 20 weeks post-transplant, the BM and spleen were harvested from all mice and immune cells were isolated from each tissue. Immune cells were analyzed for WASp expression using flow cytometry with intracellular staining. WASp expression was measured by (top) MFI and by (bottom) % WASp cells in CD34+ HSPCs, CD33+ myeloid cells, and CD19+ B cells. Data represents pooled data from 3 different donors with (n=4-7/arm). One way analysis of variance with Tukey's multiple comparisons. When examining BM CD34+ cells, CD33+ myeloid cells and splenic CD19+ B cells, IMVC-003 arms displayed higher WASp MFI (FIG. 22A-22C, top) and more % WASp+ cells compared to WAS1.6 (FIG. 22A-C, bottom). IMVC-003 was engineered with additional MK specific enhancers with the goal of increasing MK expression which explains why expression is slightly lower in other cell lineages compared to MKs.

### Example 10-IMVC-003 Restores WASp Expression and IL-2 Production in WASp KO T Cells In Vitro

In T cells, WASp sets the threshold for T cell receptor (TCR)-driven activation by regulating the dynamics of lipid raft membrane microdomains during immunological synapse formation; WASp^{-/-} T cells show impaired responses to TCR stimulation including defective cytokine production. And contribute to the clinical immunodeficiency observed in WAS patients.

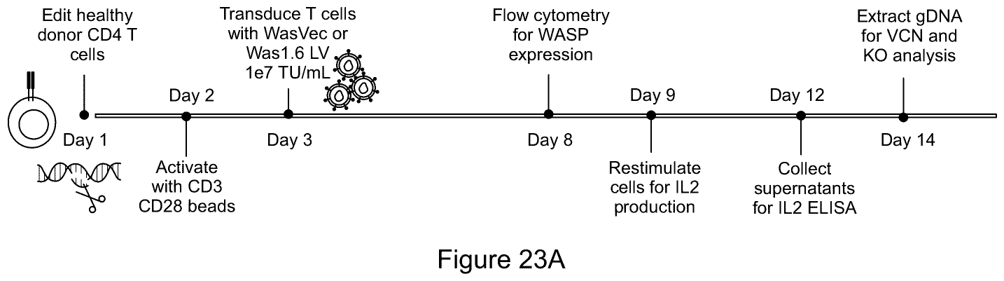

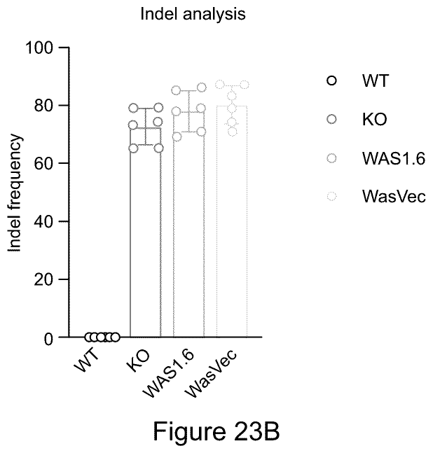

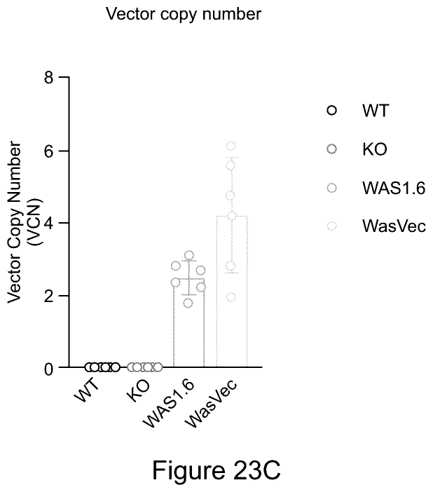

To test the activity of IMVC-003 in T cells, WASp KO T cells were generated from healthy donor CD4+ cells by electroporation of CRISPR/Cas9 RNP with a guide RNA targeting exon 1 of the WAS gene cells (see schematic of FIG. 23A). Briefly, resting healthy donor CD4 T cells from 3 independent donors were edited with CRISPR/Cas9 RNP with guide RNA targeting the endogenous WAS gene. Control (WT) T cells were mock electroporated without RNP. Cells were rested overnight and activated the following day with CD3/CD28 magnetic beads (Dynabeads). WASP KO cells were transduced with the lentiviral vectors Was 1.6 and IMVC-003. Cells were expanded and analyzed by flow cytometry 5 days post-transduction for WASp expression. Cells were re-activated on day 9 with CD3/CD28 Dynabeads and supernatants were assessed for IL2 production by enzyme-linked immunosorbent assay 72 hours post re-activation. Genomic DNA was extracted from cells on day 14 and analyzed for vector copy number and endogenous WAS gene KO.

Average KO efficiency measured by sequencing of the WAS gene ranged from 73-80% across all experimental arms (FIG. 23B). WASp KO cells were transduced with Was1.6 and IMVC-003; mean VCNs measured at Day 14 post-transduction were 2.5 and 4.2 for Was1.6 and IMVC-003, respectively, reflecting the superior gene transfer for IMVC-003 (FIG. 23C).

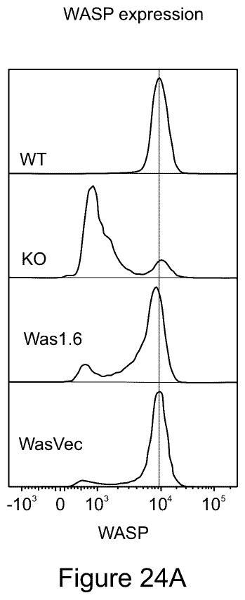

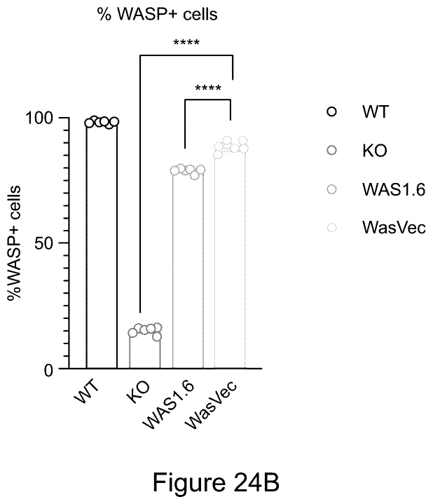

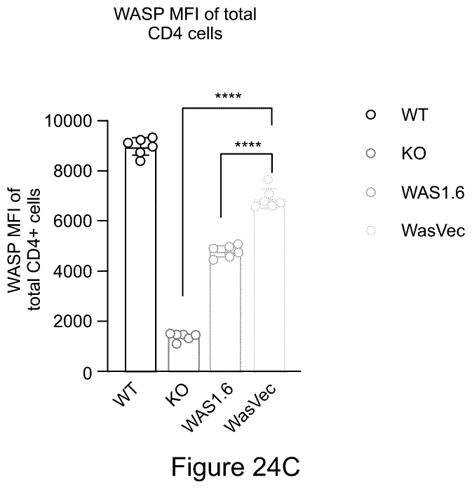

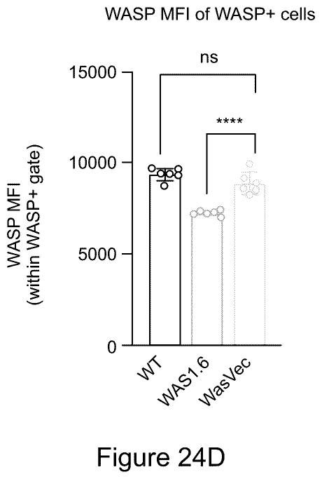

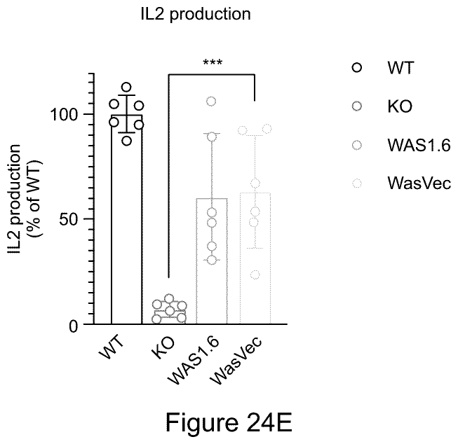

Relative to the WT (mock electroporated) control, Was1.6 and IMVC-003 both restored WASp expression. The percentage of WASp+ cells was 79% for Was1.6 and 88% for IMVC-003 (FIG. 24A). WASp expression of total CD4 cells, measured by mean fluorescence intensity (MFI) of total cells was 53% of WT levels for Was1.6 and 77% of WT levels for IMVC-003 (FIG. 24B). WASp expression per transduced cell, measured by MFI within the WASP+ gate, was 77% of WT levels for Was1.6 and 95% of WT for IMVC-003 (FIG. 24C) Collectively, these results demonstrate that IMVC-003 achieves restoration of WASp protein in 88% of T cells, and corrected T cells contain 95% of WT levels of WASp protein per cell.

T cell functionality was also tested by the ability of cells to produce IL-2 upon CD3/CD28 bead re-activation. Was1.6 and IMVC-003 restored 60% and 63% of WT levels of IL-2 production respectively. These data confirm that IMVC-003 restores functionality to WASp KO T cells. FIG. 24D represents a WASp MFI of WASP⁺ cells, representing average level of WASp per transduced (WASp⁺) cell. FIG. 24E illustrates IL2 production measured by enzyme-linked immunosorbent assay after reactivation with CD3/CD28 Dynabeads. To normalize for donor variability, experimental arms are normalized as a percentage of the WT arm for each donor. Data represents pooled data from 3 different donors with 2 replicates per donor (n=6/arm). One way analysis of variance with Tukey's multiple comparisons.

In summary, WAS1.6 gene therapy has demonstrated success in correcting T cell function, infectious complications, and autoimmunity in WAS patients (Hacein-Bey Abina et al. 2015, Labrosse et al. 2019, Magnani et al. 2022). IMVC-003 achieves levels of T cell correction equal to or superior to WAS1.6 suggesting that the level of correction achieved by IMVC-003 should similarly achieve clinical resolution of infectious complications and autoimmunity in WAS patients.

### Example 11-Dose Response Studies in Murine Lin-Cells

A prospective LV dose response was performed in wild-type murine lineage negative cells in order to select a LV dose for transplant. 100,000 WT murine lineage-negative cells were transduced at a range of doses with w1.6W LV or WasVec2.0 v2 (referred to as WASVec in the Figure) LV, cultured in BBMM culture for 14 days, followed by extraction of gDNA and analysis of VCN. Based on the dose response results, a LV dose of 6e6 TU/mL (MOI 6.6) was selected for the transplant study. This LV dose yielded an in vitro VCN of 0.8 for w1.6W and 2.7 for WasVec.

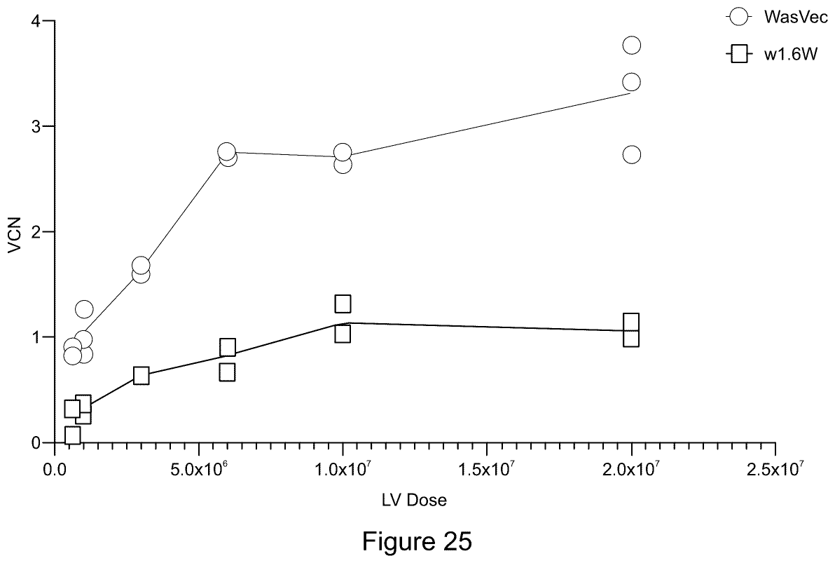

FIG. 25 is a chart illustrating results of experiments evaluating effects of the vector dose response in murine lin-cells. Briefly, an in vitro LV dose escalation was performed in murine lin-cells in order to choose an optimal LV dose for comparison of Was Vec2.0 v2 (referred to as WasVec in the Figure) and w1.6W LV. The graph in FIG. 25 represents the administered LV dose (TU/mL) and the resulting VCN after 14 days of expansion in murine myeloid differentiation media.

### Example 12-In Vivo Transplantation

10 WAS recipient mice per arm (5M and 5F) were prospectively identified for transplant, assigned a study number by car-punching for identification, and underwent irradiation on day-1 (24 hours prior to transplant). During the isolation of murine lin-cells, the quantity of male murine lin-cells obtained was only sufficient to transplant 3 of the 5 female mice in the WT arm. Therefore, two female mice (mouse 9, WT; mouse 17, WT) which underwent car-punching and irradiation did not receive transplant and were euthanized and excluded from the study.

6 study deaths (1× WT, 3× WAS NTD, and 2× w1.6W) occurred within the first 30 days post-transplant, which is the expected time frame for engraftment failure post-BMT. At d+32 to d+42 post-transplant, all mice in the study developed ulcerative dermatitis (UD), a known side effect of radiation exposure. Dermatitis was characterized by ulcerative, pruritic lesions over the dorsal neck. Topical treatment with GenOne (Gentamicin/Betamethasone) spray was initiated 5 days per week, and weekly toenail trims were performed. In two cases (Mouse 14, WAS NTD; and Mouse 19, w1.6W), the lesions became severe and euthanasia was mandated on d+53 by a veterinarian from the UCLA Division of Laboratory Medicine who was blinded to the study and treatment groups. Three additional mice with UD (Mouse 32, WasVec2.0 v2 (referred to as WASVec in the Figure); Mouse 34, WAS NTD; and Mouse 35, w1.6W) died spontaneously between d+40 and d+69. UD in all other mice resolved by d+84 with treatment. The overall attrition due to dermatitis in our cohort (5/38, 13%) is within the range of typical estimates for mice on a C57Bl/6J background (4-20%23,24). All mice which died before week 12 were excluded from analysis. One mouse (Mouse 13, WT), which was analyzed for platelet WASP expression at 12 weeks and CBC analysis at 15 weeks, was found dead at d+122 (17 weeks). This mouse was excluded from all terminal (20-week) analyses.

### Analysis of Peripheral Blood Platelets: WASP Expression

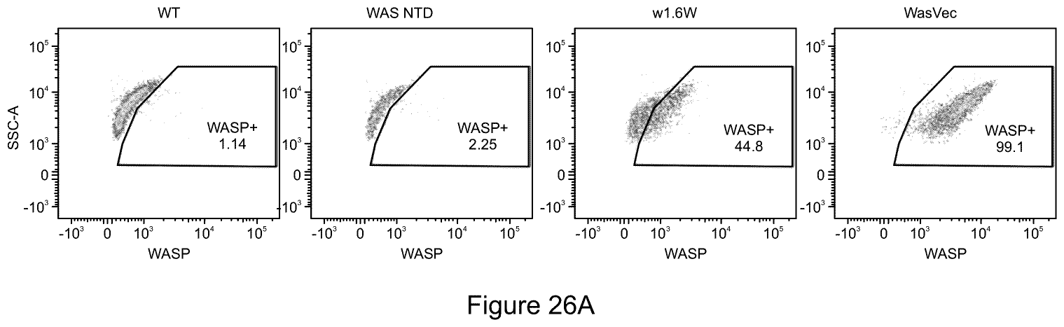

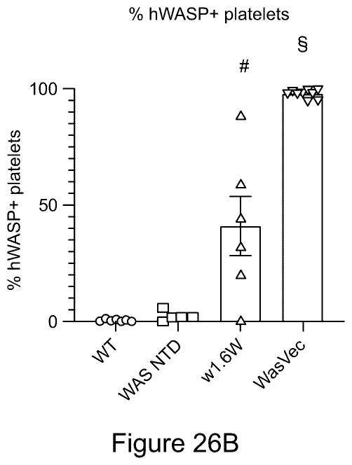

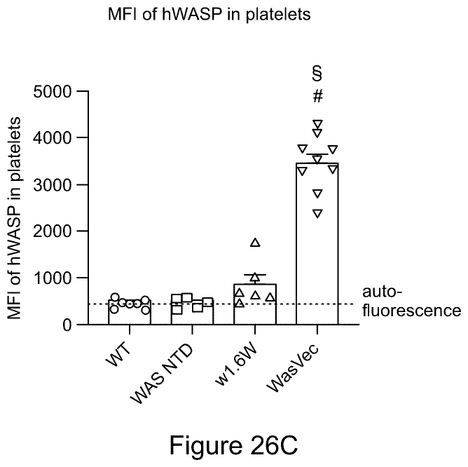

At 12 weeks post-transplant, peripheral blood was collected and platelets were stained for WASP expression by flow cytometry (FIG. 26A). Briefly, peripheral blood was stained for CD41a (platelet marker) followed by fixation and permeabilization for intracellular staining of human WASP protein. FACS plots show WASP expression in FSC^{low}SSC^{low}CD41a⁺ platelets. FIG. 26B illustrates the percentage of WASP+ platelets in each transplant arm. In mice receiving Was Vec2.0 v2 (referred to as WASVec in the Figure) transduced cells (IMVC-003), platelets were >97% WASP+(FIG. 26B). In contrast, mice receiving w1.6W transduced cells showed lower and more variable levels of WASP+ platelets (mean=41%, range 1-88%). Furthermore, the levels of WASP protein per platelet, as measured by MFI, were 7-fold higher in Was Vec2.0 v2 (referred to as WASVec in the Figure) treated mice compared to w1.6W treated mice (FIG. 26C). As expected, platelets expressing human WASP were not detected in mice receiving WT and non-transduced WAS cells. The monoclonal antibody EP2541Y used for WASP detection only detects the human WASP transgene and does not cross react with the murine WASP protein.

### CBC Analysis: Platelet Counts

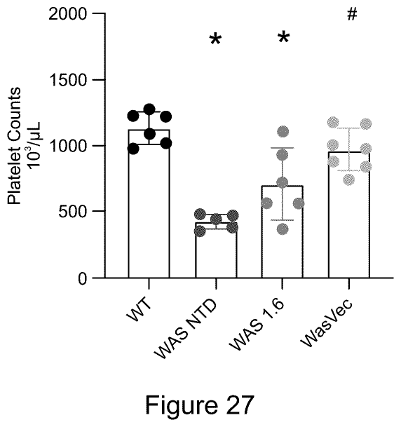

FIG. 27 illustrates the results of platelet counts from CBC analysis of peripheral blood at 15 weeks post-transplant. At 15 weeks post-transplant, peripheral blood was collected for CBC analysis. Here, we found that mice receiving non-transduced WAS cells had significantly lower platelet counts (˜2-fold lower) compared to mice receiving WT cells. Treatment with Was Vec2.0 v2 (referred to as WASVec in the Figure) transduced cells (IMVC-003) significantly increased platelet counts with 6/7 mice achieving platelet counts within the normal reference range. In contrast, only 2/6 mice treated mice receiving w1.6W transduced cells achieved normal platelet counts. Statistical Analysis: *=p<0.05 vs WT; #=p<0.05 vs WAS NTD; 1-way ANOVA with Tukey's multiple comparisons.

### CBC Analysis: Blood Lineages in Peripheral Blood

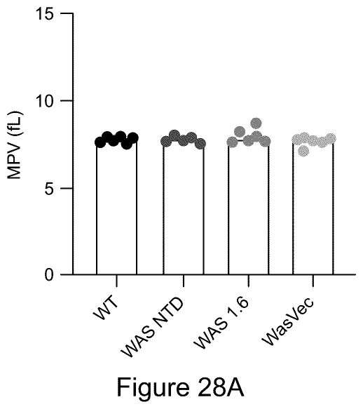

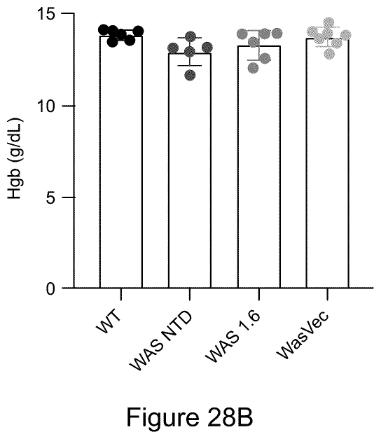

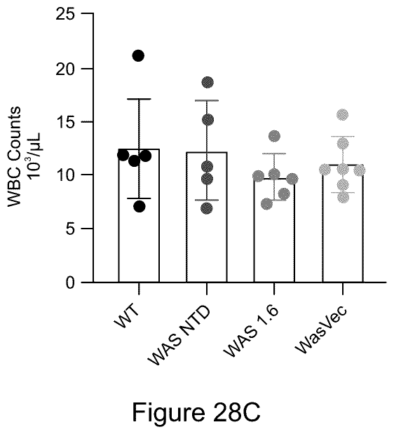

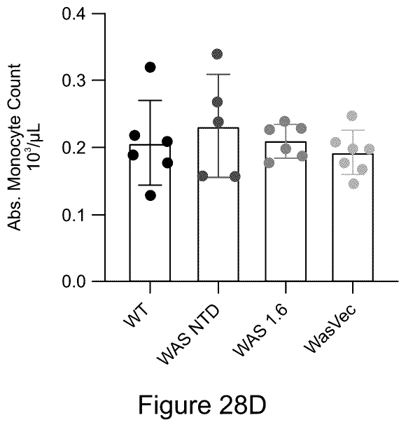

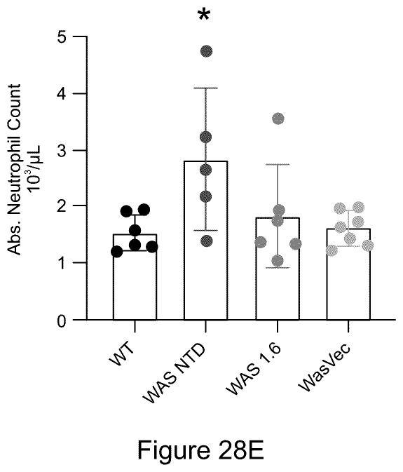

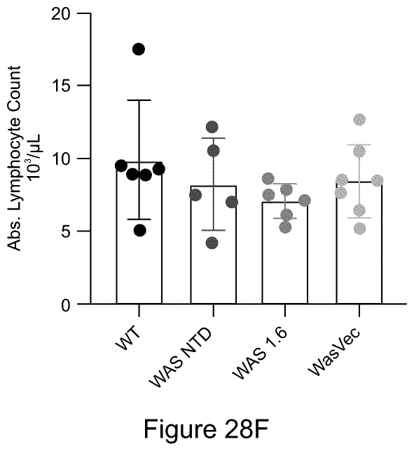

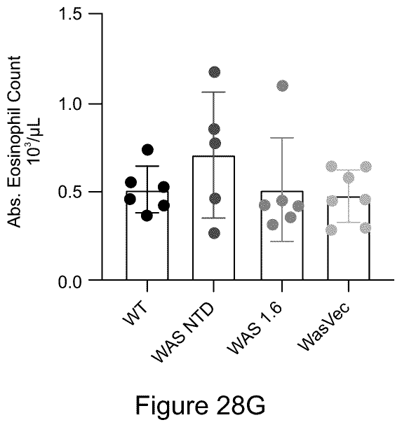

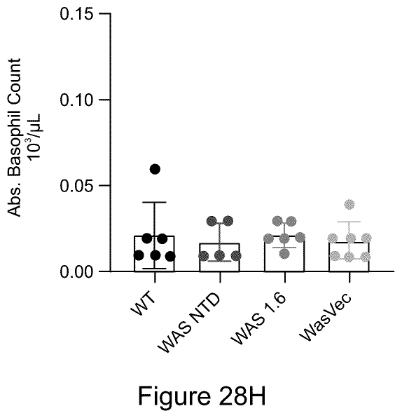

FIGS. 28A-28H depict the results of the CBC analysis of peripheral blood at 15 weeks post-transplant. Mean CBC values for mice in all 4 study arms were within normal reference ranges, with the following exceptions: Mice in all 4 study arms had elevated cosinophil counts with no significant differences among study arms. Elevated absolute neutrophil counts were observed in mice receiving non-transduced WAS cells, but were within normal range in mice receiving w1.6W and Was Vec2.0 v2 (referred to as WASVec in the Figure) transduced cells. Statistical analysis: *=p<0.05 vs WT; #=p<0.05 vs WAS NTD; 1-way ANOVA with Tukey's multiple comparisons.

### Engraftment and Vector Copy Number

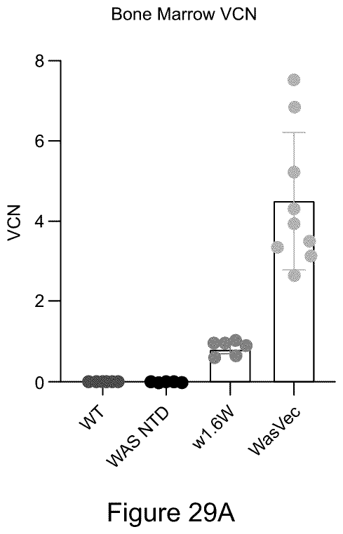

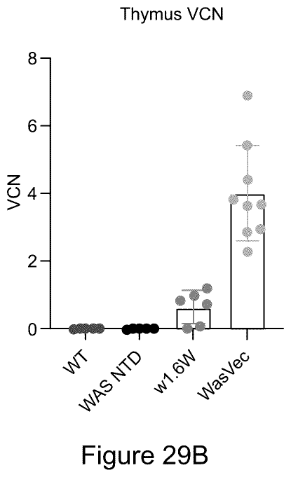

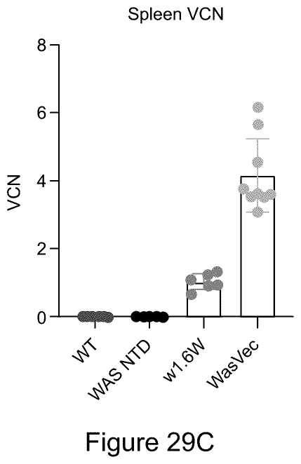

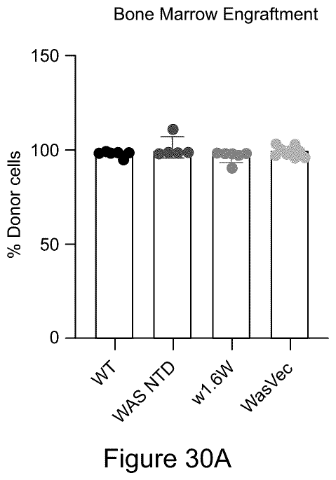

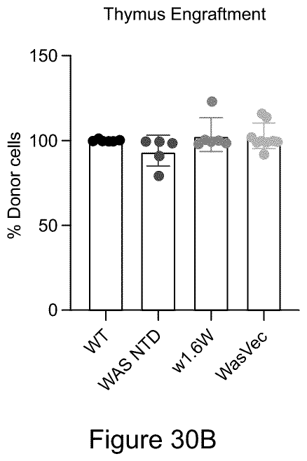

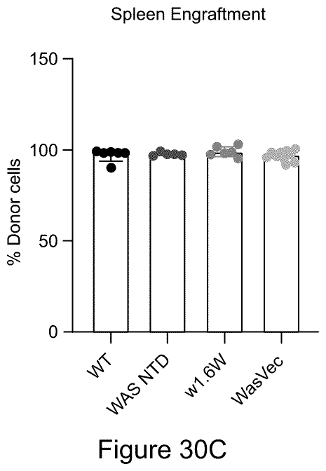

Briefly, at 20 weeks post-transplant, bone marrow, thymus, and spleen were collected and analyzed for vector copy number (FIGS. 29A-29C) and engraftment (FIGS. 30A-30C) by digital PCR. Mean bone marrow VCN was 1 for w1.6W and 4 for Was Vec2.0 v2 (referred to as WASVec in the Figure), reflecting improved gene transfer with Was Vec2.0 v2 (referred to as WASVec in the Figure) at equal TU/mL and MOI. Mean engraftment was >90% in all transplant arms in all tissues analyzed. FIGS. 29A-29C depict the results of a vector copy number (VCN) analysis in the bone marrow (A), thymus (B), and spleen (C) at 20 weeks post-transplant. FIGS. 30A-30C depict the results of engraftment as measured by ddPCR to determine the percentage of donor cells in the bone marrow (A), thymus(B), and spleen (C) at 20 weeks post-transplant.

### Lineage Distribution in Bone Marrow, Spleen, and Thymus by Lineage.

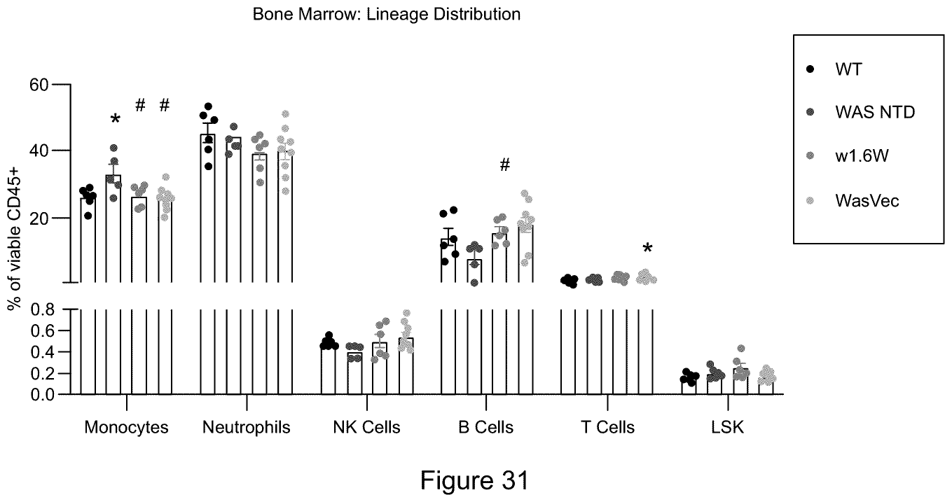

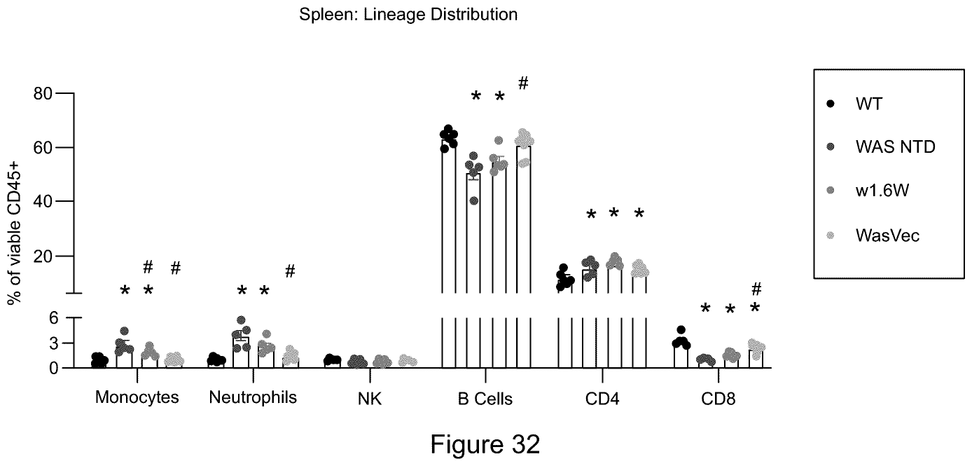

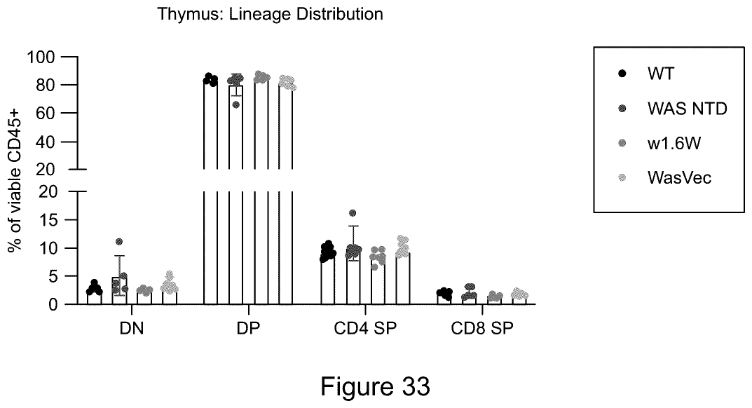

At 20 weeks post-transplant, bone marrow, thymus, and spleen were collected and analyzed for lineage distribution by flow cytometry, FIGS. 31-33 respectively. Lineage distributions across all arms were overall as expected with the following exceptions: Relative to the WT arm, mice receiving non-transduced WAS cells had mildly elevated percentage of monocytes (bone marrow and spleen) and neutrophils (spleen), and decreased percentages of B cells (spleen) and CD8 T cells (spleen), all of which were normalized in mice receiving Was Vec2.0 v2 transduced cells. Mice receiving non-transduced, w1.6W transduced, and Was Vec2.0 v2 transduced cells all had mild but statistically significant elevations in the percentage of CD4 T cells. No differences were seen among groups in thymus lineage distribution.

As shown in FIGS. 31-33, the bone marrow, spleen, and thymus lineage distribution graphs show the percentage of each defined hematopoietic lineage in either the bone marrow, spleen, or thymus. Percentages were calculated as the percentage of cells within the defined lineage relative to the number of total viable mCD45⁺ cells. LSK: Lineage negative, Sca1+, c-kit+. NK cells: Natural killer cells. Statistical analysis: *=p<0.05 vs WT; #=p<0.05 vs WAS NTD; 1-way ANOVA with Tukey's multiple comparisons.

In conclusion, WAS mice receiving IMVC-003 showed durable (20-week) multi-lineage engraftment (100%) in the bone marrow, spleen, and thymus. Mean in vivo copy number was 4.5, 4.2, and 4.0, in the BM, spleen, and thymus respectively. Mice engrafted with IMVC-003 demonstrated multi-lineage expression of the human WASP transgene in hematopoietic cells and platelets, ranging from 40-80% in hematopoietic cells and >97% in platelets.

Importantly, IMVC-003 fully resolved thrombocytopenia, with 8/9 treated mice achieving platelet counts in the normal reference range with no significant differences between mice receiving IMVC-003 or WT cells.

### Example 13-Expression of IMVC-003 vector resolved thrombocytopenia by restoring platelet expression

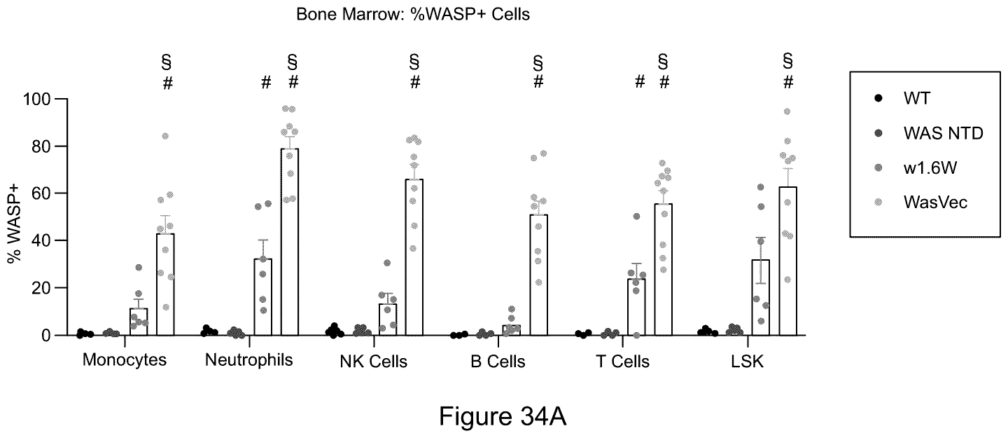

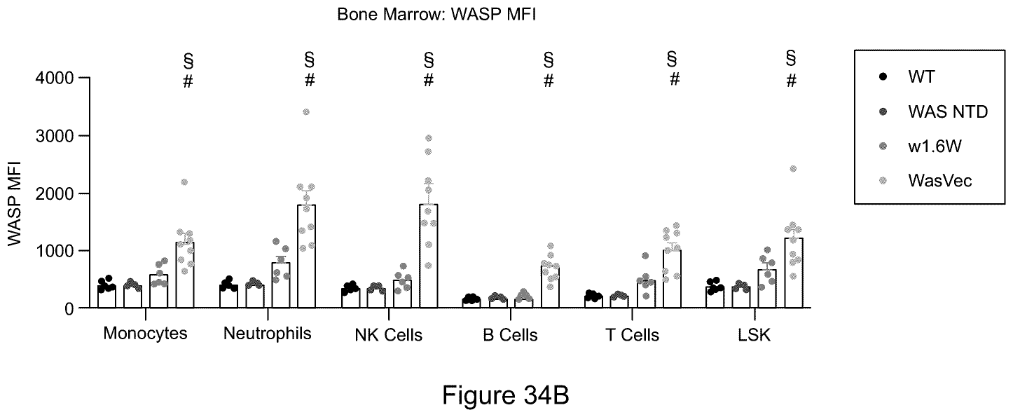

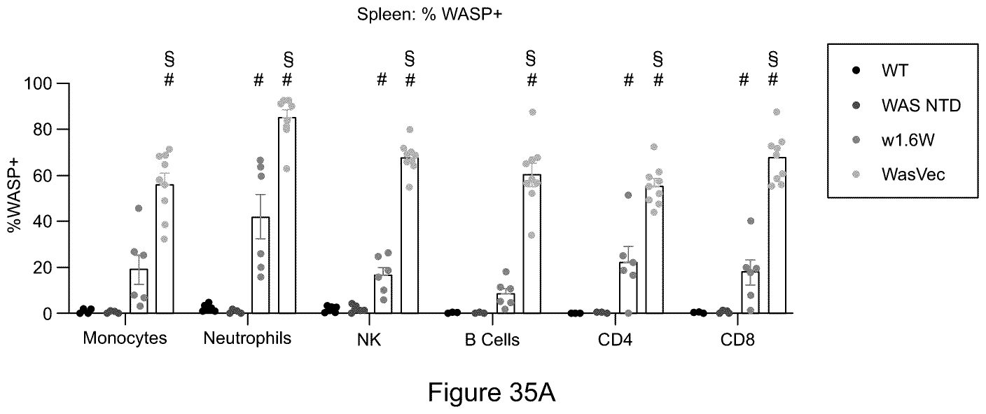

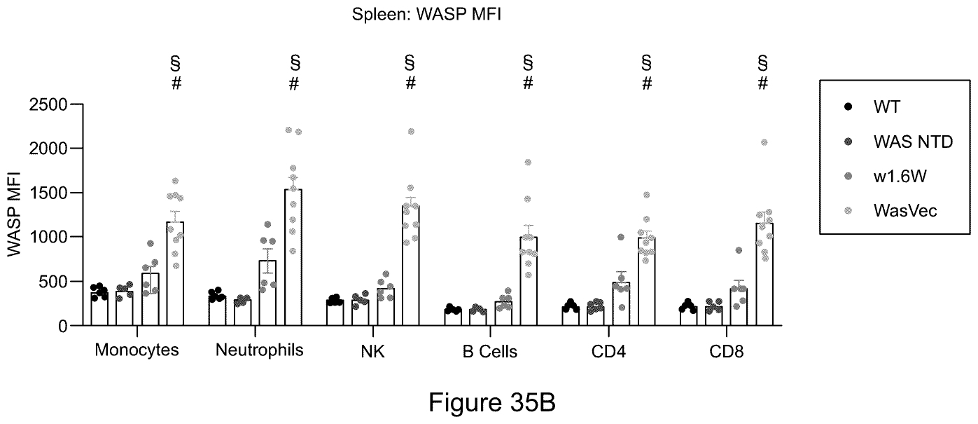

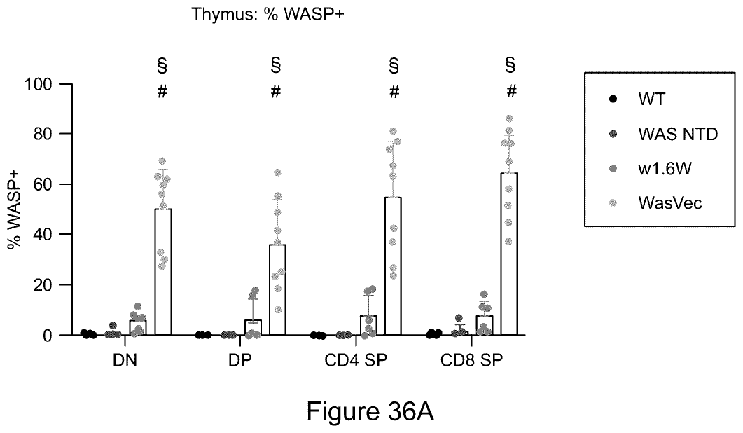

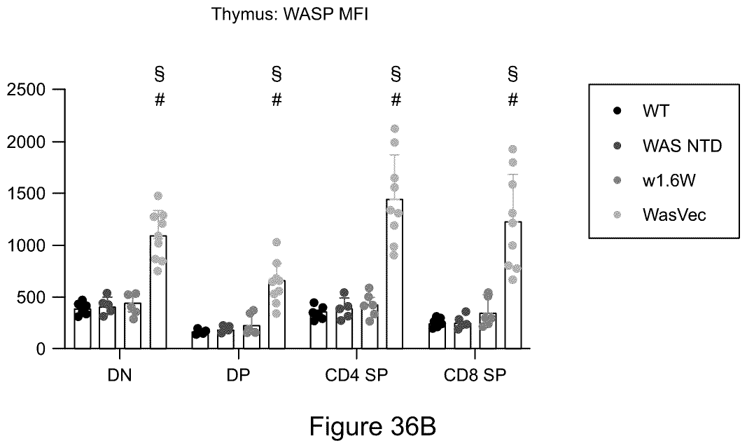

Bone marrow, thymus, and spleen were additionally analyzed for intracellular human WASP (hWASP) expression by flow cytometry. Mice receiving IMVC-003 had an average of 40-80% hWASP+ cells in all analyzed lineages. Lineage-negative, Sca1+, c-kit+(LSK) cells, representing engrafted HSC, were 60% hWASP+, indicating durable modification and engraftment of long-term HSC. In all lineages, the percentage of hWASP+ cells and the MFI of hWASP in each lineage was significantly higher in mice receiving Was Vec2.0 v2 (referred to as WASVec in the Figure)/IMVC-003 compared to mice receiving cells transduced with the w1.6WLV. As expected, cells expressing human WASP were not detected in mice receiving WT and non-transduced WAS cells. The monoclonal antibody EP2541Y used for WASP detection only detects the human WASP transgene and does not cross react with the murine WASP protein. FIG. 34A depicts the percentage of hWASP+ cells within each defined hematopoietic lineage in the bone marrow. FIG. 34B depicts the mean fluorescence intensity (MFI) of hWASP in each defined lineage in the bone marrow. Similarly, FIGS. 35A and 36A depict the percentage of hWASP+ cells within each defined hematopoietic lineage in the spleen (35A) and thymus (36A), respectively. Likewise, FIGS. 35B and 36B depict the mean fluorescence intensity (MFI) of hWASP in each defined lineage in the spleen (35B) and in the thymus (36B), respectively.

### Example 14-Identification of Minimal Enhancer Elements for WASp (WAS Protein) Expression

This example shows identification of minimal enhancer elements for WASp (WAS protein) expression, the use of which leads to superior hematopoietic stem and progenitor cell (HPSC) gene transfer and improved viral titer, while maintaining the ability to restore physiologic levels of WASp expression in WAS−/− cells.

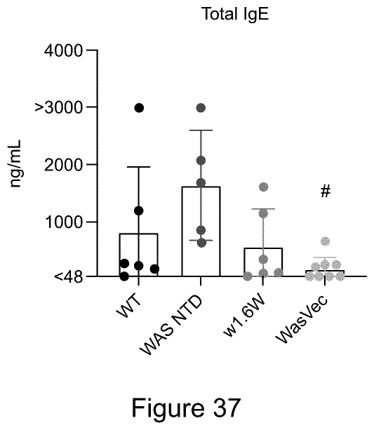

Briefly, total serum IgE was measured by ELISA at 20 weeks post-transplant (FIG. 37). WT C57Bl/6J mice typically have total serum IgE levels <100 ng/mL7. titerTreatment with Was Vec2.0v2/IMVC-003 significantly reduced mean IgE levels, with only 1/9 mice treated with WasVec2.0v2 having IgE levels >500 ng/ml (FIG. 37). *=p<0.05 vs WT; #=p<0.05 vs WAS NTD; and 1-way ANOVA with Tukey's multiple comparisons.

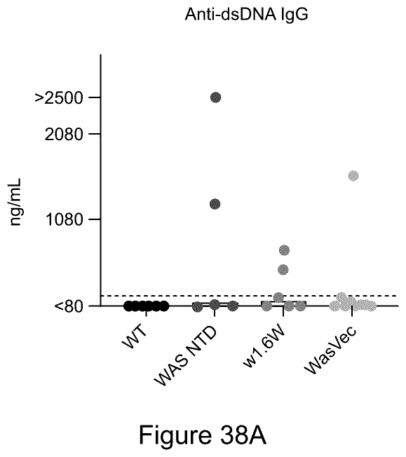

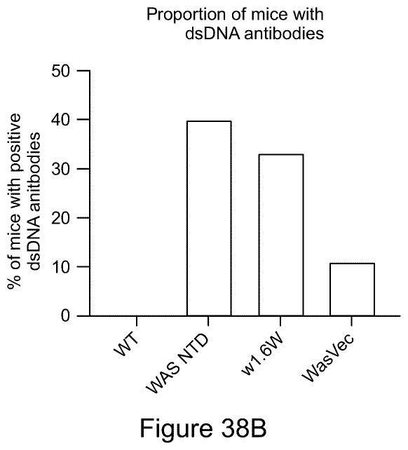

Further, serum was collected at 20 weeks and analyzed for the presence of anti-dsDNA antibodies by ELISA. In total, 5 mice within the study cohort had positive levels of anti-dsDNA antibodies (>2.5-fold x the ELISA detection limit). As depicted in FIG. 38A, concentration of serum anti-dsDNA IgG was calculated by ELISA. Anti-dsDNA antibodies were found in 0% of WAS mice receiving WT cells and 40% of WAS mice receiving WAS non-transduced developed anti-dsDNA antibodies. In contrast, only 11% of mice receiving WasVec2.0 v2 (referred to as WASVec in the Figure)/IMVC-003 developed positive anti-dsDNA antibodies, indicating a decreased autoantibody production. FIG. 38B is a chart depicting the proportion of mice in each arm with positive dsDNA antibodies.

### Example 15-Response to Vaccination with Pneumo Vax23

B-cell antibody responses to type II T-independent antigens, such carbohydrate-based Pneumococcal vaccines, are impaired in WAS mice. In order to evaluate the efficacy of IMVC-003 in restoring B cell function, mice were immunized with 2 μg of Pneumovax23 at 16 weeks post-transplant. Serum was collected at 20-weeks post-transplant for the evaluation of anti-pneumococcal IgM. Treatment with Was Vec2.0 v2 (referred to as WASVec in the Figure)/MVC-003 significantly increased anti-pneumococcal IgM, though levels were significantly lower than in mice receiving WT cells. In contrast, no significant increase in antibody response was seen in mice receiving w1.6W LV.

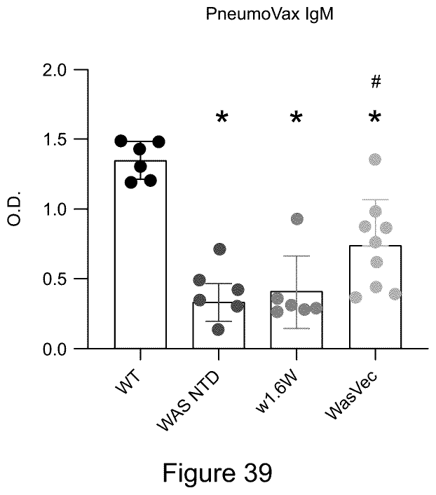

FIG. 39 depicts the results of the experiment. More specifically, mice were immunized with PneumoVax23 (2 μg/mouse i.p. injection) at 16 weeks post-transplant. Serum was collected at 20-week post-transplant (28 days post-immunization) and analyzed for the presence of anti-pneumococcal antibodies by ELISA. Graph depicts ELISA O.D. values for anti-pneumococcal IgM. Statistical analysis: *=p<0.05 vs WT; #=p<0.05 vs WAS NTD; 1-way ANOVA with Tukey's multiple comparisons

B cell dysfunction is a major hallmark of WASP, with impaired antibody responses to carbohydrate-based vaccines, and dysregulated immunoglobulin production. Treatment with IMVC-003 WASP transgene was expressed in about 60% of splenic B-cells. IMVC-003 resulted in improved antibody responses to the Pneumo Vax23 vaccine. WAS mice treated with IMVC-003 also exhibited normalization of elevated anti-dsDNA antibodies and normalization of elevated IgE levels. Thus, the use of IMVC-003 represents a valid therapeutic option for the treatment of WAS patients and may contribute to amelioration of immunodeficiency, autoimmunity, and thrombocytopenia thereby contributing to a better quality of life for this group of patients.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention. The scope of the invention will be measured by the appended claims and their equivalents. The abstract and the title are snot to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. § 112, 96.

SEQUENCES:

| SEQ ID NO | |
|---|---|
| And Description | Nucleic Acid Sequence |
| SEQ ID NO: 1 | GATATCTTGATTCCCTCAGAACCAG |
| Element 14 (E14) | GTGGAAACATGTTCTGAATAAAAGC |
| human | CAGTCCTGGGTATTCTTGCTCTACT |
| | CACCGGCTTGGTTGTGTTGGGCTGA |
| | GCCTTATAGAACACAGTGCTGGAGA |
| | CCTTCTCAGGGTCACTTCCTGTACC |
| | CTTGACTTTGAGCTGCAGCCCTCTG |
| | GTTTTCTCAGGTGAGTTTTCTTGAC |
| | TCTTTCCACTACTCTTCAGCCGATA |
| | AAGTGACCATTCCACTCATGTGCTT |
| | TCCCCTGGGGTAACTCCTAGTGGAT |
| | GCAGTGTTGGGACCCCTTAGTTTCA |
| | CTGGGCAGTGTCACAGGGCAGGCTC |
| | TTTAGAAGCAGAATTTCAGCTGGAG |
| | TTTGGGGCACAGGAGGTTTATCAGT |
| | TGATGTAAAGGGAAGGAGGAGGAAG |
| | CAGGATCGGGCAGGGGGAGAGCTGA |
| | GTATGATGCAAGCCACACAAAGCCT |
| | TGGCCAGCCCAGCGGGGAGCTCTGG |
| | AGCAGGGATGGCGTGGCAGAGTGTT |
| | TTGGGGTAAAGGGCATGAGCTTGGG |
| | GAAGTGACTTTCTGCAGACTCTGAT |
| | GGAGCTGACATCTGGAGGCTGCCTG |
| | CCCACCGCACTCCCTGCAGCTGTGC |
| | AGCGAGCCTTTCCTTGAAAAGGGGT |
| | CTCAGTGGTGCACATTTTTTTTTGA |
| | GACAGAGTCTTGCTCTGTCGCTCAG |
| | GCTGGAGTATAGTGGCATGATCTCT |
| | GCTCATTGCAACCTCTGCCTCCCGG |
| | GTTCAAGCGATTCTCACGCCTCAGC |
| | CACCCAAGTAGCTGAGATTACAGGC |
| | GTGCACCACCACACGTCTCTACTGT |
| | TTTTAGTAGAGACGGGGTTTCAGCA |
| | TGTTGACCAGGCTGGTCTCGAACTC |
| | CCGATCTCAAGTGATCCGCCCGCCT |
| | CGGCCTCCCAAAGTGCTGGGATTCC |
| | AGGTGTGAGCCACCGCACCCAGCCA |
| | GTGGTTCACATCTTTGTCTCCCACT |
| | GTCAGGAAAGGTATGGGGAGGAGCT |
| | GTGAAAAGAAATGGGTGGCCACTGG |
| | GTAAGTAACTGGGGACCCAGGGAAA |
| | ATCCTGTTACTCCCATGACTCGCCC |
| | ACATGGTCCTGAGCCAGTCACTTTA |
| | CCACTCTGTGCCTCAGTTTCCTCAA |
| | GACTCAGTTGAGGGTTGGGGAGTAT |
| | ATCCATTCATTCTTCTGACCTATAT |
| | TGACTGAGTACCTACATGTGCCAGG |
| | CACTGTGCCAAGTTATAACAGTCAA |
| | CAAGGCACACAAAACCTCGCCCTCA |
| | GGGAGCTGACATTGCCAAGAACCCC |
| | TCAAATCTACCCAGTGACCCAGACT |
| | GAGGGCAGATGTCACCCCTGGCCTT |
| | GCTTGCAATGATCATGACGCACAGT |
| | GAGCACCGTGGCCTCGTTGCATATT |
| | CAGACAGATGACAGCCTGACATGGA |
| | AGCTGAGGATCTAATCAGTGGCATC |
| | CACATGGTAACAGGAAGGAAGGAGC |
| | CACTGTGGGTCCTTGAGCAGGAGCA |
| | CGGAGATATCAGTGCAAAACTTCAG |
| | AAGGGCAATTCCAGTGGCAATCAAT |
| | AGAAAAAGACCTTTTTACTGCATGT |
| | CACCCTGCCCCTTAGGTGAGTCAAA |
| | TTAAGGCAAATAGCCGAGGCCAGGC |
| | CACCCTGTCTGTCCCCTGTTATATT |
| | TTTGGTTTTGGTTTGTTTATAGCTG |
| | AGTTATTCCCTAGTGGTGGCAGTGA |
| | TGATTGGGAATTTGGCTTTCTGATC |
| | TGACCATTTGGTGACTTCTGGGAAT |
| | GAATGAACGGATGTTGGCAGAGAAC |
| | AGCAGGAGTCAGGCATGTTATCCTG |
| | TGTCTCTTGGATCTCTTGCCCTGTG |
| | GCTAACGGTGTGAAAATGAAGCATT |
| | AAGCTCCCTCTGTGGCTGCAGTCAG |
| | ACTCCTGGGGTGAGTGAGTAATGTT |
| | TTCCCATCTCTGGAGGGTGTCTATA |
| | CATTATTTTGTAATGTCACTTGAAT |
| | TAAAGGAACTCAGTTCTGATTGGCT |
| | GATAGAGATAAATAAGCACTTATAC |
| | AAATCAAATATTCCTAAAATTTAGG |
| | AAAATGAGGAAATCAGACTTTTTAA |
| | ATGGCTGTCCTCTGTAGTCCAGGAA |
| | GAAACTGAACTTTTAGTACCGTAAA |
| | TGCACTAGTCTTGAAAAAGATTCCT |
| | CCTTCAGAAACTAATTCAGAGACTT |
| | TAAGAATCCAAGTTCTCATAATTAA |
| | GATAAATCTTTGGCAAATGACATTA |
| | GTTTAATTATTTGAGTTTAATAA |
| SEQ ID NO: 2 | GAATAACTCAGCTATAAACAAACCA |
| Element 14 core | AAACCAAAAATATAACAGGGGACAG |
| human | ACAGGGTGGCCTGGCCTCGGCTATT |
| | TGCCTTAATTTGACTCACCTAAGGG |
| | GCAGGGTGACATGCAGTAAAAAGGT |
| | CTTTTTCTATTGATTGCCACTGGAA |
| | TTGCCCTTCTGAAGTTTTGCACTGA |
| | TATCTCCGTGCTCCTGCTCAAGGAC |
| | CCACAGTGGCTCCTTCCTTCCTGTT |
| | ACCATGTGGATGCCACTGATTAGAT |
| | CCTCAGCTTCCATGTCAGGCTGTCA |
| | TCTGTCTGAATATGCAACGAGGCCA |
| | CGGTGCTCACTGTGCGTCATGATCA |
| | TTGCAAGCAAGGCCAGGGGTGACAT |
| | CTGCCCTCAGTCTGGGTCACTGGGT |
| | AGATTTGAGGGGTTCTTGGCAATGT |
| | CAGCTCCCTGAGGGCGAGGTTTTGT |
| | GTGCCTTGTTGACTGTTATAACTTG |
| | GCACAGTGCCTGGCACATGTAGGTA |
| | CTCAGTCAATATAGGTCAGAAGAAT |
| | GAATGGATATACTCCCCAACCCTCA |
| | ACTGAGTCTTGAGGAAACTGAGGCA |
| | CAGAG |
| SEQ ID NO: 3 | CCACTGGAATTGCCCTTCTGAAGTT |
| Element 14 ultra- | TTGCACTGATATCTCCGTGCTCCTG |
| core | CTCAAGGACCCACAGTGGCTCCTTC |
| human | CTTCCTGTTACCATGTGGATGCCAC |
| | TGATTAGATCCTCAGCTTCCATGTC |
| | AGGCTGTCATCTGTCTGAATATGCA |
| | ACGAGGCCACGGTGCTCACTGTGCG |
| | TCATGATCATTGCAAGCAAGGCCAG |
| | GGGTGACATCTGCCCTCAGTCTGGG |
| | TCACTGGGTA |
| SEQ ID NO: 4 | CATGACCTTATGGGACTTTCCTACT |
| Sequence of | TGGCAGTACATCTACGTATTAGTCA |
| WASVec2.0 V1 | TCGCTATTACCATGGTGATGCGGTT |
| Synthetic | TTGGCAGTACATCAATGGGCGTGGA |
| construct | TAGCGGTTTGACTCACGGGGATTTC |
| | CAAGTCTCCACCCCATTGACGTCAA |
| | TGGGAGTTTGTTTTGGCACCAAAAT |
| | CAACGGGACTTTCCAAAATGTCGTA |
| | ACAACTCCGCCCCATTGACGCAAAT |
| | GGGCGGTAGGCGTGTACGGTGGGAG |
| | GTCTATATAAGCAGAGCTCGTTTAG |
| | TGAACCGGGGTCTCTCTGGTTAGAC |
| | CAGATCTGAGCCTGGGAGCTCTCTG |
| | GCTAACTAGGGAACCCACTGCTTAA |
| | GCCTCAATAAAGCTTGCCTTGAGTG |
| | CTTCAAGTAGTGTGTGCCCGTCTGT |
| | TGTGTGACTCTGGTAACTAGAGATC |
| | CCTCAGACCCTTTTAGTCAGTGTGG |
| | AAAATCTCTAGCagtggcgcccgaa |
| | cagggacttgaaagcgaaagggaaa |
| | ccagaggagctctctcgacgcagga |
| | ctcggcttgctgaagcgcgcacggc |
| | aagaggcgaggggcggcgactggtg |
| | agtacgccaaaaattttgactagcg |
| | gaggctagaaggagagagatgggtg |
| | cgagagcgtcagtattaagcggggg |
| | agaattagatcgcgatgggaaaaaa |
| | ttcggttaaggccagggggaaagaa |
| | aaaatataaattaaaacatatagta |
| | tgggcaagcagggagctagaacgat |
| | tcgcagttaatcctggcctgttaga |
| | aacatcagaaggctgtagacaaata |
| | ctgggacagctacaaccatcccttc |
| | agacaggatcagaagaacttagatc |
| | attatataatacagtagcaaccctc |
| | tattgtgtgcatcaaaggatagaga |
| | taaaagacaccaaggaagctttaga |
| | caagatagaggaagagcaaaacaaa |
| | agtaagaccaccgcacagcaagcgg |
| | ccgctgatcttcagacctggaggag |
| | gagatatgagggacaattggagaag |
| | tgaattatataaatataaagtagta |
| | aaaattgaaccattaggagtagcac |
| | ccaccaaggcaaagagaagagtggt |
| | gcagagagaaaaaagagcagtggga |
| | ataggagctttgttccttgggttct |
| | tgggagcagcaggaagcactatggg |
| | cgcagcgtcaatgacgctgacggta |
| | caggccagacaattattgtctggta |
| | tagtgcagcagcagaacaatttgct |
| | gagggctattgaggcgcaacagcat |
| | ctgttgcaactcacagtctggggca |
| | tcaagcagctccaggcaagaatcct |
| | ggctgtggaaagatacctaaaggat |
| | caacagctcctggggatttggggtt |
| | gctctggaaaactcatttgcaccac |
| | tgctgtgccttggaatgctagttgg |
| | agtaataaatctctggaacagattt |
| | ggaatcacacgacctggatggagtg |
| | ggacagagaaattaacaattacaca |
| | agcttaatacactccttaattgaag |
| | aatcgcaaaaccagcaagaaaagaa |
| | tgaacaagaattattggaattagat |
| | aaatgggcaagtttgtggaattggt |
| | ttaacataacaaattggctgtggta |
| | tataaaattattcataatgatagta |
| | ggaggcttggtaggtttaagaatag |
| | tttttgctgtactttctatagtgaa |
| | tagagttaggcagggatattcacca |
| | ttatcgtttcagacccacctcccaa |
| | ccccgaggggacccgacaggcccga |
| | aggaatagaagaagaaggtggagag |
| | agagacagagacagatccattcgat |
| | tagtgaacggatctcgacggtatcg |
| | gttaacttttaaaagaaaagggggg |
| | attggggggtacagtgcaggggaaa |
| | gaatagtagacataatagcaacaga |
| | catacaaactaaagaattacaaaaa |
| | caaattacaaaaattcaaaatttta |
| | tcgatcacgagactagcctcgagGA |
| | ATAACTCAGCTATAAACAAACCAAA |
| | ACCAAAAATATAACAGGGGACAGAC |
| | AGGGTGGCCTGGCCTCGGCTATTTG |
| | CCTTAATTTGACTCACCTAAGGGGC |
| | AGGGTGACATGCAGTAAAAAGGTCT |
| | TTTTCTATTGATTGCCACTGGAATT |
| | GCCCTTCTGAAGTTTTGCACTGATA |
| | TCTCCGTGCTCCTGCTCAAGGACCC |
| | ACAGTGGCTCCTTCCTTCCTGTTAC |
| | CATGTGGATGCCACTGATTAGATCC |
| | TCAGCTTCCATGTCAGGCTGTCATC |
| | TGTCTGAATATGCAACGAGGCCACG |
| | GTGCTCACTGTGCGTCATGATCATT |
| | GCAAGCAAGGCCAGGGGTGACATCT |
| | GCCCTCAGTCTGGGTCACTGGGTAG |
| | ATTTGAGGGGTTCTTGGCAATGTCA |
| | GCTCCCTGAGGGCGAGGTTTTGTGT |
| | GCCTTGTTGACTGTTATAACTTGGC |
| | ACAGTGCCTGGCACATGTAGGTACT |
| | CAGTCAATATAGGTCAGAAGAATGA |
| | ATGGATATACTCCCCAACCCTCAAC |
| | TGAGTCTTGAGGAAACTGAGGCACA |
| | GAGGACCTGAAGGGTAAGGGGGTGT |
| | GGAGGTTGTGAAGGCGGGAAGGGGG |
| | GTAGCCCCTTCACCAATGTAAACAA |
| | GGATGTGGGTTCTGCGGCCACACTC |
| | TCCCCCGCCCTCCCCAGCGGCATTT |
| | CCAGCAAGTCACATGTCCTGCGCAC |
| | AGGCTGGGGGCCCCCTGCTGCTGCC |
| | TTTCTTAGAAGCCAGGACCACAGAG |
| | CCCGCACAGTGAGCTACTTGGGGAG |
| | CTATTTCTGTAGACTGAGCTTGGGG |
| | TACTGGGAGGCAGTGCTGTTGGCCT |
| | CCAACTCTCTGCTCCACGTTCCTTC |
| | CACTATTCAACACATGGCAGGAAGT |
| | CAGCCTGTAGGGCTTCACACTACAG |
| | TCTAAGCTGACTGCCATCTGTCCCC |
| | ATCCAGGCGAGGCTGGAAACGGGGG |
| | CAGCAAAAAGCCCAGGGACAAAGTC |
| | CCCTTCCCAGGCACATGTATTATGC |
| | ACTCTGCACCAAGGAAACCTCCAGA |
| | TAAGGTCTAGAGACCAAGGGCCATG |
| | TACTTCGGGGGAGAGGCCAGAGGAC |
| | TTCTGAGGTTTTACAGAGAAAAGCC |
| | AAAGGCAGCCCAGTCAGGGGAAATG |
| | TGTAGCCATAGTGCCGATAAGGAAA |
| | GGCCTTCAACCTGCCCTGGTCAGCT |
| | CTTCCTGTAAGTAGAGGCCCCTTAC |
| | CGAAGGCCCCAGTGGGAGGAAGGGT |
| | CGGGAGTActcgagTCAGCCTCAGG |
| | CTACCTAGGTGCTTTAGAAAGGAGG |
| | CCACCCAGGCCCATGACTACTCCTT |
| | GCCACAGGGAGCCCTGCACACAGAT |
| | GTGCTAAGCTCTCGCTGCCAGCCAG |
| | AGGGAGGAGGGTCTGAGCCAGTCAG |
| | AAGGAGATGGGCCCCAGAGAGTAAG |
| | AAAGGGGGAGGAGGACCCAAGCTGA |
| | TCCAAAAGGTGGGTCTAAGCAGTCA |
| | AGTGGAGGAGGGTTCCAATCTGATG |
| | GCGGAGGGCCCAAGCTCAGCCTAAC |
| | GAGGAGGCCAGGCCCACCAAGGGGC |
| | CCCTGGAGGACTTGTTTCCCTTGTC |
| | CCTTGTGGTTTTTTGCATTTCCTGT |
| | TCCCTTGCTGCTCATTGCGGAAGTT |
| | CCTCTTCTTACCCTGCACCCAGAGC |
| | CTCGCCAGAGAAGACAAGGGCAGAA |
| | AGCACCATGTCTGGCGGACCTATGG |
| | GAGGTAGACCTGGTGGAAGAGGTGC |
| | CCCCGCCGTGCAGCAGAACATCCCC |
| | AGCACCCTGCTGCAGGACCACGAGA |
| | ACCAGCGCCTGTTCGAGATGCTGGG |
| | CCGCAAGTGCCTGACCCTGGCCACC |
| | GCCGTGGTGCAGCTGTACCTGGCCC |
| | TGCCCCCCGGCGCCGAGCACTGGAC |
| | CAAGGAGCACTGCGGCGCCGTGTGC |
| | TTCGTGAAGGACAACCCCCAGAAGA |
| | GCTACTTCATCCGCCTGTACGGCCT |
| | GCAGGCCGGCCGCCTGCTGTGGGAG |
| | CAGGAGCTGTACAGCCAGCTGGTGT |
| | ACAGCACCCCCACCCCCTTCTTCCA |
| | CACCTTCGCCGGCGACGACTGCCAG |
| | GCCGGCCTGAACTTCGCCGACGAGG |
| | ACGAGGCCCAGGCCTTCCGCGCCCT |
| | GGTGCAGGAGAAGATCCAGAAGCGC |
| | AACCAGCGCCAGAGCGGCGACAGAA |
| | GGCAACTGCCTCCTCCACCTACACC |
| | AGCCAACGAGGAAAGAAGAGGCGGA |
| | CTGCCCCCCCTGCCTCTTCATCCTG |
| | GCGGAGATCAAGGTGGACCTCCTGT |
| | GGGACCACTGTCTCTTGGCCTGGCC |
| | ACCGTGGACATCCAGAACCCCGACA |
| | TCACCAGCAGCCGCTACCGCGGCCT |
| | GCCCGCCCCCGGCCCCAGCCCCGCC |
| | GACAAGAAGCGCAGCGGCAAGAAGA |
| | AGATCAGCAAGGCCGACATCGGCGC |
| | CCCCAGCGGCTTCAAGCACGTGAGC |
| | CACGTGGGCTGGGACCCCCAGAACG |
| | GCTTCGACGTGAACAACCTGGACCC |
| | CGACCTGCGCAGCCTGTTCAGCCGC |
| | GCCGGCATCAGCGAGGCCCAGCTGA |
| | CCGACGCCGAGACCAGCAAGCTGAT |
| | CTACGACTTCATCGAGGACCAGGGC |
| | GGCCTGGAGGCCGTGCGCCAGGAGA |
| | TGCGCCGCCAGGAGCCCCTGCCACC |
| | ACCTCCACCTCCATCTAGAGGCGGC |
| | AACCAGCTGCCCCGCCCTCCTATCG |
| | TTGGCGGCAACAAGGGAAGATCTGG |
| | CCCTCTGCCTCCTGTGCCTCTGGGA |
| | ATCGCTCCACCACCACCAACACCTA |
| | GAGGCCCGCCTCCACCAGGCAGAGG |
| | TGGTCCTCCGCCGCCACCTCCTCCA |
| | GCCACCGGCCGCAGCGGCCCCCTTC |
| | CTCCTCCACCACCTGGTGCTGGTGG |
| | ACCTCCAATGCCACCGCCACCGCCT |
| | CCGCCACCTCCGCCTCCAAGTTCTG |
| | GAAATGGACCTGCTCCTCCTCCTTT |
| | GCCTCCTGCTTTGGTTCCTGCTGGC |
| | GGATTGGCTCCAGGCGGAGGAAGAG |
| | GCGCACTGCTGGACCAGATCCGCCA |
| | GGGCATCCAGCTGAACAAGACCCCT |
| | GGCGCTCCTGAGAGTTCTGCTCTGC |
| | AACCGCCACCACAGTCTAGCGAAGG |
| | ACTTGTGGGAGCCCTGATGCACGTG |
| | ATGCAGAAGCGCAGCCGCGCCATCC |
| | ACAGCAGCGACGAGGGCGAGGACCA |
| | GGCCGGCGACGAGGACGAGGACGAC |
| | GAGTGGGACGACTAActgcaggaat |
| | tcgagcatcttaccgccatttattc |
| | ccatatttgttctgtttttcttgat |
| | ttgggtatacatttaaatgttaata |
| | aaacaaaatggtggggcaatcattt |
| | acatttttagggatatgtaattact |
| | agttcaggtgtattgccacaagaca |
| | aacatgttaagaaactttcccgtta |
| | tttacgctctgttcctgttaatcaa |
| | cctctggattacaaaatttgtgaaa |
| | gattgactgatattcttaactatgt |
| | tgctccttttacgctgtgtggatat |
| | gctgctttaatgcctctgtatcatg |
| | ctattgcttcccgtacggctttcgt |
| | tttctcctccttgtataaatcctgg |
| | ttgctgtctctttatgaggagttgt |
| | ggcccgttgtccgtcaacgtggcgt |
| | ggtgtgctctgtgtttgctgacgca |
| | acccccactggctggggcattgcca |
| | ccacctgtcaactcctttctgggac |
| | tttcgctttccccctcccgatcgcc |
| | acggcagaactcatcgccgcctgcc |
| | ttgcccgctgctggacaggggctag |
| | gttgctgggcactgataattccgtg |
| | gtgttgtcggggaagggcctgctgc |
| | cggctctgcggcctcttccgcgtct |
| | tcgccttcgccctcagacgagtcgg |
| | atctccctttgggccgcctccccgc |
| | ctggaattcgagctcggtaccttta |
| | agaccaatgacttacaaggcagctg |
| | tagatcttagccactttttaaaaga |
| | aaaggggggactggaagggctaatt |
| | cactcccaacgaagacaagatctgc |
| | tttttgcttgtactgggtctctctg |
| | gttagaccagatctgagcctgggag |
| | ctctctggctaactagggaacctac |
| | tgcttaagcctcaataaagcttgcc |
| | ttgagtgcttCAAGTAGTGTGTGCC |
| | CGTCTGTTGTGTGACTCTGGTAACT |
| | AGAGATCCCTCAGACCCTTTTAGTC |
| | AGTGTGGAAAATCTCTAGCagtagt |
| | agttcatgtcatcttattattcagt |
| | atttataacttgcaaagaaatgaat |
| | atcagagagtgagaggaacttgttt |
| | attgcagcttataatggttacaaat |
| | aaagcaatagcatcacaaatttcac |
| | aaataaagcatttttttcactgcat |
| | tctagttgtggtttgtccaaactca |
| | tcaatgtatcttatcatgtctggct |
| | ctagctatcccgcccctaactccgc |
| | ccatcccgcccctaactccgcccag |
| | ttccgcccattctccgccccatggc |
| | tgactaattttttttatttatgcag |
| | aggccgaggccgcctcggcctctga |
| | gctattccagaagtagtgaggaggc |
| | ttttttggaggcctagggacgtacc |
| | caattcgccctatagtgagtcgtat |
| | tacgcgcgctcactggccgtcgttt |
| | tacaacgtcgtgactgggaaaaccc |
| | tggcgttacccaacttaatcgcctt |
| | gcagcacatccccctttcgccagct |
| | ggcgtaatagcgaagaggcccgcac |
| | cgatcgcccttcccaacagttgcgc |
| | agcctgaatggcgaatgggacgcgc |
| | cctgtagcggcgcattaagcgcggc |
| | gggtgtggtggttacgcgcagcgtg |
| | accgctacacttgccagcgccctag |
| | cgcccgctcctttcgctttcttccc |
| | ttcctttctcgccacgttcgccggc |
| | tttccccgtcaagctctaaatcggg |
| | ggctccctttagggttccgatttag |
| | tgctttacggcacctcgaccccaaa |
| | aaacttgattagggtgatggttcac |
| | gtagtgggccatcgccctgatagac |
| | ggtttttcgccctttgacgttggag |
| | tccacgttctttaatagtggactct |
| | tgttccaaactggaacaacactcaa |
| | ccctatctcggtctattcttttgat |
| | ttataagggattttgccgatttcgg |
| | cctattggttaaaaaatgagctgat |
| | ttaacaaaaatttaacgcgaatttt |
| | aacaaaatattaacgcttacaattt |
| | aggtggcacttttcggggaaatgtg |
| | cgcggaacccctatttgtttatttt |
| | tctaaatacattcaaatatgtatcc |
| | gctcatgagacaataaccctgataa |
| | atgcttcaataatagcacctagatc |
| | aagagacaggatgaggatcgtttcg |
| | catgattgaacaagatggattgcac |
| | gcaggttctccggccgcttgggtgg |
| | agaggctattcggctatgactgggc |
| | acaacagacaatcggctgctctgat |
| | gccgccgtgttccggctgtcagcgc |
| | aggggcgcccggttctttttgtcaa |
| | gaccgacctgtccggtgccctgaat |
| | gaactgcaagacgaggcagcgcggc |
| | tatcgtggctggccacgacgggcgt |
| | tccttgcgcagctgtgctcgacgtt |
| | gtcactgaagcgggaagggactggc |
| | tgctattgggcgaagtgccggggca |
| | ggatctcctgtcatctcaccttgct |
| | cctgccgagaaagtatccatcatgg |
| | ctgatgcaatgcggcggctgcatac |
| | gcttgatccggctacctgcccattc |
| | gaccaccaagcgaaacatcgcatcg |
| | agcgagcacgtactcggatggaagc |
| | cggtcttgtcgatcaggatgatctg |
| | gacgaagagcatcaggggctcgcgc |
| | cagccgaactgttcgccaggctcaa |
| | ggcgagcatgcccgacggcgaggat |
| | ctcgtcgtgacccatggcgatgcct |
| | gcttgccgaatatcatggtggaaaa |
| | tggccgcttttctggattcatcgac |
| | tgtggccggctgggtgtggcggacc |
| | gctatcaggacatagcgttggctac |
| | ccgtgatattgctgaagagcttggc |
| | ggcgaatgggctgaccgcttcctcg |
| | tgctttacggtatcgccgctcccga |
| | ttcgcagcgcatcgccttctatcgc |
| | cttcttgacgagttcttctgaatta |
| | ttaacgcttacaatttcctgatgcg |
| | gtattttctccttacgcatctgtgc |
| | ggtatttcacaccgcatcaggtggc |
| | acttttcggggaaatgtgcgcggaa |
| | cccctatttgtttatttttctaaat |
| | acattcaaatatgtatccgctcatg |
| | accaaaatcccttaacgtgagtttt |
| | cgttccactgagcgtcagaccccgt |
| | agaaaagatcaaaggatcttcttga |
| | gatcctttttttctgcgcgtaatct |
| | gctgcttgcaaacaaaaaaaccacc |
| | gctaccagcggtggtttgtttgccg |
| | gatcaagagctaccaactctttttc |
| | cgaaggtaactggcttcagcagagc |
| | gcagataccaaatactgttcttcta |
| | gtgtagccgtagttaggccaccact |
| | tcaagaactctgtagcaccgcctac |
| | atacctcgctctgctaatcctgtta |
| | ccagtggctgctgccagtggcgata |
| | agtcgtgtcttaccgggttggactc |
| | aagacgatagttaccggataaggcg |
| | cagcggtcgggctgaacggggggtt |
| | cgtgcacacagcccagcttggagcg |
| | aacgacctacaccgaactgagatac |
| | ctacagcgtgagctatgagaaagcg |
| | ccacgcttcccgaagggagaaaggc |
| | ggacaggtatccggtaagcggcagg |
| | gtcggaacaggagagcgcacgaggg |
| | agcttccagggggaaacgcctggta |
| | tctttatagtcctgtcgggtttcgc |
| | cacctctgacttgagcgtcgatttt |
| | tgtgatgctcgtcaggggggcggag |
| | cctatggaaaaacgccagcaacgcg |
| | gcctttttacggttcctggcctttt |
| | gctggccttttgctcacatgttctt |
| | tcctgcgttatcccctgattctgtg |
| | gataaccgtattaccgcctttgagt |
| | gagctgataccgctcgccgcagccg |
| | aacgaccgagcgcagcgagtcagtg |
| | agcgaggaagcggaagagcgcccaa |
| | tacgcaaaccgcctctccccgcgcg |
| | ttggccgattcattaatgcagctgg |
| | cacgacaggtttcccgactggaaag |
| | cgggcagtgagcgcaacgcaattaa |
| | tgtgagttagctcactcattaggca |
| | ccccaggctttacactttatgcttc |
| | cggctcgtatgttgtgtggaattgt |
| | gagcggataacaatttcacacagga |
| | aacagctatgaccatgattacgcca |
| | agcgcgcaattaaccctcactaaag |
| | ggaacaaaagctggagctgcaagct |
| | tggccattgcatacgttgtatccat |
| | atcataatatgtacatttatattgg |
| | ctcatgtccaacattaccgccatgt |
| | tgacattgattattgactagttatt |
| | aatagtaatcaattacggggtcatt |
| | agttcatagcccatatatggAGTTC |
| | CGCGTTACATAACTTACGGTAAATG |
| | GCCCGCCTGGCTGACCGCCCAACGA |
| | CCCCCGCCCATTGACGTCAATAATG |
| | ACGTATGTTCCCATAGTAACGCCAA |
| | TAGGGACTTTCCATTGACGTCAATG |
| | GGTGGAGTATTTACGGTAAACTGCC |
| | CACTTGGCAGTACATCAAGTGTATC |
| | ATATGCCAAGTACGCCCCCTATTGA |
| | CGTCAATGACGGTAAATGGCCCGCC |
| | TGGCATTATGCCCAGTA |
| SEQ ID NO: 5 | CATGACCTTATGGGACTTTCCTACT |
| Sequence of | TGGCAGTACATCTACGTATTAGTCA |
| WASVec2.0 V2 | TCGCTATTACCATGGTGATGCGGTT |
| Synthetic | TTGGCAGTACATCAATGGGCGTGGA |
| construct | TAGCGGTTTGACTCACGGGGATTTC |
| | CAAGTCTCCACCCCATTGACGTCAA |
| | TGGGAGTTTGTTTTGGCACCAAAAT |
| | CAACGGGACTTTCCAAAATGTCGTA |
| | ACAACTCCGCCCCATTGACGCAAAT |
| | GGGCGGTAGGCGTGTACGGTGGGAG |
| | GTCTATATAAGCAGAGCTCGTTTAG |
| | TGAACCGGGGTCTCTCTGGTTAGAC |
| | CAGATCTGAGCCTGGGAGCTCTCTG |
| | GCTAACTAGGGAACCCACTGCTTAA |
| | GCCTCAATAAAGCTTGCCTTGAGTG |
| | CTTCAAGTAGTGTGTGCCCGTCTGT |
| | TGTGTGACTCTGGTAACTAGAGATC |
| | CCTCAGACCCTTTTAGTCAGTGTGG |
| | AAAATCTCTAGCagtggcgcccgaa |
| | cagggacttgaaagcgaaagggaaa |
| | ccagaggagctctctcgacgcagga |
| | ctcggcttgctgaagcgcgcacggc |
| | aagaggcgaggggcggcgactggtg |
| | agtacgccaaaaattttgactagcg |
| | gaggctagaaggagagagatgggtg |
| | cgagagcgtcagtattaagcggggg |
| | agaattagatcgcgatgggaaaaaa |
| | ttcggttaaggccagggggaaagaa |
| | aaaatataaattaaaacatatagta |
| | tgggcaagcagggagctagaacgat |
| | tcgcagttaatcctggcctgttaga |
| | aacatcagaaggctgtagacaaata |
| | ctgggacagctacaaccatcccttc |
| | agacaggatcagaagaacttagatc |
| | attatataatacagtagcaaccctc |
| | tattgtgtgcatcaaaggatagaga |
| | taaaagacaccaaggaagctttaga |
| | caagatagaggaagagcaaaaaaaa |
| | gtaagaccaccgcacagcaagcggc |
| | cgctgatcttcagacctggaggagg |
| | agatatgagggacaattggagaagt |
| | gaattatataaatataaagtagtaa |
| | aaattgaaccattaggagtagcacc |
| | caccaaggcaaagagaagagtggtg |
| | cagagagaaaaaagagcagtgggaa |
| | taggagctttgttccttgggttctt |
| | gggagcagcaggaagcactatgggc |
| | gcagcgtcaatgacgctgacggtac |
| | aggccagacaattattgtctggtat |
| | agtgcagcagcagaacaatttgctg |
| | agggctattgaggcgcaacagcatc |
| | tgttgcaactcacagtctggggcat |
| | caagcagctccaggcaagaatcctg |
| | gctgtggaaagatacctaaaggatc |
| | aacagctcctggggatttggggttg |
| | ctctggaaaactcatttgcaccact |
| | gctgtgccttggaatgctagttgga |
| | gtaataaatctctggaacagatttg |
| | gaatcacacgacctggatggagtgg |
| | gacagagaaattaacaattacacaa |
| | gcttaatacactccttaattgaaga |
| | atcgcaaaaccagcaagaaaagaat |
| | gaacaagaattattggaattagata |
| | aatgggcaagtttgtggaattggtt |
| | taacataacaaattggctgtggtat |
| | ataaaattattcataatgatagtag |
| | gaggcttggtaggtttaagaatagt |
| | ttttgctgtactttctatagtgaat |
| | agagttaggcagggatattcaccat |
| | tatcgtttcagacccacctcccaac |
| | cccgaggggacccgacaggcccgaa |
| | ggaatagaagaagaaggtggagaga |
| | gagacagagacagatccattcgatt |
| | agtgaacggatctcgacggtatcgg |
| | ttaacttttaaaagaaaagggggga |
| | ttggggggtacagtgcaggggaaag |
| | aatagtagacataatagcaacagac |
| | atacaaactaaagaattacaaaaac |
| | aaattacaaaaattcaaaattttat |
| | cgatcacgagactagcctcgagCCA |
| | CTGGAATTGCCCTTCTGAAGTTTTG |
| | CACTGATATCTCCGTGCTCCTGCTC |
| | AAGGACCCACAGTGGCTCCTTCCTT |
| | CCTGTTACCATGTGGATGCCACTGA |
| | TTAGATCCTCAGCTTCCATGTCAGG |
| | CTGTCATCTGTCTGAATATGCAACG |
| | AGGCCACGGTGCTCACTGTGCGTCA |
| | TGATCATTGCAAGCAAGGCCAGGGG |
| | TGACATCTGCCCTCAGTCTGGGTCA |
| | CTGGGTAGACCTGAAGGGTAAGGGG |
| | GTGTGGAGGTTGTGAAGGCGGGAAG |
| | GGGGGTAGCCCCTTCACCAATGTAA |
| | ACAAGGATGTGGGTTCTGCGGCCAC |
| | ACTCTCCCCCGCCCTCCCCAGCGGC |
| | ATTTCCAGCAAGTCACATGTCCTGC |
| | GCACAGGCTGGGGGCCCCCTGCTGC |
| | TGCCTTTCTTAGAAGCCAGGACCAC |
| | AGAGCCCGCACAGTGAGCTACTTGG |
| | GGAGCTATTTCTGTAGACTGAGCTT |
| | GGGGTACTGGGAGGCAGTGCTGTTG |
| | GCCTCCAACTCTCTGCTCCACGTTC |
| | CTTCCACTATTCAACACATGGCAGG |
| | AAGTCAGCCTGTAGGGCTTCACACT |
| | ACAGTCTAAGCTGACTGCCATCTGT |
| | CCCCATCCAGGCGAGGCTGGAAACG |
| | GGGGCAGCAAAAAGCCCAGGGACAA |
| | AGTCCCCTTCCCAGGCACATGTATT |
| | ATGCACTCTGCACCAAGGAAACCTC |
| | CAGATAAGGTCTAGAGACCAAGGGC |
| | CATGTACTTCGGGGGAGAGGCCAGA |
| | GGACTTCTGAGGTTTTACAGAGAAA |
| | AGCCAAAGGCAGCCCAGTCAGGGGA |
| | AATGTGTAGCCATAGTGCCGATAAG |
| | GAAAGGCCTTCAACCTGCCCTGGTC |
| | AGCTCTTCCTGTAAGTAGAGGCCCC |
| | TTACCGAAGGCCCCAGTGGGAGGAA |
| | GGGTCGGGAGTActcgagTCAGCCT |
| | CAGGCTACCTAGGTGCTTTAGAAAG |
| | GAGGCCACCCAGGCCCATGACTACT |
| | CCTTGCCACAGGGAGCCCTGCACAC |
| | AGATGTGCTAAGCTCTCGCTGCCAG |
| | CCAGAGGGAGGAGGGTCTGAGCCAG |
| | TCAGAAGGAGATGGGCCCCAGAGAG |
| | TAAGAAAGGGGGAGGAGGACCCAAG |
| | CTGATCCAAAAGGTGGGTCTAAGCA |
| | GTCAAGTGGAGGAGGGTTCCAATCT |
| | GATGGCGGAGGGCCCAAGCTCAGCC |
| | TAACGAGGAGGCCAGGCCCACCAAG |
| | GGGCCCCTGGAGGACTTGTTTCCCT |
| | TGTCCCTTGTGGTTTTTTGCATTTC |
| | CTGTTCCCTTGCTGCTCATTGCGGA |
| | AGTTCCTCTTCTTACCCTGCACCCA |
| | GAGCCTCGCCAGAGAAGACAAGGGC |
| | AGAAAGCACCATGTCTGGCGGACCT |
| | ATGGGAGGTAGACCTGGTGGAAGAG |
| | GTGCCCCCGCCGTGCAGCAGAACAT |
| | CCCCAGCACCCTGCTGCAGGACCAC |
| | GAGAACCAGCGCCTGTTCGAGATGC |
| | TGGGCCGCAAGTGCCTGACCCTGGC |
| | CACCGCCGTGGTGCAGCTGTACCTG |
| | GCCCTGCCCCCCGGCGCCGAGCACT |
| | GGACCAAGGAGCACTGCGGCGCCGT |
| | GTGCTTCGTGAAGGACAACCCCCAG |
| | AAGAGCTACTTCATCCGCCTGTACG |
| | GCCTGCAGGCCGGCCGCCTGCTGTG |
| | GGAGCAGGAGCTGTACAGCCAGCTG |
| | GTGTACAGCACCCCCACCCCCTTCT |
| | TCCACACCTTCGCCGGCGACGACTG |
| | CCAGGCCGGCCTGAACTTCGCCGAC |
| | GAGGACGAGGCCCAGGCCTTCCGCG |
| | CCCTGGTGCAGGAGAAGATCCAGAA |
| | GCGCAACCAGCGCCAGAGCGGCGAC |
| | AGAAGGCAACTGCCTCCTCCACCTA |
| | CACCAGCCAACGAGGAAAGAAGAGG |
| | CGGACTGCCCCCCCTGCCTCTTCAT |
| | CCTGGCGGAGATCAAGGTGGACCTC |
| | CTGTGGGACCACTGTCTCTTGGCCT |
| | GGCCACCGTGGACATCCAGAACCCC |
| | GACATCACCAGCAGCCGCTACCGCG |
| | GCCTGCCCGCCCCCGGCCCCAGCCC |
| | CGCCGACAAGAAGCGCAGCGGCAAG |
| | AAGAAGATCAGCAAGGCCGACATCG |
| | GCGCCCCCAGCGGCTTCAAGCACGT |
| | GAGCCACGTGGGCTGGGACCCCCAG |
| | AACGGCTTCGACGTGAACAACCTGG |
| | ACCCCGACCTGCGCAGCCTGTTCAG |
| | CCGCGCCGGCATCAGCGAGGCCCAG |
| | CTGACCGACGCCGAGACCAGCAAGC |
| | TGATCTACGACTTCATCGAGGACCA |
| | GGGCGGCCTGGAGGCCGTGCGCCAG |
| | GAGATGCGCCGCCAGGAGCCCCTGC |
| | CACCACCTCCACCTCCATCTAGAGG |
| | CGGCAACCAGCTGCCCCGCCCTCCT |
| | ATCGTTGGCGGCAACAAGGGAAGAT |
| | CTGGCCCTCTGCCTCCTGTGCCTCT |
| | GGGAATCGCTCCACCACCACCAACA |
| | CCTAGAGGCCCGCCTCCACCAGGCA |
| | GAGGTGGTCCTCCGCCGCCACCTCC |
| | TCCAGCCACCGGCCGCAGCGGCCCC |
| | CTTCCTCCTCCACCACCTGGTGCTG |
| | GTGGACCTCCAATGCCACCGCCACC |
| | GCCTCCGCCACCTCCGCCTCCAAGT |
| | TCTGGAAATGGACCTGCTCCTCCTC |
| | CTTTGCCTCCTGCTTTGGTTCCTGC |
| | TGGCGGATTGGCTCCAGGCGGAGGA |
| | AGAGGCGCACTGCTGGACCAGATCC |
| | GCCAGGGCATCCAGCTGAACAAGAC |
| | CCCTGGCGCTCCTGAGAGTTCTGCT |
| | CTGCAACCGCCACCACAGTCTAGCG |
| | AAGGACTTGTGGGAGCCCTGATGCA |
| | CGTGATGCAGAAGCGCAGCCGCGCC |
| | ATCCACAGCAGCGACGAGGGCGAGG |
| | ACCAGGCCGGCGACGAGGACGAGGA |
| | CGACGAGTGGGACGACTAActgcag |
| | gaattcgagcatcttaccgccattt |
| | attcccatatttgttctgtttttct |
| | tgatttgggtatacatttaaatgtt |
| | aataaaacaaaatggtggggcaatc |
| | atttacatttttagggatatgtaat |
| | tactagttcaggtgtattgccacaa |
| | gacaaacatgttaagaaactttccc |
| | gttatttacgctctgttcctgttaa |
| | tcaacctctggattacaaaatttgt |
| | gaaagattgactgatattcttaact |
| | atgttgctccttttacgctgtgtgg |
| | atatgctgctttaatgcctctgtat |
| | catgctattgcttcccgtacggctt |
| | tcgttttctcctccttgtataaatc |
| | ctggttgctgtctctttatgaggag |
| | ttgtggcccgttgtccgtcaacgtg |
| | gcgtggtgtgctctgtgtttgctga |
| | cgcaacccccactggctggggcatt |
| | gccaccacctgtcaactcctttctg |
| | ggactttcgctttccccctcccgat |
| | cgccacggcagaactcatcgccgcc |
| | tgccttgcccgctgctggacagggg |
| | ctaggttgctgggcactgataattc |
| | cgtggtgttgtcggggaagggcctg |
| | ctgccggctctgcggcctcttccgc |
| | gtcttcgccttcgccctcagacgag |
| | tcggatctccctttgggccgcctcc |
| | ccgcctggaattcgagctcggtacc |
| | tttaagaccaatgacttacaaggca |
| | gctgtagatcttagccactttttaa |
| | aagaaaaggggggactggaagggct |
| | aattcactcccaacgaagacaagat |
| | ctgctttttgcttgtactgggtctc |
| | tctggttagaccagatctgagcctg |
| | ggagctctctggctaactagggaac |
| | ctactgcttaagcctcaataaagct |
| | tgccttgagtgcttCAAGTAGTGTG |
| | TGCCCGTCTGTTGTGTGACTCTGGT |
| | AACTAGAGATCCCTCAGACCCTTTT |
| | AGTCAGTGTGGAAAATCTCTAGCag |
| | tagtagttcatgtcatcttattatt |
| | cagtatttataacttgcaaagaaat |
| | gaatatcagagagtgagaggaactt |
| | gtttattgcagcttataatggttac |
| | aaataaagcaatagcatcacaaatt |
| | tcacaaataaagcatttttttcact |
| | gcattctagttgtggtttgtccaaa |
| | ctcatcaatgtatcttatcatgtct |
| | ggctctagctatcccgcccctaact |
| | ccgcccatcccgcccctaactccgc |
| | ccagttccgcccattctccgcccca |
| | tggctgactaattttttttatttat |
| | gcagaggccgaggccgcctcggcct |
| | ctgagctattccagaagtagtgagg |
| | aggcttttttggaggcctagggacg |
| | tacccaattcgccctatagtgagtc |
| | gtattacgcgcgctcactggccgtc |
| | gttttacaacgtcgtgactgggaaa |
| | accctggcgttacccaacttaatcg |
| | ccttgcagcacatccccctttcgcc |
| | agctggcgtaatagcgaagaggccc |
| | gcaccgatcgcccttcccaacagtt |
| | gcgcagcctgaatggcgaatgggac |
| | gcgccctgtagcggcgcattaagcg |
| | cggcgggtgtggtggttacgcgcag |
| | cgtgaccgctacacttgccagcgcc |
| | ctagcgcccgctcctttcgctttct |
| | tcccttcctttctcgccacgttcgc |
| | cggctttccccgtcaagctctaaat |
| | cgggggctccctttagggttccgat |
| | ttagtgctttacggcacctcgaccc |
| | caaaaaacttgattagggtgatggt |
| | tcacgtagtgggccatcgccctgat |
| | agacggtttttcgccctttgacgtt |
| | ggagtccacgttctttaatagtgga |
| | ctcttgttccaaactggaacaacac |
| | tcaaccctatctcggtctattcttt |
| | tgatttataagggattttgccgatt |
| | tcggcctattggttaaaaaatgagc |
| | tgatttaacaaaaatttaacgcgaa |
| | ttttaacaaaatattaacgcttaca |
| | atttaggtggcacttttcggggaaa |
| | tgtgcgcggaacccctatttgttta |
| | tttttctaaatacattcaaatatgt |
| | atccgctcatgagacaataaccctg |
| | ataaatgcttcaataatagcaccta |
| | gatcaagagacaggatgaggatcgt |
| | ttcgcatgattgaacaagatggatt |
| | gcacgcaggttctccggccgcttgg |
| | gtggagaggctattcggctatgact |
| | gggcacaacagacaatcggctgctc |
| | tgatgccgccgtgttccggctgtca |
| | gcgcaggggcgcccggttctttttg |
| | tcaagaccgacctgtccggtgccct |
| | gaatgaactgcaagacgaggcagcg |
| | cggctatcgtggctggccacgacgg |
| | gcgttccttgcgcagctgtgctcga |
| | cgttgtcactgaagcgggaagggac |
| | tggctgctattgggcgaagtgccgg |
| | ggcaggatctcctgtcatctcacct |
| | tgctcctgccgagaaagtatccatc |
| | atggctgatgcaatgcggcggctgc |
| | atacgcttgatccggctacctgccc |
| | attcgaccaccaagcgaaacatcgc |
| | atcgagcgagcacgtactcggatgg |
| | aagccggtcttgtcgatcaggatga |
| | tctggacgaagagcatcaggggctc |
| | gcgccagccgaactgttcgccaggc |
| | tcaaggcgagcatgcccgacggcga |
| | ggatctcgtcgtgacccatggcgat |
| | gcctgcttgccgaatatcatggtgg |
| | aaaatggccgcttttctggattcat |
| | cgactgtggccggctgggtgtggcg |
| | gaccgctatcaggacatagcgttgg |
| | ctacccgtgatattgctgaagagct |
| | tggcggcgaatgggctgaccgcttc |
| | ctcgtgctttacggtatcgccgctc |
| | ccgattcgcagcgcatcgccttcta |
| | tcgccttcttgacgagttcttctga |
| | attattaacgcttacaatttcctga |
| | tgcggtattttctccttacgcatct |
| | gtgcggtatttcacaccgcatcagg |
| | tggcacttttcggggaaatgtgcgc |
| | ggaacccctatttgtttatttttct |
| | aaatacattcaaatatgtatccgct |
| | catgaccaaaatcccttaacgtgag |
| | ttttcgttccactgagcgtcagacc |
| | ccgtagaaaagatcaaaggatcttc |
| | ttgagatcctttttttctgcgcgta |
| | atctgctgcttgcaaacaaaaaaac |
| | caccgctaccagcggtggtttgttt |
| | gccggatcaagagctaccaactctt |
| | tttccgaaggtaactggcttcagca |
| | gagcgcagataccaaatactgttct |
| | tctagtgtagccgtagttaggccac |
| | cacttcaagaactctgtagcaccgc |
| | ctacatacctcgctctgctaatcct |
| | gttaccagtggctgctgccagtggc |
| | gataagtcgtgtcttaccgggttgg |
| | actcaagacgatagttaccggataa |
| | ggcgcagcggtcgggctgaacgggg |
| | ggttcgtgcacacagcccagcttgg |
| | agcgaacgacctacaccgaactgag |
| | atacctacagcgtgagctatgagaa |
| | agcgccacgcttcccgaagggagaa |
| | aggcggacaggtatccggtaagcgg |
| | cagggtcggaacaggagagcgcacg |
| | agggagcttccagggggaaacgcct |
| | ggtatctttatagtcctgtcgggtt |
| | tcgccacctctgacttgagcgtcga |
| | tttttgtgatgctcgtcaggggggc |
| | ggagcctatggaaaaacgccagcaa |
| | cgcggcctttttacggttcctggcc |
| | ttttgctggccttttgctcacatgt |
| | tctttcctgcgttatcccctgattc |
| | tgtggataaccgtattaccgccttt |
| | gagtgagctgataccgctcgccgca |
| | gccgaacgaccgagcgcagcgagtc |
| | agtgagcgaggaagcggaagagcgc |
| | ccaatacgcaaaccgcctctccccg |
| | cgcgttggccgattcattaatgcag |
| | ctggcacgacaggtttcccgactgg |
| | aaagcgggcagtgagcgcaacgcaa |
| | ttaatgtgagttagctcactcatta |
| | ggcaccccaggctttacactttatg |
| | cttccggctcgtatgttgtgtggaa |
| | ttgtgagcggataacaatttcacac |
| | aggaaacagctatgaccatgattac |
| | gccaagcgcgcaattaaccctcact |
| | aaagggaacaaaagctggagctgca |
| | agcttggccattgcatacgttgtat |
| | ccatatcataatatgtacatttata |
| | ttggctcatgtccaacattaccgcc |
| | atgttgacattgattattgactagt |
| | tattaatagtaatcaattacggggt |
| | cattagttcatagcccatatatggA |
| | GTTCCGCGTTACATAACTTACGGTA |
| | AATGGCCCGCCTGGCTGACCGCCCA |
| | ACGACCCCCGCCCATTGACGTCAAT |
| | AATGACGTATGTTCCCATAGTAACG |
| | CCAATAGGGACTTTCCATTGACGTC |
| | AATGGGTGGAGTATTTACGGTAAAC |
| | TGCCCACTTGGCAGTACATCAAGTG |
| | TATCATATGCCAAGTACGCCCCCTA |
| | TTGACGTCAATGACGGTAAATGGCC |
| | CGCCTGGCATTATGCCCAGTA |
| SEQ ID NO: 6 | CATGACCTTATGGGACTTTCCTACT |
| Sequence of | TGGCAGTACATCTACGTATTAGTCA |
| WASVec2.0 V3 | TCGCTATTACCATGGTGATGCGGTT |
| Synthetic | TTGGCAGTACATCAATGGGCGTGGA |
| construct | TAGCGGTTTGACTCACGGGGATTTC |
| | CAAGTCTCCACCCCATTGACGTCAA |
| | TGGGAGTTTGTTTTGGCACCAAAAT |
| | CAACGGGACTTTCCAAAATGTCGTA |
| | ACAACTCCGCCCCATTGACGCAAAT |
| | GGGCGGTAGGCGTGTACGGTGGGAG |
| | GTCTATATAAGCAGAGCTCGTTTAG |
| | TGAACCGGGGTCTCTCTGGTTAGAC |
| | CAGATCTGAGCCTGGGAGCTCTCTG |
| | GCTAACTAGGGAACCCACTGCTTAA |
| | GCCTCAATAAAGCTTGCCTTGAGTG |
| | CTTCAAGTAGTGTGTGCCCGTCTGT |
| | TGTGTGACTCTGGTAACTAGAGATC |
| | CCTCAGACCCTTTTAGTCAGTGTGG |
| | AAAATCTCTAGCagtggcgcccgaa |
| | cagggacttgaaagcgaaagggaaa |
| | ccagaggagctctctcgacgcagga |
| | ctcggcttgctgaagcgcgcacggc |
| | aagaggcgaggggcggcgactggtg |
| | agtacgccaaaaattttgactagcg |
| | gaggctagaaggagagagatgggtg |
| | cgagagcgtcagtattaagcggggg |
| | agaattagatcgcgatgggaaaaaa |
| | ttcggttaaggccagggggaaagaa |
| | aaaatataaattaaaacatatagta |
| | tgggcaagcagggagctagaacgat |
| | tcgcagttaatcctggcctgttaga |
| | aacatcagaaggctgtagacaaata |
| | ctgggacagctacaaccatcccttc |
| | agacaggatcagaagaacttagatc |
| | attatataatacagtagcaaccctc |
| | tattgtgtgcatcaaaggatagaga |
| | taaaagacaccaaggaagctttaga |
| | caagatagaggaagagcaaaacaaa |
| | agtaagaccaccgcacagcaagcgg |
| | ccgctgatcttcagacctggaggag |
| | gagatatgagggacaattggagaag |
| | tgaattatataaatataaagtagta |
| | aaaattgaaccattaggagtagcac |
| | ccaccaaggcaaagagaagagtggt |
| | gcagagagaaaaaagagcagtggga |
| | ataggagctttgttccttgggttct |
| | tgggagcagcaggaagcactatggg |
| | cgcagcgtcaatgacgctgacggta |
| | caggccagacaattattgtctggta |
| | tagtgcagcagcagaacaatttgct |
| | gagggctattgaggcgcaacagcat |
| | ctgttgcaactcacagtctggggca |
| | tcaagcagctccaggcaagaatcct |
| | ggctgtggaaagatacctaaaggat |
| | caacagctcctggggatttggggtt |
| | gctctggaaaactcatttgcaccac |
| | tgctgtgccttggaatgctagttgg |
| | agtaataaatctctggaacagattt |
| | ggaatcacacgacctggatggagtg |
| | ggacagagaaattaacaattacaca |
| | agcttaatacactccttaattgaag |
| | aatcgcaaaaccagcaagaaaagaa |
| | tgaacaagaattattggaattagat |
| | aaatgggcaagtttgtggaattggt |
| | ttaacataacaaattggctgtggta |
| | tataaaattattcataatgatagta |
| | ggaggcttggtaggtttaagaatag |
| | tttttgctgtactttctatagtgaa |
| | tagagttaggcagggatattcacca |
| | ttatcgtttcagacccacctcccaa |
| | ccccgaggggacccgacaggcccga |
| | aggaatagaagaagaaggtggagag |
| | agagacagagacagatccattcgat |
| | tagtgaacggatctcgacggtatcg |
| | gttaacttttaaaagaaaagggggg |
| | attggggggtacagtgcaggggaaa |
| | gaatagtagacataatagcaacaga |
| | catacaaactaaagaattacaaaaa |
| | caaattacaaaaattcaaaatttta |
| | tcgatcacgagactagcctcgagGA |
| | CCTGAAGGGTAAGGGGGTGTGGAGG |
| | TTGTGAAGGCGGGAAGGGGGGTAGC |
| | CCCTTCACCAATGTAAACAAGGATG |
| | TGGGTTCTGCGGCCACACTCTCCCC |
| | CGCCCTCCCCAGCGGCATTTCCAGC |
| | AAGTCACATGTCCTGCGCACAGGCT |
| | GGGGGCCCCCTGCTGCTGCCTTTCT |
| | TAGAAGCCAGGACCACAGAGCCCGC |
| | ACAGTGAGCTACTTGGGGAGCTATT |
| | TCTGTAGACTGAGCTTGGGGTACTG |
| | GGAGGCAGTGCTGTTGGCCTCCAAC |
| | TCTCTGCTCCACGTTCCTTCCACTA |
| | TTCAACACATGGCAGGAAGTCAGCC |
| | TGTAGGGCTTCACACTACAGTCTAA |
| | GCTGACTGCCATCTGTCCCCATCCA |
| | GGCGAGGCTGGAAACGGGGGCAGCA |
| | AAAAGCCCAGGGACAAAGTCCCCTT |
| | CCCAGGCACATGTATTATGCACTCT |
| | GCACCAAGGAAACCTCCAGATAAGG |
| | TCTAGAGACCAAGGGCCATGTACTT |
| | CGGGGGAGAGGCCAGAGGACTTCTG |
| | AGGTTTTACAGAGAAAAGCCAAAGG |
| | CAGCCCAGTCAGGGGAAATGTGTAG |
| | CCATAGTGCCGATAAGGAAAGGCCT |
| | TCAACCTGCCCTGGTCAGCTCTTCC |
| | TGTAAGTAGAGGCCCCTTACCGAAG |
| | GCCCCAGTGGGAGGAAGGGTCGGGA |
| | GTActcgagTCAGCCTCAGGCTACC |
| | TAGGTGCTTTAGAAAGGAGGCCACC |
| | CAGGCCCATGACTACTCCTTGCCAC |
| | AGGGAGCCCTGCACACAGATGTGCT |
| | AAGCTCTCGCTGCCAGCCAGAGGGA |
| | GGAGGGTCTGAGCCAGTCAGAAGGA |
| | GATGGGCCCCAGAGAGTAAGAAAGG |
| | GGGAGGAGGACCCAAGCTGATCCAA |
| | AAGGTGGGTCTAAGCAGTCAAGTGG |
| | AGGAGGGTTCCAATCTGATGGCGGA |
| | GGGCCCAAGCTCAGCCTAACGAGGA |
| | GGCCAGGCCCACCAAGGGGCCCCTG |
| | GAGGACTTGTTTCCCTTGTCCCTTG |
| | TGGTTTTTTGCATTTCCTGTTCCCT |
| | TGCTGCTCATTGCGGAAGTTCCTCT |
| | TCTTACCCTGCACCCAGAGCCTCGC |
| | CAGAGAAGACAAGGGCAGAAAGCAC |
| | CATGTCTGGCGGACCTATGGGAGGT |
| | AGACCTGGTGGAAGAGGTGCCCCCG |
| | CCGTGCAGCAGAACATCCCCAGCAC |
| | CCTGCTGCAGGACCACGAGAACCAG |
| | CGCCTGTTCGAGATGCTGGGCCGCA |
| | AGTGCCTGACCCTGGCCACCGCCGT |
| | GGTGCAGCTGTACCTGGCCCTGCCC |
| | CCCGGCGCCGAGCACTGGACCAAGG |
| | AGCACTGCGGCGCCGTGTGCTTCGT |
| | GAAGGACAACCCCCAGAAGAGCTAC |
| | TTCATCCGCCTGTACGGCCTGCAGG |
| | CCGGCCGCCTGCTGTGGGAGCAGGA |
| | GCTGTACAGCCAGCTGGTGTACAGC |
| | ACCCCCACCCCCTTCTTCCACACCT |
| | TCGCCGGCGACGACTGCCAGGCCGG |
| | CCTGAACTTCGCCGACGAGGACGAG |
| | GCCCAGGCCTTCCGCGCCCTGGTGC |
| | AGGAGAAGATCCAGAAGCGCAACCA |
| | GCGCCAGAGCGGCGACAGAAGGCAA |
| | CTGCCTCCTCCACCTACACCAGCCA |
| | ACGAGGAAAGAAGAGGCGGACTGCC |
| | CCCCCTGCCTCTTCATCCTGGCGGA |
| | GATCAAGGTGGACCTCCTGTGGGAC |
| | CACTGTCTCTTGGCCTGGCCACCGT |
| | GGACATCCAGAACCCCGACATCACC |
| | AGCAGCCGCTACCGCGGCCTGCCCG |
| | CCCCCGGCCCCAGCCCCGCCGACAA |
| | GAAGCGCAGCGGCAAGAAGAAGATC |
| | AGCAAGGCCGACATCGGCGCCCCCA |
| | GCGGCTTCAAGCACGTGAGCCACGT |
| | GGGCTGGGACCCCCAGAACGGCTTC |
| | GACGTGAACAACCTGGACCCCGACC |
| | TGCGCAGCCTGTTCAGCCGCGCCGG |
| | CATCAGCGAGGCCCAGCTGACCGAC |
| | GCCGAGACCAGCAAGCTGATCTACG |
| | ACTTCATCGAGGACCAGGGCGGCCT |
| | GGAGGCCGTGCGCCAGGAGATGCGC |
| | CGCCAGGAGCCCCTGCCACCACCTC |
| | CACCTCCATCTAGAGGCGGCAACCA |
| | GCTGCCCCGCCCTCCTATCGTTGGC |
| | GGCAACAAGGGAAGATCTGGCCCTC |
| | TGCCTCCTGTGCCTCTGGGAATCGC |
| | TCCACCACCACCAACACCTAGAGGC |
| | CCGCCTCCACCAGGCAGAGGTGGTC |
| | CTCCGCCGCCACCTCCTCCAGCCAC |
| | CGGCCGCAGCGGCCCCCTTCCTCCT |
| | CCACCACCTGGTGCTGGTGGACCTC |
| | CAATGCCACCGCCACCGCCTCCGCC |
| | ACCTCCGCCTCCAAGTTCTGGAAAT |
| | GGACCTGCTCCTCCTCCTTTGCCTC |
| | CTGCTTTGGTTCCTGCTGGCGGATT |
| | GGCTCCAGGCGGAGGAAGAGGCGCA |
| | CTGCTGGACCAGATCCGCCAGGGCA |
| | TCCAGCTGAACAAGACCCCTGGCGC |
| | TCCTGAGAGTTCTGCTCTGCAACCG |
| | CCACCACAGTCTAGCGAAGGACTTG |
| | TGGGAGCCCTGATGCACGTGATGCA |
| | GAAGCGCAGCCGCGCCATCCACAGC |
| | AGCGACGAGGGCGAGGACCAGGCCG |
| | GCGACGAGGACGAGGACGACGAGTG |
| | GGACGACTAActgcaggaattcgag |
| | catcttaccgccatttattcccata |
| | tttgttctgtttttcttgatttggg |
| | tatacatttaaatgttaataaaaca |
| | aaatggtggggcaatcatttacatt |
| | tttagggatatgtaattactagttc |
| | aggtgtattgccacaagacaaacat |
| | gttaagaaactttcccgttatttac |
| | gctctgttcctgttaatcaacctct |
| | ggattacaaaatttgtgaaagattg |
| | actgatattcttaactatgttgctc |
| | cttttacgctgtgtggatatgctgc |
| | tttaatgcctctgtatcatgctatt |
| | gcttcccgtacggctttcgttttct |
| | cctccttgtataaatcctggttgct |
| | gtctctttatgaggagttgtggccc |
| | gttgtccgtcaacgtggcgtggtgt |
| | gctctgtgtttgctgacgcaacccc |
| | cactggctggggcattgccaccacc |
| | tgtcaactcctttctgggactttcg |
| | ctttccccctcccgatcgccacggc |
| | agaactcatcgccgcctgccttgcc |
| | cgctgctggacaggggctaggttgc |
| | tgggcactgataattccgtggtgtt |
| | gtcggggaagggcctgctgccggct |
| | ctgcggcctcttccgcgtcttcgcc |
| | ttcgccctcagacgagtcggatctc |
| | cctttgggccgcctccccgcctgga |
| | attcgagctcggtacctttaagacc |
| | aatgacttacaaggcagctgtagat |
| | cttagccactttttaaaagaaaagg |
| | ggggactggaagggctaattcactc |
| | ccaacgaagacaagatctgcttttt |
| | gcttgtactgggtctctctggttag |
| | accagatctgagcctgggagctctc |
| | tggctaactagggaacctactgctt |
| | aagcctcaataaagcttgccttgag |
| | tgcttCAAGTAGTGTGTGCCCGTCT |
| | GTTGTGTGACTCTGGTAACTAGAGA |
| | TCCCTCAGACCCTTTTAGTCAGTGT |
| | GGAAAATCTCTAGCagtagtagttc |
| | atgtcatcttattattcagtattta |
| | taacttgcaaagaaatgaatatcag |
| | agagtgagaggaacttgtttattgc |
| | agcttataatggttacaaataaagc |
| | aatagcatcacaaatttcacaaata |
| | aagcatttttttcactgcattctag |
| | ttgtggtttgtccaaactcatcaat |
| | gtatcttatcatgtctggctctagc |
| | tatcccgcccctaactccgcccatc |
| | ccgcccctaactccgcccagttccg |
| | cccattctccgccccatggctgact |
| | aattttttttatttatgcagaggcc |
| | gaggccgcctcggcctctgagctat |
| | tccagaagtagtgaggaggcttttt |
| | tggaggcctagggacgtacccaatt |
| | cgccctatagtgagtcgtattacgc |
| | gcgctcactggccgtcgttttacaa |
| | cgtcgtgactgggaaaaccctggcg |
| | ttacccaacttaatcgccttgcagc |
| | acatccccctttcgccagctggcgt |
| | aatagcgaagaggcccgcaccgatc |
| | gcccttcccaacagttgcgcagcct |
| | gaatggcgaatgggacgcgccctgt |
| | agcggcgcattaagcgcggcgggtg |
| | tggtggttacgcgcagcgtgaccgc |
| | tacacttgccagcgccctagcgccc |
| | gctcctttcgctttcttcccttcct |
| | ttctcgccacgttcgccggctttcc |
| | ccgtcaagctctaaatcgggggctc |
| | cctttagggttccgatttagtgctt |
| | tacggcacctcgaccccaaaaaact |
| | tgattagggtgatggttcacgtagt |
| | gggccatcgccctgatagacggttt |
| | ttcgccctttgacgttggagtccac |
| | gttctttaatagtggactcttgttc |
| | caaactggaacaacactcaacccta |
| | tctcggtctattcttttgatttata |
| | agggattttgccgatttcggcctat |
| | tggttaaaaaatgagctgatttaac |
| | aaaaatttaacgcgaattttaacaa |
| | aatattaacgcttacaatttaggtg |
| | gcacttttcggggaaatgtgcgcgg |
| | aacccctatttgtttatttttctaa |
| | atacattcaaatatgtatccgctca |
| | tgagacaataaccctgataaatgct |
| | tcaataatagcacctagatcaagag |
| | acaggatgaggatcgtttcgcatga |
| | ttgaacaagatggattgcacgcagg |
| | ttctccggccgcttgggtggagagg |
| | ctattcggctatgactgggcacaac |
| | agacaatcggctgctctgatgccgc |
| | cgtgttccggctgtcagcgcagggg |
| | cgcccggttctttttgtcaagaccg |
| | acctgtccggtgccctgaatgaact |
| | gcaagacgaggcagcgcggctatcg |
| | tggctggccacgacgggcgttcctt |
| | gcgcagctgtgctcgacgttgtcac |
| | tgaagcgggaagggactggctgcta |
| | ttgggcgaagtgccggggcaggatc |
| | tcctgtcatctcaccttgctcctgc |
| | cgagaaagtatccatcatggctgat |
| | gcaatgcggcggctgcatacgcttg |
| | atccggctacctgcccattcgacca |
| | ccaagcgaaacatcgcatcgagcga |
| | gcacgtactcggatggaagccggtc |
| | ttgtcgatcaggatgatctggacga |
| | agagcatcaggggctcgcgccagcc |
| | gaactgttcgccaggctcaaggcga |
| | gcatgcccgacggcgaggatctcgt |
| | cgtgacccatggcgatgcctgcttg |
| | ccgaatatcatggtggaaaatggcc |
| | gcttttctggattcatcgactgtgg |
| | ccggctgggtgtggcggaccgctat |
| | caggacatagcgttggctacccgtg |
| | atattgctgaagagcttggcggcga |
| | atgggctgaccgcttcctcgtgctt |
| | tacggtatcgccgctcccgattcgc |
| | agcgcatcgccttctatcgccttct |
| | tgacgagttcttctgaattattaac |
| | gcttacaatttcctgatgcggtatt |
| | ttctccttacgcatctgtgcggtat |
| | ttcacaccgcatcaggtggcacttt |
| | tcggggaaatgtgcgcggaacccct |
| | atttgtttatttttctaaatacatt |
| | caaatatgtatccgctcatgaccaa |
| | aatcccttaacgtgagttttcgttc |
| | cactgagcgtcagaccccgtagaaa |
| | agatcaaaggatcttcttgagatcc |
| | tttttttctgcgcgtaatctgctgc |
| | ttgcaaacaaaaaaaccaccgctac |
| | cagcggtggtttgtttgccggatca |
| | agagctaccaactctttttccgaag |
| | gtaactggcttcagcagagcgcaga |
| | taccaaatactgttcttctagtgta |
| | gccgtagttaggccaccacttcaag |
| | aactctgtagcaccgcctacatacc |
| | tcgctctgctaatcctgttaccagt |
| | ggctgctgccagtggcgataagtcg |
| | tgtcttaccgggttggactcaagac |
| | gatagttaccggataaggcgcagcg |
| | gtcgggctgaacggggggttcgtgc |
| | acacagcccagcttggagcgaacga |
| | cctacaccgaactgagatacctaca |
| | gcgtgagctatgagaaagcgccacg |
| | cttcccgaagggagaaaggcggaca |
| | ggtatccggtaagcggcagggtcgg |
| | aacaggagagcgcacgagggagctt |
| | ccagggggaaacgcctggtatcttt |
| | atagtcctgtcgggtttcgccacct |
| | ctgacttgagcgtcgatttttgtga |
| | tgctcgtcaggggggcggagcctat |
| | ggaaaaacgccagcaacgcggcctt |
| | tttacggttcctggccttttgctgg |
| | ccttttgctcacatgttctttcctg |
| | cgttatcccctgattctgtggataa |
| | ccgtattaccgcctttgagtgagct |
| | gataccgctcgccgcagccgaacga |
| | ccgagcgcagcgagtcagtgagcga |
| | ggaagcggaagagcgcccaatacgc |
| | aaaccgcctctccccgcgcgttggc |
| | cgattcattaatgcagctggcacga |
| | caggtttcccgactggaaagcgggc |
| | agtgagcgcaacgcaattaatgtga |
| | gttagctcactcattaggcacccca |
| | ggctttacactttatgcttccggct |
| | cgtatgttgtgtggaattgtgagcg |
| | gataacaatttcacacaggaaacag |
| | ctatgaccatgattacgccaagcgc |
| | gcaattaaccctcactaaagggaac |
| | aaaagctggagctgcaagcttggcc |
| | attgcatacgttgtatccatatcat |
| | aatatgtacatttatattggctcat |
| | gtccaacattaccgccatgttgaca |
| | ttgattattgactagttattaatag |
| | taatcaattacggggtcattagttc |
| | atagcccatatatggAGTTCCGCGT |
| | TACATAACTTACGGTAAATGGCCCG |
| | CCTGGCTGACCGCCCAACGACCCCC |
| | GCCCATTGACGTCAATAATGACGTA |
| | TGTTCCCATAGTAACGCCAATAGGG |
| | ACTTTCCATTGACGTCAATGGGTGG |
| | AGTATTTACGGTAAACTGCCCACTT |
| | GGCAGTACATCAAGTGTATCATATG |
| | CCAAGTACGCCCCCTATTGACGTCA |
| | ATGACGGTAAATGGCCCGCCTGGCA |
| | TTATGCCCAGTA |
| SEQ ID NO: 7 | CAAACCTTGTTATCTGAGAGAAAGC |
| Element 9 slim | TAGAGCTTCCTTCTTTCAGCCCCCA |
| Human | GAGACAATGTGGCCAGGCTCCGGAG |
| | GGCTGGGAAGATGAGCAATGCTTGT |
| | GAGTCACCATGATAGGAAGCAGAAG |
| | GGTCAGGAAGTCCCTGGGAGCAAGG |
| | CTTAGGGTTAGGGTTAGGGAGAAAA |
| | CACTTGGGCCTGGAGGCTCGGGGCA |
| | GGCTTCCTAGAGGGGAGGGGTA |
| SEQ ID NO: 8 | CTAAAAGCCACCAAAACCAAGACAG |
| Hypersensitive | CAATGAAAGTAACCTCTGGTCTTCC |
| site | TAACTGCTCATTATACACTAATTAT |
| 3 (HS3) | GATGCACTAGCATGCTAAGAGACAC |
| Human | TTCCACCAGCGCCATGACAGTGCCA |
| | TGGCAACATCAAGAAGTCACCCCAC |
| | ATGGTCTAAAAAGGGGAAGAACCTT |
| | CAGTTCCCGGAATTGCCCACCCCTT |
| | TCCTGGAAAACTCATGAATAATCCA |
| | CCCCTTGTTTAGCATATAAT |
| SEQ ID NO: 9 | GATCTGGGGGAAAACCCCCTGAGGG |
| Sub-sub- | TCCCATTTCTCTGCCACTGACCCCT |
| element 1 | TTGCCTGGCAGAAAGGCTCTGCTGC |
| of element 2 | CTCCTAGCTGTGTAGCAATGGATAA |
| Human | GGCCTTTAACCTCTCTAAGCTGAAA |
| | TGTCCTCATGCATAAGGTGGAGACA |
| | GTAACTCCTCTTCCATCTGCTGGGT |
| | GTTGTCAAAACTACACAGGAGGTTT |
| | GCCAAGCACCTAAGGCACAGTAGGT |
| | GGTCAAGAAACAGGAACACAATTTG |
| | TCATGACGACAATGATTCCTTCATA |
| | TGCTAGTTACCCACGAGTCAGCCAG |
| | AATGTTGCCATAACCACTTATGAAG |
| | CCCTTCCTCATTTCTGTTGCAATTC |
| | CTCAGCCAGAGGAAAGCCCCA |
| SEQ ID NO: 10 | CACAGCCCTTGCCACTCCCCCATGG |
| Sub-element | CCCATGTAGAAACAACCATGTGGCC |
| 4 of | TTCACTCTGCCCACAGAAGCTAGCA |
| element 2 | CCAGGACCCTGGTCAGGGTTAGAGG |
| Human | TTTCTGCTGAGTCAAAGCCACATGG |
| | AGGGAGGGAGCAAGGGAGAGATGCA |
| | GAGTCATGTTTCCAGGAGGAGGTTA |
| | TCTGAGCATAACAGGGACAGGGTGG |
| | GCCACAGGATACCTCTGAGGCTCAG |
| | GTTCCCACCTCCACTCCACC |
| SEQ ID NO: 11 | AAAGGGCTGTTACAGGAGAATATGG |
| WAS promoter | ACACCCAGGCTGCACATGCACACCA |
| Human | TGGAATGCTGTATGGCAGTGGAAAT |
| | AAATGAACAGCTACCACTATAGGCA |
| | AACAGGAATCACAGCAACAGCCAAG |
| | AGTGAAGGCGTGGAGGGACGAGACC |
| | ATGCACTCACACCTGGCCTGCCTGG |
| | CTCGCACTCCGGGCAAAGGGGTCAG |
| | AACAGTGACTGGCACACACGTTAAG |
| | TGCTATGTGAGTGTTAAGATAAAAC |
| | TAGGATGTCCAGTGGGGAGAAAGCA |
| | AGCCTTTGAAGATTATGTGCTTTTA |
| | CAAACTTCAAGTGCAATGAAAACTA |
| | AACAAGATGTTGTTCAGGCATTCAT |
| | ATATGATATAAAGTTCCTTTCTTTA |
| | AAAAAGGGATGGGCTGGGCACGGTG |
| | GCTCACGCCTGTAATTCTAATACTT |
| | TGGGAGGCCGAGGCAGGTGGATCAC |
| | GAGGTCGAGAAATCGAGACCATCCT |
| | GGCCAACATGGTGAAACCCTGTCTC |
| | TACTAAAAATACAAAAAAATTAGCT |
| | GGGCGTGGTGGCGTGTGCCTGTAGT |
| | CCCAGCTACTTGGGAGGCTGAGGCA |
| | GGAGAGTCACTTGAACCCGGGAGGC |
| | AAAGGTTGCAGTGAGCCGAGATCGT |
| | GCCACCGCACTCCAGCCTGGCGACA |
| | GAGTGAGACTCCATCTCAAAAAAAA |
| | AAAGAAAAAAAAAAGTATGACAAGC |
| | AGAAAGTAATTTGGGAGCTGCGGGG |
| | AGGCAAGGGTAAGGGATGGGGAAGT |
| | GGACCAGAGGCATATGCGTCATTGG |
| | CAGTGTCTAAGCACTCACGATAGGC |
| | GTGGATCACAGGGGCTCGCTCTGTA |
| | ATTAAAAGGAAAAGGGTTTTTGTTG |
| | TGTTGTTGTTGTTGCTGTTTTTGAG |
| | ACAAGGGTCTTGCTCTGTCATCATC |
| | CAGGCTGGAGTGCAGTGGTGCAGTC |
| | TCAGCTCACTGCAACCTCCGCCTCC |
| | TGGGTTCAAGCGATTCTCCTGCCTC |
| | AGCCTCCTGAGCAGCTAGGACTACA |
| | GGTGTGTGCCACCATGCCTGGCTAA |
| | TTTTTGTATTTTTTAGTGGAAATGG |
| | GGTTTTGCCATGTTGCCCAGGCTCG |
| | TCTTGAACTCCTGACCTCAAGTGAT |
| | CCACTCGTCTCGGCCTCCCAAAGTG |
| | CTGGGATTACAGGTGTGAGCTATTG |
| | TCCCCAGCCAAAAGGAAAAGTTTTA |
| | CTGTAGTAACCCTTCCGGACTAGGG |
| | ACCTCGGGCCCTCGAGTCAGCCTCA |
| | GGCTACCTAGGTGCTTTAGAAAGGA |
| | GGCCACCCAGGCCCATGACTACTCC |
| | TTGCCACAGGGAGCCCTGCACACAG |
| | ATGTGCTAAGCTCTCGCTGCCAGCC |
| | AGAGGGAGGAGGGTCTGAGCCAGTC |
| | AGAAGGAGATGGGCCCCAGAGAGTA |
| | AGAAAGGGGGAGGAGGACCCAAGCT |
| | GATCCAAAAGGTGGGTCTAAGCAGT |
| | CAAGTGGAGGAGGGTTCCAATCTGA |
| | TGGCGGAGGGCCCAAGCTCAGCCTA |
| | ACGAGGAGGCCAGGCCCACCAAGGG |
| | GCCCCTGGAGGACTTGTTTCCCTTG |
| | TCCCTTGTGGTTTTTTGCATTTCCT |
| | GTTCCCTTGCTGCTCATTGCGGAAG |
| | TTCCTCTTCTTACCCTGCACCCAGA |
| | GCCTCGCCAGAGAAGACAAGGGCAG |
| | AAAGCACC |
| SEQ ID NO: 12 | TCAGCCTCAGGCTACCTAGGTGCTT |
| HS1pro (minimal | TAGAAAGGAGGCCACCCAGGCCCAT |
| endogenous | GACTACTCCTTGCCACAGGGAGCCC |
| promoter of the | TGCACACAGATGTGCTAAGCTCTCG |
| WAS gene) | CTGCCAGCCAGAGGGAGGAGGGTCT |
| Human | GAGCCAGTCAGAAGGAGATGGGCCC |
| | CAGAGAGTAAGAAAGGGGGAGGAGG |
| | ACCCAAGCTGATCCAAAAGGTGGGT |
| | CTAAGCAGTCAAGTGGAGGAGGGTT |
| | CCAATCTGATGGCGGAGGGCCCAAG |
| | CTCAGCCTAACGAGGAGGCCAGGCC |
| | CACCAAGGGGCCCCTGGAGGACTTG |
| | TTTCCCTTGTCCCTTGTGGTTTTTT |
| | GCATTTCCTGTTCCCTTGCTGCTCA |
| | TTGCGGAAGTTCCTCTTCTTACCCT |
| | GCACCCAGAGCCTCGCCAGAGAAGA |
| | CAAGGGCAGAAAGCACC |
| SEQ ID NO: 13 | GACCTGAAGGGTAAGGGGGTGTGGA |
| Enhancer | GGTTGTGAAGGCGGGAAGGGGGGTA |
| element | GCCCCTTCACCAATGTAAACAAGGA |
| 2 (Slim) | TGTGGGTTCTGCGGCCACACTCTCC |
| | CCCGCCCTCCCCAGCGGCATTTCCA |
| | GCAAGTCACATGTCCTGCGCACAGG |
| | CTGGGGGCCCCCTGCTGCTGCCTTT |
| | CTTAGAAGCCAGGACCACAGAGCCC |
| | GCACAGTGAGCTACTTGGGGAGCTA |
| | TTTCTGTAGACTGAGCTTGGGGTAC |
| | TGGGAGGCAGTGCTGTTGGCGATCT |
| | GGGGGAAAACCCCCTGAGGGTCCCA |
| | TTTCTCTGCCACTGACCCCTTTGCC |
| | TGGCAGAAAGGCTCTGCTGCCTCCT |
| | AGCTGTGTAGCAATGGATAAGGCCT |
| | TTAACCTCTCTAAGCTGAAATGTCC |
| | TCATGCATAAGGTGGAGACAGTAAC |
| | TCCTCTTCCATCTGCTGGGTGTTGT |
| | CAAAACTACACAGGAGGTTTGCCAA |
| | GCACCTAAGGCACAGTAGGTGGTCA |
| | AGAAACAGGAACACAATTTGTCATG |
| | ACGACAATGATTCCTTCATATGCTA |
| | GTTACCCACGAGTCAGCCAGAATGT |
| | TGCCATAACCACTTATGAAGCCCTT |
| | CCTCATTTCTGTTGCAATTCCTCAG |
| | CCAGAGGAAAGCCCCACCACCTCTG |
| | TCCTCCAGAAAGCCCTCCTCTTCCC |
| | AGAAGGCTGGCACACAGGGCTGGGG |
| | GCCAGGCTTGACTTCCCTGTGGAGG |
| | GGTCTGAGGTTGGGGGGATGACCTG |
| | CAGAGGTAGGGGAATTCAGAGAACT |
| | TGTTTTGATAGAAACTGAAACTCCT |
| | CCTCTTGCTGACGCTGCACTTGTGG |
| | GCAATCTGCACCTGTTCTCCCCTCC |
| | CAGAGGCCAGCTGGAAGGGGGTCCC |
| | CCACCATTCTACCTCTGTTCCCATC |
| | TCAAACAGGGCTCAGAAATCCTCAG |
| | AGAGCCTGGATTATCTTCCTATAGG |
| | GTCCTACCATTTGCTTTAGAGACAT |
| | CTGAGAACTGCCTACTCCCCAGGTA |
| | GCTGCCTGCCTCCCAGAAGAGCCAC |
| | AGCAAGTTCCGCTAAGGGCAAAAAA |
| | GGAAGCTAGGTGTCTACAGGGAACC |
| | TAAAAACAAACCACACTAACGTGTG |
| | TACACACAGCTGCAGAGGGAGACAG |
| | GGACCTGGTCTGCTCACAGCCCTTG |
| | CCACTCCCCCATGGCCCATGTAGAA |
| | ACAACCATGTGGCCTTCACTCTGCC |
| | CACAGAAGCTAGCACCAGGACCCTG |
| | GTCAGGGTTAGAGGTTTCTGCTGAG |
| | TCAAAGCCACATGGAGGGAGGGAGC |
| | AAGGGAGAGATGCAGAGTCATGTTT |
| | CCAGGAGGAGGTTATCTGAGCATAA |
| | CAGGGACAGGGTGGGCCACAGGATA |
| | CCTCTGAGGCTCAGGTTCCCACCTC |
| | CACTCCACCCTCCAACTCTCTGCTC |
| | CACGTTCCTTCCACTATTCAACACA |
| | TGGCAGGAAGTCAGCCTGTAGGGCT |
| | TCACACTACAGTCTAAGCTGACTGC |
| | CATCTGTCCCCATCCAGGCGAGGCT |
| | GGAAACGGGGGCAGCAAAAAGCCCA |
| | GGGACAAAGTCCCCTTCCCAGGCAC |
| | ATGTATTATGCACTCTGCACCAAGG |
| | AAACCTCCAGATAAGGTCTAGAGAC |
| | CAAGGGCCATGTACTTCGGGGGAGA |
| | GGCCAGAGGACTTCTGAGGTTTTAC |
| | AGAGAAAAGCCAAAGGCAGCCCAGT |
| | CAGGGGAAATGTGTAGCCATAGTGC |
| | CGATAAGGAAAGGCCTTCAACCTGC |
| | CCTGGTCAGCTCTTCCTGTAAGTAG |
| | AGGCCCCTTACCGAAGGCCCCAGTG |
| Human | GGAGGAAGGGTCGGGAGTA |
| SEQ ID NO: 14 | GACCTGAAGGGTAAGGGGGTGTGGA |
| 1^{st} half | GGTTGTGAAGGCGGGAAGGGGGGTA |
| of Core | GCCCCTTCACCAATGTAAACAAGGA |
| Sub-Element of | TGTGGGTTCTGCGGCCACACTCTCC |
| Enhancer | CCCGCCCTCCCCAGCGGCATTTCCA |
| Element | GCAAGTCACATGTCCTGCGCACAGG |
| 2 | CTGGGGGCCCCCTGCTGCTGCCTTT |
| Human | CTTAGAAGCCAGGACCACAGAGCCC |
| | GCACAGTGAGCTACTTGGGGAGCTA |
| | TTTCTGTAGACTGAGCTTGGGGTAC |
| | TGGGAGGCAGTGCTGTTGGC |
| SEQ ID NO: 15 | CCACCTCTGTCCTCCAGAAAGCCCT |
| Core Sub-Element | CCTCTTCCCAGAAGGCTGGCACACA |
| 2 of Enhancer | GGGCTGGGGGCCAGGCTTGACTTCC |
| Element 2 | CTGTGGAGGGGTCTGAGGTTGGGGG |
| Human | GATGACCTGCAGAGGTAGGGGAATT |
| | CAGAGAACTTGTTTTGATAGAAACT |
| | GAAACTCCTCCTCTTGCTGACGCTG |
| | CACTTGTGGGCAATCTGCACCTGTT |
| | CTCCCCTCCCAGAGGCCAGCTGGAA |
| | GGGGGTCCCCCACCATTCTACCTCT |
| | GTTCCCATCTCAAACAGGGCTCAGA |
| | AATCCTCA |
| SEQ ID NO: 16 | GAGAGCCTGGATTATCTTCCTATAG |
| Core Sub-Element | GGTCCTACCATTTGCTTTAGAGACA |
| 3 of Enhancer | TCTGAGAACTGCCTACTCCCCAGGT |
| Element 2 | AGCTGCCTGCCTCCCAGAAGAGCCA |
| Human | CAGCAAGTTCCGCTAAGGGCAAAAA |
| | AGGAAGCTAGGTGTCTACAGGGAAC |
| | CTAAAAACAAACCACACTAACGTGT |
| | GTACACACAGCTGCAGAGGGAGACA |
| | GGGACCTGGTCTGCT |
| SEQ ID NO: 17 | CTCCAACTCTCTGCTCCACGTTCCT |
| Core Sub-Element | TCCACTATTCAACACATGGCAGGAA |
| 5 of Enhancer | GTCAGCCTGTAGGGCTTCACACTAC |
| Element 2 | AGTCTAAGCTGACTGCCATCTGTCC |
| Human | CCATCCAGGCGAGGCTGGAAACGGG |
| | GGCAGCAAAAAGCCCAGGGACAAAG |
| | TCCCCTTCCCAGGCACATGTATTAT |
| | GCACTCTGCACCAAGGAAACCTCCA |
| | GATAAGGTCTAGAGACCAAGGGCCA |
| | TGTACTTCGGGGGAGAGGCCAGAGG |
| | ACTTCTGAGGTTTTACAGAGAAAAG |
| | CCAAAGGCAGCCCAGTCAGGGGAAA |
| | TGTGTAGCCATAGTGCCGATAAGGA |
| | AAGGCCTTCAACCTGCCCTGGTCAG |
| | CTCTTCCTGTAAGTAGAGGCCCCTT |
| | ACCGAAGGCCCCAGTGGGAGGAAGG |
| | GTCGGGAGTA |
| SEQ ID NO: 18 | CTAAAAGCCACCAAAACCAAGACAG |
| Enhancer Element | CAATGAAAGTAACCTCTGGTCTTCC |
| HS3 (full) | TAACTGCTCATTATACACTAATTAT |
| Human | GATGCACTAGCATGCTAAGAGACAC |
| | TTCCACCAGCGCCATGACAGTGCCA |
| | TGGCAACATCAAGAAGTCACCCCAC |
| | ATGGTCTAAAAAGGGGAAGAACCTT |
| | CAGTTCCCGGAATTGCCCACCCCTT |
| | TCCTGGAAAACTCATGAATAATCCA |
| | CCCCTTGTTTAGCATATAATTAAGA |
| | AATAACTGTAAGTATCCTTAGTCCA |
| | GCAGCCCAAGCTGCTGCTCTGCCTA |
| | TGGAGTAGCCATTCTTTATTCCTTT |
| | ACTTTCTTAATAAACTTGCTTTCAC |
| | ATTAAAAACAACAACAACAACACCA |
| | ACAACCAGCAAATATATCAGAGGCT |
| | TAGCATCCCCTATTCAAGGGGACAG |
| | TTCTGGAGGCCGAGAAAGAAGTAGA |
| | AAGGTGGAGAATAAAAGACACAAAG |
| | GAGCAGAAGAATTTTATCTTGGCCT |
| | CCATGTGGCACCTCACAGCTTGGGA |
| | ACAACA |
| SEQ ID NO: 19 | GGGATCTGTGTGCAGATTTACCTCT |
| Enhancer Element | ACCTACACCTGGCTGGGGATCATAA |
| E9 (full) | AGAAAATCAAGGGATGCGCTACCTC |
| Human | CATGAATCCAGGTTTCAGCAGAGCT |
| | AAGGGAGTGAAATTTGGGGCCATTT |
| | TACTTGTCTCAAACCTTGTTATCTG |
| | AGAGAAAGCTAGAGCTTCCTTCTTT |
| | CAGCCCCCAGAGACAATGTGGCCAG |
| | GCTCCGGAGGGCTGGGAAGATGAGC |
| | AATGCTTGTGAGTCACCATGATAGG |
| | AAGCAGAAGGGTCAGGAAGTCCCTG |
| | GGAGCAAGGCTTAGGGTTAGGGTTA |
| | GGGAGAAAACACTTGGGCCTGGAGG |
| | CTCGGGGCAGGCTTCCTAGAGGGGA |
| | GGGGTAGGAAGAGGCAGTGACAGGG |
| | CCCACAGCAATGGAGAGGAGTTGGA |
| | CTGCAGGGATGGGGTAGGGGGACAG |
| | ACGACAAGGGACACTCAGAAGACTA |
| | ATGTCTGGGAGTGGGAAAACAGTGT |
| | TTGCTCAGCCAGGAAGCTGCATCCA |
| | GCTCTTTTATCATTTGTAGAAGACC |
| | AAGTACCCAGGCCTGGGAGGAGCCC |
| | AGAGA |
| SEQ ID NO: 20 | ATGAGTGGGGGCCCAATGGGAGGAA |
| WAS cDNA | GGCCCGGGGGCCGAGGAGCACCAGC |
| Human | GGTTCAGCAGAACATACCCTCCACC |
| | CTCCTCCAGGACCACGAGAACCAGC |
| | GACTCTTTGAGATGCTTGGACGAAA |
| | ATGCTTGACGCTGGCCACTGCAGTT |
| | GTTCAGCTGTACCTGGCGCTGCCCC |
| | CTGGAGCTGAGCACTGGACCAAGGA |
| | GCATTGTGGGGCTGTGTGCTTCGTG |
| | AAGGATAACCCCCAGAAGTCCTACT |
| | TCATCCGCCTTTACGGCCTTCAGGC |
| | TGGTCGGCTGCTCTGGGAACAGGAG |
| | CTGTACTCACAGCTTGTCTACTCCA |
| | CCCCCACCCCCTTCTTCCACACCTT |
| | CGCTGGAGATGACTGCCAAGCGGGG |
| | CTGAACTTTGCAGACGAGGACGAGG |
| | CCCAGGCCTTCCGGGCCCTCGTGCA |
| | GGAGAAGATACAAAAAAGGAATCAG |
| | AGGCAAAGTGGAGACAGACGCCAGC |
| | TACCCCCACCACCAACACCAGCCAA |
| | TGAAGAGAGAAGAGGAGGGCTCCCA |
| | CCCCTGCCCCTGCATCCAGGTGGAG |
| | ACCAAGGAGGCCCTCCAGTGGGTCC |
| | GCTCTCCCTGGGGCTGGCGACAGTG |
| | GACATCCAGAACCCTGACATCACGA |
| | GTTCACGATACCGTGGGCTCCCAGC |
| | ACCTGGACCTAGCCCAGCTGATAAG |
| | AAACGCTCAGGGAAGAAGAAGATCA |
| | GCAAAGCTGATATTGGTGCACCCAG |
| | TGGATTCAAGCATGTCAGCCACGTG |
| | GGGTGGGACCCCCAGAATGGATTTG |
| | ACGTGAACAACCTCGACCCAGATCT |
| | GCGGAGTCTGTTCTCCAGGGCAGGA |
| | ATCAGCGAGGCCCAGCTCACCGACG |
| | CCGAGACCTCTAAACTTATCTACGA |
| | CTTCATTGAGGACCAGGGTGGGCTG |
| | GAGGCTGTGCGGCAGGAGATGAGGC |
| | GCCAGGAGCCACTTCCGCCGCCCCC |
| | ACCGCCATCTCGAGGAGGGAACCAG |
| | CTCCCCCGGCCCCCTATTGTGGGGG |
| | GTAACAAGGGTCGTTCTGGTCCACT |
| | GCCCCCTGTACCTTTGGGGATTGCC |
| | CCACCCCCACCAACACCCCGGGGAC |
| | CCCCACCCCCAGGCCGAGGGGGCCC |
| | TCCACCACCACCCCCTCCAGCTACT |
| | GGACGTTCTGGACCACTGCCCCCTC |
| | CACCCCCTGGAGCTGGTGGGCCACC |
| | CATGCCACCACCACCGCCACCACCG |
| | CCACCGCCGCCCAGCTCCGGGAATG |
| | GACCAGCCCCTCCCCCACTCCCTCC |
| | TGCTCTGGTGCCTGCCGGGGGCCTG |
| | GCCCCTGGTGGGGGTCGGGGAGCGC |
| | TTTTGGATCAAATCCGGCAGGGAAT |
| | TCAGCTGAACAAGACCCCTGGGGCC |
| | CCAGAGAGCTCAGCGCTGCAGCCAC |
| | CACCTCAGAGCTCAGAGGGACTGGT |
| | GGGGGCCCTGATGCACGTGATGCAG |
| | AAGAGAAGCAGAGCCATCCACTCCT |
| | CCGACGAAGGGGAGGACCAGGCTGG |
| | CGATGAAGATGAAGATGATGAATGG |
| | GATGACTGA |
| SEQ ID NO: 21 | ATGTCTGGCGGACCTATGGGAGGTA |
| Codon-optimized | GACCTGGTGGAAGAGGTGCCCCCGC |
| WAS CDNA | CGTGCAGCAGAACATCCCCAGCACC |
| Synthetic | CTGCTGCAGGACCACGAGAACCAGC |
| | GCCTGTTCGAGATGCTGGGCCGCAA |
| | GTGCCTGACCCTGGCCACCGCCGTG |
| | GTGCAGCTGTACCTGGCCCTGCCCC |
| | CCGGCGCCGAGCACTGGACCAAGGA |
| | GCACTGCGGCGCCGTGTGCTTCGTG |
| | AAGGACAACCCCCAGAAGAGCTACT |
| | TCATCCGCCTGTACGGCCTGCAGGC |
| | CGGCCGCCTGCTGTGGGAGCAGGAG |
| | CTGTACAGCCAGCTGGTGTACAGCA |
| | CCCCCACCCCCTTCTTCCACACCTT |
| | CGCCGGCGACGACTGCCAGGCCGGC |
| | CTGAACTTCGCCGACGAGGACGAGG |
| | CCCAGGCCTTCCGCGCCCTGGTGCA |
| | GGAGAAGATCCAGAAGCGCAACCAG |
| | CGCCAGAGCGGCGACAGAAGGCAAC |
| | TGCCTCCTCCACCTACACCAGCCAA |
| | CGAGGAAAGAAGAGGCGGACTGCCC |
| | CCCCTGCCTCTTCATCCTGGCGGAG |
| | ATCAAGGTGGACCTCCTGTGGGACC |
| | ACTGTCTCTTGGCCTGGCCACCGTG |
| | GACATCCAGAACCCCGACATCACCA |
| | GCAGCCGCTACCGCGGCCTGCCCGC |
| | CCCCGGCCCCAGCCCCGCCGACAAG |
| | AAGCGCAGCGGCAAGAAGAAGATCA |
| | GCAAGGCCGACATCGGCGCCCCCAG |
| | CGGCTTCAAGCACGTGAGCCACGTG |
| | GGCTGGGACCCCCAGAACGGCTTCG |
| | ACGTGAACAACCTGGACCCCGACCT |
| | GCGCAGCCTGTTCAGCCGCGCCGGC |
| | ATCAGCGAGGCCCAGCTGACCGACG |
| | CCGAGACCAGCAAGCTGATCTACGA |
| | CTTCATCGAGGACCAGGGCGGCCTG |
| | GAGGCCGTGCGCCAGGAGATGCGCC |
| | GCCAGGAGCCCCTGCCACCACCTCC |
| | ACCTCCATCTAGAGGCGGCAACCAG |
| | CTGCCCCGCCCTCCTATCGTTGGCG |
| | GCAACAAGGGAAGATCTGGCCCTCT |
| | GCCTCCTGTGCCTCTGGGAATCGCT |
| | CCACCACCACCAACACCTAGAGGCC |
| | CGCCTCCACCAGGCAGAGGTGGTCC |
| | TCCGCCGCCACCTCCTCCAGCCACC |
| | GGCCGCAGCGGCCCCCTTCCTCCTC |
| | CACCACCTGGTGCTGGTGGACCTCC |
| | AATGCCACCGCCACCGCCTCCGCCA |
| | CCTCCGCCTCCAAGTTCTGGAAATG |
| | GACCTGCTCCTCCTCCTTTGCCTCC |
| | TGCTTTGGTTCCTGCTGGCGGATTG |
| | GCTCCAGGCGGAGGAAGAGGCGCAC |
| | TGCTGGACCAGATCCGCCAGGGCAT |
| | CCAGCTGAACAAGACCCCTGGCGCT |
| | CCTGAGAGTTCTGCTCTGCAACCGC |
| | CACCACAGTCTAGCGAAGGACTTGT |
| | GGGAGCCCTGATGCACGTGATGCAG |
| | AAGCGCAGCCGCGCCATCCACAGCA |
| | GCGACGAGGGCGAGGACCAGGCCGG |
| | CGACGAGGACGAGGACGACGAGTGG |
| | GACGACTAA |
| SEQ ID NO: 22 | AGTTCCGCGTTACATAACTTACGGT |
| CMV promoter | AAATGGCCCGCCTGGCTGACCGCCC |
| Viral (from CMV) | AACGACCCCCGCCCATTGACGTCAA |
| | TAATGACGTATGTTCCCATAGTAAC |
| | GCCAATAGGGACTTTCCATTGACGT |
| | CAATGGGTGGAGTATTTACGGTAAA |
| | CTGCCCACTTGGCAGTACATCAAGT |
| | GTATCATATGCCAAGTACGCCCCCT |
| | ATTGACGTCAATGACGGTAAATGGC |
| | CCGCCTGGCATTATGCCCAGTACAT |
| | GACCTTATGGGACTTTCCTACTTGG |
| | CAGTACATCTACGTATTAGTCATCG |
| | CTATTACCATGGTGATGCGGTTTTG |
| | GCAGTACATCAATGGGCGTGGATAG |
| | CGGTTTGACTCACGGGGATTTCCAA |
| | GTCTCCACCCCATTGACGTCAATGG |
| | GAGTTTGTTTTGGCACCAAAATCAA |
| | CGGGACTTTCCAAAATGTCGTAACA |
| | ACTCCGCCCCATTGACGCAAATGGG |
| | CGGTAGGCGTGTACGGTGGGAGGTC |
| | TATATAAGCAGAGCTCGTTTAGTGA |
| | ACCG |
| SEQ ID NO: 23 | GGGTCTCTCTGGTTAGACCAGATCT |
| 5′R/U5 | GAGCCTGGGAGCTCTCTGGCTAACT |
| Viral - HIV | AGGGAACCCACTGCTTAAGCCTCAA |
| | TAAAGCTTGCCTTGAGTGCTTCAAG |
| | TAGTGTGTGCCCGTCTGTTGTGTGA |
| | CTCTGGTAACTAGAGATCCCTCAGA |
| | CCCTTTTAGTCAGTGTGGAAAATCT |
| | CTAGC |
| SEQ ID NO: 24 | TCGACGCAGGACTCGGCTTGCTGAA |
| Packaging Signal | GCGCGCACGGCAAGAGGCGAGGGGC |
| (PSI) | GGCGACTGGTGAGTACGCCAAAAAT |
| Viral-HIV | TTTGACTAGCGGAGGCTAGAAGGAG |
| | AGAGATGGGTGCGAGAGCGTCAGTA |
| | TTAAGCGGGGGAG |
| SEQ ID NO: 25 | TCCTTGGGTTCTTGGGAGCAGCAGG |
| RRE of lentiviral | AAGCACTATGGGCGCAGCGTCAATG |
| vector | ACGCTGACGGTACAGGCCAGACAAT |
| Viral-HIV | TATTGTCTGGTATAGTGCAGCAGCA |
| | GAACAATTTGCTGAGGGCTATTGAG |
| | GCGCAACAGCATCTGTTGCAACTCA |
| | CAGTCTGGGGCATCAAGCAGCTCCA |
| | GGCAAGAATCCTGGCTGTGGAAAGA |
| | TACCT |
| SEQ ID NO: 26 | CCCATATTTGTTCTGTTTTTCTTGA |
| WPRE | TTTGGGTATACATTTAAATGTTAAT |
| Synthetic | AAAACAAAATGGTGGGGCAATCATT |
| | TACATTTTTAGGGATATGTAATTAC |
| | TAGTTCAGGTGTATTGCCACAAGAC |
| | AAACATGTTAAGAAACTTTCCCGTT |
| | ATTTACGCTCTGTTCCTGTTAATCA |
| | ACCTCTGGATTACAAAATTTGTGAA |
| | AGATTGACTGATATTCTTAACTATG |
| | TTGCTCCTTTTACGCTGTGTGGATA |
| | TGCTGCTTTAATGCCTCTGTATCAT |
| | GCTATTGCTTCCCGTACGGCTTTCG |
| | TTTTCTCCTCCTTGTATAAATCCTG |
| | GTTGCTGTCTCTTTATGAGGAGTTG |
| | TGGCCCGTTGTCCGTCAACGTGGCG |
| | TGGTGTGCTCTGTGTTTGCTGACGC |
| | AACCCCCACTGGCTGGGGCATTGCC |
| | ACCACCTGTCAACTCCTTTCTGGGA |
| | CTTTCGCTTTCCCCCTCCCGATCGC |
| | CACGGCAGAACTCATCGCCGCCTGC |
| | CTTGCCCGCTGCTGGACAGGGGCTA |
| | GGTTGCTGGGCACTGATAATTCCGT |
| | GGTGTTGTCGGGGAAGGGCCTGCTG |
| | CCGGCTCTGCGGCCTCTTCCGCGTC |
| | TTCGCCTTCGCCCTCAGACGAGTCG |
| | GATCTCCCTTTGGGCCGCCTCCCCG |
| | CCTGGA |
| SEQ ID NO: 27 | TTTTTAAAAGAAAAGGGGGGAC |
| 3′ PPT of | |
| lentiviral | |
| vector | |
| Viral | |
| SEQ ID NO: 28 | TGGAAGGGCTAATTCACTCCCAACG |
| 3′ delta U3/R/U5 | AAGACAAGATCTGCTTTTTGCTTGT |
| Synthetic | ACTGGGTCTCTCTGGTTAGACCAGA |
| | TCTGAGCCTGGGAGCTCTCTGGCTA |
| | ACTAGGGAACCTACTGCTTAAGCCT |
| | CAATAAAGCTTGCCTTGAGTGCTTC |
| | AAGTAGTGTGTGCCCGTCTGTTGTG |
| | TGACTCTGGTAACTAGAGATCCCTC |
| | AGACCCTTTTAGTCAGTGTGGAAAA |
| | TCTCTAGC |
| SEQ ID NO: 29 | ATCCCGCCCCTAACTCCGCCCAGTT |
| SV40 origin of | CCGCCCATTCTCCGCCCCATGGCTG |
| replication | ACTAATTTTTTTTATTTATGCAGAG |
| Viral | GCCGAGGCCGCCTCGGCCTCTGAGC |
| | TATTCCAGAAGTAGTGAGGAGGCTT |
| | TTTTGGAGGCCTAGG |
| SEQ ID NO: 30 | ATTGAACAAGATGGATTGCACGCAG |
| KANr | GTTCTCCGGCCGCTTGGGTGGAGAG |
| (kanamycin | GCTATTCGGCTATGACTGGGCACAA |
| resistance | CAGACAATCGGCTGCTCTGATGCCG |
| cassette) | CCGTGTTCCGGCTGTCAGCGCAGGG |
| Bacterial | GCGCCCGGTTCTTTTTGTCAAGACC |
| | GACCTGTCCGGTGCCCTGAATGAAC |
| | TGCAAGACGAGGCAGCGCGGCTATC |
| | GTGGCTGGCCACGACGGGCGTTCCT |
| | TGCGCAGCTGTGCTCGACGTTGTCA |
| | CTGAAGCGGGAAGGGACTGGCTGCT |
| | ATTGGGCGAAGTGCCGGGGCAGGAT |
| | CTCCTGTCATCTCACCTTGCTCCTG |
| | CCGAGAAAGTATCCATCATGGCTGA |
| | TGCAATGCGGCGGCTGCATACGCTT |
| | GATCCGGCTACCTGCCCATTCGACC |
| | ACCAAGCGAAACATCGCATCGAGCG |
| | AGCACGTACTCGGATGGAAGCCGGT |
| | CTTGTCGATCAGGATGATCTGGACG |
| | AAGAGCATCAGGGGCTCGCGCCAGC |
| | CGAACTGTTCGCCAGGCTCAAGGCG |
| | AGCATGCCCGACGGCGAGGATCTCG |
| | TCGTGACCCATGGCGATGCCTGCTT |
| | GCCGAATATCATGGTGGAAAATGGC |
| | CGCTTTTCTGGATTCATCGACTGTG |
| | GCCGGCTGGGTGTGGCGGACCGCTA |
| | TCAGGACATAGCGTTGGCTACCCGT |
| | GATATTGCTGAAGAGCTTGGCGGCG |
| | AATGGGCTGACCGCTTCCTCGTGCT |
| | TTACGGTATCGCCGCTCCCGATTCG |
| | CAGCGCATCGCCTTCTATCGCCTTC |
| | TTGACGAGTTCTTCTGA |
| SEQ ID NO: 31 | AGATCAAAGGATCTTCTTGAGATCC |
| COLE1 | TTTTTTTCTGCGCGTAATCTGCTGC |
| (resistance | TTGCAAACAAAAAAACCACCGCTAC |
| cassette) | CAGCGGTGGTTTGTTTGCCGGATCA |
| Bacterial | AGAGCTACCAACTCTTTTTCCGAAG |
| | GTAACTGGCTTCAGCAGAGCGCAGA |
| | TACCAAATACTGTTCTTCTAGTGTA |
| | GCCGTAGTTAGGCCACCACTTCAAG |
| | AACTCTGTAGCACCGCCTACATACC |
| | TCGCTCTGCTAATCCTGTTACCAGT |
| | GGCTGCTGCCAGTGGCGATAAGTCG |
| | TGTCTTACCGGGTTGGACTCAAGAC |
| | GATAGTTACCGGATAAGGCGCAGCG |
| | GTCGGGCTGAACGGGGGGTTCGTGC |
| | ACACAGCCCAGCTTGGAGCGAACGA |
| | CCTACACCGAACTGAGATACCTACA |
| | GCGTGAGCTATGAGAAAGCGCCACG |
| | CTTCCCGAAGGGAGAAAGGCGGACA |
| | GGTATCCGGTAAGCGGCAGGGTCGG |
| | AACAGGAGAGCGCACGAGGGAGCTT |
| | CCAGGGGGAAACGCCTGGTATCTTT |
| | ATAGTCCTGTCGGGTTTCGCCACCT |
| | CTGACTTGAGCGTCGATTTTTGTGA |
| | TGCTCGTCAGGGGGGCGGAGCCTAT |
| | GGAAAAACGCCAGCAACGCG |
| SEQ ID NO: 32 | GACCTGAAGGGTAAGGGGGTGTGGA |
| uCore E2 | GGTTGTGAAGGCGGGAAGGGGGGTA |
| element | GCCCCTTCACCAATGTAAACAAGGA |
| Human | TGTGGGTTCTGCGGCCACACTCTCC |
| | CCCGCCCTCCCCAGCGGCATTTCCA |
| | GCAAGTCACATGTCCTGCGCACAGG |
| | CTGGGGGCCCCCTGCTGCTGCCTTT |
| | CTTAGAAGCCAGGACCACAGAGCCC |
| | GCACAGTGAGCTACTTGGGGAGCTA |
| | TTTCTGTAGACTGAGCTTGGGGTAC |
| | TGGGAGGCAGTGCTGTTGGCCTCCA |
| | ACTCTCTGCTCCACGTTCCTTCCAC |
| | TATTCAACACATGGCAGGAAGTCAG |
| | CCTGTAGGGCTTCACACTACAGTCT |
| | AAGCTGACTGCCATCTGTCCCCATC |
| | CAGGCGAGGCTGGAAACGGGGGCAG |
| | CAAAAAGCCCAGGGACAAAGTCCCC |
| | TTCCCAGGCACATGTATTATGCACT |
| | CTGCACCAAGGAAACCTCCAGATAA |
| | GGTCTAGAGACCAAGGGCCATGTAC |
| | TTCGGGGGAGAGGCCAGAGGACTTC |
| | TGAGGTTTTACAGAGAAAAGCCAAA |
| | GGCAGCCCAGTCAGGGGAAATGTGT |
| | AGCCATAGTGCCGATAAGGAAAGGC |
| | CTTCAACCTGCCCTGGTCAGCTCTT |
| | CCTGTAAGTAGAGGCCCCTTACCGA |
| | AGGCCCCAGTGGGAGGAAGGGTCGG |
| | GAGTA |
| SEQ ID NO: 33 | agttccgcgttacataacttacggt |
| WASVec1.0 | aaatggcccgcctggctgaccgccc |
| Synthetic | aacgacccccgcccattgacgtcaa |
| Construct | taatgacgtatgttcccatagtaac |
| | gccaatagggactttccattgacgt |
| | caatgggtggagtatttacggtaaa |
| | ctgcccacttggcagtacatcaagt |
| | gtatcatatgccaagtacgccccct |
| | attgacgtcaatgacggtaaatggc |
| | ccgcctggcattatgcccagtacat |
| | gaccttatgggactttcctacttgg |
| | cagtacatctacgtattagtcatcg |
| | ctattaccatggtgatgcggttttg |
| | gcagtacatcaatgggcgtggatag |
| | cggtttgactcacggggatttccaa |
| | gtctccaccccattgacgtcaatgg |
| | gagtttgttttggcaccaaaatcaa |
| | cgggactttccaaaatgtcgtaaca |
| | actccgccccattgacgcaaatggg |
| | cggtaggcgtgtacggtgggaggtc |
| | tatataagcagagctcgtttagtga |
| | accggggtctctctggttagaccag |
| | atctgagcctgggagctctctggct |
| | aactagggaacccactgcttaagcc |
| | tcaataaagcttgccttgagtgctt |
| | caagtagtgtgtgcccgtctgttgt |
| | gtgactctggtaactagagatccct |
| | cagacccttttagtcagtgtggaaa |
| | atctctagcagtggcgcccgaacag |
| | ggacttgaaagcgaaagggaaacca |
| | gaggagctctctcgacgcaggactc |
| | ggcttgctgaagcgcgcacggcaag |
| | aggcgaggggcggcgactggtgagt |
| | acgccaaaaattttgactagcggag |
| | gctagaaggagagagatgggtgcga |
| | gagcgtcagtattaagcgggggaga |
| | attagatcgcgatgggaaaaaattc |
| | ggttaaggccagggggaaagaaaaa |
| | atataaattaaaacatatagtatgg |
| | gcaagcagggagctagaacgattcg |
| | cagttaatcctggcctgttagaaac |
| | atcagaaggctgtagacaaatactg |
| | ggacagctacaaccatcccttcaga |
| | caggatcagaagaacttagatcatt |
| | atataatacagtagcaaccctctat |
| | tgtgtgcatcaaaggatagagataa |
| | aagacaccaaggaagctttagacaa |
| | gatagaggaagagcaaaacaaaagt |
| | aagaccaccgcacagcaagcggccg |
| | ctgatcttcagacctggaggaggag |
| | atatgagggacaattggagaagtga |
| | attatataaatataaagtagtaaaa |
| | attgaaccattaggagtagcaccca |
| | ccaaggcaaagagaagagtggtgca |
| | gagagaaaaaagagcagtgggaata |
| | ggagctttgttccttgggttcttgg |
| | gagcagcaggaagcactatgggcgc |
| | agcgtcaatgacgctgacggtacag |
| | gccagacaattattgtctggtatag |
| | tgcagcagcagaacaatttgctgag |
| | ggctattgaggcgcaacagcatctg |
| | ttgcaactcacagtctggggcatca |
| | agcagctccaggcaagaatcctggc |
| | tgtggaaagatacctaaaggatcaa |
| | cagctcctggggatttggggttgct |
| | ctggaaaactcatttgcaccactgc |
| | tgtgccttggaatgctagttggagt |
| | aataaatctctggaacagatttgga |
| | atcacacgacctggatggagtggga |
| | cagagaaattaacaattacacaagc |
| | ttaatacactccttaattgaagaat |
| | cgcaaaaccagcaagaaaagaatga |
| | acaagaattattggaattagataaa |
| | tgggcaagtttgtggaattggttta |
| | acataacaaattggctgtggtatat |
| | aaaattattcataatgatagtagga |
| | ggcttggtaggtttaagaatagttt |
| | ttgctgtactttctatagtgaatag |
| | agttaggcagggatattcaccatta |
| | tcgtttcagacccacctcccaaccc |
| | cgaggggacccgacaggcccgaagg |
| | aatagaagaagaaggtggagagaga |
| | gacagagacagatccattcgattag |
| | tgaacggatctcgacggtatcggtt |
| | aacttttaaaagaaaaggggggatt |
| | ggggggtacagtgcaggggaaagaa |
| | tagtagacataatagcaacagacat |
| | acaaactaaagaattacaaaaacaa |
| | attacaaaaattcaaaattttatcg |
| | atcacgagactagcctcgagcaaac |
| | cttgttatctgagagaaagctagag |
| | cttccttctttcagcccccagagac |
| | aatgtggccaggctccggagggctg |
| | ggaagatgagcaatgcttgtgagtc |
| | accatgataggaagcagaagggtca |
| | ggaagtccctgggagcaaggcttag |
| | ggttagggttagggagaaaacactt |
| | gggcctggaggctcggggcaggctt |
| | cctagaggggaggggtactaaaagc |
| | caccaaaaccaagacagcaatgaaa |
| | gtaacctctggtcttcctaactgct |
| | cattatacactaattatgatgcact |
| | agcatgctaagagacacttccacca |
| | gcgccatgacagtgccatggcaaca |
| | tcaagaagtcaccccacatggtcta |
| | aaaaggggaagaaccttcagttccc |
| | ggaattgcccacccctttcctggaa |
| | aactcatgaataatccaccccttgt |
| | ttagcatataatgacctgaagggta |
| | agggggtgtggaggttgtgaaggcg |
| | ggaaggggggtagccccttcaccaa |
| | tgtaaacaaggatgtgggttctgcg |
| | gccacactctcccccgccctcccca |
| | gcggcatttccagcaagtcacatgt |
| | cctgcgcacaggctgggggccccct |
| | gctgctgcctttcttagaagccagg |
| | accacagagcccgcacagtgagcta |
| | cttggggagctatttctgtagactg |
| | agcttggggtactgggaggcagtgc |
| | tgttggcgatctgggggaaaacccc |
| | ctgagggtcccatttctctgccact |
| | gacccctttgcctggcagaaaggct |
| | ctgctgcctcctagctgtgtagcaa |
| | tggataaggcctttaacctctctaa |
| | gctgaaatgtcctcatgcataaggt |
| | ggagacagtaactcctcttccatct |
| | gctgggtgttgtcaaaactacacag |
| | gaggtttgccaagcacctaaggcac |
| | agtaggtggtcaagaaacaggaaca |
| | caatttgtcatgacgacaatgattc |
| | cttcatatgctagttacccacgagt |
| | cagccagaatgttgccataaccact |
| | tatgaagcccttcctcatttctgtt |
| | gcaattcctcagccagaggaaagcc |
| | ccacacagcccttgccactccccca |
| | tggcccatgtagaaacaaccatgtg |
| | gccttcactctgcccacagaagcta |
| | gcaccaggaccctggtcagggttag |
| | aggtttctgctgagtcaaagccaca |
| | tggagggagggagcaagggagagat |
| | gcagagtcatgtttccaggaggagg |
| | ttatctgagcataacagggacaggg |
| | gggccacaggatacctctgaggctc |
| | aggttcccacctccactccaccctc |
| | caactctctgctccacgttccttcc |
| | actattcaacacatggcaggaagtc |
| | agcctgtagggcttcacactacagt |
| | ctaagctgactgccatctgtcccca |
| | tccaggcgaggctggaaacgggggc |
| | agcaaaaagcccagggacaaagtcc |
| | ccttcccaggcacatgtattatgca |
| | ctctgcaccaaggaaacctccagat |
| | aaggtctagagaccaagggccatgt |
| | acttcgggggagaggccagaggact |
| | tctgaggttttacagagaaaagcca |
| | aaggcagcccagtcaggggaaatgt |
| | gtagccatagtgccgataaggaaag |
| | gccttcaacctgccctggtcagctc |
| | ttcctgtaagtagaggccccttacc |
| | gaaggccccagtgggaggaagggtc |
| | gggagtactcgagtcagcctcaggc |
| | tacctaggtgctttagaaaggaggc |
| | cacccaggcccatgactactccttg |
| | ccacagggagccctgcacacagatg |
| | tgctaagctctcgctgccagccaga |
| | gggaggagggtctgagccagtcaga |
| | aggagatgggccccagagagtaaga |
| | aagggggaggaggacccaagctgat |
| | ccaaaaggtgggtctaagcagtcaa |
| | gtggaggagggttccaatctgatgg |
| | cggagggcccaagctcagcctaacg |
| | aggaggccaggcccaccaaggggcc |
| | cctggaggacttgtttcccttgtcc |
| | cttgtggttttttgcatttcctgtt |
| | cccttgctgctcattgcggaagttc |
| | ctcttcttaccctgcacccagagcc |
| | tcgccagagaagacaagggcagaaa |
| | gcaccatggtgagcaagggcgagga |
| | gctgttcaccggggtggtgcccatc |
| | ctggtcgagctggacggcgacgtaa |
| | acggccacaagttcagcgtgtccgg |
| | cgagggcgagggcgatgccacctac |
| | ggcaagctgaccctgaagttcatct |
| | gcaccaccggcaagctgcccgtgcc |
| | ctggcccaccctcgtgaccaccttc |
| | ggctacggcctgatgtgcttcgccc |
| | gctaccccgaccacatgaagcagca |
| | cgacttcttcaagtccgccatgccc |
| | gaaggctacgtccaggagcgcacca |
| | tcttcttcaaggacgacggcaacta |
| | caagacccgcgccgaggtgaagttc |
| | gagggcgacaccctggtgaaccgca |
| | tcgagctgaagggcatcgacttcaa |
| | ggaggacggcaacatcctggggcac |
| | aagctggagtacaactacaacagcc |
| | acaacgtctatatcatggccgacaa |
| | gcagaagaacggcatcaaggtgaac |
| | ttcaagatccgccacaacatcgagg |
| | acggcagcgtgcagctcgccgacca |
| | ctaccagcagaacacccccatcggc |
| | gacggccccgtgctgctgcccgaca |
| | accactacctgagctaccagtccgc |
| | cctgagcaaagaccccaacgagaag |
| | cgcgatcacatggtcctgctggagt |
| | tcgtgaccgccgccgggatcactct |
| | cggcatggacgagctgtacaagtga |
| | ctgcaggaattcgagcatcttaccg |
| | ccatttattcccatatttgttctgt |
| | ttttcttgatttgggtatacattta |
| | aatgttaataaaacaaaatggtggg |
| | gcaatcatttacatttttagggata |
| | tgtaattactagttcaggtgtattg |
| | ccacaagacaaacatgttaagaaac |
| | tttcccgttatttacgctctgttcc |
| | tgttaatcaacctctggattacaaa |
| | atttgtgaaagattgactgatattc |
| | ttaactatgttgctccttttacgct |
| | gtgtggatatgctgctttaatgcct |
| | ctgtatcatgctattgcttcccgta |
| | cggctttcgttttctcctccttgta |
| | taaatcctggttgctgtctctttat |
| | gaggagttgtggcccgttgtccgtc |
| | aacgtggcgtggtgtgctctgtgtt |
| | tgctgacgcaacccccactggctgg |
| | ggcattgccaccacctgtcaactcc |
| | tttctgggactttcgctttccccct |
| | cccgatcgccacggcagaactcatc |
| | gccgcctgccttgcccgctgctgga |
| | caggggctaggttgctgggcactga |
| | taattccgtggtgttgtcggggaag |
| | ggcctgctgccggctctgcggcctc |
| | ttccgcgtcttcgccttcgccctca |
| | gacgagtcggatctccctttgggcc |
| | gcctccccgcctggaattcgagctc |
| | ggtacctttaagaccaatgacttac |
| | aaggcagctgtagatcttagccact |
| | ttttaaaagaaaaggggggactgga |
| | agggctaattcactcccaacgaaga |
| | caagatctgctttttgcttgtactg |
| | ggtctctctggttagaccagatctg |
| | agcctgggagctctctggctaacta |
| | gggaacctactgcttaagcctcaat |
| | aaagcttgccttgagtgcttcaagt |
| | agtgtgtgcccgtctgttgtgtgac |
| | tctggtaactagagatccctcagac |
| | ccttttagtcagtgtggaaaatctc |
| | tagcagtagtagttcatgtcatctt |
| | attattcagtatttataacttgcaa |
| | agaaatgaatatcagagagtgagag |
| | gaacttgtttattgcagcttataat |
| | ggttacaaataaagcaatagcatca |
| | caaatttcacaaataaagcattttt |
| | ttcactgcattctagttgtggtttg |
| | tccaaactcatcaatgtatcttatc |
| | atgtctggctctagctatcccgccc |
| | ctaactccgcccatcccgcccctaa |
| | ctccgcccagttccgcccattctcc |
| | gccccatggctgactaatttttttt |
| | atttatgcagaggccgaggccgcct |
| | cggcctctgagctattccagaagta |
| | gtgaggaggcttttttggaggccta |
| | gggacgtacccaattcgccctatag |
| | tgagtcgtattacgcgcgctcactg |
| | gccgtcgttttacaacgtcgtgact |
| | gggaaaaccctggcgttacccaact |
| | taatcgccttgcagcacatccccct |
| | ttcgccagctggcgtaatagcgaag |
| | aggcccgcaccgatcgcccttccca |
| | acagttgcgcagcctgaatggcgaa |
| | tgggacgcgccctgtagcggcgcat |
| | taagcgcggcgggtgtggtggttac |
| | gcgcagcgtgaccgctacacttgcc |
| | agcgccctagcgcccgctcctttcg |
| | ctttcttcccttcctttctcgccac |
| | gttcgccggctttccccgtcaagct |
| | ctaaatcgggggctccctttagggt |
| | tccgatttagtgctttacggcacct |
| | cgaccccaaaaaacttgattagggt |
| | gatggttcacgtagtgggccatcgc |
| | cctgatagacggtttttcgcccttt |
| | gacgttggagtccacgttctttaat |
| | agtggactcttgttccaaactggaa |
| | caacactcaaccctatctcggtcta |
| | ttcttttgatttataagggattttg |
| | ccgatttcggcctattggttaaaaa |
| | atgagctgatttaacaaaaatttaa |
| | cgcgaattttaacaaaatattaacg |
| | cttacaatttaggtggcacttttcg |
| | gggaaatgtgcgcggaacccctatt |
| | tgtttatttttctaaatacattcaa |
| | atatgtatccgctcatgagacaata |
| | accctgataaatgcttcaataatag |
| | cacctagatcaagagacaggatgag |
| | gatcgtttcgcatgattgaacaaga |
| | tggattgcacgcaggttctccggcc |
| | gcttgggtggagaggctattcggct |
| | atgactgggcacaacagacaatcgg |
| | ctgctctgatgccgccgtgttccgg |
| | ctgtcagcgcaggggcgcccggttc |
| | tttttgtcaagaccgacctgtccgg |
| | tgccctgaatgaactgcaagacgag |
| | gcagcgcggctatcgtggctggcca |
| | cgacgggcgttccttgcgcagctgt |
| | gctcgacgttgtcactgaagcggga |
| | agggactggctgctattgggcgaag |
| | tgccggggcaggatctcctgtcatc |
| | tcaccttgctcctgccgagaaagta |
| | tccatcatggctgatgcaatgcggc |
| | ggctgcatacgcttgatccggctac |
| | ctgcccattcgaccaccaagcgaaa |
| | catcgcatcgagcgagcacgtactc |
| | ggatggaagccggtcttgtcgatca |
| | ggatgatctggacgaagagcatcag |
| | gggctcgcgccagccgaactgttcg |
| | ccaggctcaaggcgagcatgcccga |
| | cggcgaggatctcgtcgtgacccat |
| | ggcgatgcctgcttgccgaatatca |
| | tggtggaaaatggccgcttttctgg |
| | attcatcgactgtggccggctgggt |
| | gtggcggaccgctatcaggacatag |
| | cgttggctacccgtgatattgctga |
| | agagcttggcggcgaatgggctgac |
| | cgcttcctcgtgctttacggtatcg |
| | ccgctcccgattcgcagcgcatcgc |
| | cttctatcgccttcttgacgagttc |
| | ttctgaattattaacgcttacaatt |
| | tcctgatgcggtattttctccttac |
| | gcatctgtgcggtatttcacaccgc |
| | atcaggtggcacttttcggggaaat |
| | gtgcgcggaacccctatttgtttat |
| | ttttctaaatacattcaaatatgta |
| | tccgctcatgaccaaaatcccttaa |
| | cgtgagttttcgttccactgagcgt |
| | cagaccccgtagaaaagatcaaagg |
| | atcttcttgagatcctttttttctg |
| | cgcgtaatctgctgcttgcaaacaa |
| | aaaaaccaccgctaccagcggtggt |
| | ttgtttgccggatcaagagctacca |
| | actctttttccgaaggtaactggct |
| | tcagcagagcgcagataccaaatac |
| | tgttcttctagtgtagccgtagtta |
| | ggccaccacttcaagaactctgtag |
| | caccgcctacatacctcgctctgct |
| | aatcctgttaccagtggctgctgcc |
| | agtggcgataagtcgtgtcttaccg |
| | ggttggactcaagacgatagttacc |
| | ggataaggcgcagcggtcgggctga |
| | acggggggttcgtgcacacagccca |
| | gcttggagcgaacgacctacaccga |
| | actgagatacctacagcgtgagcta |
| | tgagaaagcgccacgcttcccgaag |
| | ggagaaaggcggacaggtatccggt |
| | aagcggcagggtcggaacaggagag |
| | cgcacgagggagcttccagggggaa |
| | acgcctggtatctttatagtcctgt |
| | cgggtttcgccacctctgacttgag |
| | cgtcgatttttgtgatgctcgtcag |
| | gggggcggagcctatggaaaaacgc |
| | cagcaacgcggcctttttacggttc |
| | ctggccttttgctggccttttgctc |
| | acatgttctttcctgcgttatcccc |
| | tgattctgtggataaccgtattacc |
| | gcctttgagtgagctgataccgctc |
| | gccgcagccgaacgaccgagcgcag |
| | cgagtcagtgagcgaggaagcggaa |
| | gagcgcccaatacgcaaaccgcctc |
| | tccccgcgcgttggccgattcatta |
| | atgcagctggcacgacaggtttccc |
| | gactggaaagcgggcagtgagcgca |
| | acgcaattaatgtgagttagctcac |
| | tcattaggcaccccaggctttacac |
| | tttatgcttccggctcgtatgttgt |
| | gtggaattgtgagcggataacaatt |
| | tcacacaggaaacagctatgaccat |
| | gattacgccaagcgcgcaattaacc |
| | ctcactaaagggaacaaaagctgga |
| | gctgcaagcttggccattgcatacg |
| | ttgtatccatatcataatatgtaca |
| | tttatattggctcatgtccaacatt |
| | accgccatgttgacattgattattg |
| | actagttattaatagtaatcaatta |
| | cggggtcattagttcatagcccata |
| | tatgg |

### INCORPORATION BY REFERENCE

Various references such as patents, patent applications, and publications are cited herein, the disclosures of which are hereby incorporated by reference herein in their entireties.

What is claimed is:

1. A recombinant vector comprising:
a nucleic acid sequence of an enhancer comprising a nucleic acid sequence of enhancer element 14 or an effective fragment thereof, wherein the nucleic acid sequence of enhancer element 14 comprises, or consists of, SEQ ID NO:1;
a nucleic acid sequence of a promoter or an effective fragment thereof; and
a nucleic acid that encodes a gene product operably linked to said nucleic acid sequence of an enhancer and said nucleic acid sequence of a promoter.

2. The vector of claim 1, wherein the nucleic acid sequence of enhancer element 14 or an effective fragment thereof is a nucleic acid sequence of a core fragment of element 14 comprising, or consisting of, SEQ ID NO:2.

3. The vector of claim 1, wherein the nucleic acid sequence of enhancer element 14 or an effective fragment thereof is a nucleic acid sequence of an ultra-core fragment of element 14 comprising, or consisting of, SEQ ID NO:3.

4. The vector of any one of claims 1-3, wherein the enhancer comprises the first half of enhancer element 2 core sub-element 1 of SEQ ID NO: 14, and/or enhancer element 2 core sub-element 5 of SEQ ID NO:17.

5. The vector of any one of claims 1-4, wherein said vector does not comprise the nucleic acid sequence of sub-sub-element 1 of element 2 of SEQ ID NO:9 or an effective fragment thereof.

6. The vector of any one of claims 1-5, wherein said vector does not comprise the nucleic acid sequence of sub-element 4 of enhancer element 2 of SEQ ID NO: 10 or an effective fragment thereof.

7. The vector of any one of claims 1-6, wherein said vector does not comprise the nucleic acid sequence of enhancer element 9 slim of SEQ ID NO:7 or an effective fragment thereof.

8. The vector of any one of claims 1-7, wherein said vector does not comprise the nucleic acid sequence of hypersensitive site 3 (HS3) core of SEQ ID NO:8 or an effective fragment thereof.

9. The vector of any one of claims 1-8, wherein said nucleic acid sequence of an enhancer consists of, or substantially consists of: (i) a nucleic acid sequence of enhancer element 14 or an effective fragment thereof, wherein the nucleic acid sequence of enhancer element 14 comprises, or consists of, SEQ ID NO:1, and (ii) a nucleic acid sequence of uCore E2 element of SEQ ID NO:32.

10. A recombinant vector comprising:
a nucleic acid sequence of an enhancer comprising, or consisting of, the first half of enhancer element 2 core sub-element 1 of SEQ ID NO:14, and enhancer element 2 core sub-element 5 of SEQ ID NO:17;
a nucleic acid sequence of a promoter or an effective fragment thereof; and
a nucleic acid that encodes a gene product operably linked to said nucleic acid sequence of an enhancer and said nucleic acid sequence of a promoter;
wherein said vector does not comprise: (i) the nucleic acid sequence of sub-sub-element 1 of element 2 of SEQ ID NO:9 or an effective fragment thereof; (ii) the nucleic acid sequence of sub-element 4 of enhancer element 2 of SEQ ID NO: 10 or an effective fragment thereof; (iii) the nucleic acid sequence of enhancer element 9 slim of SEQ ID NO:7 or an effective fragment thereof, and/or (iv) the nucleic acid sequence of hypersensitive site 3 (HS3) core of SEQ ID NO:8 or an effective fragment thereof.

11. The vector of claim 10, wherein said vector comprises a nucleic acid sequence of enhancer element 14 or an effective fragment thereof, wherein the nucleic acid sequence of enhancer element 14 comprises, or consists of, SEQ ID NO:1.

12. The vector of claim 10, wherein said vector comprises a nucleic acid sequence of a core fragment of element 14 comprising, or consisting of, SEQ ID NO:2.

13. The vector of claim 10, wherein said vector comprises a nucleic acid sequence of an ultra-core fragment of element 14 comprising, or consisting of, SEQ ID NO:3.

14. The vector of any one of claim 10, wherein said nucleic acid sequence of an enhancer consists of, or substantially consists of: (i) a nucleic acid sequence of enhancer element 14 or an effective fragment thereof, wherein the nucleic acid sequence of enhancer element 14 comprises, or consists of, SEQ ID NO:1, and (ii) a nucleic acid sequence of uCore E2 element of SEQ ID NO:32.

15. The vector of any one of claims 1-14, wherein the gene product is Wiskott-Aldrich Syndrome protein (WASp).

16. The vector of claim 15, wherein the nucleic acid that encodes the WASp is a WAS cDNA, optionally wherein WAS cDNA comprises or consist of SEQ ID NO:20.

17. The vector of claim 15, wherein the nucleic acid that encodes the WASp is a codon optimized WAS, optionally wherein codon optimized WAS comprises or consist of SEQ ID NO:21.

18. The vector of claim 17, wherein the codon optimized WAS is selected from the group consisting of jCAT codon optimized WAS, GeneArt optimized WAS, and IDT optimized WAS.

19. The vector of any one of claims 1-18, wherein the promoter is a human promoter.

20. The vector of any one of claims 1-19, wherein the promoter is the endogenous promoter of the WAS gene.

21. The vector of any one of claims 1-18, wherein the promoter is a WAS gene promoter of SEQ ID NO:11.

22. The vector of any one of claims 1-18, wherein the promoter or the effective fragment thereof is an effective fragment of the endogenous promoter of the WAS gene which has maximum length of 600 bp and contains the nucleic acid sequence of HS1pro of SEQ ID NO:12.

23. The vector of any one of claims 1-18, wherein the promoter or the effective fragment thereof is an effective fragment of the endogenous promoter of the WAS gene consisting of the sequence of HS1pro (SEQ ID NO:12).

24. The vector of any one of claims 1-23, which is a recombinant lentiviral vector.

25. The vector of claim 24, wherein said vector is incapable of reconstituting a wild-type lentivirus through recombination.

26. The vector of any one of claims 1-25, wherein said vector comprises a packaging signal.

27. The vector of any one of claims 1-26, wherein said vector comprises a Rev Responsive Element (RRE).

28. The vector of any one of claims 1-27, wherein said vector comprises a central polypurine tract.

29. The vector of any one of claims 1-28, wherein said vector comprises a posttranscriptional regulatory element.

30. The vector of claim 29, wherein the posttranscriptional regulatory element is a Woodchuck Post-transcriptional Regulatory Element (WPRE).

31. A vector comprising or consisting of a nucleic acid sequence having at least 70%, at least 75%, at least 80%, at least 85%, at least 90%, at least 95%, at least 97%, at least 98%, at least 99%, or 100% nucleic acid sequence identity to SEQ ID NO:4.

32. A vector comprising or consisting of a nucleic acid sequence having at least 70%, at least 75%, at least 80%, at least 85%, at least 90%, at least 95%, at least 97%, at least 98%, at least 99%, or 100% nucleic acid sequence identity to SEQ ID NO:5.

33. A vector comprising or consisting of a nucleic acid sequence having at least 70%, at least 75%, at least 80%, at least 85%, at least 90%, at least 95%, at least 97%, at least 98%, at least 99%, or 100% nucleic acid sequence identity to SEQ ID NO:6.

34. The vector of any one of claims 1-33, which upon introduction into a cell, optionally a megakaryocyte, leads to expression of the gene product, optionally a physiologic or high expression of the gene product.

35. A viral particle comprising the vector of any one of claims 1-34.

36. A cell transduced with a vector according to any one of claims 1-34 or with a viral particle of claim 35.

37. The cell of claim 36, wherein the cell is a stem cell or a progenitor cell.

38. The cell of claim 36 or 37, wherein the cell is a CD34+ hematopoietic stem and/or progenitor cell.

39. The cell of any one of claims 36-38, wherein said cell is a cell derived from bone marrow, umbilical cord blood, and/or peripheral blood.

40. The cell of claim 36, wherein said cell is a dendritic cell, a CD4⁺ T cell, or a peripheral blood B or T cell.

41. The cell of any one of claims 36-40, wherein the cell is a human cell.

42. A pharmaceutical composition comprising a vector of any one of claims 1-34, a viral particle of claim 35, or a cell of any one of claims 36-41, wherein the pharmaceutical composition further comprises a pharmaceutically acceptable carrier.

43. A method of treating a disease or disorder associated with a deficient expression of a gene product in a subject in need thereof, said method comprising: transducing a stem cell and/or progenitor cell derived from said subject with a vector of any one of claims 1-34; and transplanting said transduced stem and/or progenitor cell into said subject, where said cell or derivative thereof expresses said gene product.

44. A method of treating Wiskott-Aldrich Syndrome (WAS) in a subject in need thereof, said method comprising: transducing a stem cell and/or progenitor cell derived from said subject with a vector according to any one of claims 1-34, wherein the gene product expressed by the vector is WASp; and transplanting said transduced stem and/or progenitor cell into said subject, where said cell or derivative thereof expresses said WASp.

45. The method of any one of claim 43 or 44, wherein the stem cell and/or a progenitor cell is a human hematopoietic stem and/or progenitor cell.

46. The method of claim 45, wherein the human hematopoietic stem cell and/or a progenitor cell is derived from bone marrow and/or a CD34+ cell.

47. A method of treating or preventing a disease or disorder associated with a deficient expression of the gene product in a subject in need thereof, said method comprising: administering to said subject a vector of any one of claims 1-34, a viral particle of claim 35, a cell of claims of any one of claims 36-41 or a pharmaceutical composition of claim 42.

48. A method of treating or preventing WAS in a subject in need thereof, said method comprising: administering to said subject a vector of any one of claims 1-34, a viral particle of claim 35, a cell of claims of any one of claims 36-41 or a pharmaceutical composition of claim 42, wherein the gene product expressed by the vector is WASp.

49. The method of any one of claims 43-48, wherein the subject is a human.

50. The method of any one of claims 43-49, wherein the vector is a lentiviral vector.

51. The method of any one of claims 43-50, wherein the vector is administered in a dose in the range of about 1×10⁵ TU/ml to about 1×10⁸ TU/ml.

52. A recombinant nucleic acid comprising a nucleic acid sequence of enhancer element 14 comprising, or consisting of, SEQ ID NO:1 or an effective fragment thereof.

53. The nucleic acid of claim 52, wherein the nucleic acid comprises a nucleic acid sequence of a core fragment of element 14 comprising, or consisting of, SEQ ID NO:2.

54. The nucleic acid of claim 52, wherein the nucleic acid comprises a nucleic acid sequence of ultra-core fragment of element 14 comprising, or consisting of, SEQ ID NO:3.

55. The nucleic acid of any one of claims 52-54, wherein the nucleic acid comprises a nucleic acid sequence of the first half of enhancer element 2 core sub-element 1 of SEQ ID NO:14, and/or enhancer element 2 core sub-element 5 of SEQ ID NO:17.

56. The nucleic acid of any one of claims 52-55, wherein the nucleic acid comprises or consists of a combination of (i) a nucleic acid sequence of enhancer element 14 comprising, or consisting of, SEQ ID NO:1 or an effective fragment thereof, optionally comprising or consisting of SEQ ID NO:2 or SEQ ID NO:3, and (ii) a nucleic acid sequence of uCore E2 element of SEQ ID NO:32.

57. The nucleic acid of any one of claims 52-56, wherein the nucleic acid comprises a nucleic acid sequence of any human promoter or an effective fragment thereof.

58. The nucleic acid of any one of claims 52-57, wherein the nucleic acid comprises a nucleic acid sequence of an endogenous promoter of the WAS gene.

59. The nucleic acid of any one of claims 52-58, wherein the nucleic acid comprises a nucleic acid sequence of a WAS gene promoter comprising, or consisting of, SEQ ID NO:11.

60. The nucleic acid of any one of claims 52-56, wherein the promoter or the effective fragment thereof is an effective fragment of the endogenous promoter of the WAS gene which has maximum length of 600 bp and contains the nucleic acid sequence of HS1pro of SEQ ID NO:12.

61. The nucleic acid of any one of claims 52-56, wherein the promoter or the effective fragment thereof is an effective fragment of the endogenous promoter of the WAS gene consisting of the sequence of HS1pro (SEQ ID NO:12).

62. The nucleic acid of any one of claims 52-61, wherein the nucleic acid does not comprise: (i) the nucleic acid sequence of sub-sub-element 1 of element 2 of SEQ ID NO:9 or an effective fragment thereof; (ii) the nucleic acid sequence of sub-element 4 of enhancer element 2 of SEQ ID NO: 10 or an effective fragment thereof; (iii) the nucleic acid sequence of enhancer element 9 slim of SEQ ID NO:7 or an effective fragment thereof; and/or (v) the nucleic acid sequence of hypersensitive site 3 (HS3) of SEQ ID NO:8 or an effective fragment thereof.

63. The nucleic acid of any one of claims 52-62, wherein the nucleic acid comprises a transgene.

64. The nucleic acid of claim 63, wherein the transgene encodes WASp.

65. The nucleic acid of any one of claims 52-64, wherein said nucleic acid is comprised within an expression cassette.

66. The nucleic acid of claim 65, wherein said expression cassette, when transduced into a cell, is capable of expressing a gene product encoded by a transgene operably linked to the nucleic acid sequence of enhancer element 14, optionally capable of expressing the gene product in the cell at a physiologic or high expression level.

67. The nucleic acid of claim 65, wherein the transgene encodes WASp, and said expression cassette, when transduced into a cell, is capable of expressing WASp, optionally capable of expressing WASp in the cell at a physiologic or high level.
